(19) United States
(12) Patent Application Publication
(10) Pub. No.: US 2003/0233670 A1
(43) Pub. Date: Dec. 18, 2003

(54) Gene sequences and uses thereof in plants

(76) Inventors: Michael D. Edgerton, St. Louis, MO (US); Paul S. Chomet, Mystic, CT (US); Lucille B. Laccetti, Groton, CT (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/310,154

(22) Filed: Dec. 4, 2002

Related U.S. Application Data
(60) Provisional application No. 60/337,358, filed on Dec. 4, 2001.

(51) Int. Cl. A01H 1/00; C12N 15/82; C12Q 1/68; C07H 21/04; C12N 9/24; C12P 21/02

(52) U.S. Cl. 800/278; 435/6; 435/69.1; 435/200; 435/320.1; 435/419; 536/23.2

(57) ABSTRACT

The invention provides polynucleotides and proteins encoded by the polypeptides. The disclosed polynucleotides and polypeptides find use in production of transgenic plants to produce plants having improved properties. The invention further provides methods of producing fertile transgenic plants, preferably maize, with desirable phenotypes and progeny of any generation derived from the fertile transgenic plants.

### CROSS-REFERENCE TO RELATED APPLICATIONS

This application claims priority under 35 U.S.C. §119(e) of U.S. Provisional Application No. 60/337,358 filed Dec. 4, 2001, the disclosure of which application is incorporated herein by reference in its entirety.

### INCORPORATION OF SEQUENCE LISTING

Two copies of the sequence listing (Copy 1 and Copy 2) and a computer readable form (CRF) of the sequence listing, all on CD-ROMs, each containing the file named pa_{—}00432.rpt which is 3,660,811 bytes (measured in MS-Windows 2000) and was created on Nov. 26, 2002, are herein incorporated by reference.

### FIELD OF THE INVENTION

Disclosed herein are inventions in the field of molecular biology, genetics, and plant biochemistry and plant biology. More specifically disclosed are nucleic acid sequences from a variety of organisms, including plants, cyanobacteria, bacteria, yeast and other fungi. Further disclosed are arrays, oligonucleotide fragments and DNA constructs of such molecules. Also disclosed are proteins and polypeptides which are encoded by such nucleic acid molecules. Also disclosed are transgenic organisms, preferably maize, with expression of such proteins.

### BACKGROUND OF THE INVENTION

Maize, Zea mays L., is one of the major crops grown worldwide as a primary source for animal feed, human food and industrial purposes. Maize plants with improved agronomic traits, such as yield or pest resistance, improved quality traits such as oil, protein or starch quality or quantity, or improved processing characteristics, such as extractability of desirable compounds, are desirable for both the farmer and consumer of maize and maize derived products.

The ability to breed or develop transgenic plants with improved traits depends in part on identification of genes. Whereas the unqiue sequences disclosed herein that were identified in the maize genome may be useful both as mapping tools to assist in plant breeding and in transgenic maize plants, unique sequences identified in species other than maize will primarily be useful to confer novel phenotypes in transgenic maize.

Disclosed herein are DNA sequences useful for breeding or to express transgenes in maize to effect traits such as nitrogen metabolism, steroid biosynthesis and signaling, beta-carotene synthesis, cell division and cell cycle, pest resistance, including disease, insect and virus resistance, light perception and associated signaling processes, growth regulator perception and associated signaling processes, cold tolerance, heat tolerance, grain qualtiy, such as modifications in oil, amino acid or protein, or starch compositon, resistance to abiotic stresses, such as water limiting conditions and oxidative stress, nutrient availability and utilization, such as phosphorous or nitrogen, and factors generally effecting plant growth reproduction and vigor.

### SUMMARY OF THE INVENTION

The present invention provides isolated polynucleotides which comprise a nucleic acid sequence selected from the group consisting of SEQ ID NO: 1 through SEQ ID NO: 368 or complements thereof. The invention further provides recombinant DNA constructs comprising a polynucleotide selected from the group consisting of SEQ ID NO: 1 through SEQ ID NO: 368 or complements thereof. Furthermore, the invention provides transformed cells or organisms comprising a polynucleotide of the present invention, prefereably where the transformed cell or organism is a plant cell or plant and most preferably, wherein the transformed organism is a plant selected from the group consisting of cotton, wheat, soybean, maize, rice, canola, teosinte and Arabidopsis.

The present invention provides substantially purified polypeptides encoded by the polynucleotides provided herein, where the polypeptides comprise an amino acid sequence selected from the group consisting of SEQ ID NO: 369 through SEQ ID NO: 736 In addition, the invention provides polypeptides having at least 70% amino acid sequence identity with a polypeptide provided herein.

The present invention provides a recombinant DNA construct comprising a polynucleotide selected from the group consisting of:

(a) a polynucleotide comprising a nucleic acid sequence selected from the group consisting of SEQ ID NO: 1 through SEQ ID NO: 368;

(b) a polynucleotide encoding a polypeptide having an amino acid sequence selected from the group consisting of SEQ ID NO: 369 through SEQ ID NO: 736;

(c) a polynucleotide comprising a nucleic acid sequence complementary to a nucleic acid sequence selected from the group consisting of SEQ ID NO: 1 through SEQ ID NO: 368;

(d) a polynucleotide having at least 60% sequence identity to a polynucleotide of (a), (b) or (c);

(e) a polynucleotide encoding a polypeptide having at least 70% sequence identity to a polypeptide having an amino acid sequence selected from the group consisting of SEQ ID NO: 369 through SEQ ID NO: 736

(f) an oligonucleotide comprising from about 15 to 100 nucleotide bases, wherein the oligonucleotide hybridizes under low stringency conditions to a polynucleotide of (a), (b) or (c); and

(g) a polynucleotide comprising a promoter functional in a plant cell, operably joined to a coding sequence for a polypeptide having at least 70% sequence identity to a polypeptide having an amino acid sequence selected from the group consisting of SEQ ID NO: 369 through SEQ ID NO: 736, wherein the encoded polypeptide is a functional homolog of the polypeptide having an amino acid sequence selected from the group consisting of SEQ ID NO: 369 through SEQ ID NO: 736; and

(h) a polynucleotide comprising a promoter functional in a plant cell, operably joined to a coding sequence for a polypeptide having an amino acid sequence selected from the group consisting of SEQ ID NO: 369 through SEQ ID NO: 736, wherein transcription of the coding sequence produces an RNA molecule having sufficient complementarity to a polynucleotide encoding the polypeptide to result in decreased expression of the polypeptide when the construct is expressed in a plant cell.

Of particular interest are recombinant DNA constructs, as described above, wherein the construct comprises a promoter region functional in a plant cell operably joined to a polynucleotide of the present invention. The polynucleotide may be oriented with respect to the promoter such that transcription of the polynucleotide produces an mRNA encoding the polypeptide. Alternatively, the present invention provides recombinant constructs wherein the polynucleotide is oriented with respect to the plant promoter such that transcription from the polynucleotide produces an RNA complementary to the mRNA encoding the polypeptide.

The constructs of the present invention are useful for production of transgenic plants having at least one improved property as the result of increased or decreased expression of a polypeptide described herein. Of particular interest are uses of the disclosed polynucleotides to provide plants having improved yield resulting from improved utilization of key biochemical compounds, such as nitrogen, phosphorous and carbohydrate, or resulting from improved responses to environmental stresses, such as cold, heat, drought, salt, and attack by pests or pathogens. Polynucleotides of the present invention may also be used to provide plants having improved growth and development, and ultimately increased yield, as the result of modified expression of plant growth regulators or modification of cell cycle or photosynthesis pathways. Other traits of interest that may be modified in plants using polynucleotides of the present invention include shade avoidance, flavonoid content, seed oil and protein quantity and quality, herbicide tolerance and rate of homologous recombination. Such improved properties and polynucleotides useful to obtain such properties are set forth in more detail herein, for example in section D and Table 1.

Thus, the present invention also comprises a transformed plant comprising a recombinant construct of the present invention. Of particular interest in the present invention are transformed maize and soybean plants.

The present invention provides a method of producing a plant having an improved property, wherein the method comprises transforming a plant with a recombinant construct comprising a promoter region functional in a plant cell operably joined to a polynucleotide comprising coding sequence for a polypeptide associated with said property, and growing said transformed plant. Desired properties and the polynucleotides useful to obtain such properties are described in detail herein. The polypeptides useful for providing improved properties may be polypeptides whose sequences are provided herein, or functional homologs of such polypeptides.

In the methods of the present the polynucleotide may be oriented with respect to the promoter such that transcription of the polynucleotide produces an mRNA encoding the polypeptide, and wherein the mRNA is translated to express the polypeptide in the plant. Alternatively, the polynucleotide may be oriented with respect to the promoter such that transcription from the polynucleotide produces an RNA complementary to the mRNA encoding the polypeptide, and wherein the level of the polypeptide in the plant is decreased as the result of the presence of the complementary RNA. Expression of complementary RNA is of particular interest where the polynucleotide is native to the target plant host.

A further aspect of the present invention relates to the discovery that transformation by insertion into the corn genome of heterologous genes, wherein the transcription of said heterologous genes is not known to produce a phenotype in corn, can be used as a reliable generator of modification of the corn genome to produce unexpected yet desired phenotypes. Thus, this invention provides methods for introducing into a maize line an enhanced phenotype as compared to a phenotype in parental units of said maize line. The method comprises generating a population of transgenic plants comprising a variety of heterologous DNA for the transcription of which there is no known phenotype in maize. In one aspect of the invention the population is generated for a plurality of transgenic events for a plurality of unique transgenic DNA constructs. Each transgenic event comprises introducing into the genome of a parental units a single transgenic DNA construct comprising a promoter operably linked to heterologous DNA for the transcription of which there is no known phenotype in corn. The transgenic DNA construct is introduced into the parental genome in sufficient quantity to produce transgenic cells which can be cultured into plants of transgenic maize having said enhanced phenotype. The transgenic cells are cultured into transgenic plants producing progeny transgenic seed. The population of transgenic plants are screened for observable phenotypes. Seed is collected from transgenic plants which are selected as having an unexpected enhanced phenotype. Optionally, the method comprises repeating a cycle of germinating transgenic seed, growing subsequent generation plants from said transgenic seed, observing phenotypes of said subsequent generation plants and collecting seeds from subsequent generation plants having an enhanced phenotype.

In preferred aspects of the method a large population is screened by generating at least 2 transgenic events for at least 20 unique transgenic DNA constructs, more preferably upwards of 10 or more transgenic events, say up to 100 or more transgenic events or upwards of 50 or more unique transgenic DNA constructs, say 100 or more or even 500 or more unique transgenic constructs.

Other preferred aspects of the method employ DNA constructs where the heterologous DNA is operably linked to a selected promoter, e.g. a promoter region comprising a rice actin promoter and rice actin intron. The DNA construct may be introduced into a random location in the genome or into a preselected site in the genome.

Yet another aspect of the invention provides a method comprising crossing transgenic plants from the population of transgenic plants with at least one other maize line to produce a hybrid population of transgenic plants, observing phenotypes in the hybrid population and selecting seed from transgenic plants having unexpected enhanced phenotypes in the hybrid population.

### BRIEF DESCRIPTION OF THE DRAWINGS

FIG. 1: Map of plasmid pMON64154. The plasmid comprises a 9215 base pair sequence comprising an nptII expression cassette and a GATEWAY™ cloning site (Invitrogen, Carlsbad, Calif.) flanked at the 5′ end by a rice actin 1 promoter, actin1 exon1, and actin1 intron 1 sequence and at the 3′ end by an Agrobacterium tumefaciens nopaline synthase-potato proteinase inhibitor II (pinII) 3′UTR.

FIG. 2: Map of plasmid pMON72472. The plasmid comprises a 9708 base pair sequence comprising an nptII expression cassette and a GATEWAY™ cloning site (Invitrogen, Carlsbad, Calif.) flanked at the 5′ end by a rice actin 1 promoter, actin1 exon1, and actin1 intron 1 sequence and at the 3′ end by an Agrobacterium tumefaciens nopaline synthase-potato proteinase inhibitor II (pinII) 3′UTR. The GATEWAY cloning and nptII cassettesa are flanked by the Agrobacterium tumefaciens left border and right border sequences.

### DETAILED DESCRIPTION OF THE INVENTION

Following are definitions for terms as specifically used herein.

“Heterologous” refers to a segment of DNA that is present in a non-natural DNA construct, e.g. regulatory DNA as well as DNA coding for a protein.

“Transgenic DNA construct” means a segment of DNA which is introduced into the genome of a parental maize line. While a transgenic DNA construct can comprise any segment of DNA that is heterologous to the insertion site, in preferred aspects of the invention the transgenic DNA construct will be designed to provide a specific function, e.g. suppress or over express a selected protein. Useful transgenic DNA constructs comprise gene regulatory segment operably linked to a protein coding segment. A gene regulatory segment can more specifically comprise promoter elements, enhancers, silencers, introns and untranslated regions. An especially useful gene regulatory segment for use in maize comprises a rice actin promoter with a rice actin intron as described more specifically below. Protein coding segment can be any coding segment that may be of interest for investigation into its effect in a transgenic plant. Exemplary protein coding segments include DNA segments encoding all or a part of any protein such as a cytochrome p450, a transporter, a lipase, a kinase, a receiver domain, a synthase, a transcription factor, a reductase, a phosphatase, a ribonuclease, an anhydrase and the like.

A “non-predetermined location in genomic DNA” means a random locus in a maize chromosome in which a transgenic DNA construct is inserted by chance.

“Transformation” means a method of introducing a transgenic DNA construct into a genome and can include any of the well-known and demonstrated methods including electroporation as illustrated in U.S. Pat. No. 5,384,253, microprojectile bombardment as illustrated in U.S. Pat. Nos. 5,015,580; 5,550,318; 5,538,880; 6,160,208; 6,399,861 and 6,403,865, Agrobacterium mediated transformation as illustrated in U.S. Pat. Nos. 5,635,055; 5,824,877; 5,591,616; 5,981,840 and 6,384,301, and protoplast transformation as illustrated in U.S. Pat. No. 5,508,184, all of which are incorporated herein by reference.

“Tissue from a parental maize line” means tissue which is specifically adapted for a selected method of transformation and can include cell culture or embryonic callus.

“Yield” as used herein means the production of shelled corn kernels per unit of production area, e.g. in bushels per acre or metric tons per hectare, often reported on a moisture adjusted basis, e.g. at 15.5% moisture. As a bushel of corn is defined by law in the State of Iowa as 56 pounds by weight, a useful conversion factor for corn yield is: 100 bushels per acre is equivalent to 6.272 metric tons per hectare.

A. Nucleic Acids

Nucleic acid molecules of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989), and by Haynes et al., Nucleic Acid Hybridization, A Practical Approach, IRL Press, Washington, DC (1985). Departures from complete complementarity are therefore permissible, as long as such departures do not completely preclude the capacity of the molecules to form a double-stranded structure. Thus, in order for a nucleic acid molecule or fragment of a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50 ° C. to moderately stringent conditions of about 2.0×SSC at 40 ° C. to high stringency conditions of about 0.2×SSC at 50° C.

As used herein “sequence identity” refers to the extent to which two optimally aligned polynucleotide or peptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in reference sequence segment, i.e. the entire reference sequence or a smaller defined part of the reference sequence. “Percent identity” is the identity fraction times 100.

This invention provides nucleic acid molecules comprising DNA sequence representing genes or fragments of genes such as substantial parts of genes providing the protein coding sequence part of the gene. More specifically the nucleic acid molecules of this invention comprise nucleic acid molecules provided herein as SEQ ID NO: 1 through SEQ ID NO: 368.

A subset of the nucleic acid molecules of the present invention includes nucleic acid molecules that are marker molecules. Another subset of the nucleic molecules of this invention includes fragments of genes consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO: 1 through SEQ ID NO: 368.

Nucleic acid molecules of the invention may encode significant portions of, or indeed most if not all of, the protein encoded by the corresponding gene.

Nucleic acid sequences can be screened for the presence of protein encoding sequence which is homologous to genes of other organisms with known protein encoding sequence using any of a variety of search algorithms. Such search algorithms can be homology-based or predictive-based. Homology-based searches (e.g., GAP2, BLASTX supplemented by NAP and TBLASTX) detect conserved sequences during comparisons of DNA sequences or hypothetically translated protein sequences to public and/or proprietary DNA and protein databases. Existence of a gene is inferred if significant sequence similarity extends over the majority of the target gene. Since homology-based methods may overlook genes unique to the source organism, for which homologous nucleic acid molecules have not yet been identified in databases, gene prediction programs are also used. Gene prediction programs generally use “signals” in the sequence, such as splice sites or “content” statistics, such as codon bias; to predict gene structures (Stormo, G., Genome Research 10: 394-397 (2000)).

With respect to nucleotide sequences, degeneracy of the genetic code provides the possibility to substitute at least one base of the base sequence of a gene with a different base without causing the amino acid sequence of the polypeptide produced from the gene to be changed. Hence, the DNA of the present invention may also have any base sequence that has been changed from SEQ ID NO: 1 to SEQ ID NO: 368 by substitution in accordance with degeneracy of genetic code. References describing codon usage include: Carels et al., J. Mol. Evol. 46: 45 (1998) and Fennoy et al., Nucl. Acids Res. 21 (23): 5294 (1993).

B. Protein and Polypeptide Molecules

This invention also provides protein and polypeptide molecules, including those encoded by nucleic acid molecules of the present invention. Of particular interest, are polypeptides whose sequences are provided herein as SEQ ID NO: 369 through SEQ ID NO: 736. As used herein, the terms “protein” and “polypeptide” include any molecule that comprises five or more amino acids. It is well known in the art that proteins and polypeptides may undergo modification, including post-translational modifications, such as, but not limited to, disulfide bond formation, glycosylation, phosphorylation, or oligomerization. Thus, the “proteins” and “polypeptides” of this invention include molecules that are modified by any biological or non-biological process. The terms “amino acid” and “amino acids” refer to all naturally occurring L-amino acids. This definition is meant to include norleucine, ornithine, homocysteine, and homoserine.

A further aspect of the invention comprises polypeptides which differ in one or more amino acids from those of a protein sequence provided herein as the result of one or more conservative amino acid substitutions, deletions or insertions, but having the same function as a polypeptide provided herein. Such functional homologs are also of interest in the present invention and find use in methods of producing plants having improved properties.

It is well known in the art that one or more amino acids in a native sequence can be substituted with another amino acid(s), the charge and polarity of which are similar to that of the native amino acid, resulting in a silent change. For instance, valine is a conservative substitute of alanine and threonine is a conservative substitute for serine. Conservative substitutions for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine.

Also of interest are polypeptides which have the same function as polypeptides of this invention and a modified activity. Variations in protein activity can be achieved by mutagenesis; screening methods for obtaining a specified protein or enzymatic activity of interest are disclosed in U.S. Pat. No. 5,939,250. An alternative approach to the generation of variants uses random recombination techniques such as “DNA shuffling” as disclosed in U.S. Pat. Nos. 5,605,793; 5,811,238; 5,830,721; 5,837,458 and International Applications WO 98/31837, WO 99/65927. An alternative method of molecular evolution involves a staggered extension process (StEP) for in vitro mutagenesis and recombination of nucleic acid molecule sequences, as disclosed in U.S. Pat. No. 5,965,408 and International Application WO 98/42832.

C. Plant Transformation Constructs

Nucleic acid molecules of the present invention may be used in transformation, e.g. where exogenous genetic material may be transferred into a plant cell and the plant cell regenerated into a whole plant. As used herein, an “exogenous coding region” or “selected coding region” is a coding region not normally found in the host genome in an identical context. By this, it is meant that the coding region may be isolated from a different species than that of the host genome, or alternatively, isolated from the host genome, but is operably linked to one or more regulatory regions which differ from those found in the unaltered, native gene. An exogenous nucleic acid molecule can have a naturally occurring or non-naturally occurring nucleotide sequence. One skilled in the art understands that an exogenous nucleic acid molecule can be a nucleic acid molecule derived from a different or the same species into which it is introduced. Such exogenous genetic material may be transferred into either monocotyledons and dicotyledons including but not limited to the plants, soy, cotton, canola, maize, wheat and rice.

Exogenous genetic material may be transferred into a plant cell by the use of a DNA vector or construct designed for such a purpose. Preferred exogenous genetic material comprise a nucleic acid molecule of the present invention. Vectors have been engineered for transformation of constructs comprising one or more nucleic acid molecules into plant genomes. Vectors have been designed to replicate in both E. coli and A. tumefaciens and have all of the features required for transferring large inserts of DNA into plant chromosomes. Exogenous genetic material may be transferred into a host cell by the use of a DNA vector or construct designed for such a purpose.

An important aspect of the present invention is the introduction into plants of a gene provided herein to affect the expression of a particular protein or polypeptide product. The design and construction of vectors which may be employed in conjunction with plant transformation techniques according to the present invention will be known to those of skill of the art in light of the present disclosure (see for example, Sambrook et al., 1989; Gelvin et al., 1994; Clark 1997). The techniques of the current invention are thus not limited to any particular DNA, and may be used to introduce one or more desirable genes into a plant, the expression of which will confer a desirable phenotype on a plant.

In cases where over expression of heterologous DNA may not be satisfactory, effective or desirable in producing an observed enhanced phenotype, it is contemplated that a person or ordinary skill in the art would look to protein pathways for an alternate route to the desired enhanced phenotype. Such alternate route may include insertion of heterologous DNA coding for a protein which is upstream or downstream of the protein originally associated with the observed enhanced phenotype. Another alternate route may include insertion of heterologous DNA which is effective in suppression of a competitive protein. When suppression of protein expression is the intended objective, the heterologous DNA can be designed to produce a gene silencing effect, e.g. by an antisense or RNAi mechanism. Anti-sense suppression of genes in plants by introducing by transformation of a construct comprising DNA of the gene of interest in an anti-sense orientation is disclosed in U.S. Pat. Nos. 5,107,065; 5,453,566; 5,759,829; 5,874,269; 5,922,602; 5,973,226; 6,005,167; WO 99/32619; WO 99/61631; WO 00/49035; WO 02/02798; all of which are incorporated herein by reference. Interfering RNA suppression of genes in a plant by introducing by transformation of a construct comprising DNA encoding a small (commonly less than 30 base pairs) double-stranded piece of RNA matching the RNA encoded by the gene of interest is disclosed in U.S. Pat. Nos. 5,190,931; 5,272,065; 5,268,149; WO 99/61631; WO 01/75164; WO 01/92513, all of which are incorporated herein by reference.

GATEWAY™ cloning technology (Invitrogen Life Technologies, Carlsbad, Calif.) is preferably used for construction of vectors. GATEWAY™ technology uses phage lambda base site-specific recombination for vector construction, instead of restriction endonucleases and ligases. The GATEWAY™ method produces a high frequency of inserts in a plasmid in the correct orientation relative to other elements in the plasmid such as promoters, enhancers, and the such. Routine cloning of any desired DNA sequence into a vector comprising operable plant expression elements is thereby facilitated. Using the GATEWAY™ cloning technology, a desired DNA sequence, such as a coding sequence, may be amplified by PCR with the phage lambda attB1 sequence added to the 5′ primer and the attB2 sequence added to the 3′ primer. Alternatively, nested primers comprising a set of attB1 and attB2 specific primers and a second set of primers specific for the selected DNA sequence can be used. Sequences, such as coding sequences, flanked by attB1 and attB2 sequences can be readily inserted into plant expression vectors using GATEWAY™ methods.

In certain embodiments, the present inventors contemplate the transformation of a recipient cell with more than one transformation construct. For example, a gene of interest for plant improvement may be employed in combination with a marker gene. One may employ the separate genes on either the same or different DNA segments for transformation. In the latter case, the different vectors may be delivered concurrently to recipient cells if cotransformation into a single chromosomal location is desired. Using microprojectile bombardment and Agrobacterium mediated transformation, a certain percentage of cells in which the marker has been introduced will also receive the other gene(s) of interest, and the selectable marker and gene(s) of interest will be cointegrated at a single locus in the host genome. Two or more transgenes can also be introduced in a single transformation event using either distinct selected-protein encoding vectors, or using a single vector incorporating two or more gene coding sequences. Of course, any two or more transgenes of any description, such as those conferring, for example, herbicide, insect, disease (viral, bacterial, fungal, nematode) or drought resistance, male sterility, drydown, standability, prolificacy, starch quantity or properties, oil quantity and quality, or those increasing yield or nutritional quality may be employed as desired.

In other embodiments of the invention, it is contemplated that one may wish to employ replication-competent viral vectors for plant transformation. Such vectors include, for example, wheat dwarf virus (WDV) “shuttle” vectors, such as pW1-11 and pW1-GUS (Ugaki et al., 1991). These vectors are capable of autonomous replication in maize cells as well as E. coli, and as such may provide increased sensitivity for detecting DNA delivered to transgenic cells. A replicating vector also may be useful for delivery of genes flanked by DNA sequences from transposable elements such as Ac/Ds, or Mu. It has been proposed that transposition of these elements within the maize genome requires DNA replication (Laufs et al., 1990). It also is contemplated that transposable elements would be useful for producing transgenic plants lacking elements necessary for selection and maintenance of the plasmid vector in bacteria, e.g. antibiotic resistance genes, or other selectable markers, and origins of DNA replication. It also is proposed that use of a transposable element such as Ac, Ds, or Mu would actively promote integration of the desired DNA and hence increase the frequency of stably transformed cells.

Vectors used for plant transformation may include, for example, plasmids, cosmids, YACs (yeast artificial chromosomes), BACs (bacterial artificial chromosomes), PACs (plant artificial chromosomes), or any other suitable cloning system. It is contemplated that utilization of cloning systems with large insert capacities will allow introduction of large DNA sequences comprising more than one selected gene. Introduction of such sequences may be facilitated by use of bacterial or yeast artificial chromosomes (BACs or YACs, respectively), or even plant artificial chromosomes (PACs). For example, the use of BACs for Agrobacterium-mediated transformation was disclosed by Hamilton et al. (1996).

Particularly useful for transformation are expression cassettes which have been isolated from such vectors. DNA segments used for transforming plant cells will, of course, generally comprise the cDNA, gene or genes which one desires to introduced into and have expressed in the host cells. These DNA segments can further include structures such as promoters, enhancers, 3′ untranslated regions (such as polyadenylation sites), polylinkers, or even regulatory genes as desired. The DNA segment or gene chosen for cellular introduction may encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

1. Regulatory Elements

A number of promoters that are active in plant cells have been described in the literature. Such promoters would include but are not limited to the nopaline synthase (NOS) and octopine synthase (OCS) promoters that are carried on tumor-inducing plasmids of Agrobacterium tumefaciens, the caulimovirus promoters such as the cauliflower mosaic virus (CaMV) 19S and 35S promoters and the figwort mosaic virus (FMV) 35S promoter, the enhanced CaMV35S promoter (e35S), the light-inducible promoter from the small subunit of ribulose bisphosphate carboxylase (ssRUBISCO, a very abundant plant polypeptide). It is particularly contemplated that the rice actin 1 promoter will be useful in the practice of the present invention. All of these promoters have been used to create various types of DNA constructs that have been expressed in plants. See, for example PCT publication WO 84/02913 (Rogers et al., Monsanto, herein incorporated by reference in its entirety).

Promoter hybrids can also be constructed to enhance transcriptional activity (Hoffman, U.S. Pat. No. 5,106,739), or to combine desired transcriptional activity, inducibility and tissue specificity or developmental specificity. Promoters that function in plants include but are not limited to promoters that are inducible, viral, synthetic, constitutive as described (Poszkowski et al., 1989; Odell et al., 1985), and temporally regulated, spatially regulated, and spatio-temporally regulated (Chau et al., 1989). Other promoters that are tissue-enhanced, tissue-specific, or developmentally regulated are also known in the art and envisioned to have utility in the practice of this invention.

Promoters may be obtained from a variety of sources such as plants and plant DNA viruses and include, but are not limited to the CaMV35S and FMV35S promoters and promoters isolated from plant genes such as ssRUBISCO genes. As described below, it is preferred that the particular promoter selected should be capable of causing sufficient expression to result in the production of an effective amount of the gene product of interest.

The promoters used in the DNA constructs (i.e. chimeric/recombinant plant genes) of the present invention may be modified, if desired, to affect their control characteristics. Promoters can be derived by means of ligation with operator regions, random or controlled mutagenesis, etc. Furthermore, the promoters may be altered to contain multiple “enhancer sequences” to assist in elevating gene expression. Examples of such enhancer sequences have been reported by Kay et al. (1987).

By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from the CaMV 35S promoter, octopine synthase genes (Ellis et al., 1987), the maize alcohol dehydrogenase gene, the maize shrunken 1 gene and promoters from non-plant eukaryotes (e.g., yeast; Ma et al., 1988).

Where an enhancer is used in conjunction with a promoter for the expression of a selected protein, it is believed that it will be preferred to place the enhancer between the promoter and the start codon of the selected coding region. However, one also could use a different arrangement of the enhancer relative to other sequences and still realize the beneficial properties conferred by the enhancer. For example, the enhancer could be placed 5′ of the promoter region, within the promoter region, within the coding sequence (including within any other intron sequences which may be present), or 3′ of the coding region.

In addition to introns with enhancing activity, other types of elements can influence gene expression. For example, untranslated leader sequences predicted to enhance gene expression as well as “consensus” and preferred leader sequences have been identified (Joshi, 1987). Preferred leader sequences are contemplated to include those which have sequences predicted to direct optimum expression of the attached coding region, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants, and in maize in particular, will be most preferred, for example, sequences derived from the small subunit of ribulose bisphosphate carboxylase (RUBISCO).

It also is contemplated that expression of one or more transgenes may be eliminated upon expression of a transgene. In particular, by operably linking a promoter to a particular coding sequence in antisense orientation, accumulation of the respective protein encoded by the sense transcript could be eliminated or decreased. This could allow, for example, elimination of a particular gene product which would contribute to the adverse effects of osmotic stress or attack by pests, disease, or other conditions.

It also is contemplated that it may be useful to target DNA within a cell. For example, it may be useful to target introduced DNA to the nucleus as this may increase the frequency of transformation. Particular DNA sequences which are capable of targeting DNA to the nucleus are known, e.g., the Agrobacterium tumefaciens virD2 gene (Tinland et al., 1995). Within the nucleus itself, it would be useful to target a gene in order to achieve site specific integration. For example, it would be useful to have a gene introduced through transformation replace an existing gene in the cell. Furthermore, it would be useful to target a transgene to integrate into the genome at a predetermined site from which it is known that gene expression occurs. Several site specific recombination systems exist which are known, including cre-lox (U.S. Pat. No. 4,959,317) and FLP-FRT (U.S. Pat. No. 5,527,695). Both of these cited site specific recombination systems have been shown to function in plants (Albert et al., 1995; Lyznik et al., 1996).

2. 3′ Untranslated Regions (3′ UTR)

Transformation constructs prepared in accordance with the invention will typically include a 3′ end untranlated sequence DNA sequence that follows the coding sequence and typically contains a polyadenylation sequence. One type of 3′ untranslated sequence which may be used is a 3′ UTR from the nopaline synthase gene of Agrobacterium tumefaciens (nos 3′ end; Bevan et al., 1983). Where a 3′ end other than a nos 3′ UTR is used in accordance with the invention, the most preferred 3′ ends are contemplated to be those from a gene encoding the small subunit of a ribulose-1,5-bisphosphate carboxylase-oxygenase (rbcS), and more specifically, from a rice rbcS gene (PCT Publication WO 00/70066), the 3′ UTR for the T7 transcript of Agrobacterium tumefaciens (Dhaese et al., 1983), the 3′ end of the protease inhibitor I or II genes from potato (An et al., 1989) or tomato (Pearce et al., 1991), and the 3′ region isolated from Cauliflower Mosaic Virus (Timmermans et al., 1990). Alternatively, one also could use a gamma coixin, oleosin 3 or other 3′ UTRs from the genus Coix (PCT Publication WO 99/58659).

3. Transit or Signal Peptides

Sequences that are joined to the coding sequence of an expressed gene, which are removed post-translationally from the initial translation product and which facilitate the transport of the protein into or through intracellular or extracellular membranes, are termed transit sequences (usually into vacuoles, vesicles, plastids and other intracellular organelles) and signal sequences (usually to the endoplasmic reticulum, golgi apparatus, peroxisomes or glyoxysomes, and outside of the cellular membrane). By facilitating the transport of the protein into compartments inside and outside the cell, these sequences may increase the accumulation of a gene product protecting the protein from intracellular proteolytic degradation. These sequences also allow for additional mRNA sequences from highly expressed genes to be attached to the coding sequence of the genes. Since mRNA being translated by ribosomes is more stable than naked mRNA, the presence of translatable mRNA 5′ of the gene of interest may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

A particular example of such a use concerns the direction of a protein conferring herbicide resistance, such as a glyphosate resistant EPSPS protein, to a particular organelle such as the chloroplast, rather than to the cytoplasm. This is exemplified by the use of the rbcS transit peptide, the chloroplast transit peptide described in U.S. Pat. No. 5,728,925, or the optimized transit peptide described in U.S. Pat. No. 5,510,471, which confer plastid-specific targeting of proteins. In addition, it may be desirable to target certain genes responsible for male sterility to the mitochondria, or to target certain genes for resistance to phytopathogenic organisms to the extracellular spaces, or to target proteins to the vacuole. A further use concerns the direction of enzymes involved in amino acid biosynthesis or oil synthesis to the plastid. Such enzymes include dihydrodipicolinic acid synthase which may contribute to increasing the lysine content of a seed.

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. An intracellular targeting DNA sequence may be operably linked 5′ or 3′ to the coding sequence depending on the particular targeting sequence. The resultant transit, or signal, peptide will transport the protein to a particular intracellular, or extracellular destination, respectively, and will then be post-translationally removed.

4. Marker Genes

Characteristics useful for selectable markers in plants have been outlined in a report on the use of microorganisms (Advisory Committee on Novel Foods and Processes, July 1994). These include:

(i) stringent selection with minimum number of nontransformed tissues;

(ii) large numbers of independent transformation events with no significant interference with regeneration of plants;

(iii) application to a large number of species; and

(iv) availability of an assay to score the tissues for presence of the marker.

As mentioned, several antibiotic resistance markers satisfy these criteria, including those conferring resistance to kanamycin (nptII), hygromycin B (aph IV) and gentamycin (aac3 and aacC4) as well as herbicides such as glufosinate (bar orpat) and glyphosate (EPSPS).

A number of selectable marker genes are known in the art and can be used in the present invention (see for example Wilmink and Dons, 1993). Particularly preferred selectable marker genes for use in the present invention would include genes that confer resistance to compounds such as antibiotics like kanamycin (Dekeyser et al., 1989), and herbicides like glyphosate (Della-Cioppa et al., 1987). Other selection devices can also be implemented including but not limited to tolerance to phosphinothricin, bialaphos, and positive selection mechanisms (Joersbo et al., 1998) and would still fall within the scope of the present invention.

By employing a selectable or screenable marker gene as, or in addition to, the gene of interest, one can provide or enhance the ability to identify transformants. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening”′ (e.g., the green fluorescent protein). Of course, many examples of suitable marker genes are known to the art and can be employed in the practice of the invention.

Included within the terms selectable or screenable marker genes also are genes which encode a “secretable marker” whose secretion can be detected as a means of identifying or selecting for transformed cells. Examples include marker genes which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR—S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

a. Selectable Markers

Many selectable marker coding regions may be used in connection with the present invention including, but not limited to, nptII (Potrykus et al., 1985) which provides kanamycin resistance and can be selected for using kanamycin, G418, paromomycin, etc.; bar, which confers bialaphos or phosphinothricin resistance; a glyphosate resistant EPSP synthase protein (Hinchee et al, 1988 U.S. Pat. No. 5,633,435) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (EP 0 154 204); a methotrexate resistant DHFR (Thillet et al., 1988), a dalapon dehalogenase that confers resistance to the herbicide dalapon (U.S. Pat. No. 5,780,708); or a mutated anthranilate synthase that confers resistance to 5-methyl tryptophan or other anthranilate synthase inhibiting compounds (U.S. Pat. No. 6,118,047). The cloning of the bar gene has been described (Murakami et al., 1986; Thompson et al., 1987) as has the use of the bar gene in the context of U.S. Pat. No. 5,550,318).

b. Screenable Markers

Screenable markers that may be employed include a β-glucuronidase or uidA gene (Jefferson et al., 1986; the protein product is commonly referred to as GUS), isolated from E. coli, which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an (x-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; β-galactosidase gene, which encodes an enzyme for which there are chromogenic substrates; a luciferase (lux) gene (Ow et al., 1986), which allows for bioluminescence detection; an aequorin gene (Prasher et al, 1985) which may be employed in calcium-sensitive bioluminescence detection; or a gene encoding for green fluorescent protein (Sheen et al., 1995; Haseloff et al., 1997; Reichel et al, 1996; Tian et al, 1997; PCT Publication WO 97/41228).

The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which has the genotype r-g, b, P1. Alternatively, any genotype of maize can be utilized if the C1 and R alleles are introduced together.

It further is proposed that R gene regulatory regions may be employed in chimeric constructs in order to provide mechanisms for controlling the expression of chimeric genes. More diversity of phenotypic expression is known at the R locus than at any other locus (Coe et al., 1988). It is contemplated that regulatory regions obtained from regions 5′ to the structural R gene would be valuable in directing the expression of genes for, e.g., insect resistance, herbicide tolerance or other protein coding regions. For the purposes of the present invention, it is believed that any of the various R gene family members may be successfully employed (e.g., P, S, Lc, etc). However, the most preferred will generally be Sn (particularly Sn:bol3). Sn is a dominant member of the R gene complex and is functionally similar to the R and B loci in that Sn controls the tissue specific deposition of anthocyanin pigments in certain seedling and plant cells, therefore, it's phenotype is similar to R.

Other screenable markers provide for visible light emission as a screenable phenotype. A screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is contemplated as a particularly useful reporter gene (Sheen et al., 1995; Haseloff et al., 1997; Reichel et al., 1996; Tian et al., 1997; PCT Publication WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP-NPTII gene fusion (PCT Publication WO 99/60129). This could allow, for example, selection of transformed cells followed by screening of transgenic plants or seeds. In a similar manner, it is possible to utilize other readily available fluorescent proteins such as red fluorescent protein (CLONTECH, Palo Alto, Calif.).

Exogenous Genes for Modification of Plant Phenotypes

A particularly important advance of the present invention is that it provides DNA sequences useful for producing desirable phenotypes in plants, preferably crop plants such as soybean, cotton, canola, sunflower and wheat, and most preferably in maize.

The choice of a selected gene for expression in a plant host cell in accordance with the invention will depend on the purpose of gene expression. One of the major purposes of transformation of crop plants is to add commercially desirable, agronomically important or end-product traits to the plant. Such traits include, but are not limited to, herbicide resistance or tolerance, insect resistance or tolerance, disease resistance or tolerance (viral, bacterial, fungal, nematode), stress tolerance and/or resistance, as exemplified by resistance or tolerance to drought, heat, chilling, freezing, excessive moisture, salt stress and oxidative stress, increased yield, food or feed content and value, physical appearance, male sterility, drydown, standability, prolificacy, starch quantity and quality, oil quantity and quality, protein quality and quantity, amino acid composition, and the like. The present invention provides various strategies for crop improvement and genes associated with the strategies. The genes and strategies are listed in Table 1 below and described in more detail herein.

TABLE 1

| | | PEP | | | | |
|---|---|---|---|---|---|---|
| SEQ | | SEQ | | | | |
| ID | | ID | | DONOR | CODING | |
| NO | PHE_ID | NO | GENE NAME | ORGANISM | SEQUENCE | STRATEGY |
| 1 | PHE0000022 | 369 | Zm AAP6 | Zea mays | 96-1547 | Amino acid transporters |
| 2 | PHE0000023 | 370 | Os ProT | Oryza sativa | 144-1562 | Amino acid transporters |
| 3 | PHE0000279 | 371 | sorghum proline | Sorghum | 16-1341 | Amino acid transporters |
| | | | permease | bicolor | | |
| 4 | PHE0000280 | 372 | rice AA transporter | Oryza sativa | 61-1485 | Amino acid transporters |
| 5 | PHE0000281 | 373 | K/H specific amino | Zea mays | 63-1427 | Amino acid transporters |
| | | | acid permease | | | |
| 6 | PHE0000402 | 374 | rice amino acid | Oryza sativa | 89-1426 | Amino acid transporters |
| | | | transporter-like | | | |
| | | | protein | | | |
| 7 | PHE0000403 | 375 | corn amino acid | Zea mays | 116-1453 | Amino acid transporters |
| | | | permease | | | |
| 8 | PHE0000404 | 376 | rice proline transport | Oryza sativa | 313-1731 | Amino acid transporters |
| | | | protein | | | |
| 9 | PHE0000450 | 377 | rice amino acid | Oryza sativa | 110-1450 | Amino acid transporters |
| | | | permease | | | |
| 10 | PHE0000108 | 378 | ASH1 | Arabidopsis | 61-801 | Protection against |
| | | | | thaliana | | osmotic stress |
| 11 | PHE0000109 | 379 | rice ASH1-like1 | Oryza sativa | 136-1008 | Protection against |
| | | | | | | osmotic stress |
| 12 | PHE0000110 | 380 | rice MtN2-like | Oryza sativa | 425-464, 546-582, | Protection against |
| | | | | | 672-783, | osmotic stress |
| | | | | | 812-898, | |
| | | | | | 988-1149, | |
| | | | | | 1556-1675, | |
| | | | | | 1776-1952 | |
| 10 | PHE0000108 | 378 | 61-801 | ASH1 | Arabidopsis | Modification of sugar |
| | | | | | thaliana | transport and/or |
| | | | | | | metabolism |
| 11 | PHE0000109 | 379 | 136-1008 | rice ASH1- | Oryza sativa | Modification of sugar |
| | | | | like1 | | transport and/or |
| | | | | | | metabolism |
| 12 | PHE0000110 | 380 | 425-464, 546-582, | rice MtN2-like | Oryza sativa | Modification of sugar |
| | | | 672-783, 812-898, | | | transport and/or |
| | | | 988-1149, 1556-1675, | | | metabolism |
| | | | 1776-1952 | | | |
| 13 | PHE0000262 | 381 | cytochrome P450-like | Zea mays | 29-1495 | Plant steroid |
| | | | protein | | | biosynthesis pathway |
| | | | | | | and signal transduction |
| | | | | | | genes |
| 14 | PHE0000263 | 382 | cytochrome P450 | Zea mays | 141-1637 | Plant steroid |
| | | | | | | biosynthesis pathway |
| | | | | | | and signal transduction |
| | | | | | | genes |
| 15 | PHE0000264 | 383 | cytochrome P450-like | Zea mays | 104-1657 | Plant steroid |
| | | | | | | biosynthesis pathway |
| | | | | | | and signal transduction |
| | | | | | | genes |
| 16 | PHE0000265 | 384 | CYP90 protein | Zea mays | 81-1589 | Plant steroid |
| | | | | | | biosynthesis pathway |
| | | | | | | and signal transduction |
| | | | | | | genes |
| 17 | PHE0000266 | 385 | cytochrome P450 | Zea mays | 92-1648 | Plant steroid |
| | | | DWARF3 | | | biosynthesis pathway |
| | | | | | | and signal transduction |
| | | | | | | genes |
| 18 | PHE0000267 | 386 | cytochrome P450 | Zea mays | 134-1543 | Plant steroid |
| | | | | | | biosynthesis pathway |
| | | | | | | and signal transduction |
| | | | | | | genes |
| 19 | PHE0000268 | 387 | rice receptor protein | Oryza sativa | 183-476, 706-735, | Plant steroid |
| | | | kinase | | 2796-6734 | biosynthesis pathway |
| | | | | | | and signal transduction |
| | | | | | | genes |
| 20 | PHE0000084 | 388 | rice cyclin H | Oryza sativa | 235-1227 | Cyclin dependent kinase |
| | | | | | | activating kinase |
| 21 | PHE0000085 | 389 | rice cdc2+/CDC28- | Oryza sativa | 173-1447 | Cyclin dependent kinase |
| | | | related protein kinase | | | activating kinase |
| 22 | PHE0000086 | 390 | Cdk-activating kinase 1 | Glycine max | 14-1240 | Cyclin dependent kinase |
| | | | | | | activating kinase |
| 23 | PHE0000284 | 391 | menage a trois-like | Zea mays | 164-745 | Cyclin dependent kinase |
| | | | | | | activating kinase |
| 24 | PHE0000362 | 392 | CDC28-related | Zea mays | 198-1484 | Cyclin dependent kinase |
| | | | protein kinase | | | activating kinase |
| 25 | PHE0000083 | 393 | PDR5 | Saccharomyces | 1552-6087 | Cercosporin resistance |
| | | | | cerevisiae | | |
| 26 | PHE0000089 | 394 | CHL1 | Arabidopsis | 85-1857 | Chlorate/Nitrate |
| | | | | thaliana | | transporter |
| 27 | PHE0000090 | 395 | NTR1 | Oryza sativa | 144-1898 | Chlorate/Nitrate |
| | | | | | | transporter |
| 28 | PHE0000228 | 396 | Synechocystis cobA | Synechocystis | 70-801 | Siroheme synthesis |
| | | | | sp. PCC 6803 | | |
| 29 | PHE0000229 | 397 | Xylella tetrapyrrole | Xylella | 1-774 | Siroheme synthesis |
| | | | methylase | fastidiosa | | |
| 30 | PHE0000230 | 398 | maize | Zea mays | 15-1286 | Siroheme synthesis |
| | | | uroporphyrinogen III | | | |
| | | | methyltransferase | | | |
| 31 | PHE0000190 | 399 | LEA3 | Zea mays | 171-755 | Cold induced genes |
| 32 | PHE0000191 | 400 | non-specific lipid | Zea mays | 70-456 | Cold induced genes |
| | | | transfer protein | | | |
| 33 | PHE0000234 | 401 | soy LEA protein | Glycine max | 6-704 | Cold induced genes |
| 34 | PHE0000235 | 402 | dehydrin-like protein | Glycine max | 33-710 | Cold induced genes |
| 35 | PHE0000236 | 403 | glycine-rich protein | Zea mays | 91-558 | Cold induced genes |
| 36 | PHE0000237 | 404 | dehydrin 3 | Zea mays | 84-584 | Cold induced genes |
| 37 | PHE0000238 | 405 | probable lipase | Zea mays | 98-967 | Cold induced genes |
| 38 | PHE0000239 | 406 | yeast GRE1 | Saccharomyces | 1024-1527 | Cold induced genes |
| | | | | cerevisiae | | |
| 39 | PHE0000240 | 407 | yeast STF2 | Saccharomyces | 683-934 | Cold induced genes |
| | | | | cerevisiae | | |
| 40 | PHE0000241 | 408 | yeast SIP18 | Saccharomyces | 376-855 | Cold induced genes |
| | | | | cerevisiae | | |
| 41 | PHE0000242 | 409 | yeast YBM6 | Saccharomyces | 744-1130 | Cold induced genes |
| | | | | cerevisiae | | |
| 42 | PHE0000243 | 410 | yeast HSP12 | Saccharomyces | 282-611 | Cold induced genes |
| | | | | cerevisiae | | |
| 43 | PHE0000038 | 411 | corn cycD2.1 | Zea mays | 125-1156 | Corn cyclins |
| 44 | PHE0000043 | 412 | rice cycB1 | Oryza sativa | 148-1407 | Corn cyclins |
| 45 | PHE0000044 | 413 | rice cycC1 | Oryza sativa | 97-870 | Corn cyclins |
| 46 | PHE0000045 | 414 | rice cycB2 | Oryza sativa | 74-1336 | Corn cyclins |
| 47 | PHE0000046 | 415 | rice cycA1 | Oryza sativa | 97-1623 | Corn cyclins |
| 48 | PHE0000047 | 416 | rice cycB5 | Oryza sativa | 292-361, 1019-1347, | Corn cyclins |
| | | | | | 1447-1572, | |
| | | | | | 1657-1908, | |
| | | | | | 2059-2217, | |
| | | | | | 2315-2493, | |
| | | | | | 3276-3432 | |
| 49 | PHE0000050 | 417 | corn cycA2 | Zea mays | 107-1222 | Corn cyclins |
| 50 | PHE0000051 | 418 | corn cycB2 | Zea mays | 137-1408 | Corn cyclins |
| 51 | PHE0000052 | 419 | corn cycB5 | Zea mays | 82-1518 | Corn cyclins |
| 52 | PHE0000053 | 420 | corn cycB4 | Zea mays | 254-1579 | Corn cyclins |
| 53 | PHE0000054 | 421 | corn cycD3.2 | Zea mays | 220-1380 | Corn cyclins |
| 54 | PHE0000055 | 422 | corn cycDx.1 | Zea mays | 218-1180 | Corn cyclins |
| 55 | PHE0000056 | 423 | corn cycD1.1 | Zea mays | 288-1334 | Corn cyclins |
| 56 | PHE0000082 | 424 | corn cycB3 | Zea mays | 88-1425 | Corn cyclins |
| 57 | PHE0000101 | 425 | corn cycD3.1 | Zea mays | 250-1422 | Corn cyclins |
| 58 | PHE0000105 | 426 | corn cycD1.2 | Zea mays | 229-1275 | Corn cyclins |
| 59 | PHE0000106 | 427 | corn cycA1 | Zea mays | 107-1633 | Corn cyclins |
| 60 | PHE0000107 | 428 | corn cycD1.3 | Zea mays | 206-1252 | Corn cyclins |
| 61 | PHE0000382 | 429 | corn cycB1 | Zea mays | 114-1385 | Corn cyclins |
| 62 | PHE0000014 | 430 | rice cycD2 | Oryza sativa | 13-324, 623-709, | Cytokinins |
| | | | | | 813-911, | |
| | | | | | 1003-1204, | |
| | | | | | 1314-1438, | |
| | | | | | 1529-1774 | |
| 63 | PHE0000015 | 431 | rice GCR1 | Oryza sativa | 312-500, 1123-1154, | Cytokinins |
| | | | | | 1384-1553, | |
| | | | | | 2048-2163, | |
| | | | | | 2724-2825, | |
| | | | | | 2946-3002, | |
| | | | | | 3331-3474, | |
| | | | | | 3930-4000, | |
| | | | | | 4118-4223 | |
| 64 | PHE0000016 | 432 | kn 1 | Zea mays | 181-1257 | Cytokinins |
| 65 | PHE0000115 | 433 | Receiver domain | Zea mays | 277-1002 | Cytokinins |
| | | | (RR3-like) 7 | | | |
| 66 | PHE0000116 | 434 | Receiver domain | Zea mays | 188-2245 | Cytokinins |
| | | | (ARR2-like) 1 | | | |
| 67 | PHE0000117 | 435 | Receiver domain | Zea mays | 112-2238 | Cytokinins |
| | | | (TOC1-like) 2 | | | |
| 68 | PHE0000118 | 436 | Receiver domain | Zea mays | 84-1976 | Cytokinins |
| | | | (TOC1-like) 3 | | | |
| 69 | PHE0000119 | 437 | Receiver domain | Zea mays | 39-1931 | Cytokinins |
| | | | (ARR2-like) 4 | | | |
| 70 | PHE0000120 | 438 | Receiver domain | Zea mays | 61-1812 | Cytokinins |
| | | | (RR11-like) 5 | | | |
| 71 | PHE0000121 | 439 | Receiver domain | Zea mays | 391-1116 | Cytokinins |
| | | | (RR3-like) 6 | | | |
| 72 | PHE0000122 | 440 | Receiver domain | Zea mays | 335-1066 | Cytokinins |
| | | | (RR3-like) 8 | | | |
| 73 | PHE0000123 | 441 | Receiver domain 9 | Zea mays | 55-759 | Cytokinins |
| 74 | PHE0000124 | 442 | ZmRR2 | Zea mays | 154-624 | Cytokinins |
| 75 | PHE0000125 | 443 | Receiver domain | Zea mays | 374-722, 791-2019 | Cytokinins |
| | | | (TOC1-like) 10 | | | |
| 76 | PHE0000272 | 444 | corn GCR1 | Zea mays | 74-1036 | Cytokinins |
| 77 | PHE0000231 | 445 | nucellin-like protein | Zea mays | 122-1594 | Alteration of oil content |
| | | | | | | by effecting embryo size |
| 78 | PHE0000232 | 446 | nucellin-like protein | Zea mays | 76-1605 | Alteration of oil content |
| | | | | | | by effecting embryo size |
| 79 | PHE0000233 | 447 | nucellin-like protein | Zea mays | 195-1628 | Alteration of oil content |
| | | | | | | by effecting embryo size |
| 80 | PHE0000269 | 448 | soy E2F-like | Glycine max | 80-1117 | Cell cycle regulation by |
| | | | | | | E2F |
| 81 | PHE0000270 | 449 | nuclear matrix | Zea mays | 243-3371 | Cell cycle regulation by |
| | | | constituent protein | | | E2F |
| 82 | PHE0000271 | 450 | OsE2F1 | Oryza sativa | 93-1403 | Cell cycle regulation by |
| | | | | | | E2F |
| 83 | PHE0000067 | 451 | yeast eIF-5A | Saccharomyces | 569-1042 | Reduction of senescence |
| | | | | cerevisiae | | |
| 84 | PHE0000068 | 452 | yeast deoxyhypusine | Saccharomyces | 173-1336 | Reduction of senescence |
| | | | synthase | cerevisiae | | |
| 85 | PHE0000069 | 453 | yeast L5 | Saccharomyces | 987-1880 | Reduction of senescence |
| | | | | cerevisiae | | |
| 86 | PHE0000070 | 454 | yeast ornithine | Saccharomyces | 576-1976 | Reduction of senescence |
| | | | decarboxylase | cerevisiae | | |
| 87 | PHE0000071 | 455 | rice exportin 4-like | Oryza sativa | 501-750, 1257-1417, | Reduction of senescence |
| | | | | | 1735-1800, | |
| | | | | | 3104-3218, | |
| | | | | | 3318-3427, | |
| | | | | | 3525-3620, | |
| | | | | | 7587-7744, | |
| | | | | | 7828-7915, | |
| | | | | | 8565-8669, | |
| | | | | | 8774-8878, | |
| | | | | | 9421-9450, | |
| | | | | | 9544-9656, | |
| | | | | | 9732-9819, | |
| | | | | | 9961-10180, | |
| | | | | | 11034-11164, | |
| | | | | | 12058-12204, | |
| | | | | | 12770-12898, | |
| | | | | | 12975-13073, | |
| | | | | | 13221-13259, | |
| | | | | | 14674-14823 | |
| 88 | PHE0000072 | 456 | yeast S- | Saccharomyces | 415-1605 | Reduction of senescence |
| | | | adenosylmethionine | cerevisiae | | |
| | | | decarboxylase | | | |
| 89 | PHE0000073 | 457 | corn S- | Zea mays | 268-1365 | Reduction of senescence |
| | | | adenosylmethionine | | | |
| | | | decarboxylase 1 | | | |
| 90 | PHE0000074 | 458 | corn S- | Zea mays | 581-1780 | Reduction of senescence |
| | | | adenosylmethionine | | | |
| | | | decarboxylase 2 | | | |
| 91 | PHE0000204 | 459 | deoxyhypusine | Glycine max | 26-1129 | Reduction of senescence |
| | | | synthase | | | |
| 92 | PHE0000291 | 460 | deoxyhypusine | Zea mays | 54-1163 | Reduction of senescence |
| | | | synthase | | | |
| 93 | PHE0000292 | 461 | corn eIF-5A | Zea mays | 85-564 | Reduction of senescence |
| 94 | PHE0000286 | 462 | oryzacystatin | Oryza sativa | 108-527 | Endogenous insecticides |
| 95 | PHE0000287 | 463 | Similar to cysteine | Oryza sativa | 18-767 | Endogenous insecticides |
| | | | proteinase inhibitor | | | |
| 96 | PHE0000288 | 464 | cysteine proteinase | Sorghum | 135-461 | Endogenous insecticides |
| | | | inhibitor | bicolor | | |
| 97 | PHE0000001 | 465 | esk2-like-cellulose | Zea mays | 113-3061 | Freezing tolerance- |
| | | | synthase | | | Eskimo 2 |
| 98 | PHE0000227 | 466 | soy omega-3 fatty | Glycine max | 138-1496 | Fatty acid desaturases |
| | | | acid desaturase | | | |
| 99 | PHE0000258 | 467 | AtFAD7 | Arabidopsis | 132-1472 | Fatty acid desaturases |
| | | | | thaliana | | |
| 100 | PHE0000259 | 468 | AtFAD8 | Arabidopsis | 61-1368 | Fatty acid desaturases |
| | | | | thaliana | | |
| 101 | PHE0000260 | 469 | desB | Synechocystis | 643-1719 | Fatty acid desaturases |
| | | | | sp. PCC 6803 | | |
| 102 | PHE0000186 | 470 | maize ferritin 2 | Zea mays | 3-758 | Ferritin |
| 103 | PHE0000187 | 471 | maize ferritin 1 | Zea mays | 34-795 | Ferritin |
| 104 | PHE0000188 | 472 | E. coli cytoplasmic | Escherichia | 245-742 | Ferritin |
| | | | ferritin | coli | | |
| 105 | PHE0000102 | 473 | AnFPPS (farnesyl- | Emericella | 146-1186 | Farnesylpyrophosphate |
| | | | pyrophosphate | nidulans | | synthase |
| | | | synthetase) | | | |
| 106 | PHE0000103 | 474 | OsFPPS | Oryza sativa | 42-1103 | Farnesylpyrophosphate |
| | | | | | | synthase |
| 107 | PHE0000104 | 475 | corn FPPS 2 | Zea mays | 313-1377 | Farnesylpyrophosphate |
| | | | | | | synthase |
| 108 | PHE0000277 | 476 | wheat G28-like | Triticum | 65-877 | Fungal Resistance |
| | | | | aestivum | | |
| 109 | PHE0000261 | 477 | yeast glutamate | Saccharomyces | 33-1790 | Gamma-aminobutyric |
| | | | decarboxylase | cerevisiae | | acid synthesis |
| 110 | PHE0000019 | 478 | AOX1a | Oryza sativa | 4531-4851, 5011-5139, | Heat generation |
| | | | | | 6072-6560, | |
| | | | | | 6663-6722 | |
| 111 | PHE0000020 | 479 | alxA | Emericella | 2189-2442, 2492-2783, | Heat generation |
| | | | | nidulans | 2843-3352 | |
| 112 | PHE0000095 | 480 | HSF1 | Saccharomyces | 1017-3518 | Heat tolerance |
| | | | | cerevisiae | | |
| 113 | PHE0000096 | 481 | Zm HSP101 | Zea mays | 436-1773, 1878-2159, | Heat tolerance |
| | | | | | 2281-2621, | |
| | | | | | 2711-2990, | |
| | | | | | 3079-3276, | |
| | | | | | 3371-3670 | |
| 114 | PHE0000097 | 482 | HSP104 | Saccharomyces | 334-3060 | Heat tolerance |
| | | | | cerevisiae | | |
| 115 | PHE0000098 | 483 | E. coli clpB | Escherichia | 557-3130 | Heat tolerance |
| | | | | coli | | |
| 116 | PHE0000099 | 484 | Synechocystis clpB | Synechocystis | 316-2931 | Heat tolerance |
| | | | | sp. PCC 6803 | | |
| 117 | PHE0000100 | 485 | Xylella clpB | Xylella | 187-2769 | Heat tolerance |
| | | | | fastidiosa | | |
| 118 | PHE0000192 | 486 | soy HSF | Glycine max | 23-1114 | Heat tolerance |
| 119 | PHE0000193 | 487 | soy HSF | Glycine max | 93-992 | Heat tolerance |
| 120 | PHE0000133 | 488 | G protein b subunit | Zea mays | 90-1229 | Heterotrimeric G |
| | | | | | | proteins |
| 121 | PHE0000273 | 489 | soy mlo-like | Glycine max | 15-1532 | Heterotrimeric G |
| | | | | | | proteins |
| 122 | PHE0000274 | 490 | soy mlo-like | Glycine max | 48-1841 | Heterotrimeric G |
| | | | | | | proteins |
| 123 | PHE0000275 | 491 | rice G alpha 1 | Oryza sativa | 106-1248 | Heterotrimeric G |
| | | | | | | proteins |
| 124 | PHE0000276 | 492 | soy G-gamma subunit | Glycine max | 210-536 | Heterotrimeric G |
| | | | | | | proteins |
| 125 | PHE0000062 | 493 | sRAD54 - with NLS | Synechocystis | 437-3556 | Homologous |
| | | | | sp. PCC 6803 | | recombination |
| 126 | PHE0000063 | 494 | T4 endonuclease VII | coliphage T4 | 603-1148 | Homologous |
| | | | (gp49) - with NLS | | | recombination |
| 127 | PHE0000169 | 495 | maize p23 | Zea mays | 106-708 | HSP90 |
| 128 | PHE0000170 | 496 | maize cyclophilin | Zea mays | 99-1757 | HSP90 |
| 129 | PHE0000171 | 497 | yeast HSP82 | Saccharomyces | 333-2462 | HSP90 |
| | | | | cerevisiae | | |
| 130 | PHE0000172 | 498 | yeast SIT1 | Saccharomyces | 361-2130 | HSP90 |
| | | | | cerevisiae | | |
| 131 | PHE0000173 | 499 | yeast CNS1 | Saccharomyces | 762-1919 | HSP90 |
| | | | | cerevisiae | | |
| 132 | PHE0000174 | 500 | yeast HCH1 | Saccharomyces | 193-654 | HSP90 |
| | | | | cerevisiae | | |
| 133 | PHE0000298 | 501 | rice p23 co-chaperone | Oryza sativa | 68-706 | HSP90 |
| 134 | PHE0000299 | 502 | corn p23 co- | Zea mays | 71-565 | HSP90 |
| | | | chaperone | | | |
| 135 | PHE0000300 | 503 | rice p23 co-chaperone | Oryza sativa | 124-642 | HSP90 |
| 136 | PHE0000301 | 504 | corn p23 co- | Zea mays | 90-617 | HSP90 |
| | | | chaperone | | | |
| 137 | PHE0000436 | 505 | rice cns1-like | Oryza sativa | 121-1242 | HSP90 |
| 138 | PHE0000437 | 506 | corn HCH1-like 1 | Zea mays | 42-1100 | HSP90 |
| 139 | PHE0000438 | 507 | corn HOP-like 1 | Zea mays | 88-1830 | HSP90 |
| 140 | PHE0000439 | 508 | corn HOP-like 2 | Zea mays | 65-1261 | HSP90 |
| 141 | PHE0000440 | 509 | rice CHIP-like 1 | Oryza sativa | 121-939 | HSP90 |
| 142 | PHE0000441 | 510 | corn CHIP-like 2 | Zea mays | 115-939 | HSP90 |
| 143 | PHE0000442 | 511 | corn HSP90 1 | Zea mays | 55-2478 | HSP90 |
| 144 | PHE0000443 | 512 | rice HSP90 1 | Oryza sativa | 68-2500 | HSP90 |
| 145 | PHE0000444 | 513 | corn HSP90 2 | Zea mays | 63-2423 | HSP90 |
| 146 | PHE0000445 | 514 | sorghum HSP90 1 | Sorghum | 138-2285 | HSP90 |
| | | | | bicolor | | |
| 147 | PHE0000446 | 515 | rice HSP90 2 | Oryza sativa | 78-2174 | HSP90 |
| 148 | PHE0000215 | 516 | invW | Oryza sativa | 1108-1489, 1813-2684, | Invertase |
| | | | | | 6105-6266, | |
| | | | | | 6417-6658, | |
| 149 | PHE0000248 | 517 | Zm lipoxygenase | Zea mays | 89-2821 | Jasmonate |
| 150 | PHE0000249 | 518 | corn allene oxide | Zea mays | 111-1556 | Jasmonate |
| | | | synthase | | | |
| 151 | PHE0000250 | 519 | corn COI1-like | Zea mays | 139-1911 | Jasmonate |
| 152 | PHE0000252 | 520 | corn COI1-like | Zea mays | 130-1923 | Jasmonate |
| 153 | PHE0000253 | 521 | COI1-like | Zea mays | 389-2368 | Jasmonate |
| 154 | PHE0000256 | 522 | corn 1- | Zea mays | 61-1011 | Jasmonate |
| | | | aminocyclopropane- | | | |
| | | | 1-carboxylate oxidase | | | |
| 155 | PHE0000257 | 523 | rice 1- | Oryza sativa | 2-1465 | Jasmonate |
| | | | aminocyclopropane-1 | | | |
| | | | carboxylate synthase | | | |
| 156 | PHE0000432 | 524 | corn 12- | Zea mays | 128-1240 | Jasmonate |
| | | | oxophytodienoate | | | |
| | | | reductase 1 | | | |
| 157 | PHE0000433 | 525 | corn 12-oxo- | Zea mays | 166-1242 | Jasmonate |
| | | | phytodienoate | | | |
| | | | reductase-like 3 | | | |
| 158 | PHE0000434 | 526 | corn 12- | Zea mays | 92-1210 | Jasmonate |
| | | | oxophytodienoate | | | |
| | | | reductase-like 4 | | | |
| 159 | PHE0000435 | 527 | corn hydroperoxide | Zea mays | 83-1594 | Jasmonate |
| | | | lyase | | | |
| 160 | PHE0000484 | 528 | soy JMT-like protien 1 | Glycine max | 26-1135 | Jasmonate |
| 161 | PHE0000485 | 529 | corn JMT-like protein 1 | Zea mays | 39-1184 | Jasmonate |
| 162 | PHE0000486 | 530 | corn JMT-like protein 2 | Zea mays | 63-1208 | Jasmonate |
| 163 | PHE0000077 | 531 | yeast | Saccharomyces | 1695-2894 | Nitric oxide |
| | | | flavohemoglobin - | cerevisiae | | |
| | | | chloroplastic | | | |
| 164 | PHE0000039 | 532 | nph1 | Zea mays | 415-3150 | Photomorphogenic |
| | | | | | | responses |
| 165 | PHE0000176 | 533 | RNAse S | Zea mays | 85-771 | Phosphate uptake |
| 166 | PHE0000177 | 534 | maize ecto-apyrase | Zea mays | 210-2312 | Phosphate uptake |
| 167 | PHE0000178 | 535 | PHO5 | Saccharomyces | 1-1404 | Phosphate uptake |
| | | | | cerevisiae | | |
| 168 | PHE0000179 | 536 | high affinity | Glycine max | 105-1703 | Phosphate uptake |
| | | | phosphate | | | |
| | | | translocator | | | |
| 169 | PHE0000180 | 537 | high affinity | Zea mays | 128-1750 | Phosphate uptake |
| | | | phosphate | | | |
| | | | translocator | | | |
| 170 | PHE0000181 | 538 | Xylella citrate | Xylella | 256-1545 | Phosphate uptake |
| | | | synthase | fastidiosa | | |
| 171 | PHE0000182 | 539 | E. coli citrate | Escherichia | 309-1592 | Phosphate uptake |
| | | | synthase | coli | | |
| 172 | PHE0000183 | 540 | rice citrate synthase | Oryza sativa | 105-1523 | Phosphate uptake |
| 173 | PHE0000184 | 541 | citrate synthase | Zea mays | 56-1564 | Phosphate uptake |
| 174 | PHE0000185 | 542 | citrate synthase | Glycine max | 153-1691 | Phosphate uptake |
| 175 | PHE0000302 | 543 | putative purple acid | Oryza sativa | 22-1038 | Phosphate uptake |
| | | | phosphatase precursor | | | |
| 176 | PHE0000303 | 544 | acid phosphatase type 5 | Zea mays | 143-1186 | Phosphate uptake |
| 177 | PHE0000304 | 545 | aleurone ribonuclease | Oryza sativa | 47-814 | Phosphate uptake |
| 178 | PHE0000305 | 546 | putative ribonuclease | Zea mays | 55-888 | Phosphate uptake |
| 179 | PHE0000306 | 547 | S-like RNase | Zea mays | 15-770 | Phosphate uptake |
| 180 | PHE0000307 | 548 | ribonuclease | Zea mays | 95-781 | Phosphate uptake |
| 181 | PHE0000027 | 549 | SbPhytochrome A | Sorghum | 238-3633 | Phytochrome |
| | | | | bicolor | | |
| 182 | PHE0000028 | 550 | OsPhytochrome B | Oryza sativa | 67-3582 | Phytochrome |
| 183 | PHE0000029 | 551 | SbPhytochrome B | Sorghum | 429-2640, 3333-4140, | Phytochrome |
| | | | | bicolor | 5819-6112, | |
| | | | | | 7491-7713 | |
| 184 | PHE0000030 | 552 | OsPhytochrome C | Oryza sativa | 1036-3100, 3205-4021, | Phytochrome |
| | | | | | 4418-4711, | |
| | | | | | 5272-5509 | |
| 185 | PHE0000031 | 553 | SbPhytochrome C | Sorghum | 303-3710 | Phytochrome |
| | | | | bicolor | | |
| 186 | PHE0000032 | 554 | Positive Factor 1 | Oryza sativa | 35-676 | Phytochrome |
| 187 | PHE0000033 | 555 | GT-2 | Oryza sativa | 58-2271 | Phytochrome |
| 188 | PHE0000034 | 556 | biliverdin IXa | Synechocystis | 9-992 | Phytochrome |
| | | | reductase | sp. PCC 6803 | | |
| 189 | PHE0000049 | 557 | OsPhytochrome A | Oryza sativa | 4626-6690, 6913-7729, | Phytochrome |
| | | | | | 8011-8307, | |
| | | | | | 8410-8617 | |
| 190 | PHE0000057 | 558 | corn mt NDK - | Zea mays | 60-725 | Phytochrome |
| | | | LIB189022Q1E1E9 | | | |
| 191 | PHE0000058 | 559 | corn cp NDK - | Zea mays | 103-816 | Phytochrome |
| | | | 700479629 | | | |
| 192 | PHE0000059 | 560 | corn NDK - | Zea mays | 49-495 | Phytochrome |
| | | | LIB3597020Q1K6C3 | | | |
| 193 | PHE0000060 | 561 | corn NDK - | Zea mays | 162-608 | Phytochrome |
| | | | 700241377 | | | |
| 194 | PHE0000061 | 562 | OsGAI | Oryza sativa | 216-2093 | Phytochrome |
| 195 | PHE0000064 | 563 | corn NDPK - fC- | Zea mays | 91-624 | Phytochrome |
| | | | zmemLIB3957015Q1 | | | |
| | | | K6H6 | | | |
| 196 | PHE0000111 | 564 | PAS domain kinase | Zea mays | 358-2613 | Phytochrome |
| 197 | PHE0000126 | 565 | corn HY5-like | Zea mays | 32-541 | Phytochrome |
| 198 | PHE0000127 | 566 | scarecrow 1 (PAT1- | Zea mays | 295-1929 | Phytochrome |
| | | | like) | | | |
| 199 | PHE0000128 | 567 | scarecrow 2 | Zea mays | 153-1934 | Phytochrome |
| 200 | PHE0000283 | 568 | scarecrow 6 | Zea mays | 520-2145 | Phytochrome |
| 201 | PHE0000293 | 569 | gibberellin response | Zea mays | 131-2020 | Phytochrome |
| | | | modulator | | | |
| 202 | PHE0000294 | 570 | scarecrow-like protein | Zea mays | 266-1948 | Phytochrome |
| 203 | PHE0000308 | 571 | helix-loop-helix | Zea mays | 202-756 | Phytochrome |
| | | | protein (PIF3-like) | | | |
| 204 | PHE0000318 | 572 | scarecrow 17 | Zea mays | 441-2102 | Phytochrome |
| 205 | PHE0000361 | 573 | PAT1-like scarecrow 9 | Zea mays | 191-1900 | Phytochrome |
| 206 | PHE0000427 | 574 | corn SPA1-like 1 | Zea mays | 227-3139 | Phytochrome |
| 207 | PHE0000428 | 575 | corn PIF3-like | Zea mays | 173-856 | Phytochrome |
| 208 | PHE0000429 | 576 | soy Athb-2-like 1 | Glycine max | 78-932 | Phytochrome |
| 209 | PHE0000430 | 577 | corn SUB1-like 1 | Zea mays | 44-1954 | Phytochrome |
| 210 | PHE0000431 | 578 | soy GH3 protein | Glycine max | 42-1820 | Phytochrome |
| 211 | PHE0000065 | 579 | TOR1 | Saccharomyces | 302-7714 | TOR pathway |
| | | | | cerevisiae | | |
| 212 | PHE0000152 | 580 | 14-3-3-like protein 2 | Glycine max | 85-861 | TOR pathway |
| 213 | PHE0000153 | 581 | 14-3-3-like protein D | Glycine max | 42-824 | TOR pathway |
| 214 | PHE0000154 | 582 | 14-3-3 protein 1 | Glycine max | 49-834 | TOR pathway |
| 215 | PHE0000155 | 583 | Rice FAP1-like | Oryza sativa | 654-1862, 2310-2426, | TOR pathway |
| | | | protein | | 3407-3492, | |
| | | | | | 3590-3752, | |
| | | | | | 3845-3890, | |
| | | | | | 4476-4522, | |
| | | | | | 4985-5191, | |
| | | | | | 5306-5392, | |
| | | | | | 5473-5640 | |
| 216 | PHE0000156 | 584 | rice TAP42-like | Oryza sativa | 199-1338 | TOR pathway |
| 217 | PHE0000157 | 585 | rice eIF-4E | Oryza sativa | 58-741 | TOR pathway |
| 218 | PHE0000158 | 586 | BMH1 | Saccharomyces | 79-882 | TOR pathway |
| | | | | cerevisiae | | |
| 219 | PHE0000311 | 587 | GF14-c protein | Oryza sativa | 81-848 | TOR pathway |
| 220 | PHE0000312 | 588 | 14-3-3-like protein | Oryza sativa | 6-785 | TOR pathway |
| 221 | PHE0000313 | 589 | rice eIF-(iso)4F | Oryza sativa | 96-713 | TOR pathway |
| 222 | PHE0000314 | 590 | rice eIF-4F | Oryza sativa | 46-726 | TOR pathway |
| 223 | PHE0000315 | 591 | sorghum eIF-(iso)4F | Sorghum | 78-707 | TOR pathway |
| | | | | bicolor | | |
| 224 | PHE0000316 | 592 | sorghum eIF-4F | Sorghum | 9-668 | TOR pathway |
| | | | | bicolor | | |
| 225 | PHE0000317 | 593 | rice F1P37-like | Oryza sativa | 73-1128 | TOR pathway |
| 226 | PHE0000040 | 594 | Zm Hb1-hemoglobin | Zea mays | 172-669 | Plant hemoglobins |
| 227 | PHE0000400 | 595 | soy G559-like | Glycine max | 301-1560 | Plant hemoglobins |
| 228 | PHE0000091 | 596 | Zm SET domain 2 | Zea mays | 101-1009 | Polycomb proteins for |
| | | | | | | apomixis |
| 229 | PHE0000092 | 597 | Zm SET domain 1 | Zea mays | 528-1544 | Polycomb proteins for |
| | | | | | | apomixis |
| 230 | PHE0000114 | 598 | Su(var) 3-9-like | Zea mays | 71-814 | Polycomb proteins for |
| | | | | | | apomixis |
| 231 | PHE0000175 | 599 | corn EZA1-like | Zea mays | 34-2706 | Polycomb proteins for |
| | | | | | | apomixis |
| 232 | PHE0000282 | 600 | SET-domain protein- | Zea mays | 478-3045 | Polycomb proteins for |
| | | | like | | | apomixis |
| 233 | PHE0000075 | 601 | retinoblastoma-related | Zea mays | 37-2634 | Retinoblastoma (Rb) like |
| | | | protein 1 | | | genes |
| 234 | PHE0000076 | 602 | C1 protein | Wheat dwarf | 49-843 | Retinoblastoma (Rb) like |
| | | | | virus | | genes |
| 235 | PHE0000006 | 603 | RAP2.8/G9 | Arabidopsis | 81-1136 | Root mass |
| | | | | thaliana | | |
| 236 | PHE0000007 | 604 | rice G9-like 1 | Oryza sativa | 336-1430 | Root mass |
| 237 | PHE0000008 | 605 | rice G9-like 2 | Oryza sativa | 572-1522 | Root mass |
| 238 | PHE0000012 | 606 | rs81 | Zea mays | 1-747 | Root mass |
| 239 | PHE0000013 | 607 | rs288 | Zea mays | 1-864 | Root mass |
| 240 | PHE0000244 | 608 | corn SVP-like | Zea mays | 177-860 | Short vegetative phase |
| 241 | PHE0000245 | 609 | corn SVP-like | Zea mays | 93-791 | Short vegetative phase |
| 242 | PHE0000246 | 610 | soy SVP-like | Glycine max | 96-713 | Short vegetative phase |
| 243 | PHE0000247 | 611 | soy jointless-like | Glycine max | 60-674 | Short vegetative phase |
| 244 | PHE0000451 | 612 | wheat SVP-like 1 | Triticum | 149-736 | Short vegetative phase |
| | | | | aestivum | | |
| 245 | PHE0000452 | 613 | corn SVP-like 3 | Zea mays | 75-749 | Short vegetative phase |
| 246 | PHE0000453 | 614 | corn SVP-like 5 | Zea mays | 304-774, 956-1219 | Short vegetative phase |
| 247 | PHE0000087 | 615 | STURDY | Arabidopsis | 329-1267, 1353-1562 | Lodging resistance |
| | | | | thaliana | | |
| 248 | PHE0000088 | 616 | patatin-like protein | Nostoc | 451-2184 | Lodging resistance |
| | | | | PCC7120 | | |
| 249 | PHE0000220 | 617 | corn RNase PH | Zea mays | 86-805 | Virus resistance |
| 250 | PHE0000221 | 618 | SKI2 | Saccharomyces | 1351-5211 | Virus resistance |
| | | | | cerevisiae | | |
| 251 | PHE0000222 | 619 | SKI3 | Saccharomyces | 793-5091 | Virus resistance |
| | | | | cerevisiae | | |
| 252 | PHE0000223 | 620 | SKI4 | Saccharomyces | 323-1201 | Virus resistance |
| | | | | cerevisiae | | |
| 253 | PHE0000224 | 621 | SKI6 | Saccharomyces | 1007-1747 | Virus resistance |
| | | | | cerevisiae | | |
| 254 | PHE0000225 | 622 | SKI7 | Saccharomyces | 279-2519 | Virus resistance |
| | | | | cerevisiae | | |
| 255 | PHE0000226 | 623 | rice SKI7-like | Oryza sativa | 464-884, 1132-1287, | Virus resistance |
| | | | | | 2103-2252, | |
| | | | | | 2353-2487, | |
| | | | | | 2957-3288, | |
| | | | | | 3399-3509, | |
| | | | | | 3596-4095, | |
| | | | | | 4350-4518, | |
| | | | | | 4783-5022, | |
| | | | | | 5097-5228, | |
| | | | | | 5315-5449 | |
| 256 | PHE0000309 | 624 | SKI4-like protein | Zea mays | 36-632 | Virus resistance |
| 257 | PHE0000310 | 625 | putative 3 | Zea mays | 238-1098 | Virus resistance |
| | | | exoribonuclease | | | |
| 258 | PHE0000159 | 626 | rice chloroplastic | Oryza sativa | 41-1261 | Yield associated genes |
| | | | fructose-1,6- | | | |
| | | | bisphosphatase | | | |
| 259 | PHE0000160 | 627 | E. coli fructose-1,6- | Eseherichia | 208-1206 | Yield associated genes |
| | | | bisphosphatase | coli | | |
| 260 | PHE0000161 | 628 | Synechocystis | Synechocystis | 1-1164 | Yield associated genes |
| | | | fructose-1,6- | sp. PCC 6803 | | |
| | | | bisphosphatase F-I | | | |
| 261 | PHE0000162 | 629 | Synechocystis | Synechocystis | 480-1523 | Yield associated genes |
| | | | fructose-1,6- | sp. PCC 6803 | | |
| | | | bisphosphatase F-II | | | |
| 262 | PHE0000163 | 630 | Rice TBP1 | Oryza sativa | 26-1315 | Yield associated genes |
| 263 | PHE0000164 | 631 | Yeast RPT5 | Saccharomyces | 883-2187 | Yield associated genes |
| | | | | cerevisiae | | |
| 264 | PHE0000165 | 632 | Yeast RRP5 | Saccharomyces | 331-5520 | Yield associated genes |
| | | | | cerevisiae | | |
| 265 | PHE0000166 | 633 | Rice CBP-like gene | Oryza sativa | 277-436, 479-1524 | Yield associated genes |
| | | | | | 1790-2065, | |
| | | | | | 2150-2425, | |
| | | | | | 3134-3262, | |
| | | | | | 3380-3580, | |
| | | | | | 3683-3825, | |
| | | | | | 3905-4190, | |
| | | | | | 4294-4433, | |
| | | | | | 4711-4789, | |
| | | | | | 4874-4929, | |
| | | | | | 5754-5946 | |
| 266 | PHE0000167 | 634 | rice BAB09754 | Oryza sativa | 616-903, 1848-1940, | Yield associated genes |
| | | | | | 2046-2165, | |
| | | | | | 2254-2355, | |
| | | | | | 2443-2693, | |
| | | | | | 2849-2994, | |
| | | | | | 3165-3363, | |
| | | | | | 3475-4141, | |
| | | | | | 4438-4770, | |
| | | | | | 5028-5309 | |
| 267 | PHE0000168 | 635 | LIB3061-001-H7 FLI | Zea mays | 309-1037 | Yield associated genes |
| 268 | PHE0000295 | 636 | ubiquitin-conjugating | Zea mays | 114-599 | Yield associated genes |
| | | | enzyme-like protein | | | |
| 269 | PHE0000296 | 637 | unknown protein | Zea mays | 90-785 | Yield associated genes |
| | | | recognized by | | | |
| | | | PF01169 | | | |
| 270 | PHE0000297 | 638 | 26S protease | Oryza sativa | 57-1343 | Yield associated genes |
| | | | regulatory subunit 6A | | | |
| | | | homolog | | | |
| 271 | PHE0000454 | 639 | fC-zmhuLIB3062- | Zea mays | 113-853 | Yield associated genes |
| | | | 044-Q1-K1-B8 | | | |
| 272 | PHE0000455 | 640 | corn E4/E8 binding | Zea mays | 253-2259 | Yield associated genes |
| | | | protein-like | | | |
| 273 | PHE0000009 | 641 | F9L1.31/G975 | Arabidopsis | 58-654 | Wax biosynthesis |
| 274 | PHE0000010 | 642 | rice G975 | Oryza sativa | 201-283, 516-1161 | Wax biosynthesis |
| 275 | PHE0000011 | 643 | glossy15 | Zea mays | 385-1722 | Wax biosynthesis |
| 276 | PHE0000079 | 644 | CUT1 | Oryza sativa | 372-1082, 1176-1946 | Wax biosynthesis |
| 277 | PHE0000278 | 645 | corn G975 | Zea mays | 41-679 | Wax biosynthesis |
| 278 | PHE0000024 | 646 | 700456686H1 | Zea mays | 441-2390 | Alteration of |
| | | | | | | development |
| 279 | PHE0000025 | 647 | ZmGRF1 | Zea mays | 55-1470 | Growth regulating factor 1 |
| 280 | PHE0000026 | 648 | OsGRF1 | Oryza sativa | 193-1380 | Growth regulating factor 1 |
| 281 | PHE0000289 | 649 | Zm-GRF1 (GA | Zea mays | 96-1202 | Growth regulating factor 1 |
| | | | responsive factor) | | | |
| 282 | PHE0000290 | 650 | ZmSE001-like | Zea mays | 253-2115 | Alteration of |
| | | | | | | development |
| 283 | PHE0000216 | 651 | thylakoid carbonic | Nostoc | 49-843 | Carbonic anhydrase |
| | | | anhydrase, ecaA | PCC7120 | | |
| 284 | PHE0000217 | 652 | bicarbonate | Chlamydomon | 156-1232 | Carbonic anhydrase |
| | | | transporter, LIP-36G1 | as reinhardtii | | |
| 285 | PHE0000218 | 653 | bicarbonate | Synechococcus | 271-1674 | Carbonic anhydrase |
| | | | transporter, cp | sp. PCC 7942 | | |
| | | | stromal IctB | | | |
| 286 | PHE0000219 | 654 | thylakoid carbonic | Chlamydomon | 62-994 | Carbonic anhydrase |
| | | | anhydrase, cah3 | as reinhardtii | | |
| 287 | PHE0000251 | 655 | corn TIR1-like | Zea mays | 113-1906 | F-box proteins |
| 288 | PHE0000254 | 656 | F-box protein | Glycine max | 123-1304 | F-box proteins |
| 289 | PHE0000255 | 657 | F-box protein | Glycine max | 228-1916 | F-box proteins |
| 290 | PHE0000357 | 658 | maize tubby-like | Zea mays | 519-1958 | F-box proteins |
| 291 | PHE0000358 | 659 | maize tubby-like | Zea mays | 517-1269 | F-box proteins |
| 292 | PHE0000359 | 660 | soy HMG CoA | Glycine max | 80-1441 | HMG_CoA synthase |
| | | | synthase | | | |
| 293 | PHE0000360 | 661 | yeast HMGS | Saccharomyces | 220-1695 | HMG_CoA synthase |
| | | | | cerevisiae | | |
| 294 | PHE0000319 | 662 | maize MnSOD | Zea mays | 249-947 | Oxidative stress |
| 295 | PHE0000320 | 663 | sodB | Escherichia | 94-675 | Oxidative stress |
| | | | | coli | | |
| 296 | PHE0000321 | 664 | Synechococcus sp. | Synechococcus | 142-747 | Oxidative stress |
| | | | PCC 7942 sodB | sp. PCC 7942 | | |
| 297 | PHE0000322 | 665 | maize catalase-1 | Zea mays | 208-1683 | Oxidative stress |
| 298 | PHE0000323 | 666 | maize catalase-3 | Zea mays | 30-1511 | Oxidative stress |
| 299 | PHE0000324 | 667 | ascorbate peroxidase | Zea mays | 197-1063 | Oxidative stress |
| 300 | PHE0000325 | 668 | corn GDI | Zea mays | 57-1397 | Oxidative stress |
| 301 | PHE0000326 | 669 | soy GDI | Glycine max | 45-1418 | Oxidative stress |
| 302 | PHE0000327 | 670 | corn rho GDI | Zea mays | 463-1203 | Oxidative stress |
| 303 | PHE0000328 | 671 | basic blue copper | Zea mays | 13-408 | Oxidative stress |
| | | | protein | | | |
| 304 | PHE0000329 | 672 | plantacyanin | Zea mays | 109-489 | Oxidative stress |
| 305 | PHE0000330 | 673 | basic blue copper | Glycine max | 83-463 | Oxidative stress |
| | | | protein | | | |
| 306 | PHE0000331 | 674 | Similar to blue copper | Zea mays | 323-868 | Oxidative stress |
| | | | protein precursor | | | |
| 307 | PHE0000332 | 675 | lamin | Zea mays | 62-646 | Oxidative stress |
| 308 | PHE0000333 | 676 | allyl alcohol | Zea mays | 56-1105 | Oxidative stress |
| | | | dehydrogenase | | | |
| 309 | PHE0000334 | 677 | allyl alcohol | Glycine max | 103-1128 | Oxidative stress |
| | | | dehydrogenase | | | |
| 310 | PHE0000335 | 678 | allyl alcohol | Zea mays | 6-1079 | Oxidative stress |
| | | | dehydrogenase | | | |
| 311 | PHE0000336 | 679 | quinone | Zea mays | 47-1051 | Oxidative stress |
| | | | oxidoreductase | | | |
| 312 | PHE0000337 | 680 | E. nidulans cysA | Emericella | 384-1961 | Oxidative stress |
| | | | | nidulans | | |
| 313 | PHE0000338 | 681 | serine | Synechocystis | 801-1547 | Oxidative stress |
| | | | acetyltransferase | sp. PCC 6803 | | |
| 314 | PHE0000339 | 682 | Synechocystis thiol- | Synechocystis | 36-638 | Oxidative stress |
| | | | specific antioxidant | sp. PCC 6803 | | |
| | | | protein | | | |
| 315 | PHE0000340 | 683 | yeast TSA2 | Saccharomyces | 108-698 | Oxidative stress |
| | | | | cerevisiae | | |
| 316 | PHE0000341 | 684 | yeast mTPx | Saccharomyces | 730-1512 | Oxidative stress |
| | | | | cerevisiae | | |
| 317 | PHE0000342 | 685 | yeast nTPx | Saccharomyces | 103-750 | Oxidative stress |
| | | | | cerevisiae | | |
| 318 | PHE0000343 | 686 | yeast TPx III | Saccharomyces | 657-1187 | Oxidative stress |
| | | | | cerevisiae | | |
| 319 | PHE0000344 | 687 | corn type 2 | Zea mays | 37-522 | Oxidative stress |
| | | | peroxiredoxin | | | |
| 320 | PHE0000345 | 688 | soy putative 2-cys | Glycine max | 160-939 | Oxidative stress |
| | | | peroxiredoxin | | | |
| 321 | PHE0000346 | 689 | soy peroxiredoxin | Glycine max | 104-745 | Oxidative stress |
| 322 | PHE0000347 | 690 | heat shock protein 26, | Zea mays | 117-836 | Oxidative stress |
| | | | plastid-localized | | | |
| 323 | PHE0000348 | 691 | heat shock protein | Zea mays | 79-732 | Oxidative stress |
| | | | hsp22 precursor, | | | |
| | | | mitochondrial | | | |
| 324 | PHE0000349 | 692 | heat shock protein | Zea mays | 112-735 | Oxidative stress |
| 325 | PHE0000350 | 693 | low molecular weight | Zea mays | 28-690 | Oxidative stress |
| | | | heat shock protein | | | |
| 326 | PHE0000351 | 694 | 18 kDa heat shock | Zea mays | 103-597 | Oxidative stress |
| | | | protein | | | |
| 327 | PHE0000352 | 695 | heat shock protein | Zea mays | 229-690 | Oxidative stress |
| | | | 16.9 | | | |
| 328 | PHE0000353 | 696 | HSP21-like protein | Zea mays | 73-696 | Oxidative stress |
| 329 | PHE0000354 | 697 | Opt1p | Saccharomyces | 508-2904 | Oxidative stress |
| | | | | cerevisiae | | |
| 330 | PHE0000355 | 698 | SVCT2-like permease | Zea mays | 220-1779 | Oxidative stress |
| 331 | PHE0000356 | 699 | SVCT2-like permease | Zea mays | 34-1632 | Oxidative stress |
| 332 | PHE0000385 | 700 | H+ transporting | Zea mays | 176-2836 | ATP synthesis |
| | | | ATPase | | | |
| 333 | PHE0000386 | 701 | cation-transporting | Zea mays | 222-2168 | ATP synthesis |
| | | | ATPase | | | |
| 334 | PHE0000387 | 702 | yeast DRS2 (ALA1- | Saccharomyces | 170-4237 | ATP synthesis |
| | | | like) | cerevisiae | | |
| 335 | PHE0000388 | 703 | S. pombe ALA1-like | Schizosaccharo | 56-3832 | ATP synthesis |
| | | | | myces pombe | | |
| 336 | PHE0000389 | 704 | rice ALA1-like 1 | Oryza sativa | 47-1538,1619-1925, | ATP synthesis |
| | | | | | 3116-3824, | |
| | | | | | 3920-4043, | |
| | | | | | 4143-4362, | |
| | | | | | 4590-5048, | |
| | | | | | 5937-6153 | |
| 337 | PHE0000469 | 705 | yeast YKL091c | Saccharomyces | 110-1042 | ATP/ADP transporters |
| | | | | cerevisiae | | |
| 338 | PHE0000470 | 706 | corn Ssh1-like protein 1 | Zea mays | 57-1037 | ATP/ADP transporters |
| 339 | PHE0000471 | 707 | corn Ssh1-like protein 3 | Zea mays | 89-841 | ATP/ADP transporters |
| 340 | PHE0000472 | 708 | corn Ssh1-like protein 4 | Zea mays | 309-1196 | ATP/ADP transporters |
| 341 | PHE0000473 | 709 | soy Ssh1-like protein | Glycine max | 209-976 | ATP/ADP transporters |
| | | | 2 [ssh2] | | | |
| 342 | PHE0000017 | 710 | Zm-AAA1 - AAA | Zea mays | 184-2214 | AAA-type ATPase |
| | | | ATPase | | | |
| 343 | PHE0000018 | 711 | Zm-AAA2 - AAA | Zea mays | 104-2533 | AAA-type ATPase |
| | | | ATPase | | | |
| 344 | PHE0000395 | 712 | soy phantastica | Glycine max | 275-1345 | Plant architecture |
| 345 | PHE0000396 | 713 | soy phantastica 2 | Glycine max | 178-1260 | Plant architecture |
| 346 | PHE0000397 | 714 | maize rough sheath 1 | Zea mays | 92-1144 | Plant architecture |
| 347 | PHE0000398 | 715 | soy lg3-like 1 | Glycine max | 103-1026 | Plant architecture |
| 348 | PHE0000399 | 716 | soy rough sheath1- | Glycine max | 144-1076 | Plant architecture |
| | | | like 1 | | | |
| 349 | PHE0000401 | 717 | soy G1635-like 1 | Glycine max | 28-888 | Plant architecture |
| 350 | PHE0000411 | 718 | corn monosaccharide | Zea mays | 331-2565 | Carbohydrate |
| | | | transporter 2 | | | transporters |
| 351 | PHE0000412 | 719 | corn monosaccharide | Zea mays | 75-1643 | Carbohydrate |
| | | | transporter 1 | | | transporters |
| 352 | PHE0000413 | 720 | soy monosaccharide | Glycine max | 132-1685 | Carbohydrate |
| | | | transporter 3 | | | transporters |
| 353 | PHE0000414 | 721 | corn monosaccharide | Zea mays | 141-1670 | Carbohydrate |
| | | | transporter 3 | | | transporters |
| 354 | PHE0000415 | 722 | soy monosaccharide | Glycine max | 160-1899 | Carbohydrate |
| | | | transporter 1 | | | transporters |
| 355 | PHE0000416 | 723 | corn monosaccharide | Zea mays | 74-1690 | Carbohydrate |
| | | | transporter 6 | | | transporters |
| 356 | PHE0000418 | 724 | corn monosaccharide | Zea mays | 146-1744 | Carbohydrate |
| | | | transporter 4 | | | transporters |
| 357 | PHE0000419 | 725 | soy monosaccharide | Glycine max | 63-1505 | Carbohydrate |
| | | | transporter 2 | | | transporters |
| 358 | PHE0000420 | 726 | soy sucrose | Glycine max | 63-1595 | Carbohydrate |
| | | | transporter | | | transporters |
| 359 | PHE0000421 | 727 | corn sucrose | Zea mays | 76-1599 | Carbohydrate |
| | | | transporter 2 | | | transporters |
| 360 | PHE0000422 | 728 | corn monosaccharide | Zea mays | 201-1763 | Carbohydrate |
| | | | transporter 8 | | | transporters |
| 361 | PHE0000423 | 729 | corn monosaccharide | Zea mays | 93-1634 | Carbohydrate |
| | | | transporter 7 | | | transporters |
| 362 | PHE0000390 | 730 | rice chloroplastic | Oryza sativa | 136-1311 | Carbon assimilation |
| | | | sedoheptulose-1,7- | | | |
| | | | bisphosphatase | | | |
| 363 | PHE0000391 | 731 | rice cytosolic | Oryza sativa | 171-1187 | Carbon assimilation |
| | | | fructose-1,6- | | | |
| | | | bisphosphatase | | | |
| 364 | PHE0000392 | 732 | Wheat sedoheptulose- | Triticum | 14-1192 | Carbon assimilation |
| | | | 1,7-bisphosphatase | aestivum | | |
| 365 | PHE0000393 | 733 | dual function | Ralstonia | 80-1399 | Carbon assimilation |
| | | | SBPase/FBPase | eutropha | | |
| 366 | PHE0000394 | 734 | sedoheptulose-1,7- | Chlorella | 90-1238 | Carbon assimilation |
| | | | bisphosphatase | sorokiniana | | |
| 367 | PHE0000425 | 735 | soy isoflavone | Glycine max | 45-1607 | Flavonoids |
| | | | synthase | | | |
| 368 | PHE0000426 | 736 | soy ttg1-like 2 | Glycine max | 52-1059 | Flavonoids |

Details of the above listed strategies for crop improvement are provided below.

1. Amino Acid Transporters

Free amino acids are the most important translocatable organic nitrogen forms in plants. Amino acid transporters are actively involved in amino acid movement from cell to cell and further to vascular tissues for long distance translocation. It is expected that genetic modification of the transporter activity in plants in a given tissue and stages of development may direct more translocation of free amino acids loaded into vascular bundle tissue from source tissues and further transport into sink tissues, such as developing vegetative tissues or seeds, for protein biosynthesis. Increased seed protein content may be expected by enhancing organic nitrogen that is loaded in the developing seeds.

Nucleic acid and polypeptide sequences for use in generation of transgenic crop plants having improved properties as described in the above strategy are provided in Table 1.

2. Protection Against Osmotic Stress and/or Modification of Sugar Transport and/or Metabolism—ASH1/GCE1

Water stress is a major cause of crop loss. A screen was conducted to identify genes which, when overexpressed, protect Saccharomyces cerevisiae from high osmotic conditions. It is expected that these genes, when transferred to crop plants will confer resistance to water-stress conditions such as drought. Two genes from Arabidopsis thaliana, designated ASH1 and ASH2, for Arabidopsis Suppresor of hog1 (osmosensitive mutant) were identified in the S. cerevisiae screen. BLAST analysis reveals that ASH1 is closely related to MTN3, a member of a group of genes identified as induced by Rhizobium during root nodule development. ASH2 encodes a hypothetical protein of unknown function and contains an N-terminal domain motif (motif Y) that appears to be plant specific and is found in approximately 19 Arabidopsis proteins.

In an unrelated study, the Arabidopsis ASH1 gene was identified as being involved in carbon/nitrogen signaling. A number of Arabidopsis mutants that are insensitive to glucose inhibition of chlorophyll accumulation in the absence of nitrogen (GCE mutants) were identified. Such mutants develop green cotyledons and true leaves on nitrogen free 3% glucose only medium, while wild type plants under the same conditions develop purple cotyledons. In eight different glucose insensitive plants, the mutation responsible for the mutant phenotype, GCE1 mutation, was mapped to the same ASH1 gene identified in the above yeast osmosensitivity screen.

Bioinformatic analysis of ASH1/GCE1 reveals that it encodes a member of a relatively well conserved family of proteins represented in Arabidopsis by 16 family members. In addition, closely related homologs were identified in a number of plant species, including soy (Glycine max), rice (Oryza sativa), corn (Zea mays), cotton (Gossypium hirsutum), leek (Allium porrum) and wheat (Triticum aestivum). Analysis of the structure of the ASH1/GCE1 gene product indicates the presence of a number of transmembrane domains and regions of homology to known transporters, including sugar transporters and sodium:solute transporters. This evidence, in combination with the observed phenotypes and the report of the possible involvement of a related petunia gene, NEC1, in sugar metabolism or transport (Ge et al., 2000), indicate that ASH1/GCE1 may contribute to plant yield because of its roles in osmoprotection and carbon partitioning. Thus, expression of ASH1/GCE1 and/or its crop homologs in transgenic plants is expected to be useful for production of transgenic plants having protection against osmotic stress and/or improved yield as the result of altered sugar transport and/or metabolism.

Nucleic acid and polypeptide sequences for use in generation of transgenic crop plants having improved properties as described in the above strategy are provided in Table 1.

3. Plant Steroid Biosynthesis Pathway and Signal Transduction Genes

Brassinosteroids are believed to be plant growth regulators. Over forty related brassinosteroid (BR) compounds have been identified in plant extracts (Clouse 1998). BR compounds have been shown to affect stem elongation (Clouse, 1998; Cutler, 1991), cell division,(Clouse, 1993) reproductive and vascular development (Kauschmann 1996), membrane polarization and proton pumping, source/sink relationships and modulation of stress (Artica, 1995; Mandava, 1988; Sakurai, 1997; Sasse; 1990).

Using ¹²⁵I-labeled BRs, it was demonstrated that BRs accumulate in the elongating zones of mung bean epicotyls and the apex of cucumber seedling (Xu, 1994). Both BR and auxin promote elongation. Synergisms and additive effects were observed when BRs were combined with auxins and gibberellins (Mandava, 1988) as measured by elongation. BR effects are inhibited by cytokinin, absiscic acid and ethylene as measured by BR-induced elongation. (Iwasaki, 1991; Sasse, 1990). Treatment with exogenous BR was shown to increase ethylene production in stem tissue (Arteca, 1995). The inhibitory effect of high concentrations of BR on expanding root cells was attributed to the overproduction of ethylene (Arteca, 1995). BR effects on elongation are accompanied by proton extrusion and hyperpolarization of cell membranes (Cao, 1995; Bajguz, 1996;Bloch 1983; Hartmann 1987).

An indirect induction of ATPase activity by BR may affect sucrose transport (Petzold, 1992). Goetz et al. (2000) reported a link between steroid signaling and carbohydrate metabolism. There was an increase in the activity of cell-wall-bound invertase in cell-suspension cultures treated with BR and an increased capacity for sugar uptake into cells.(Goetz, 2000).

Tobacco field trials with exogenously applied BR resulted in significantly greater biomass as well as significantly higher yield (Sairam, 1994). Plants treated with BR compounds have also been reported to have enhanced tolerance to both cold and heat. Membrane electrolyte leakage was reduced during chilling and there was a reduction in malondialdhyde content and superoxide dismutase activity (Patterson,1993). ATP and proline levels were shown to increase after applicatin of BRs (Wang, 1993). An application of BR increased thermotolerance in heat-stressed wheat and produced a change in the spectrum of heat shock proteins. Drought stress and salt stress were both ameliorated by treatment with BR (Sairman, 1994; Schuler, 1991; Singh, 1993; Takeuchi, 1992).

The biosynthetic pathway of BRs has been elucidated by feeding studies with radiolabeled precursors of brassinolide the most biologically active form of BR. BR mutants have been produced and primarily result in dwarf plants (Bach, 1997; Benveniste 1986; Campbell 1980; Choe 1999a & b; Crowley 1998; Gachotte 1995; Husselstein 1999; Klahre 1998; Lai 1994; Lees 1999; Ourisson 1994;)

BR-insensitive mutants were identified in Arabidopsis, pea and tomato. One such gene, BAS-1, is believed to regulate BR levels and light responsiveness in Arabidopsis. The BAS-1 gene was found to be the control point between cytochrome P450 photoreceptor systems and BR signal transduction. Seedlings with reduced BAS1 expression displayed reduced sensitivity to light and hyper-responsiveness to BR in a light dependent manner (Neff, 1999). The Arabidopsis receptor kinase BRI1 was identified as a leucine rich BR signal receptor (He 2000; Li, 1997; Schumacher, 2000) A mutant, designated FACKEL, was isolated from Arabidopsis that displayed distorted embryos, multiple shoot meristems and stunted roots. The mutant gene (FIC) was cloned and is similar to the yeast C-14 sterol reductase (Lorenz 1992; Marcireau 1992). The mutant could not be rescued by exogenously applied BR. The FIC gene product is believed to be required for cell division and expansion of the developing embryo (Farese 1998; Jang 2000; Schrick 2000). Identification of mutants with the above described phenotypes indicates rate limiting steps in steroid biosynthesis and signal pathways.

Disruption of the OsBR11 rice orthologue of BRI1, an LLR-kinase and putative brassinosteroid receptor (He et al., 2000; Friedrichsen et al., 2000), has demonstrated that OsBRI1 functions in various growth and developmental processes in rice, including (1) internode elongation, by inducing the formation of the intercalary meristem and the longitudinal elongation of internode cells; (2) bending of the lamina joint; and (3) skotomorphogenesis (Yamamuro et al., 2000).

It is expected that overexpressed levels of these genes in Zea mays L will result in plants with higher endogenous levels of plant steroids improving plant vigor, yield and stress tolerance.

Nucleic acid and polypeptide sequences for use in generation of transgenic crop plants having improved properties as described in the above strategy are provided in Table 1.

4. Cyclin Dependent Kinase Activating Kinase

Overexpression of cyclins in plants has been shown to accelerate growth of A. thaliana without inducing neoplasia (Doerner et al., 1996; Cockcroft et al., 2000). This can bring about a number of potentially desirable phentotypic changes that may enhance crop productivity or grain composition. For example, longer roots may increase the ability of a plant to perform under conditions of limited water availability and increased leaf area may allow a plant to produce more grain or leaf mass. Increases in embryo size relative to endosperm mass can also bring about overall increases in grain oil content.

An alternative to overexpression of cyclins to bring about phenotypic changes in plants is the overexpression of cyclin dependant kinase activating kinases (CDK-activating kinases or CAKs). CDK-activating kinase is a complex of cdk7, cyclin H and MAT1 (menage a trois 1) and has been proposed to function in the control of cell cycle progression, DNA repair and RNA polymerase II transcription (Nigg, 1996). CAK exists in at least two distinct states in the cell; i.e., as a free complex and as a component of the RNA polymerase II general transcription factor TFIIH. CAK phosphorylates many substrates in vitro, including several CDKs, the general transcription factors TFIIE, TFIIF and TBP, and the carboxy-terminal domain of the largest subunit of RNA pol II (Morgan, 1995; Hoeijmakers et al., 1996; Nigg, 1996). Little is known about factors that goven substrate preference of CAK, but it does appear that specificity is modulated by association of CAK with MAT1 and TFIIH (Yankulov and Bentley, 1997).

CDK-activating kinase has been reported in plants, although many of the details surrounding the composition and role in the cell remain unclear. CAK in rice seems to act in a manner similar to that described in mammalian systems. A rice homologue of cdk7, R2, has been reported to complement a CAK mutation in yeast and phosphorylate the rice CDK (Cdc2Os1) and the carboxy-terminal domain (CTD) of the largest subunit of RNA polymerase II of Arabidopsis (Yamaguchi et al., 1998). This protein has also been shown to interact with rice cyclin H in a manner similar to that of mammalian cdk7 (Yamaguchi et al., 2000). R2 expression levels also appear to correlate with progression through G1 and S phase (Sauter, 1997; Lorbiecke and Sauter, 1999), although this link is not entirely clear (Umeda et al., 1999). In contrast to rice, an Arabidopsis CAK cloned by complementation in yeast is unrelated to the ckd7-like protein R2 and was also unable to phosphorylate the CTD of RNA polymerase II (Umeda et al., 1998), suggesting that activation of CDKs and RNA polymerase II transcription are separable in plants. Yeast appears to use an unrelated CAK called CAK1 (Kaldis et al., 1996; Kladis, 1999; Ross et al., 2000).

It is expected that alteration of expression of CAK transgenes in crop plants, particularly maize and maize grain, may increase plant resistance to abiotic stresses or alter the nutritional content of the grain.

Nucleic acid and polypeptide sequences for use in generation of transgenic crop plants having improved properties as described in the above strategy are provided in Table 1.

5. Cercosporin Resistance

Cercospora zeae-maydis, the causative agent of grey leaf spot disease in maize, produces the toxin cercosporin. It is expected that methods of increasing tolerance of maize to cercosporin will increase plant resistance to gray leaf spot disease. The yeast gene, PDR5, is induced by cercosporin and encodes an ABC (ATP-Binding Casette) efflux pump involved in multiple drug resistance. The specific disruption of this pump is sufficient to confer hypersensitivity to cercosporin in yeast. It is expected that overexpression of the PDR5 gene in corn will result in resistance to grey-leaf spot disease in maize.

Nucleic acid and polypeptide sequences for use in generation of transgenic crop plants having improved properties as described in the above strategy are provided in Table 1.

6. Chlorate/Nitrate Transport

The Arabidopsis gene, CHL1 (AtNRT1) encodes a nitrate transporter. The CHL1 gene product possesses dual-affinity properties for both the high-affinity and low-affinity transport systems within Arabidopsis roots.

It is well known that an increase in the amount of nitrogen made available to the plant correlates with an increase in the total amount of nitrogen measured within the plant. Ta and Weiland (1992) have reported that more nitrogen is specifically mobilized to the grain if nitrogen is provided in the soil later in development. It has been further reported (Reed et al.(1980)) that half of the nitrogen in the ear is accumulated prior to the remobilization of reduced nitrogen from the vegetative tissues. Since nitrogen transported into the ear is not from the remobilization of vegetative reduced nitrogen, it is believed that grain nitrogen is derived from nitrate stored within the plant or directly from nitrate uptake by the roots. Also, cob development is complete approximately 12 days after pollination, therefore most of the reduced nitrogen within the ear is partitioned to the grain after this time. Lastly, in some maize genotypes, the rate of nitrate uptake declines after approximately 30 days post-anthesis (Reed et al. 1980).

Expression of the CHL1 gene in maize may increase the level of nitrogen/nitrate in the plant during vegetative growth. This will provide the kernel with increased levels of reduced nitrogen as a result of the remobilization of vegetative reduced nitrogen. Furthermore, expression of CHL1 in maize may increase the rate of nitrate uptake during the first 30 days post-anthesis when the need for nitrate appears to be critical for grain-filling. Additionally, this approach could expand the window in which nitrate uptake occurs post-anthesis, primarily between 30 days post-anthesis through maturity. It is expected that this will provide more nitrate to the kernel during the entire grain-filling process. Photosynthetic activity may might also be preserved through the prolonged period of nitrogen uptake by delaying the re-mobilization of vegetative reduced nitrogen, thereby postponing senescence and allowing for the production of higher grain yields.

The CHL1 gene is expressed using the rice actin promoter in order to constitutively express CHL1 throughout the plant. The gene is also expressed with the root-specific maize rs81 promoter (U.S. Pat. No. 6,207,879) to provide for expression in the endodermal layer of the root. Lastly, the CHL1 is also expressed under control of a senescence-specific promoter to increase the level of nitrate/nitrogen within the plant and/or kernel during the grain-filling period.

A rice nitrate transporter, NTR1, has been functionally characterized (Lin et al, 2000). This gene is more similar in sequence to barley oligopeptide transporters than to the A. thaliana chlorate transporter. However, the rice transporter also has been shown to transport nitrate and not peptides in xenopus oocytes.

Nucleic acid and polypeptide sequences for use in generation of transgenic crop plants having improved properties as described in the above strategy are provided in Table 1.

7. Siroheme Synthesis

cobA, cysG, and upm1 are examples of S-adenosyl-L-methionine-dependent uroporphyrinogen III (uro'gen) methyl transferases (SUMT) that produce bright red fluorescent porphyriniod compounds when overexpressed in E. coli, yeast, and CHO cells. This property enabled visual selection of transformed E. coli colonies (Rossner and Scott 1995) and automated sorting of transformed yeast and CHO cells (Wildt and Deuschle 1999). The fluorescence is the result of intracellular accumulation of di- and tri-methylated uro'gen (dihydrosirohydrochlorin and trimethylpyrorocorphin), both of which are compounds found in porphyrin synthesis pathways (i.e., chlorophyll and cobalamin).

The siroheme porphyrin is an essential prosthetic group in maize nitrate reductase and is also synthesized via this methylation (Sakakibara, Takei, and Sugiyama 1996). It is expected that expression of a gene involved in siroheme synthesis will contribute to enhanced nitrogen metabolism by a plant.

The first step in the biosynthesis of Uro'gen III is the dimerization of two molecules of alanine to give the pyrrole porphobilinogen. This molecule is acted upon by two enzymes, PBG-deaminase and Uro'gen III synthase, to give Uro'gen-III. Transmethylation of Uro'gen III by a SAM-dependent methyltransferase (MT) gives precorrin-2 (PC-2), which is subsequently converted into siroheme after dehydrogenation and ferrochelation. Trimethylpyrrocorphin is an overmethylated PC-2 derivative that accumulates in cells that overexpress Uro'gen-III methyltransferases.

Cells transformed with either cobA (from P. freudenreichii) or green fluorescent protein genes yield roughly equivalent fluorescent signals. With absorbance peaks at 384 nm and 500 nm along with an emission band at 605 nm, the fluorescent porphyriniods generated by the cobA uro'gen methyl transferase have a good spectral signature for detection in plant material. Excitation at either 384 or 500 nm avoids strong chlorophyll absorbance and the resulting red emission will be readily detected as it has a substantial Stokes shift (from the 500 nm absorbance origin) and yet does not overlap with chlorophyll autofluorescence in the far red (Haseloff, 1999) Red emitting markers are generally desirable since these signals have a lower autofluorescent background.

The carboxy terminus of the maize sumt (genbank D83391; Monsanto ZEAMA-04OCT00-CLUSTER29_{—}1), Arabidopsis upm1 (L47479), and E. coli cysG (X14202) are significantly homologous to the bacterial cobA genes of P. freudenreichii (U13043), A. nidulans (X70966), and P. denitrificans (M59236) (Sakakibara et al 1996). The E. coli cysG gene is multifunctional (producing enzymes for the subsequent porphyrin oxidation and Fe⁺² chelation steps) but the SUMT activity has been located to this same region of homology (Warren et al. 1994). Given that the cobA gene should not effect expression or activity of the endogenous maize SUMT (as needed for producing the siroheme required for nitrate reductase), the transformed marker uro'gen methyl transferase is derived from one of these or similar bacterial sources. Plant siroheme synthases have been reported to be localized in the chloroplast (Leustek et al., 1997), and therefore these genes are expressed with a chloroplast transit peptide.

It is expected that expression of cobA, cysG, and upm1 like proteins will provide useful fluorescent screenable markers in maize.

Nucleic acid and polypeptide sequences for use in generation of transgenic crop plants having improved properties as described in the above strategy are provided in Table 1.

8. Cold Induced Genes

The inability of corn plants (Zea mays L.) to endure low temperatures and freezing is a major determinant of the geographical areas where the crop is grown. Even in climates considered suitable, crop failure or decreases in yield frequently occur due to cold temperatures. Injury to the plant's cell membrane is an important site of cold damage (Troyer, 1999).

Ice formation is initiated in the intercellular spaces of the plant's cells because the extracellular fluid has a higher freezing point or lower solute concentration than intracellular fluid. Unfrozen water moves from inside the cell to the intercellular spaces down the chemical potential gradient. The plasma membrane is damaged due to dehydration causing lysis, segregation of proteins within the membrane (phase transition), and fracture lesions. The cell's cold-induced production of reactive oxygen species contributes to the membrane damage (McKersie et al., 1997). At prolonged exposure to below freezing temperatures, intercellular ice can cause cell rupture (Olien and Smith, 1977).

Many plants acclimate to cold upon exposure to prolonged low nonfreezing temperatures because the expression of certain cold induced genes stabilize membranes against freeze-induced injury. Transforming Z. mays L. to overexpress these genes results in plants that are more cold tolerant.

It is believed that expression of genes involved in dessication tolerance in a plant may contribute to increased tolerance to chilling, freezing, dessication, water stress, or other abiotic stresses. Three classes of Late Embryogenic Abundant (LEA) proteins have been assigned based on structural similarities (see Dure et al., 1989). All three classes of LEAs have been demonstrated in maturing (i.e. desiccating) seeds. Within these 3 types of LEA proteins, the Type-II (dehydrin-type) have generally been implicated in drought and/or desiccation tolerance in vegetative plant parts (i.e. Mundy and Chua, 1988; Piatkowski et al., 1990; Yamaguchi-Shinozaki et al., 1992). Expression of a Type-III LEA (HVA-1) in tobacco was found to influence plant height, maturity and drought tolerance (Fitzpatrick, 1993). Expression of HVA1 in rice was shown to increase resistance to salt and water stress ( U.S. Pat. No. 5,981,842). Expression of genes from all three LEA groups may therefore confer drought tolerance in a transgenic plant.

Highly hydrophilic proteins such as the dehydrins have recently been found to play a role in water stress responses in a wide variety of eukaryotes and prokaryotes (Garay-Arroya et al., 2000). Garay-Arroya et al. developed an algorithm that identified all known plant LEA proteins (except for the glycine poor group 5 LEAs). This algorithm also identified hydrophilic LEA-like proteins in yeast and E. coli. Eight of the 12 yeast genes they identified were also shown to be induced by osmotic stress. Given the reported osmotic and temperature stress resistances associated with overexpression of LEA and COR genes, it seems plausible that overexpression of these microbial genes in corn may improve corn's response to cold or drought stress. Five genes from yeast are overexpressed in corn.

A total of 65 putative dehydrins were identified. Sequence analysis showed that these proteins fell into 6 or 7 distinct groups. Based on this analysis, five additional dehydrins have been selected for overexpression in maize in order to improve tolerance to drought or cold temperatures.

In addition, several group 3 LEA proteins are introduced into maize including barley HVA1 (X78205), a maize LEA3 protein (fC-zmenLIB3061034Q1K1C6-FLI), and the maize group 1 LEA protein EMB5 (700623908_FLI)

Nucleic acid and polypeptide sequences for use in generation of transgenic crop plants having improved properties as described in the above strategy are provided in Table 1.

9. Corn Cyclins

Cyclins play a key regulatory role in passage of cells through the cell cycle. Interaction of a cyclin with a cyclin-dependent kinase (CDK) activates the kinase, resulting in the specific phosphorylation of other molecules and advancement of the cell cycle. Degradation sequences within the protein free the CDK for association with another cyclin. There are several different classes of cyclins, with synthesis and degradation regulated such that the availability of a particular cyclin is restricted to a particular phase of the cell cycle. In plants, three classes of cyclins have been identified: cycA, cycB, and cycD.

Three reports examine the consequences of ectopic cyclin expression on plant growth. Doerner et al. (1996) generated Arabidopsis plants expressing Arabidopsis cycB1 in roots. In the roots of normal plants this cyclin is expressed in the meristem, with transcripts accumulating prior to cytokinesis and declining once cell division has occurred. CycB1 is also induced by exogenous treatment with IAA. Transgenic plants containing a cylcin B1 gene driven by the Arabidopsis cdc2a promoter exhibited an increase in root growth rate. Increased growth rate was due to an increase in cell number rather than cell size, and the overall morphology of the roots was normal. Cockcroft et al (2000) examined the consequences of expressing Arabidopsis cycD2 driven by the 35S promoter in transgenic tobacco plants. Plants expressing the Arabidopsis protein were similar in appearance to wild type plants, but had an increased growth rate, reaching the flowering stage 9-14 days earlier than non-transgenic plants. Examination of vegetative meristems indicated normal cell organization but an increased rate of leaf primordia initiation due to a more rapid rate of cell division. In contrast, overexpression of a cytokinin-regulated cyclin, Arabidopsis cycD3, in transgenic plants resulted in disorganized meristems, an abnormal appearance, and delayed flowering (Riou-Khamlichi et al., 1999). Unlike callus from wild type plants, callus generated from the cycD3 expressing transgenic plants was able to thrive in the absence of exogenous cytokinin, suggesting that for certain processes cycD3 is a component of a cytokinin response pathway.

The variable results of constitutive cycD expression reported in the literature may be due to different roles played by the cycDs tested, or may be due to unidentified experimental differences. Corn cycD genes are tested for the consequences of their constitutive expression in corn. cycD2-like genes are preferably expressed in plants. A cycD gene from rice is also introduced into maize. That gene is be placed under the regulation of an endosperm-specific promoter, with the specific goal of enhancing cell division in the seed. PCT publications WO 98/42851 and WO 00/17364 disclose altering the expression of cycD in plants to modulate plant growth.

Yield is impacted in cyclin overexpressers by a variety of means, including early flowering, improved establishment, increased root mass, or increased seed mass. The key desired phenotype is normal morphology but enlargement of selected organs (roots, ears) or increased growth rate resulting in improved yield. Other assays include yield and altered growth rate or developmental pattern

Nucleic acid and polypeptide sequences for use in generation of transgenic crop plants having improved properties as described in the above strategy are provided in Table 1.

10. Cytokinins

Cytokinins promote and sustain plant cell division (Riou-Khamlichi et al., 1999), modulate developmental processes such as apical dominance (Napoli et al., 1999) and senescence (Gan and Amasino, 1995), play a role in nitrogen (Yu et al, 1998) and carbon (Miyazawa et al., 1999) metabolism and interact with most other phytohormone signaling pathways (Cary et al., 1995; Kusnetsov et al., 1998; Miyazawa et al., 1999). Manipulation of cytokinin levels or signaling pathways can thus be expected to improve yield by increasing kernel cell numbers, delaying senescence, increasing the number of ears a corn plant sets, antagonizing abscisic acid signaling, improving nitrogen use efficiency or increasing carbon flow into amyloplasts. However, the diverse roles that cytokinins play in plant growth and development also suggest that temporal and spatial control will be required to manipulate cytokinin signaling for plant improvement.

Enhancement of cytokinin activity in early embryos and/or endosperm can be predicted to have a number of effects that will lead to increases in corn yield. Cytokinin-mediated cell division may increase overall yield by increasing endosperm and/or embryo size. A rapid increase in cell number driven by increased cytokinin activity may also lead to an earlier establishment of newly fertilized kernels as strong sinks. Sink strength might also be enhanced by increasing the rate at which amyloplasts acumulate starch. Increased cytokinin activity is expected to block kernel abortion either through antagonism of abscisic acid or sucrose signals associated with low water potentials (Cheikh and Jones, 1994; Ikeda et al., 1999; Zinselmeier et al., 1999) or through the increases in sink strength mentioned above.

Grain quality may be altered through either of two methods. Enhanced nitrogen accumulation or use may occur due to cytokinin induction of nitrate reductase. This may lead to an increase in grain protein levels or improved performance under water limiting conditions where nitrate reductase activity is reduced (Foyer et al., 1998). Increased starch content or altered grain carbon distribution could also occurr due to cytokinin induction of amyloplast starch accumulation, ADP-glucose pyrophosphorylase, starch synthase or starch branching enzymes (Miyazawa et al., 1999).

Increases in cytokinin levels have been shown to reduce apical dominance and delay senescence in tobacco plants engineered to conditionally overexpress a cytokinin synthase (Gan and Amasino, 1995; Faise et al., 1997; McKenzie et al., 1998). This indicates that altered cytokinin activity could be used to increase yield by promoting the growth of multiple ears per plant or prolonging the fill period prior to grain dry down. It has also been reported that senescence is delayed in tobacco by expressing an upstream activator of cytokinin biosynthesis, knotted1 (Ori et al., 1999).

Cytokinin activity can be manipulated in several different manners. Cytokinin levels can be elevated by increasing cytokinin production, increasing the rate of zeatin release from less active O-glycosyl derivatives, decreasing the rate of degradation, decreaseing the rate of inactivation by glycosylation or altering transport of the molecule. Alternatively, cytokinin signaling can by manipulated by overexpression of putative receptors, and upstream or downstream signaling components. A list of genes involved in cytokinin activity is provided below.

Genes Related to Cytokinin Biosynthesis and Activity

| Gene | Accession | Function | Source | Reference |
|---|---|---|---|---|
| Isopentyl transferase | AB025109 | cytokinin | A. tumefaciens | Ebinuma et al., |
| (ipt) | | biosynthesis | | 1997 |
| Beta-glucosidase (Zm- | CAA52293 | cytokinin-beta- | Z. mays | Brzobohaty et al., |
| p60) | | glucosidase | | 1993 |
| Cytokinin oxidase | AAC27500 | cytokinin | Z. mays | Morris et al., 1999 |
| (CKO) | | degradation | | |
| zeatin O- | AF116858 | Cytokinin | P. vulgaris | Martin et al., 1999a |
| xylosyltransferase | | inactivation | | |
| (ZOX) | | | | |
| zeatin O- | AAD04166 | Cytokinin | P. lunatus | Martin et al., 1999b |
| glucosyltransferase | | inactivation | | |
| (ZOG) | | | | |
| Purine permease1 | AAF64547 | Cytokinin | A. thaliana | Gillissen et al., |
| (PUP1) | | transporter | | 2000 |
| Cytokinin independent | BAA13416 | Histidine kinase | A. thaliana | Kakimoto, 1996 |
| 1 (CKI1) | | | | |
| G-protein coupled | U95142 | G protein | A. thaliana | Plakidou-Dymock |
| receptor 1 (GCR1) | | coupled receptor | | et al., 1998 |
| Cyclin D3 (CycD3) | T05420 | Cyclin D3 | A. thaliana | Riou-Khamlichi et |
| | | | | al., 1999 |
| Knotted1 (KN1) | S14283 | Homeobox TF | Z. mays | Ori et al., 1999 |

Expression patterns may be varied when alteration of cytokinin activity is to be used to enhance crop performance. Constitutive overexpression of A. tumefaciens iospentyl transferase (ipt) was reported to lead to a variety undesirable effects including decreased root production, shoots that fail to elongate, and small rounded leaves (McKenzie et al., 1998). In contrast, expression of ipt driven by a senescence specific promoter has been shown to significantly delay senescence without inducing other deleterious phenotypes (Gan and Amasino, 1995).

It is expected that the effects of altered cytokinin activity will depend on the tissue specificity and developmental timing of gene expression. Expression in the endosperm early in development should drive increases in cell number, alterations in carbon metabolism and increase sink strength. For example, the basal endosperm transfer layer promoter (bet1) is expressed in the basal endosperm transfer layer between day 9 and day 25 post-pollination (Hueros et al., 1995). The window of bet1 promoter activity corresponds roughly with a normal spike in cytokinin level seen in kernel development (Cheik and Jones, 1994). Two additional genes are known to be expressed specifically early in kernel development. The Esr genes are expressed between 4 and 7 days post-pollination in a region surrounding the embyro (Opsahl-Ferstad et al., 1997; Bonello et al., 2000) and discolored-1 (dsc1) is also expressed over the same time point (Scanlon and Myers, 1998). Expression patterns for dsc1 have not been reported, but dsc1 is known to be essential for both endosperm development and embryo development independent of the endosperm suggesting that it is expressed in both the embryo and endosperm. Senescence-induced promoters have been identified in A. thaliana, but none are currently known in maize (Weaver et al., 1998).

Isopentyl transferase (ipt) is one of many related bacterial genes that catalyze cytokinin biosynthesis from AMP and delta-2-isopentenyl pyrophosphate (McGaw, 1987). Ipt genes have been isolated from many bacterial species including Agrobacterium tumefaciens (GenBank Accession No. AB025109), A. vitis (GenBank Accession No. AAB41870), A. rhizogenes (GenBank Accession No. P14011), Ralstonia solanacearum (GenBank Accession No. P14333), Erwinia herbicola (GenBank Accession No. Q47851), Pseudomonas syringae (GenBank Accession No. P06619) and Anabaena PCC7120 (GenBank Accession No. CAB44665). The ipt gene is directly tested for the ability to improve yield under both normal and stress conditions with the bet1 promoter.

Beta-glucosidase (Zm-p60 or Glu1) is a gene that has been reported to cleave less active cytokinin-O-glucosides and kinetin-N3-glucoside to release active cytokinin (Brzobohaty et al., 1993). However, maize beta-glucosidase has also been reported to hydrolyze a broad range of glycoconjugates making it possible that additional effects would be observed from overexpression of maize beta-glucosidase (Cicek, 2000).

Zeatin O-xylosyltransferase (zox1) and zeatin O-glucosyltransferase (zog1) utilize UDPX or UDPG as sugar donors and catalyze the formation of O-xylosylzeatin or O-glucosylzeatin from zeatin. These compounds are thought to be less active storage or possibly transport cytokinin forms (Martin et al., 1999a and 1999b). The two proteins (ZOX1 and ZOG1) are highly related sharing 86.6% identity over 449 amino acids. Inactivation or inhibition of zeatin O-transferases could result in an increase in available cytokinins.

Purine permase 1 (AtPUP1) is a newly identified member of a 15 member gene family of transporters in A. thaliana (Gillissen et al., 2000). AtPUP1 has been demonstrated to be an energy dependent transporter of adenine. Competitive inhibition of adenine uptake by cytosine, hypoxantine, kinetin, zeatin and caffeine suggests that it may also be able to transport these substances. However the gene is not expressed in roots, which are thought to transport cytokinins to the shoots. No clear physiological role has been demonstrated for any member of the purine permease gene family and it is not clear that AtPUP1 is an endogenous cytokinin permease. Cytokinin permease overexpressing plants are expected to share some phenotypes with ipt overexpressing transformants, but may also appear to have cytokinin deficiencies.

Cytokinin independent 1 (CKI1) was identified in a screen of an A. thaliana TDNA mutant collection for cytokinin independent development on media lacking cytokinins. The protein was found to be a histidine kinase with a typical bacterial receiver domain. A. thaliana callus transformed with CKI1 driven by a 35S promoter was able to develop without addition of exogenous cytokinins (Kakimoto, 1996).

G-protein coupled receptor 1 (GCR1) is a member of the seven transmembrane receptor superfamily most closely related to D. discoideum cAMP receptors. Anti-sense suppression of GCR1 leads to decreased root and shoot sensitivity to cytokinins (Plakidou-Dymock et al., 1998). It has been suggested that GCR1 acts as a cytokinin receptor.

Cyclin D3 is thought to mediate cytokinin stimulation of cell division. A. thaliana seedlings treated with zeatin were found to accumulate transcripts of the D-type cyclin CycD3 as did the A. thaliana mutants amp1 and pt, both of which have elevated cytokinin levels. Overexpression of CycD3 in adult A. thaliana plants led to leaf curling, increased leaf number, late flowering and delayed senescence. In addition, callus derived from these lines was able to grow independently of cytokinins. None of the cycD3 transgenic lines accumulated cytokinins, consistant with a downstream role for cycD3 (Riou-Karnlichi et al., 1999).

D-type cyclins have been identified in corn, sorghum and rice. Both the corn and sorghum genes appear to be more closely related to A. thaliana CycD2 than CycD3. However the rice cyclin (GenBank Accession No. OJ990727_{—}05.9A26.C3) is most similar to A. thaliana CycD3. Additional cyclin D3-like genes have been reported from many different dicots including Nicotiana tabacum (GenBank Accession No. ABO 15222), Lycopersicon esculentum (GenBank Accession No. AJ002589), Pisum sativum (GenBank Accession No. AB008188), Medicago sativa (GenBank Accession No. AJ132929) and Antirrhinum majus (GenBank Accession No. AJ250398).

Knotted 1 (kn1) is a maize transcription factor of the homeobox family. Ectopic expression of kn1 in maize results in a non-cell autonomous alteration in cell fate determination (Vollbrecht et al., 1991). Overexpression of the protein in tobacco results in phenotypes resembling ipt overexpression (Sinha et al., 1993; Ori et al., 1999), e.g., development of meristems on vegetative and inflorescence tissues (Williams-Carrier et al., 1997). The similarities in phenotype between plants ectopically expressing kn1 and ipt have lead to the suggestion that kn1 is acting to promote cytokinin activity. Similar phenotypes have also been observed with plants overexpressing A. thaliana kn1-like protein suggesting that different knotted-like proteins will have similar effects (Lincoln et al., 1994). Overexpression of kn-1 in barley, however, did not lead to development of vegetative meritems on leaves, suggesting that leaf cells of dictos retain a more flexible, less determined state, and therefore, react to signals differently.

Nucleic acid and polypeptide sequences for use in generation of transgenic crop plants having improved properties as described in the above strategy are provided in Table 1.

11. Alteration of Oil Content by Affecting Embryo Size

Corn oil occurs mainly in the cells of the scutellum portion of the embryo (Watson 1987, Weber 1987). Consistent with this distribution pattern is the fact that commercially available high oil corn varieites usually have a larger than normal embryo size (Roth 1998). Scutellum usually occupies more than 90% of the dry weight of the maize embryo (Watson 1987). It is anticipated that by enlarging the size of scutellum, the oil content of the corn kernel could be increased.

A number of maize embryo mutants have been characterized (Clark and Sheridan 1991; Sheridan and Clark 1993). However, the genetic element(s) responsible for those phenotypes is known for few of them. A 3.8 kb genomic fragment was isolated from a maize kernel mutant named dsc1 via Mutator tagging (Scanlon and Myers, 1998). dsc1 displays reduced endosperm size. Embryo growth in homozygous dsc1 kernels was found to be delayed, but proceeds normally to stage 1 in which the scutellum, coleoptile and shoot apex are clearly defined. Compared with the wild type kernels, the size of scutellum in dsc1 mutants was greatly reduced. Embryo growth stopped after stage 1 and embryonic tissues degraded.

The 3.8 kb genomic fragment was cloned and sequenced (Scanlon and Myers 1998). This fragment was shown to co-segregate with the mutant phenotype. It was further determined that this fragment is part of the maize gene responsible for the mutant phenotype and not a sequence closely linked to that locus. Northern blot assay revealed that dsc1 is expressed from 5 to 7 days after pollination (Scanlon and Myers 1998). Genetic analysis showed that the product of dsc1 plays an essential function in the development of the embryo and endosperm, and that dsc1 function is necessary for embryo development beyond stage 1 (Scanlon and Myers 1998). It is believed that overexpression of dsc1 in maize during embryo development will increase the size of the scutellum and the oil content.

A maize DNA sequence was identified by BLAST searching of a Monsanto proprietary database with the dsc1 DNA sequence. The identified sequence encodes a protein that is related to barley nucellin, an aspartic protease-like protein that may be involved in cell death in the nucellus of barley (Chen and Foolad, 1997). This suggests that dsc1 may encode an aspartic protease-like protein that may play a similar role in corn. This class of proteins is also related to tobacco CND41, a chloroplast localized protease with DNA-binding activity that has been suggested to play a role in regulation of gene expression in the chloroplast (Nakano et al., 1997; Murakami et al., 2000).

Nucleic acid and polypeptide sequences for use in generation of transgenic crop plants having improved properties as described in the above strategy are provided in Table 1.

12. Cell Cycle Regulation by E2F

The retinoblastoma gene was one of the first tumor suppressor genes to be identified in mammals. The Rb tumor suppressor gene functions as a negative regulator of cell proliferation and normally acts to inhibit unregulated cell division in mammalian cells. Unlike mammalian cells, little is known how plants regulate G1 progression (Ach et al. 1997). cDNAs have been isolated in maize that encode Rb-related proteins and have been shown to be highly expressed in the shoot apex (Ach et al. 1997). The Rb-like proteins have been shown to interact with type D-Cyclins (Ach et al. 1997), are a family of proteins that regulate the activity of cyclin dependant kinases (CDK; Riou-Khamlichi et al. 1999) and control progression through the cell cycle (Cooper, 1995). It is suggested that G1 regulation in plant cells is controlled by mechanisms similar to the ones found in mammalian cells (Ach et al. 1997). The presence of CDKs and other cyclins suggest that the mechanism for regulation has been conserved in eukaryotic evolution (Ach et al. 1997). Recent evidence suggests that G1 regulation in plants involves Rb-related proteins. Rb has also been shown to interact with other proteins such as the wheat dwarf virus C1 protein (Xie et al. 1995). Inactivation of the Rb-related proteins either contitutively or at specific times in development is expected to increase cell division rates, resulting in bigger plants, higher yields, increased turgor and a higher degree of seed set in times of water stress.

Association of Rb with E2F is central to cell cycle regulation in animal cells (Ramirez-Parra et al. 1999). E2F is a family of transcription factors that is regulated by the formation of complexes with Rb proteins. E2F activates genes involved in cell proliferation and division, the activity of which is thought to induce expression of genes involved in DNA metabolism and cell cycle progression. The E2F transcription factor is overexpressed transgenically in order to increase transcription of cell cycle related genes at a higher rate. E2F transcription factors have been isolated from several plant species; Daucus. carota (GenBank Accession No. AJ251585), Triticum sp. (GenBank Accession No., GenBank Accession No. AJ238590) and Oryza sativa (GenBank Accession No. AB041725). Plant E2F proteins have been shown to be expressed in proliferating cells (Albani et al. 2000, Ramirez-Parra et al. 1999). The E2F protein was also shown to be induced during the G₁/S transition of the cell cycle (Albani et al. 2000). High levels of constitutive expression of the E2F transcription factor is expected to initiate transcription of cell cycle progression genes.

DP proteins have been demonstrated to form heterodimers with E2F and promote binding of E2F to DNA (Helin et al, 1993; Ramirez-Parra and Gutierrez, 2000). The specific subunit composition of the E2F/DP heterodimer appears to affect binding site selectivity and will likely alter the resulting group of genes induced (Tao et al, 1997). Overexpression of specific DP proteins may produce similar effects to overexpression of E2Fs and can potentially lead to improvements in crop performance. Effects of E2F and DP overexpression are reported to vary widely depending upon the specific protein isoforms and cell types involved (Chen et al., 2000).

Nucleic acid and polypeptide sequences for use in generation of transgenic crop plants having improved properties as described in the above strategy are provided in Table 1.

13. Reduction of Senescence

Senescence is associated with at least two agronomically important stages of corn development, i.e., leaf senescence and endosperm development (Young and Gallie, 2000). Delayed leaf senescence or stay green is associated with longer grain filling periods and increased kernel weight (Prioul, 1992). Delaying endosperm senescence may have the affect of increasing grain weight or quality by increasing endosperm size or cell number.

Eukaryotic initiation factor 5A (eIF-5A) transcripts have been shown to accumulate in unfertilized egg cells in maize (Dresselhaus, 1999) and may play a role in cell growth or division upon fertilization. eIF-5A was originally isolated as a putative translation factor from a polyribosome-bound fraction (Kemper et al., 1976; Benne et al., 1978) and was suggested to be involved in the formation of the first peptide bond. Subsequent studies, however, indicated that translational initiation is not directly affected by a loss of eIF-5A function (Kang and Hershey, 1994; Zuk and Jacobson, 1998) and that eIF-5A is not required to assemble translation initiation complexes. Translation initiation factors are usually present substoichiometrically to ribosomes and associate with them only during initiation. In contrast, eIF-5A is present in excess over ribosomes (Hershey, 1-994) and largely bound to cytoplasmic, puromycin-sensitive structures that might represent rough endoplasmic reticulum-bound polysomes (Shi et al., 1997). Therefore, if eIF-5A functions in translation, it is probably at some step subsequent to initiation.

EIF-5A is activated by a post-translational modification of a specific lysine residue to hypusine (N-(4-amino-2-hydroxybutyl)lysine) in an enzyme-catalyzed two-step mechanism (Park et al., 1993; 1997). In the first step, the aminobutyl moiety of the polyamine spermidine is transferred by deoxyhypusine synthase (EC 1.1.1.249) in an NAD+-dependent reaction to the epsilon-amino group of a specific lysine residue in the EIF-5A precursor protein to form deoxyhypusine. In the second step, deoxyhypusine hydroxylase (EC 1.14.99.29) catalyzes the hydroxylation of the deoxyhypusine residue to hypusine. Activated EIF-5A is the only protein in which the unusual amino acid hypusine has been detected to date.

The hypusine modification of eIF-5A is conserved in all organisms studied to date and is required for cell growth. However, the presence of high levels of hypusine-modified eIF-5A is associated with high levels of cell division both during development and in human cancers (Chen et al., 1997b). The build up of unmodified eIF-5A is associated with senescence in animal systems (Chen et al., 1997a). Inhibitors of hypusine modification have been used to show that there is a direct link between eIF-5A modification state and cell cycle (Hanauske-Abel et al., 1994; Park et al., 1997). This active role played by eIF-5A in cell growth and senescence suggests that modification of the activity state of eIF-5A may be used to enhance agronomically important traits such as kernel growth rates and stay green.

Deoxyhypusine synthase has been cloned from tobacco and Senecio vernalis (Ober and Hartmann (1999a, b)). Both of the tobacco and S. vernalis enzymes are able to efficiently aminobutylate putrescine to form homospermidine. Homospermidine is the first pathway-specific intermediate in the synthesis of pyrrolizidine alkaloids. However, homospermidine is synthesized by homospermidine synthase, an enzyme thought to have evolved from deoxyhypusine synthase, in plants that contain pyrrolizidine alkaloids. The role of homospermidine in other plants in unclear.

Elevated putrescine levels have been associated with induction of apoptosis in mammalian cells (Tome et al., 1997) and cells selected for growth on ornithine decarboxylase inhibitors accumulate more eIF-5A (Tome et al., 1996). This has lead to the suggestion that variations in spermidine levels may lead to alterations in eIF-5A activity. This suggestion is supported by the observation that many cancer cells overexpress ornithine decarboxylase and S-adenosylmethionine decarboxylase (Ravanko et al., 2000).

Expression of ornithine decarboxylase is highly regulated in eukaryotic cells. The protein is inhibited by a specific antizyme that can only be translated when high levels of polyamines allow frameshifting (Matsufuji et al., 1990). The activity of the antizyme is, in turn inhibited by an antizyme inhibitor (Murakami et al., 1996). Forced expression of the antizyme has been shown to inactivate ornithine decarboxylase and suppress cellular polyamine levels (Murakami et al., 1994).

Increasing spermidine levels by overexpression of ornithine decarboxylase or S-adenomethionine decarboxylase may alter cellular growth patterns and retard senescence, possibly by increasing the levels of hypusine-modified eIF-5A. This same effect might also be achived by overexpressing the ornithine decarboxylase antizyme inhibitor.

eIF-5A appears to be an RNA-binding protein (Liu et al., 1997), and probably the hypusine modification and the C-terminal domain contribute to the interaction (Liu et al., 1997; Kim et al., 1998; Peat et al., 1998). Although it is not yet know with which RNAs eIF-5A interacts, it is possibile that eIF-5A functions as an export adapter for these RNAs. However, assuming that the essential function of elF-5A is conserved between eukaryotes and archaea, and given that archaea have no nuclei, it is possible that nuclear export of other macromolecules is not the primary function of eIF-5A. Furthermore, if eIF-5A was an export adapter, then it is expected that it would be actively imported into nuclei. However, nuclear accumulation of eIF-5A appears to occur solely by passive diffusion, as it is insensitive to reagents that block facilitated nuclear import. In addition, the hypusine modification is essential for eIF-5A function, probably involved in the interaction with its functional targets, and required for the RNA-binding activity of eIF-5A. Hypusine is also involved in the interaction with exportin 4 (Exp4). It is not known whether the hypusine in an eIF-5A-Exp4-complex is still available for RNA binding and export. Nucleocytoplasmic transport was reviewed by Mattaj and Englemeier (1998).

The eIF-5A protein shuttles between the nucleous and the cytoplasm in a hypusine-dependant manner. eIF-5A interacts with another nucleocytoplasmic shuttle protein, L5. This transport appears to be mediated by a specific export protein, exportin 4 (EXP4). L5 is an RNA binding protein that interacts with 5S RNA and modulates transport and assembly of the 60S ribosome particle. eIF-5A is also an RNA binding protein. Together, this suggests that one of the functions of eIF-5A may be to modulate the rate of 5S RNA assembly into 60S ribosome particles. Accumulation of unmodified eIF-5A would lead to accumulation of eIF-5A and it's putative cargo in the nucleus. However, it is also likely that eIF-5A interacts with additional proteins and RNAs.

eIF-5A is exported from the nucleus by a specific exporter, exportin 4 (Lipowsky et al., 2000). Exportin 4 is the most distant family member of the exportin protein identified so far. Nevertheless, it functions according to the same principles as the other exportins i.e. it binds its export cargo preferentially in a nuclear environment in the presence of RanGTP, forming a trimeric eIF-5A-Exp4-RanGTP export complex. This complex is subsequently transferred out of the nucleus. Cytoplasmic disassembly of the complex and concomitant cargo release are brought about by the concerted action of RanGAP and RanBP1 (or RanBP2, respectively), and also result in the hydrolysis of the Ran-bound GTP. The ‘empty’ exportin can then re-enter the nucleus and participate in another round of export.

The hypusine modification is apparently part of the signal that allows eIF-5A to access the Exp4 pathway. Recombinant eIF-5A that lacks the modification binds to Exp4 35 times more weakly than the fully modified protein, i.e. the recognition of the modification appears to contribute ˜8 KJ/mol binding energy. Deoxyhypusine can partially, but not fully, substitute for hypusine, indicating that the hydroxyl moiety of hypusine also contributes to Exp4 binding.

Exp4 may mask hypusine and other crucial parts of the eIF-5A molecule in order to prevent an interaction of eIF-5A with potential targets in the nucleus. Thus, eIF-5A might be actively exported from nuclei, not because it is a transporter for other macromolecules, but instead because its function is restricted to the cytoplasm. The available data is consistent with eIF-5A being involved in some aspect of translation or cytoplasmic degradation of mRNA or with eIF-5A functioning as an RNA chaperonin or in some other facet of RNA metabolism. eIF-5A accumulates in the absence of Exp4 in nucleoli, the sites where ribosomes are assembled. Untimely binding of eIF-5A to pre-ribosomal particles may interfere with ribosome biogenesis. Exp4-mediated export of eIF-5A would ameliorate such an effect.

L5 is a nucleocytoplasmic shuttle protein that is involved in the intracellular transport of 5 S rRNA (Steitz et al., 1988; Guddat et al., 1990; Rudt and Pieler, 1996). After transcription, 5 S rRNA transiently binds the La antigen, a 50-kDa protein that acts in the termination of polymerase III transcripts, in the nucleus (Rinke and Steitz, 1982; Gottlieb and Steitz, 1989). Furthermore, in the nucleus, 5 S rRNA also binds either its own transcription factor IIIA or ribosomal protein L5, forming 7 or 5 S ribonucleoprotein particles, respectively. In particular, it has been suggested that the 5 S rRNA·L5 complex (5 S ribonucleoprotein particle) acts as a precursor to ribosome assembly by delivering 5 S rRNA from the nucleoplasm to the nucleolar assembly site of 60 S ribosomal subunits (Steitz et al., 1988). Studies in Xenopus oocytes have shown that 5 S rRNA can be exported from the nucleus to the cytoplasm for subsequent accumulation at distinct cytoplasmic storage sites by either transcription factor IIIA or L5 (Guddat et al., 1990, Allison et al., 1995). As a consequence of increased ribosomal subunit synthesis, stored 5 S rRNA must be reimported from the cytoplasm into the oocyte nucleus. In contrast to nuclear export, however, the nuclear import of 5 S rRNA appears to be exclusively mediated by L5 protein (Allison et al., 1991; Rudt and Pieler, 1996). Although cytoplasmic storage sites for 5 S rRNA have not been observed in mammalian cells, the data so far raised in Xenopus oocytes demonstrated that L5 protein is an intracellular 5 S rRNA transport factor.

The network of nucleic acid-protein interactions involving TFIIIA, L5, 5 S rRNA, and the 5 S rRNA gene suggests a model in which 5 S rRNA synthesis is coupled to the accumulation of the L5 ribosomal protein. In the proposed regulatory loop, an increase in the concentration of L5 would result in displacement of the equilibrium between each of the relevant RNPs (5 and 7 S) and its constituent components in opposite directions, resulting in the formation of additional 5 S RNP and the release of free TFIIIA from 7 S RNPs. The TFIIIA released from 7 S RNPs would be available for binding to and nucleating transcription complex formation on additional 5 S rRNA genes. Thus, 5 S rRNA synthesis would be responsive to levels of L5 expression, even though there is no reason to believe L5 is directly involved in 5 S rRNA synthesis in any way (Pittman et al., 1999).

eIF-5A has been shown to directly interact with ribosomal protein L5 and both proteins are required for export of the HIV rev protein (Schatz et al., 1998). L5 has also been shown to interact with mdm2 (Marechal et al., 1994). Mdm2 interacts with p53 to inactivate the protein and promote tumor growth in mammalian cells (Momand et al., 1992). This suggests that eIF-5A and L5 may also interact to modulate in the export or assembly of 5S RNA into 60S ribosomal subunits.

The three dimensional structures of eIF-5A from two archaea species have been elucidated using X-ray crystallography (Kim et al., 1998; Peat et al., 1998). They show eIF-5A to be composed of two compact domains that are linked by a flexible hinge. N-terminal domain I contains the hypusine modification site in an extended, protruding and highly conserved loop. The modification is unlikely to have a major effect on the eIF-5A structure. Instead, its absolute conservation indicates that the hypusine mediates essential interactions with other macromolecules. Hypusine is a 2-fold positively charged amino acid and resembles nucleic acid-binding polyamines such as spermine and spermidine. Domain II is similar to the RNA-binding motif found in the prokaryotic cold shock protein CspA, which has been suggested to function as an RNA chaperone (Jiang et al., 1997). Domain II and the hypusine-containing loop from domain I may thus constitute a bipartite RNA-binding site (Kim et al., 1998; Peat et al., 1998). An RNA-binding activity of eIF-5A was detected in vitro and was found to depend on the hypusine modification (Liu et al., 1997). However, it is still unclear whether this RNA-binding activity reflects a genuine function of eIF-5A and if so what the physiological RNA ligand(s) of eIF-5A may be.

Eubacteria lack a hypusine-modified eIF-5A equivalent. However, the sequence similarity between eubacterial EF-P and archaebacterial/eukaryotic eIF-5A is significant enough to assume that the two represent homologous proteins (Kyrpides and Woese, 1998). EF-P is essential for viability in Escherichia coli (Aoki et al., 1997) and present in all eubacterial genomes examined. eIF-5A/EF-P can thus be considered a universally conserved and essential protein; it is apparently the only known protein that falls into this category whose function has remained elusive.

Loss of eIF-5A function is ultimately lethal (Schnier et al., 1991; Kang and Hershey, 1994; Sasaki et al., 1996; Zuk and Jacobson, 1998; Jansson et al., 2000). Saccharomyces cerevisiae cells stop cell division, but continue to enlarge in size. One specific defect is an impaired degradation of mRNA, particularly of short-lived messages (Zuk and Jacobson, 1998). The defect apparently occurs because of reduced mRNA decapping and degradation by the Xrn1p exonuclease and may be consistent with the assumed RNA-binding activity of eIF-5A. However, it is unclear whether RNA turnover is the primary function that makes eIF-5A indispensable for viability in all organisms. Upon eIF-5A inactivation in yeast, the overall rate of translation is reduced by ˜30%, but is not immediately abolished (Kang and Hershey, 1994; Zuk and Jacobson, 1998), suggesting that translational initiation and elongation can proceed in the absence or at very low concentrations of eIF-5A. The intermediate effect on the overall translation rate and the requirement of eIF-5A for viability could possibly be explained by a failure of eIF-5A-deficient cells to synthesize a subset of proteins or to synthesize them in a biologically active form.

Overexpression of deoxyhypusine synthase is expected to increase the level of hypusine modification found in eIF-5A and thus inhibit senescence and/or promote growth. Likewise overexpression of the eIF-5A exporter exportin 4 is expected to maintain eIF-5A transport levels when the lower affinity unmodified eIF-5A form is present. Overexpression of eIF-5A is expected to increase the levels of unmodified protein and slow growth or the protein may be modified by the endogenous deoxyhypusine synthase to promote growth. Overexpression of L5 can be expected to increase the transcription rate of 5S RNA and may overcome the affects of increasing amounts of unmodified eIF-5A which accumulate during sensecence.

Nucleic acid and polypeptide sequences for use in generation of transgenic crop plants having improved properties as described in the above strategy are provided in Table 1.

14. Endogenous Insecticides

One method by which plants may defend themselves from herbivorous insects is by producing bioactive secondary metabolites. For example, maize seedlings attacked by beet armyworm larvae (Spodoptera exigua) produce a mixture of terpenoid and indole volatiles that serve to attract parasitic wasps (Turlings and Fritzsche, 1999). This response is induced in plants by the production of volicitin, N-(17-hydroxylinolenoyl)-L-glutamine by the herbivore during feeding (Pare et al., 1998).

Genes involved in the production of both terpenoids (Shen et al., 2000) and indole (Frey et al., 2000) in response to volicitin have recently been identified in corn. Both terpenoids and indole are induced in response to volicitin, suggesting that overexpression of these genes may lead to increases in terpenoids or indole. Increased production of these compounds may lead to the build up of other insecticidial metabolites such as hydroxamic acids (Gierl and Frey, 1999).

A gene encoding a 33-kDa cysteine proteinase was recently identified in corn lines resistant to numerous lepidopteran species including fall armyworm (Spodoptera frugiperda), southwestern corn borer (Diatraea grandiosella), European corn borer (Ostinia nubilalis), sugarcane borer (D. saccharalis), tobacco budworm (Heliothis virescens) and corn earworm (Helicoverpa zea) (Pechan et al., 1999; Pechan et al., 2000). Larvae reared on Black Mexican Sweet (BMS) callus that was transformed to express active 33-kDa cysteine proteinase were markedly smaller than those reared on control callus. Low concentrations of the 33-kDa cysteine proteinase were constitutively present in the whorl and the protein accumulated greatly in response to larval feeding, suggesting that this protein may also be part of the response to volicitin. Overexpression of this protein may reduce feeding damage due to lepidopteran insects.

Sesquiterpene cyclase mutants lack the sesquiterpenoid naphthalene, 1,2,4α-5,8,8α-hexahydro-4,7-dimethyl-1-(1-methylethyl)-(1,4α,8α), which is a volatile component of the set of terpenoids produced by maize in response to volicitin (Shen et al., 2000). In contrast, biochemical analysis has defined hydroxymethyl glutaryl (HMG)-CoA reductase and terpenoid synthase as two key regulatory enzymes of terpenoid biosynthesis (Bohlmann et al., 1998). HMG-CoA reductase catalyzes the committed reaction of the general terpenoid synthesis pathway. Terpenoid synthase catalyzes the divergent reactions that produce the vast variety of terpenoids seen in plants.

Overexpression of either terpenoid synthase or HMG-CoA reductase may lead to the overexpression of volatile leading to increased pest resistance in a transgenic plant (Turlings et al., 1990, Shen et al., 2000).

IGL (indole-3-glycerol phosphate lyase) is similar to the maize bx1 gene and a newly identified corn gene called TSA-like that is believed to be the corn tryptophan synthase alpha subunit. Overexpression of either bx1 or IGL should increase levels of indole and possibly also levels of DIMBOA. Accumulation of hydroxamic acids such as DIMBOA should lead to detectable changes in insect growth rates.

The mir1 33-kD cysteine proteinase protein belongs to the papain family of cysteine proteases. The protein is very similar to three other maize cysteine proteases, mir2, mir3 and CPPIC and the rice protease oryzain alpha with the exception of it's C-terminus which lacks a conserved region found in the other four proteases. CPPIC is found as a complex with cystatin (Yamada et al., 1999). All of these proteins are predicted to be secreted due to N-terminal signal sequences. The C-terminal truncation of mir1 may act to activate the protein by reducing interaction with cystatin or to increase the free concentration of cystatin, which then leads to the reduction in insect feeding and growth rates. It is expected that overexpression of cystatin in maize will also lead to a reduction in insect feeding.

Nucleic acid and polypeptide sequences for use in generation of transgenic crop plants having improved properties as described in the above strategy are provided in Table 1.

15. Freezing Tolerance—Eskimo 2

eskimo2 (esk2) is a mutant in Arabidopsis thaliana ecotype which accumulates high levels of free proline and is constitutively freeze-tolerant in the absence of cold acclimation. Esk2 was cloned based on map position and corresponds to the gene F28A21.190 (g4539397) annotated by the public Arabidopsis sequencing project. ESK2 is annotated as a cellulose synthase-like protein because it has a strong similarity to a presumed cellulose synthase (U58283) gene isolated from a cotton fiber library. Currently, two cellulose synthase genes have been cloned from Arabidopsis, RSW1 (AF027172) and IRX3 (AF088917 and AF091713). Esk2 is a new gene that shows 60% identity with either of the two cellulose synthase genes previously isolated from Arabidopsis. The constitutive freezing tolerance phenotype shown by esk2-2 mutants suggests that esk2 may mediate freezing tolerance in Arabidopsis. The N-terminal 200 amino acid sequence is strongly similar to the PHD domain of a class of transcription factors.

Rajashekar and Burke (1996) and Rajashekar and Lafta (1996) reported that cold acclimation and abscisic acid treatment induces changes in cell wall properties. These changes are correlated with increases in freezing tolerance. Marshall and Dumbroff (1999) showed that cell wall adjustment in response to slow drought and pacloputrazol treatment is associated with turgor regulation and drought tolerance in white spruce. Consistent with a possible role in structural polymer biosynthesis, xylem structure in both esk2-1 and esk2-2 plants appears to be affected by the esk2 mutation. In contrast to the published irx3 mutant, which has less rigid stems than those of wild-type, stems of esk2-1 appear to be stronger. By sequence analysis, there are about 16 genes encoding cellulose synthase-like proteins in Arabidopsis. It is unclear if all cellulose synthases or specific ones that involve the modification of cell wall properties related to abiotic stress tolerance. Expression of the gene esk2 in corn is expected to contribute to increasing cold tolerance and water use efficiency in a plant.

Nucleic acid and polypeptide sequences for use in generation of transgenic crop plants having improved properties as described in the above strategy are provided in Table 1.

16. Fatty Acid Desaturases

One promising approach to improving cold or heat tolerance in plants is to alter the composition of membrane lipid fatty acids (Somerville, 1995). It is expected that increasing the desaturation of fatty acids will allow membranes to remain fluid and functional at lower temperatures. This idea is supported by several lines of evidence. First, comparison of the membrane lipid fatty acid composition of chill-sensitive and. chill-tolerant species indicated that chill-tolerant species tend to have greater amounts of desaturation in the chloroplast membrane lipid, phosphatidylglycerol. Also, when chill-tolerant plants are acclimated to cold temperatures, the amount of desaturated fatty acids increases. Second, mutants of Arabidopsis and Synechocystis defective in various desaturases are more chill-sensitivite than wild type. Finally, introduction of transgenes that increase membrane lipid desaturation has resulted in plants that have increased cold tolerance, while transgenes that decrease membrane lipid desaturation resulted in plants that are more chill sensitive. It has recently been shown that decreasing trienoic fatty acids in chloroplast membrane lipids in transgenic tobacco and Arabidopsis resulted in greater heat tolerance (Murakami et al. 2000).

In rice, there is a strong correlation among different varieties between high levels of 18:3 fatty acids and cold tolerance (Bertin et al. 1998). Biosynthesis of 18:3 fatty acids is catalyzed by omega 3 desaturases. Arabidopsis has three genes that encode omega 3 desaturases. FAD3 encodes a microsomal form, and FAD7 and FAD8 encode proteins targeted to the chloroplast. FAD8 mRNA accumulation is induced by low temperature. Expression of an omega 3 desaturase from Arabidopsis, FAD7, in tobacco caused increased accumulation of 18:3 fatty acids and increased cold tolerance (Kodama et al. 1995).

It is believed that the overexpression of either Arabidopsis FAD7 or FAD8 genes in corn will contribute to cold tolerance. It is also believed that down regulation of FAD7 by antisense or cosupression may contribute to heat tolerance. Use of fatty acid desaturase transgenes to confer chilling tolerance is disclosed in PCT Publication No. WO 94/18337.

Fatty acid composition of leaf tissue is determined in transgenic plants expressing FAD7 or FAD8. High and low temperature tolerances are tested if the desired modifications in fatty acid composition are obtained. Cold tolerance is tested by germination and rate of seedling growth at an inhibitory temperature. Chlorophyll and anthocyanin accumulation at a chilling temperature are also determined. Heat tolerance is determined by seedling growth at high temperature, and by chlorophyll accumulation at high temperature. Effects of high temperature on fertility are either determined by growth in a hot greenhouse or at an appropriate field site.

Nucleic acid and polypeptide sequences for use in generation of transgenic crop plants having improved properties as described in the above strategy are provided in Table 1.

17. Ferritin

During an abiotic or biotic stress response, plants undergo a structural change within the cell wall to maintain their defenses against other stresses or pathogen attacks. Membrane-mediated responses occur, which include release and accumulation of reactive oxygen species and lipoxygenase enzymes. Hyperoxidation of membrane phospholipidsis is triggered, resulting in production of toxic compounds that disrupt cell membranes and may cause cell collapse and death. The maintenance of cell wall integrity during stress is a key to maintaining plant productivity. As a result of abiotic stress, oxidative stress at the cellular level causes much damage (Buchanan et al., 2000). It is believed that antioxidant enzymes are critical for prevention of oxidative stress in plants (Allen, 1995).

Overexpression of ferritin in plants results in an increase of resistance to abiotic and biotic oxidative stresses. Ferritins are multimeric proteins containing 24 subunits and have been found to maintain cell wall composition. The ferritin gene family occurs in plants, animals and microorganisms and is responsible for storage and distribution of iron within cells. Ferritin synthesis in plants is regulated at the transcriptional level as opposed to translational control as in animals. Plant ferritins are localized in plastids whereas in animals, they are found in the cytoplasm (Gaymard et al. 1996).

Ferritin gene expression has been demonstrated in a variety of plants including maize, Arabidopsis, cowpea, soybean, bean and pea. Most available evidence shows that the mature protein is located in plastids and its production is under transcriptional control. Briat et al. (1999) suggested that an A. thaliana library of expressed sequence tags might contain sequences derived from divergent ferritin sequences. This information implies that there is a large family of ferritin genes in each higher plant genome (Wardrop et al., 1999). Briat et al. (1999) further reported on a family of homologous ferritin sequences in maize. These disclosed sequences varied mostly in the control regions of the genes.

Different ferritin subunits are expressed under different physiological conditions (Fobis-Loisy et al., 1995; Savino et al. 1997). In maize, two different nuclear ferritin genes have been identified; they were found to express protein under different physiological conditions and are named ZmFer1 and ZmFer2. The two different ferritin genes that have been identified are differentially expressed in response to abscisic acid or iron-mediated oxidative stress (Savino et al, 1997). A cellular pathway involving abscisic acid (ABA) regulates ZmFer2, but the ZmFer1 gene is regulated via an ABA-independent pathway (Petit et al., 2000). ZmFer2 mRNA accumulates in response to both iron and ABA treatments, whereas ZmFer1 mRNA level is only increased by iron treatment (Briat et al, 1999). Petit et al. (2001) demonstrated in plantlets from which the roots were removed and maize cell suspension that the ZmFer1 gene is regulated by both iron and redox signals. Hydrogen peroxide treatments induce an increase in ZmFer1 mRNA levels and treatment with N-acetylcysteine (NAC), an antioxidant agent, inhibited iron-induced accumulation of the ZmFer1 transcript. This implies that ZmFer1 accumulates in response to iron via an oxidative step in the pathway as disclosed by Savino et al. (1997). A similar result was observed in Arabidopsis, where an increase of AtFer1 transcript in response to iron was inhibited by the antioxidant N-acetylcysteine (NAC). These results indicate that an oxidative pathway, independent of abscisic acid, could be responsible for the iron induction of ferritin synthesis in A. thaliana.

The production of reactive oxygen species (ROS) occurs during oxidative stress due to the release of free iron ions from ferritin degradation. Oxidative stress is caused by drought, heat, cold stress and pathogen attack. Overproduction of ferritin causes more iron to be bound and therefore eliminates damage that may be caused by oxygen free radicals (WO 98/46775). Overexpression of ferritin in plants, specifically alfalfa ferritin expressed into tobacco, was reported to make transgenic plants more resistant to oxidative stress. Transgenic plants sprayed with paraquat and fusaric acid, which cause oxidative stress, had reduced necrotization relative to nontransgenic plants.

Overexpression of ferritin promotes cellular productivity during limited water conditions to prevent formation of oxygen radicals. Iron is also important in plant-specific pathways like nitrogen fixation and phytohormone synthesis. Iron starvation results in physiological changes in plants (Allen, 1995). Ferritin is localized to the plastids in plants and is degraded during germination for iron utilization for growth of the seedling. Iron deficiency leads to chlorosis of leaf tissue, which is an indicator that sufficient chlorophyll is not present, and that photosynthesic activity has been reduced. Iron toxicity is linked to high reactivity to oxygen species and production of hydroxyl radicals, both of which can damage cell components (Savino et al., 1997). This can result in activation of ferritins by iron. Ferritin mRNA accumulation was inhibited in the presence of an antioxidant in maize plantlets that were de-rooted and it also accumulates in response to iron overload. A five-fold increase in ABA concentration in roots and leaves was observed in response to an iron overload in maize plantlets and ferritin mRNA accumulated in response to exogenous ABA treatment (Briat et. al, 1999).

Nucleic acid and polypeptide sequences for use in generation of transgenic crop plants having improved properties as described in the above strategy are provided in Table 1.

18. Farnesylpyrophosphate Synthase

FPPS, farnesylpyrophosphate synthase, plays a role in glyphosate resistance in yeast.

Expression of FPPS in corn may be useful to develop improved glyphosate formulations, increase glyphosate resistance, screen for compounds that synergize with glyphosate, broad spectrum disease control, or broad spectrum insect control.

Nucleic acid and polypeptide sequences for use in generation of transgenic crop plants having improved properties as described in the above strategy are provided in Table 1.

19. Fungal Resistance

Cold field temperatures after planting corn seed in the soil can delay germination and emergence of the seedling from the soil. If emergence of the corn seedling from the soil is significantly delayed, fungal infection of the seed may occur, thus severely damaging or destroying it. One approach to introduce cold tolerance into corn seedlings is to make the seed or seedling more resistant to fungal infection. Such a seed/seedling may be better adapted to survive without fungal infection until soil temperature increases.

Mendel Biotechnology has identified genes (G28, G378, G19 and G188) that confer enhanced fungal resistance in Arabidopsis plants when overexpressed with a constitutive promoter. The gene sequence for G28 corresponds to AtERF1 (Fujimoto, et al. 2000), which is described as an Ethylene Responsive Element Binding Factor (EREBP). In general, these genes are thought to be involved in various stress responses. A homolog of G28 from wheat is provided herein.

It is expected that constituitive overexpression in corn of the G28 and G378 genes and their homologs will enhance fungal resistance, and may indirectly enhance cold tolerance of corn plants by allowing the plants to resist fungal infection when emergence from the soil is delayed by cold temperatures.

Nucleic acid and polypeptide sequences for use in generation of transgenic crop plants having improved properties as described in the above strategy are provided in Table 1.

20. Gamma-Aminobutyric Acid Synthesis

Gamma-aminobutyric acid (GABA) is a non-protein amino acid produced ubiquitously in plants. GABA is synthesized primarily through the alpha decarboxylation of L-glutamate catalyzed by the enzyme glutamate decarboxylase (GAD). GABA is well known in animals as a stress-induced inhibitory neurotransmitter but its exact role in plant metabolism is unclear. Much research has shown that high levels of GABA accumulate rapidly in plant tissues that are exposed to a wide variety of stresses. Examples of the greatest reported GABA accumulation are: acidosis, 300% of control in 15 seconds (Crawford, 1994); mechanical damage, 1800% of control in 1 min. (Ramputh, 1996); cold, 2000% of control in 5 minutes. (Wallace, 1984); anoxia, 1000% of control in 24 hours. (Aurisano, 1995); heat, 1800% of control in 24 hours (Mayer, 1990); drought, 1000% of control in 3 days (Thompson, 1996); salt, 300% of control in 5 days (Bolarin, 1995); and viral, 130% of control in 13 days (Cooper, 1974). Many of these stresses are unrelated which implies a linkage between the perception of the stress, GABA accumulation and diverse mitigation of each stress. This implies a signal transduction pathway and a role for GABA as a signaling molecule. Studies have shown that signaling molecules must follow certain criteria. They alter gene expression (activate enzymes), alter growth (metabolism), and have receptor molecules (Kinnersly, 2000).

Several plant GAD genes have been cloned (Baum, 1993; Johnson, 1997; Turano, 1998; Yunand Oh, 1998; Zik, 1998). Recent studies have found that plant GADs have 22 to 25 additional amino acids at the C-terminal when compared with mammalian GADs and these domains have been shown to be sufficient for the binding of calmodulin (CaM) and Ca2+(Arazi, 1995; Gallepo, 1995; Ling, 1994; Snneden, 1996). Another study found that CaM binding is required for GABA metabolism and normal plant development in plants (Baum, 1996). It is well recognized that Ca2+/CaM complexes stimulate enzymes to initiate a cascade of biochemical and molecular events in plant cells (Zielinski, 1998) and that many environmental stresses increase Ca2+levels in cells (Sanders, 1999). Cloned cDNAs encoding GAD have been used to demonstrate that Ca2+/CaM play a role in regulating GAD activity that was stimulated nearly 100 fold (Snedden, 1996). Another study investigated the role of Ca2+/CaM in cold-shock stimulated GABA synthesis of rice (Oryza sativa) isolated mesophyll cells. In the same study, a fluorescent indicator demonstrated an increase in Ca2+ levels within 2 sec. of the cold shock with GABA increases within a minute. A Ca2+ ionophore stimulated similar Ca2+ levels and GABA synthesis in the absence of the cold while Ca2+ channel blockers and CaM antagonists inhibited cold shock stimulated GABA accumulation (Cholewa, 1997).

It has been suggested that GABA is an inducer of “stress ethylene”. Direct evidence of GABA's effect on ethylene production was obtained in vitro with Stellaria longipes, when ethylene evolved and stem elongation was promoted by high concentrations of GABA (Kathiresan, 1998). In a separate study with sunflower cotyledons, there was a 14-fold increase in ethylene production with exogenously applied GABA (Kathiresan, 1997).

GABA has been found to alter growth by mineral acquisition in experiments with duckweed (Lemma minor L.). Plant growth and mineral content were increased when duckweed plants were cultured in media containing 1 and 10 mM GABA. Plants treated with 10 mM GABA had significantly higher levels of all macronutrients and manganese, zinc and boron than in untreated plants (Kinnersley, 2000). In another study, wheat plants treated with foliar applied GABA yielded 35% more grain with plant tissue containing 32% higher manganese levels than untreated plants (Kinnersley, 1998). Manganese is an essential element for activating enzymes in the lignin biosynthetic pathway (lignin is a complex polymer in cell walls that acts as a physical barrier to fungal hyphae, wounding, etc). In tomatoes, exogenous GABA treatments increased in plant nutrients and yield as compared to untreated plants (Kinnersley, 1998). These observations suggest that GABA mediates a root-specific response and intercellular GABA transport by stress-induced GABA transporter proteins has been discussed (Shelp, 1999).

GABA has been identified as playing a role in stress metabolism. GABA is synthesized as described above by GAD enzymes catalyzing the reaction L-Glu+H+→GABA+CO2. GABA is metabolized through a reversible transamination (GABA+pyruvate=succinic semialdehyde+Ala) catalyzed by GABA transaminase. The product succinic semialdehyde is oxidized to succinate (succinic semialdehyde+NAD+H2O→succinate+NADPH) in an irreversible reaction catalyzed by succinate dehydrogenase.

GABA metabolism takes place in the mitochondria and the above three reactions constitute a pathway known as the GABA shunt. It is important to plant metabolism that intramitochondrial concentrations of the citric acid cycle (TCA cycle) intermediates remain constant over time. Anaplerotic reactions (filling up reactions) are metabolic processes that ensure this constancy. Since the TCA cycle performs an anabolic role, intermediates are drained from the cycle for biosynthesis of amino acids. A crucial role of anabolic reactions is in stress related metabolism. Amino acids form the buidling blocks for heat shock proteins, cold-induced proteins, anaerobic proteins, pathogen-related proteins, etc. Depletion of TCA cycle intermediates during times of stress has been demonstrated to be relieved by anaplerotic carbon fixation of oxaloacetic acid that is synthesized from phosphoenolpyruvic acid (PEP) by PEP carboxylase (Plaxton, 1996). When PEP carboxylase is limited, because PEP is required to support stress-induced phenylpropanoid biosynthesis and its byproduct lignin, the GABA shunt may provide carbon through GABA catabolism to succinic acid (Shelp, 1999).

The final criteria for GABA being a signaling molecule came with the discovery of glutamate receptor-like genes in plants. Glutamate receptor genes (GLR) have domains in common with the GABA receptors in animals. Mammalian GABA receptors function by modulating CA2+ ion channels in response to stress. Animal mitochondria have an important role in cellular Ca2+ signaling by releasing large quanties of Ca2+ (Babcock, 1997). Plant mitochondria are a likely store of Ca2+ and a possible site of GABA receptors (GLR's). Studies have shown that stress induced release of Ca2+ from maize mitochondria (Subbaiah, 1998). Plant enzymes that catabolize GABA are found in the mitochondria exclusively (Breitkreuz, 1995). The GABA-like receptor gene from Arabidopsis thaliana, GLR2, is reported to regulate the utilization of Ca2+. Plant GLR's contain six signature domains characteristic of animal ionotropic glutamate receptors (iGLRs). These include three transmembrane, one pore forming and two putative glutamate binding domains located in the C-terminal. Arabidopsis GLR's have a high degree of sequence homology to the iGLR's (Lam, 1998). Hydrophobicity analysis and transmembrane analysis models confirm this similarity (Lam, 1998; Chiu et al., 1999).

Overexpressed levels of GAD1 and GAD2 genes in Zea mays L. may result in plants that have higher concentrations of GABA and therefore are more tolerant to diverse stress and are higher yielding. Overexpressed levels of GLR2a, GLR2b, GLR5, GLR6 and GLR7 genes in Z. mays may result in a higher concentration of GABA receptors for signaling stress tolerance.

Overexpression of yeast glutamate decarboxylase in yeast has been demonstrated to increase tolerance to both hydrogen peroxide and diamide (Coleman et al., 2001). Expression of this gene in maize both in a native form and with mitochondrial signal peptide is expected to increase stress tolerance.

Nucleic acid and polypeptide sequences for use in generation of transgenic crop plants having improved properties as described in the above strategy are provided in Table 1.

21. Heat Generation

The inability of maize plants (Zea mays L.) to endure low temperatures and freezing is a major determinant of where the crop is grown. Even in climates considered suitable, crop failure or decreases in yield frequently occur due to cold temperatures. Injury to the plant's cell membrane is the primary site of cold damage (Troyer et al., 1999).

Ice formation is initiated in the intercellular spaces of the plant's cells, because the extracellular fluid has a higher freezing point or lower solute concentration than intracellular fluid. Liquid water moves down the chemical potential gradient from inside the cell to the intercellular spaces. The plasma membrane is damaged due to dehydration causing lysis, segregation of proteins within the membrane (phase transition) and fracture lesions. The cell's cold-induced production of reactive oxygen species contributes to the membrane damage (Makensie and Bowley, 1997). At prolonged exposure to below freezing temperatures, intercellular ice can cause cell rupture (Olien and Smith, 1977).

The natural ability of some plants to generate heat was first reported over 200 years ago by the French naturalist Jean-Baptist de Lamarck upon observing that the European arum lily became warm when flowering. More recent observations conducted on the skunk cabbage (Symplocarpus foetidus) reported that it is able to hold its temperature between 15 and 22 degrees C. when air temperatures are below freezing. It is postulated that the plant converts electrochemical energy from the production of ATP to the production of heat (Seymour, 1997).

Plant mitochondria produce cellular ATP from ADP as electron transfer in the respiratory chain induces the transport of protons across the mitochondrial membrane providing the driving force for oxidative phosphorylation. This electron transport chain is located in the mitochondria's inner membrane and contains four respiratory electron carrier complexes. Electrons are generated from the oxidation of NADH and succinic acid. Each of the four complexes are capable of rapid and reversible oxidation and reduction components and thus acts as an electron carrier. The terminal complex passes electrons to molecular oxygen and this final electron acceptor is reduced to water. The transfer of electrons to molecular oxygen yielding water is catalyzed by oxidases. Plant mitochondria contain two terminal oxidases: cytochrome oxidase located in complex IV and an alternative pathway which branches from the cytochrome pathway at the ubiquinone pool (electron carrier—dissolved quinol in the mitochondria's inner membrane lipid bilayer). This pathway passes electrons to alternative oxidase and reduces molecular oxygen to water in a four electron step. ATP production is not a byproduct of this pathway (Day et al., 1991; Day et al., 1995; Day and Wiskich, 1995, Maia et al., 1998, Rhoads and McIntosh, 1991).

The pathway was first discovered in the arum lily family (Araceae) where heat produced during anthesis attracts pollinators to aromatic compounds in the inflorescence. For a few hours during the two to four day period during the onset of flowering the rate of respiration increases from 20 μL to 100 μL of oxygen consumed per 100 mg fresh weight per hour to 1,000 μL to 1,500 μL of oxygen consumed per 100 mg of fresh tissue per hour (Chivasa et al., 1999; Li el al., 1996; Low and Merida, 1996).

Alternative oxidase was first cloned from the voodoo lily (Sauromatum guttatum). Antibodies were used to isolate a nuclear encoded 42-kDa polypeptide cDNA clone designated AOX1. AOX1 clones have since been isolated from numerous other species (Cruz-Hernandez and Gomez-Lin, 1995; Elthone et al, 1989; Kumar and Soil, 1992; Laloi et al., 1997, Nagano et al., 1995, Scandalios, 1990; Simons et al., 1999).

AOX1 is upregulated by stress including oxidative stress, cold, drought and wounding. AOX1 gene expression responds to harmful active oxygen species such as superoxide, peroxide, or a hydroxyl radical that are generated during periods of biotic and abiotic stress (Allen, 1995; Low and Merida, 1996; Scandalios, 1990; Wagner, 1995; Wagner and Moore, 1997; Yukiok et al., 1998). Alternative oxidase is cyanide resistant with a non-heme binuclear iron center (cyanide is produced during wounding and shuts down the cytochrome (heme) pathway (Albury et al., 1996; Siedowet et al., 1995)). The level of alternative oxidase present in the mitochondria determines the maximum possible partitioning of electrons to the alternative pathway (Hiser et al., 1996; Sedow and Ulmbach, 1995; Siedow et al., 1995; Vanlerberghe and McIntosh, 1999). Alternative oxidase is controlled to its most active or reduced state by the levels of reduced ubiquinone and by the oxidation of TCA cycle substrates isocitrate and malate (Vanlerberghe et al, 1995; Vanlergerghe and McIntosh, 1999).

Transgenic tobacco and potato with overexpressing AOX1 showed increased alternative oxidase activity in the mitochondria when the cytochrome pathway was inhibited by cyanide (Hiser et al., 1996; Vanlerberghe et al., 1994). Overexpressed levels of AOX1 in transgenic tobacco lowered the reactive oxygen species that are harmful to cells. Antisense suppression of AOX1 resulted in significantly higher levels of ROS than in wild type, whereas overexpression lowered the levels of reactive oxygen species (Maxwell et al., 1990). It is expected that overexpression of alternative oxidase in Z. mays will lead to increased tolerance of the plant to oxidative, drought and cold stresses.

In mammals, mitochondrial uncoupling proteins (UCP) dissipate the proton gradient formed through respiration without ATP synthesis. Mammals are able to maintain their body temperatures in cold environments as energy is readily converted to heat (Seymour, 1997). Nuclear encoded mitochondrial uncoupling proteins contain six transmembrane domains and are mainly expressed in the inner mitochondrial membrane of brown adipose tissue in mammals. A cDNA encoded peptide, identified using a yeast two hybrid protein-interaction trap was used to isolate a full-length cDNA from a potato (S. tuberosum) cDNA library that is 44% homologus to the mammalian UCP1 protein at the amino acid sequence level. The StUCP has a molecular mass of 32K and consists of three 100-residue domains. Each domain contains two transmembrane regions and a mitochondrial transporter motif. The protein was found to be expressed in leaves and tubers at low levels, at moderate levels in stems and fruit and at high levels in roots and flowers (Laloi et al., 1997). A similar protein was cloned from Arabidopsis thaliana termed AtPUMP (H) and a novel isoform cDNA cloned in A. thaliana (AtUCP2; Watanabe et al., 1999).

It was hypothesized that fatty acid mediated anion transport across the inner mitochondrial membrane was responsible for proton uncoupling. According to this hypothesis, the uncoupling protein is a pure anion porter and does not transport protons. It is designed to enable fatty acids to behave as cycling protonophores and anions are transported through the fatty acid domain of UCP (Garlid et al, 1996). A second report concluded that the UCP1 was able to transport protons in the absence of fatty acids (Gonzalez-Barroso et al., 1998). In another study, linoleic acid induced the activity of tomato fruit (L. esculentum). Tomato is a climacteric fruit known to express both PUMP and AOX proteins (Jezek et al., 1996; Jezek et al., 1997). An increase in respiration was observed from 151 to 411 nmol of oxygen atom/min/mg of mitochondrial protein. This increase in respiration was linked to PUMP activation. Exponential flux-voltage characteristics were studied and it was concluded that PUMP can efficiently divert energy from oxidative phosphorylation (Jarmuszkiewicz et al., 1998).

Expression of the S. tuberosum UCP protein was strongly induced by cold treatment after 1 to 2 days exposure to 4 degrees C. and is postulated to be important in plant thermogenesis (Laloi et al., 1997; Vercesi et al., 1995). It is expected that overexpression of a PUMP or UCP-like protein in Z. mays will increase plant tolerance to cold stress.

Higher plant mitochondria have been reported to contain an alternative terminal oxidase in the electron transport chain that is recognized to be a part of the plant's ability to regulate its energy/carbon balance in response to a stressful environment (McIntosh, 1994; Vanlerberghe et al., 1995). Alternative oxidase genes that have been sequenced encode a highly similar protein with two alpha-helical membrane-spanning regions in the center and a possible alpha-helix that is surface exposed. There are N- and C-terminal hydrophilic regions that are exposed to the mitochondrial matrix (Rhoads and McIntosh, 1991; Siedow et al., 1992). It is postulated that the conserved cysteine residues in the N-terminal region form a labile disulfide-bridge. The oxidation (less active form) or reduction (most active form) of this bridge controls alternative oxidase activity. Sequence data suggests that that the protein contains four potential small C-terminal alpha-helices and two contain a totally conserved Glu-X-X-His motif with a binuclear iron center (Albury et al., 1998; Saisho et al., 1997; Siedow et al., 1995). Using PCR analysis, three isoforms of the alternative oxidase gene have been identified in soybean and two or more copies of the gene have been identified in tobacco (McCabe et al., 1998; Whelan et al., 1996). The isoforms are differentially expressed during plant development; i.e., AOX1 is expressed cotyledons and leaves, AOX3 is expressed in the leaves and AOX2 is expressed in the aging plant. The structure of the S. guttatum AOX1 gene (Finnegan et al., 1997 and Rhoads and McIntosh, 1993) and its promoter (McKersie and Bowley, 1997) have been described. Salicylic acid-responsive elements are located in the region-525 to -568. Salicylic acid triggers increased expression of alternative oxidase (Raskin et al., 1989; Rhoads and McIntosh, 1992) and is a by-product of plant's response to stress. The expression of the AOX1 gene also responds to reactive oxygen species (ROS) as signals are sent to the nucleus to activate AOX1 (Allen, 1995; Low and Merida, 1996; McIntosh, 1994; Scandalios, 1990; Simons et al., 1999; Wagner, 1995; Wagner and Moore, 1997). Hydrogen peroxide is reported to be an intermediate in this signalling process (McIntosh et al., 1993).

The actual electron flux to the alternative pathway is controlled via it's redox state (reduced, most active). The ubiquinone pool is dependent upon the activity an NADH-dehydrogenase and succinate-dehydrogenase supplying electrons for ubiquinone reduction and the terminal oxidases' activity. Q electrode experiments have determined that the level of reduced ubiquinone determines alternative oxidase activity (Ojajanegara et al., 1999; Moore and Siedow, 1991; Ribas-Carbo et al, 1995; Siedow and Bickett, 1981). Therefore, the high levels of TCA cycle electron carrier byproducts determine the active alternative pathway, as the cytochrome pathway shuts down with high levels of reduced ubiquinone. In this manner, the alternative oxidase contributes to a higher level of carbon skeletons supplied for the plant's use during times of stress by favoring electron transport and oxidation of the pyridine nucleotide pool of the TCA cycle (Crawford et al., 1989; Issakidis et al., 1994; Scheibe, 1991). Transgenic tobacco containing incresed levels of alternative oxidase protein were used to determine the mechanisms regulating AOX1 activity. Reduction of AOX1 to its most active or reduced form was generated by the oxidaton of TCA cycle substrates isocitrate and malate (isocitrate dehydrogenase and malate dehydrogenase) (Umbach and Siedow, 1993).

Transgenic tobacco plants containing sense and antisense DNA constructs were used to produce plants with increased and decreased levels of AOX1. Suspension cells, grown in heterotrophic batch culture confirmed that genetic alteration of the level of AOX1 was sufficient to alter the level of electron transport through the alternative pathway (Vanlerberghe and McIntosh, 1999). The alternative pathway may function as a mechanism to decrease the formation of reactive oxygen species (ROS) produced during respiratory electron transport while the plant is experiencing abiotic or biotic stress. The ROS-sensitive probe 2′,7′-dichlorofluorescein diacetate proved that the overexpression of AOX1 resulted in cells with lower reactive oxygen species as compared to wild-type (Maxwell et al., 1999).

It is expected that expression of alternative oxidase-type proteins as well as UCP-like proteins in transgenic plants will increase the tolerance of the plants to chilling and freezing conditions, as well as increase resistance and oxidative stress.

Nucleic acid and polypeptide sequences for use in generation of transgenic crop plants having improved properties as described in the above strategy are provided in Table 1.

22. Heat Tolerance

Heat stress can reduce corn yields. Shaw (1983) reported that each 1° C. increase in temperature over 25° C. results in a 3 to 4% decrease in grain yield. The deleterious effects of heat on yield may be due to a reduction in the rate and duration of endosperm cell division in the early phases of endosperm development (Jones et al., 1985), a reduction in kernel growth rates during the fill period or a reduction in the fill period (Wilhelm et al., 1999). Long term analysis of weather patterns and corn yield has suggested that reductions in corn yield due to heat stress are most severe when high temperatures occur in conjunction with low levels of rainfall (Carlson, 1990). Coupled with predicted climate changes and increases in the frequency of climatic extremes (Easterling et al., 2000), heat tolerance can be expected to increase in importance as a factor in determining corn yield.

Overexpression of proteins shown to improve heat tolerance in other plants may increase heat tolerance in corn and lead to an improvement in corn yield. These effects may only be manifest in years where temperatures are high and may best be used in conjunction with other yield enhancing genes such as those that confer tolerance to low soil moisture levels.

Screening for improved germination rate at 32° C. in A. thaliana plants overexpressing endogenous transcription factors has identified two genes encoding transcription factors, G682 and G464, which increase heat tolerance of transgenic plants relative to wild type controls. G682 (GenBank Accession No. CAB80915) encodes a 77 amino acid protein with a single C-terminal myb domain. G464 is derived from Indole Acetic Acid Induced 2 (IAAI2) (GenBank Accession No. Q38830), but G464 encodes a C-terminal truncation of IAAI2 due to a 4 nucleotide deletion in the sequence. Neither of these genes appears to have a monocot orthologue. However, sequences were identified for two corn IAA/AUX family members.

Overexpression of HSF1 and HSF3, transcription factors that regulate expression of heat shock genes in A. thaliana, has also been shown to improve thermotolerance in A. thaliana (Lee et al., 1995; Prandl et al., 1998). In both cases, overexpression of the heat shock factors is associated with constitutive expression of heat shock proteins. Tomato HSF A1 and A2 can functionally replace yeast HSF1 (Boscheinen et al., 1997), suggesting that the yeast protein may be able to function in plants.

Heat shock proteins act to ameliorate problems caused by protein misfolding and aggregation at higher temperatures (Saibil, 2000). Modification of HSP levels has been reported to alter thermotolerance in several different plant systems. Overexpression of HSP101 has been report to improve thermotolerance in A. thaliana (Queitsch et al., 2000). Likewise overexpression of HSP17.7 has been reported to improve thermotolerance in carrot cells (Malik et al., 1999). Antisense inhibition of HSP70 has also been reported to decrease thermotolerance in A. thaliana (Lee and Schoffl, 1995). In these latter two cases, expression of other heat shock genes was modified by altering the expression of the target gene suggesting a complex relationship between heat shock protein levels and their synthesis.

HSP 101 or clpB proteins are highly conserved and have been suggested to rescue aggregated proteins (Glover and Lundquist, 1998). HSP101 has been cloned from maize (Nieto-Sotelo et al., 1999) and shown to complement a yeast HSP104 deletion (Nieto-Sotelo et al, 2000). Corn HSP101 appears to be somewhat more closely related to bacterial clpB than HSP104. This suggests that overexpression of microbial HSP101 orthologues may also improve corn thermotolerance.

Nucleic acid and polypeptide sequences for use in generation of transgenic crop plants having improved properties as described in the above strategy are provided in Table 1.

23. Heterotrimeric G Proteins

Heterotrimeric G-proteins have been suggested to play a role in a number of different plant environmental and developmental responses including gibberillic acid (GA) signaling, stomatal movement and disease resistance responses. Altering the activity of these proteins may improve corn productivity by increasing tolerance to high density planting, altering plant height, altering water relations parameters or increasing resistance to pathogens.

The isolation of a rice dwarf mutation which is less responsive to GA and contains a mutation in the alpha subunit of a heterotrimeric G protein suggests that these proteins are involved in GA signaling (Fujisawa et al., 1999; Ashikari et al., 1999; Ueguchi-Tanaka et al., 2000). This conclusion is consistent with studies of GA induced alpha-amylase expression in barley aleurone (Jones et al., 1998).

Studies have suggested that heterotrimeric G-proteins can activate inward rectifying K+ channels that are involved in guard cell movement (Li and Assmann, 1993; Wu and Assmann, 1994). The effects of G protein on guard cell movement may be indirect, as they have been shown to bind to and modulate the polymeric state of tubulin (Wang et al., 1990; Roychowdhury and Rasenick, 1994; Ravindra et al., 1996). Alterations in the cellular tubulin pool have been shown to be required for stomatal closure (Huang et al., 2000; Marcus et al., 2001).

Several different subunits from heterotrimeric G proteins have been cloned from plants, including two distinct types of G-alpha subunit (Ma et al., 1990; Lee and Assman, 1999). Plant G-alpha (Weiss et al., 1993), G-beta (Peskan and Oelmulle, 2000) and G-gamma (Mason and Botella, 2000) subunits have been reported to be ubiquitously expressed with the highest levels found in younger tissues. Tobacco alpha and beta subunits have also been reported to be induced by auxin (Kaydamov et al., 2000).

Two potential G protein coupled receptors have been identified in plants. GCR1 is most closely related to D. discoideum cAMP receptors. Anti-sense suppression of GCR1 leads to decreased root and shoot sensitivity to cytokinins (Plakidou-Dymock et al., 1998). A second putative receptor gene, the barley Mlo gene, encodes a protein that confers powdery mildew resistance and is predicted to contain seven transmembrane domains (Buschges et al., 1997). Mlo defines a large gene family in A. thatiana (Devoto et al., 1999). However, neither GCR1 nor Mlo has been shown to directly couple with heterotrimeric G proteins. A membrane bound protein that is capable of interacting with G protein alpha subunits has been identified in maize microsomes, but not cloned (Wise et al., 1994).

The sequence of an A. thaliana G-protein gamma subunit has recently been reported (Mason et al., 2000). G gamma subunits are typically modified by both prenylation and palmitoylation at conserved cysteine residues located near the C-terminus (Hirschman and Jenness, 1999). This modification is required for membrane localization of the G beta-gamma dimer and assembly of a functional heterotrimeric G protein. The soy and corn G protein gamma subunit sequences of the present invention have conserved cysteine residues in their C-termini that likely are involved in membrane localization of the G beta-gamma dimer and assembly of a functional heterotrimeric G protein plants. Construction of C-terminal mutants lacking these cysteine residues either by truncation or point mutation creates a protein that acts as a dominant negative effector of the activity of heterotrimeric G-proteins or at least the G protein beta-gamma subunits. It is expected that expression of truncated or mutated G proteins may result in altered plant height or growth rates.

Nucleic acid and polypeptide sequences for use in generation of transgenic crop plants having improved properties as described in the above strategy are provided in Table 1.

24. Homologous Recombination

Transgene introgression by repeated backcrossing is limited by the rate of homologous recombination. Enhancing the rate of homologous recombination is expected to speed the introgression process and improve the quality of the resulting plant. The speed of introgression is determined by balancing the number of generations the process will take against the cost of genotyping and phenotyping exponentially increasing numbers of individuals. Increasing the rate of homologous recombination is expected to reduce the number of individuals that need to be examined to identify a plant containing the transgene and only a small segment of DNA from the donor parent. Thus, lines displaying enhanced recombination rates can be used to: 1) speed the introgression process by reducing the number of generations needed to identify a line with only the desired introgressed segment; 2) reduce the cost of the process by reducing the number of individuals screened; and 3) improve the quality of the process by increasing the frequency with which lines with only very small introgressed segments are identified.

Increased rates of homologous recombination can also be expected to enhance the conventional breeding process. Elevated recombination rates will allow rare recombinants between closely linked genes in phase repulsion to be identified more easily. This is likely to result in a more rapid rate of plant enhancement and may also allow the more extensive use of exotic germplasm as donor sources.

Increased homologous recombination rates may also improve the usefulness of gene targeting in plants or other species.

Genes involved in homologous recombination in bacterial systems have been shown to enhance recombination rates when overexpressed in plants. E. coli resolvase, ruvC, was shown to increase the rate of somatic homologous recombination in transgenic tobacco (Shalev et al., 1999). Homologous recombination in mammalian cells has been shown to be mediated by sister chromatid exchange (Sonoda et al., 1999). Expresssion of E. coli recA has been shown to increase the frequency of sister chromatid exchange and intrachromosomal recombination in transgenic tobacco (Reiss et al., 1996, 2000).

Recombination in eukaryotes is mediated by a group of proteins refered to as the RAD52 epistasis group. These genes were first identified in Saccharomyces cerevisiae and consist of RAD50, RAD51, RAD52, RAD54, RAD55, RAD57, RAD59, RDH54/TID1, MRE11, and XRS2 (Sung et al., 2000). The RAD52 group of proteins are well conserved across eukaryotes. Homologous recombination during mieosis is initiated by the formation of double stranded breaks. Breaks have been shown to cluster in intergenic regions containing transcriptional promoters in yeast (Wu and Lichten, 1994; Baudat and Nicolas, 1997). Spo11 has been suggested to catalyze double-stranded break formation during mieosis (Keeney et al, 1997). Double-stranded breaks in DNA are bound by a set of annealing exonucleases that includes recBCD (Arnold and Kowalczykowski, 2000), RecE/RecT and Red alpha/Red beta nuclease in E. coli (Muyrers et al, 2000). In E. coli these enzyme complexes convert the double-stranded break into a coated single-stranded region due to their 3′ to 5′ exonuclease activity and promote recA loading onto the single-stranded DNA and D-loop formation with homologous double-stranded DNA.

The Mre11-Rad50-Xrs2 protein complex has been suggested to convert double-stranded DNA breaks into tailed single-stranded breaks in yeast (Bressan et al., 1999). RAD52 binds directly to single stranded and double-stranded breaks in DNA and promotes homologeous recombination (Van Dyck et al., 1999; Parsons et al., 2000). In yeast the activity of RAD52 is stimulated by the presence of replication protein A (Sugiyama et al., 1998) as well as heterodimers of RAD55/RAD57 (Gasior et al., 1998). RAD51 is orthologous to bacterial RecA (Shinohara and Ogawa, 1999). RecA and RAD51 polymerize on single-stranded DNA and promote D-loop formation. RAD51 appears to differ from RecA in that it prefers tailed duplex DNA over single stranded DNA and can promote strand invasion from either the 5′ or 3′ direction (Mazin et al., 2000). RAD51 activity is also promoted by RAD54, a member of the SNF2/SWI2 family of DNA binding proteins. Double stranded DNA-activated ATPase activity in RDH54 generates unconstrained negative and positive supercoils in DNA. Efficient D-loop formation occurs with even topologically relaxed DNA, suggesting that via specific protein-protein interactions, the negative supercoils produced by RAD54 are used by RAD51 for making DNA joints (Petukhova et al., 2000).

Resolvases belonging to four distinct structural classes have been identified in prokaryotes (Aravind et al., 2000). RuvC-like proteins are found in most bacteria and include the mitochondrial resolvase Cce1 from Saccharomyces cerevisiae and its Schizosaccharomyces pombe ortholog (Ezekiel and Zassenhaus, 1993; Oram et al., 1998). The group of genes defined by E. coli YqgF is also included in the family of RuvC-like proteins. RuvC functions as a Holliday junction resolvase, by selectively nicking Holliday junctions to produce symmetrical incisions in the branched DNA structure. It typically acts in conjunction with two additional proteins, RuvA and RuvB. The second family of resolvases was identified following the cloning and biochemical characterization of an aracheal resolvase from Pyrococcus furiosus (Komori et al., 1999). Members of this family share structural similarity with RecB and □ exonuclease. The third family of resolvases in typified by bacteriophage T4 endonuclease VII (Mizuuchi et al,. 1983). The final resolvase family is characterized by the E. coli RusA protein (Chan et al., 1997). No eukaryotic nuclear resolvases have been identified to date.

It is expected that increaseing the rate of branch migration or resolution increases the available concentration of RAD51 or other proteins required for homologous recombination.

Homologous recombination rates in plants may be stimulated by: 1) Increasing the rate of double stranded break formation; or 2) Increasing the rate of entry into the homologous recombination pathway versus the ku-dependent non-homologous end-joining repair pathway. Double-stranded break formation may be stimulated by overexpression of Spo11. Stimulating the homologous recombination pathway may be achived by increasing the activity or concentration of proteins involved in the initial steps of the homologous recombination pathway such as RAD52, RAD51 or RAD54.

Nucleic acid and polypeptide sequences for use in generation of transgenic crop plants having improved properties as described in the above strategy are provided in Table 1.

25. HSP90

Hsp90 proteins have been shown to play a role in protecting organisms from heat shock, possibly by accelerating the rate of reactivation of a select group of proteins involved in signal transduction. This has been demonstrated in A. thaliana where a mutant that fails to accumulate HSP90 has been shown to be thermosensitive and this sensitivity is reduced by overexpression of HSP90 (Ludwig-Muller et al., 2000). The protein is essential in all eukaryotes studied to date ((Nathan et al., 1997; 1999). However, little is know about the effects of overexpression of Hsp90 on thermotolerance. Overexpression of an HSP90-class chaperone or its regulators may result in increased thermotolerance. It is expected that impairment of the HSP90 pathway will increase thermotolerance as HSP90 has been reported to act as a repressor of the heat shock response in yeast (Duina et al., 1998). Likewise, alteration of HSP90 function may alter salt tolerance (Imai and Yahara, 2000) in corn.

HSP90 has also been reported to act as an evolutionary “capacitor”, buffering organisms from the effects of allelic variation in its regulatory substrates under non-stress conditions and exposing these variations for use in selection under stress conditions (Rutherford and Lindquist, 1998). Supporting this notion, HSP90 has been demonstrated to bind to a number of important signaling molecules including receptor and non-receptor tyrosine kinases, glucocorticoid-type transcription factors and various elements of MAP kinase signaling cascades and the cell cycle. This suggests that alteration of HSP90 function may have effects on plant performance other than heat tolerance. In particular, reducing HSP90 function may uncover phenotypic variation that can be used for selection in conventional breeding programs.

Corn has been reported to contain two Hsp90 family members, Hsp81 and Hsp82. Hsp81 is only mildly heat inducible in leaf tissue, but is strongly expressed in the absence of heat shock during the pre-meiotic and meiotic prophase stages of pollen development and in embryos, as well as in heat-shocked embryos and tassels. Hsp82 shows strong heat inducibility at heat-shock temperatures (37-42 degrees C.) and in heat shocked embryos and tassels but is only weakly expressed in the absence of heat shock (Marrs et al., 1993).

Hsp90 functions in concert with Hsp70, Sti1/Hop and p23 in an ATP-driven cycle that refolds or protects proteins (Young and Hartl, 2000). This interaction appears to be facilitated by tetratricopeptide repeats in HSP90 ligands (Young et al., 1998).

Hsp90 plays a role in intracellular transport of many of its ligands, a role that is likely to require Hsp90 cofactors FKBP52 or cdc37 (Pratt et al, 1999). Overexpression of either of these classes of proteins is also likely to alter Hsp90 function

Two yeast genes, Cns1 and Hch1 have also been shown to function in concert with hsp90 although their biochemical roles are not well understood (Nathan et al, 1999). Mammalian hsp90 has been shown to function in yeast, making it likely that other components of this signal transduction cascade are conserved as well (Picard et al, 1990).

CHIP (co-chaperone) has been identified as an additional HSP90 regulatory protein. Overexpression of CHIP was shown to alter the fate of the HSP90 substrate glucocorcoid receptor towards ubquitinination (Connell et al., 2001). Overexpression of CHIP in corn may lead to an alteration the level of activity associated with HSP90 substrates and either an increase in thermotolerace or an uncovering of additional variation that may be used in a breeding program.

Nucleic acid and polypeptide sequences for use in generation of transgenic crop plants having improved properties as described in the above strategy are provided in Table 1.

26. Invertase

Kernel abortion under stress conditions has an adverse effect on yield. Zinselmeier et al. (1999) show that when the sugar stream is disrupted, as under low water potential, young embryos abort. The block appears to be at the first step in sucrose utilization, catalyzed by invertase. It is expected that improvement of the kinetic efficiency of invertase under low water conditions will lead to decreased kernel abortion, thereby increasing grain yield. Invertases from different species are tested to identify an invertase with better kinetics under low water conditions. Gene sources may include corn, sorghum, rice and Coix. The source of invertases need not be limited to higher plants (Gerday et al., 2000) and may include microbes, fungi, algae, and insect. This approach to yield increase requires both an invertase and a sink/pedicel-specific promoter. Transgenic, constitutive expression of invertase in tomato (Dickinson et al. 1991) and tobacco (von Schaewen et al, 1990) resulted in detrimental phenotypes. Corn invertase sequences are used to isolate native promoters to drive the heterologous genes. Alternatively, a glutamine synthase 1-2 (pedicel-specific) promoter which drives pedicel specific expression may be used. Other seed specific promoters such as a cytokinin oxidase (early endosperm) promoter or the bet1 promoter, specific to the basal endosperm transfer layer, are useful.

The effect of expression of invertase is expected to be production of a greater number of mature kernels per ear under drought conditions. A drought will be imposed several days before and after pollination followed by well-watered conditions for the remaining life of the plant. A kernel count per ear will be made after kernel maturation.

Nucleic acid and polypeptide sequences for use in generation of transgenic crop plants having improved properties as described in the above strategy are provided in Table 1.

27. Jasmonate

Jasmonates have been identified as natural compounds occurring throughout the plant kingdom which affect many developmental processes such as senescence, germination, tuber formation, pollen fertility, fruit ripening, insect and disease resistance, drought, cold and salinity tolerance (Creelman, 1997). Jasmonates are linolenic acid (18:3) derived plant growth regulators or phytohormones. The biosynthesis pathway of jasmonates is known and many of it's enzymes have been cloned or purified (Farmer, 1998). The natural jasmonates are (−)-jasmonic acid (JA), its stereoisomer, (+)-7-isoJA and the volatile methyl ester of (−)-JA The biosynthesis of jasmonates begins with a conversion of linolenic acid to 13-hydroperoxylinolenic acid by lipoxygenase (LOX). Plant membranes contain a high concentration of linoleic acid and it has been postulated that increased levels of JA may result from the release of linolenic acid from phospholipids catalyzed by phospholipase. Phospholipases prefer oxygenated fatty acids before further metabolism (Banas, 1992). The lipoxygenase (LOX) pathway oxidizes alpha-linolenic acid and releases it from membrane bound phospholipids for the synthesis of JA. AtLOX1 and AtLOX2 have been identified in Arabidopsis thaliana (Melan, 1993). AtLOX2 is localized in the chloroplast and is required for the wound-induced synthesis of jasmonates in transgenic Arabidopsis leaves (Bell, 1995).

Linolenic acid is a substrate for allene oxide synthase (CYP74/AOS) which catalyses the formation of 12-oxo-phytodienic acid (JA precursor) (Laudert, 1996). Wounded transgenic tobacco and Arabidopsis thaliana plants overexpressing CYP74 accumulated significantly more JA than wounded, untransformed control plants (Laubert, 2000; Kubigsteltig, 1999). Allene oxide cyclase converts the unstable allene epioxide precursor to 12-oxo-phytodienoic acid (OPDA). Jasmonic acid is synthesized from OPDA by reduction of the double bond of OPDA by OPDA-reductase (OPR). Isoenzymes OPR1, OPR2 were cloned from A. thaliana (Biegen, 1998). A third isoenzyme OPR3 has been recently identified as a brassinosteroid-upregulated gene that is responsive to a variety of stimuli (Mussig, 2000). A recent study identified OPR3 as the isoenzyme involved in JA biosynthesis (Schaller, 2000). Additionally, OPR3 has been recently confirmed as being a potential link between brassinosteroid-action and jasmonic acid synthesis as OPR3 expression increased with the application of brassinolide (Mussig, 2000).

The natural accumulation of JA varies as a function of tissue and cell type, developmental stage and in response to environmental stimuli. Higher levels are found in zones of cell division such as floral and reproductive structures compared to zones of cell elongation in more mature tissue, such as the stem. Jasmonate levels are rapidly and transiently increased by mechanical perturbations such as wind, touch or wounding. Levels also increase in response to turgor reduction (Creelan, 1995). Foliar application of methyl-JA (MJ) reduces both glycolipid and phospholidid content of strawberry leaves as compared to nonstressed controls. MJ was effective in lessening the decline in the unsaturated:saturated fatty acid ratio and in maintaining a higher percentage of unsaturated membrane lipids under water stress conditions in both glycolipids and phospholipids. Transpiration levels were reduced, peroxidation levels were reduced and ascorbic acid levels remained constant in treated plants compared to controls (Wang 1999). Reduction of chilling injury and osmotic streses by JA has been reported in rice, peanut seedlings and cucumbers (Lee, 1996; Pan, 1995; Wang, 1994). Exogenous treatment of barley seedlings with JA diminished the inhibitory effect of high salt concentration on growth and photosynthesis (Tsonev, 1997).

Overexpressed levels of the CYP74, OPR3, and atLOX2 genes in Zea mays L. is expected to result in plants with higher endogenous levels of jasmonates and an improved tolerance to abiotic and biotic stresses.

Jasmonic acid signaling has been identified as playing a role in reducing salicylic acid (SA) concentrations and abolishing ozone-induced, SA dependent cell death. Ozone accumulates naturally in the extracellular spaces and spontaneously dissociates generating reactive oxygen species, thereby damaging the cell during exposure to stress. Ozone reacts primarily with the plasma membrane and causes alterations in lipid composition that produce a higher concentration of linoleic acid. High levels of exogenously applied JA decreased ozone-induced concentrations of hydrogen peroxide in tobacco (Rao, 2000). The oxidative burst that accompanies a plant's response to stress may induce JA by producing oxidized fatty acids. JA in turn induces genes involved in defense responses. The role of reactive oxygen species and JA as mediators in response to pathogen attack and wounding is well documented (Low, 1996; Seo, 1997; Wasternack, 1997).

The production of ethylene in mesocarp tissue of winter squash is induced by mechanical wounding. A cDNA fragment WSACS2 was isolated from the wounded tissue. The sequence of WSACS2 was highly similar to the wound-inducible ACC synthase that catalyzes ethylene production. Methyl jasmonate stimulated WSACS2 synthesis in wounded tissue (Watanabe, 1998). This is consistent with reports that JA and ethylene synergistically activate defense genes (Xu, 1994; Schweizer 1997; Watanabe 1998).

It is believed that the jasmonate pathway is involved in plant responses to biotic stresses. The gene PDF1.2 is involved in fungal resistance in A. thaliana (Staswick, 1998) PDF expression is induced by JA, ethylene and oxygen radical generators such as paraquat and rose bengal (Penninckx, 1998

Leaf tissues of barley responded to JA application by inducing gene expression of JIPs or jasmonate-induced proteins (JIPs). JIP60 was identified as a ribosome-inactivating protein present in stressed leaf tissue. JIP's down regulate genes that encode “late embryogenesis” (LEA) proteins that are thought to mitigate changes in the leaf due to drought stress (Gorschen, 1997; Reinbothe, 1997).

The atEXT1 gene, encoding an extension protein, is also induced by MJ in response to wounding. Hydroxyproline-rich glycoproteins (extensins) are abundant in plant cell walls. It is believed that extensins are synthesised in response to wounding or pathogen attack and their function is to reinforce the cell wall by intermolecular cross-linking of tyrosines on adjacent monomers. The atEXT1 gene is normally expressed in the root, but not in the leaf. There are promoter motifs that are activated by salicylic acid, abscisic acid and methyl jasmonate during leaf wounding stress, resulting in the expression of extensins (Merkouropoulos, 1999).

COI1 has been recently identified as a gene required for jasmonate response in Arabidopsis. The COI1 protein contains 16 leucine-rich repeats (LLRs) and an F-box motif similar to other signaling pathway proteins. The F-box proteins function as receptors that selectively recruit repressor proteins required for the removal of substrates by ubiquitination. This study speculates that COI1 recruits regulators of defense response for this function (Xie, 1998).

Overexpression of jasmonate signaling and induced genes (COI1, atEXT1, JIP60, PDF1.2, WSACS2) in Z. mays is expected to result in plants with higher levels of these proteins during periods of abiotic and biotic stress resulting in greater stress tolerance.

Nucleic acid and polypeptide sequences for use in generation of transgenic crop plants having improved properties as described in the above strategy are provided in Table 1.

28. Nitric Oxide

Biochemical, physiological and molecular experiments suggest that nitric oxide (NO) may play a role in agronomically important traits in corn such as kernel maturation, leaf sensecence, disease resistance, root growth or photomorphogenic responses. Removal of endogenous NO by overexpression of NO detoxifying enzymes can be expected to elucidate the role(s) NO plays in the expression of agronomic traits in corn. Overexpression of enzymes activated by NO may also be expected to yield similar results. In both cases agronomic traits may also be improved by either a reduction in nitrosative stress or an amplification of NO signaling.

Nitric oxide has been shown to play a major role as a signaling molecule in animal systems (Ignarro, 2000). In contrast, many different roles for NO in plant sigaling have been suggested, but the proposed plant signaling pathways are not well understood (Wojtaszek, 2000). For example, nitric oxide has been reported to mediate photomorphogenic responses in wheat, lettuce, potato and A. thaliana (Beligni and Lamattina, 2000), promote root elongation in corn (Gouvea, 1997), and to promote ripening in strawberry and avocado (Leshem and Pinchasov, 2000). Involvement of NO in the tobacco defense response is perhaps the best-documented role played by nitric oxide in plant signaling (Klessig et al., 2000; Foissner et al., 2000).

Plants have been reported to contain nitric oxide synthase (Barroso et al., 1999), based on immunological cross reactivity with nitric oxide synthase (NOS) from other species (Lo et al., 2000). Some alternative NO generating proteins also appear to exist in plants. A. thaliana contains a protein (CAB51217) that is related to the br-1, a snail nitric oxide synthase that does not share homology with mammalian NOS (Huang et al., 1997). Corn nitric reductase has also been reported to generate NO (Yamasaki and Sakihama, 2000).

Flavohemoglobins, chimeric flavohemoproteins composed of a single polypeptide having both a single heme and FAD (Poole and Hughes, 2000), have been reported to provide protection from nitrosative stress in yeast (Liu et al., 2000) and E. coli (Membrillo-Hernandez et al., 1999). Overexpression of proteins of this class can be expected to provide protection from NO when expressed in specific subcellular compartments and also to quench NO signaling pathways.

It is expected that alteration of expression of genes involved in NO signaling pathways may affect kernel maturation, thereby contributing to enhanced yield, contribute to increased disease resistance, increase root growth, thereby enhancing water stress resistance, or effect photomorphogenic responses leading to, for example, reduced shade avoidance and an increase of yield by use of increased planting densities.

Nucleic acid and polypeptide sequences for use in generation of transgenic crop plants having improved properties as described in the above strategy are provided in Table 1.

29. Photomorphogenic Responses

Transcriptional profiling was used to identify a gene, nonphototropic hypocotyl 1 (NPH1), which encodes a blue light receptor and is involved in a plant's photomorphogenic response. In Arabidopsis, NPH1 acts as classic Pr-Pfr form of phytochrome by autophosphorylating and de-phosphorylating. In Pfr form (phosphorylated), NPH1 is capable of phosphorylating down-stream signal transduction components, such as NPH2, 3, 4 and auxin response genes. NPH1 is also known to respond to light by reducing the elongation rate of the stem and other organs.

The cDNA of NPH1 was isolated from maize. It is expected that light insensitive overexpression of NPH1 in maize will improve plant architecture and increase hybrid vigor. Overexpression may also produce plants that have improved light perception.

Current studies have shown that NPH1 contains the LOV1, and LOV2 domains and a Ser/Thr protein kinase domain at its C-terminal. The NPH1 kinase domain contains the 11 sequence motifs typical of protein kinases and falls into the AGC (PVPK1) family of Ser/Thr protein kinase within the protein kinase C group. Among the 11 protein kinase sub-domains, sub-domain VIII appears to play a major role in the recognition of peptide substrates. Many protein kinases are known to be activated by phosphorylation of residues in the activation loop between sub-domain VII and VIII. The group C protein kinases tend to phosphorylate substrates at serine/thereonine residues lying very close to the argunine and lysine residues. Using site directed mutagenesis, three mutant NPH1 genes were made by changing serine residues at 849 and/or 851 into aspartic acid residues (Mut1-NPH1, Mut2-NPH1 and Mut1Mut2-NPH1, respectively).

Nucleic acid and polypeptide sequences for use in generation of transgenic crop plants having improved properties as described in the above strategy are provided in Table 1.

30. Phosphate Uptake

Phosphorus makes up about 0.2% of a plant's dry weight and is a key macronutrient for the production of nucleic acids, phospholipids and ATP. Phosphate is also involved in controlling key enzyme reactions and metabolic pathway regulation. Total phosphorous in the soil is usually high but more than 80% of soil phosphorous is present in unavailable forms for plant uptake, because it is adsorbed to soil particles, or held in inorganic or organic forms. Inorganic phosphate interacts with other elements such as aluminum and iron in acid soils, and calcium and magnesium in alkaline soils. The majority of applied fertilizer phosphorous may be fixed in the soil, forcing farmers to apply many times more than is necessary for crop production, leading to phosphorous run off from agricultural fields that is deleterious to ecosystems. The available phosphorous is taken up by the plant by diffusion. The rate of diffusion is slow which creates a zone around the root that is phosphorous depleted (Holford, 1997).

Plants have consequently developed highly specialized mechanisms in response to consistently low levels of available phosphate in the rhizosphere. Responses for enhanced phosphate uptake include secretion of organic acids and phosphatases into the rhizosphere, and the activation of induced novel genes, phosphate transporters, RNases and apyrases (Raghothama, 1999).

An Arabidopsis thaliana mutant lacking a phosphate starvation induced (psi) phosphatase has been cloned and represents evidence that plant roots excrete phosphatases in response to inorganic phosphate starvation (Trull and Deikman, 1998). Phosphate starvation induced phosphatases are acid phosphatases that release phosphate from forms bound in organic matter and the phosphate ester pool of the plant (Huang, 2000). An Arabidopsis psi gene (AtACP5) was cloned and its activity is highly induced under phosphate limiting conditions. AtACP5 was purified from plants starved of phosphate for 10 days (Del Pozo et al., 1999). AtACP5 is a member of a class of dinuclear metallo purple phosphatases (Doi, 1998), which are responsible for the scavenging of active oxygen species. AtACP5 is also induced by stimuli such as ABA, high salt and H2O2. Purple phosphatases have been found in both the aerial parts and roots of the plant (Haran, 2000). It is expected that overexpression of AtACP5 in Zea mays L. will result in more available phosphate for uptake by the resulting in a more vigorous, higher yielding plant.

Maize is reported to have at least three acid phosphatases (El-Metainy and Omar, 1981) that may represent the reported plasmalemma, vacuolar and nuclear forms (Deltour et al., 1981).

Yeast acid phosphatase, pho5 (GenBank Accession Number X0 1079), may also perform the same role as the plant enzymes when secreted.

Plants also release organic acids in response to phosphate starvation. Organic acids lower the soil pH and release phosphate from inorganic complexes such as calcium and magnesium phosphates. Plants produce and secrete citrate, malate and oxalate for this purpose. Overexpression of citrate synthase (ACS) in Nicotiana tabacum enhanced use of insoluble phosphorous compounds upon secretion of greater levels of citrate. Furthermore, citrate-overproducing plants yielded significantly greater leaf and fruit biomass when grown in phosphorous-limiting soils (Lopez-Bucio, 2000). Citrate is a chelator of aluminum, calcium, and iron. Therefore, overexpression of citrate synthase also helps plants alleviate aluminum toxicity in acid soils. Organic acids such as citrate are known to enhance the availability of reduced or ferrous iron required by plants. Increased secretion of citrate also provides a carbon source for microorganisms in the soil, thereby increasing microbial release of phosphate from organic sources (Raghothana, 1999). It is expected that overexpression of citrate synthase in Z. mays will result in a higher concentration of phosphate available to the plant when soil phosphate availability is low. Overexpression of both mitochondrial and cytoplasmic forms of citrate synthase has been reported to improve phosphate uptake in model plants (Lopez-Bucio et al., 2000; Koyama et al., 2000). A mitochondrial form of rice citrate synthase (GenBank Accession Number AF302906) has been identified.

Another class of proteins that has been shown to increase under phosphorous limiting growth conditions is ribonucleases (RNases). S-like RNases release inorganic phosphate from RNA molecules in the intracellular matrix of the cell and are also secreted into the rhizosphere to release inorganic phosphate from RNA in soil organisms. RNases are involved in the turnover and degradation of RNA and it has been shown that they are induced in response to diverse stresses such as seed germination, water stress, wounding, pathogen infection, xylogenesis and phosphate starvation (Raghothana, 1999). RNases have been grouped according to their sequence and function. The function of S-RNases is gametophytic self-incompatibility whereas S-like RNases have been reported to be induced by diverse stresses. A barley (Hordeum vulgare L.) S-like RNase gene, RSH1, was recently cloned (Gausing, 2000). In cultivated tomato (Lycopersicon esculentum), two ribonucleases genes, extracellular located RNase LE and intracellular located RNase LX, are induced in response to phosphate depletion of the cultivation medium. The Arabidopsis thaliana ribonuclease gene, RNS1-3, is also induced in response to phosphate starvation (Bariola, 1994). S-like RNases have also been induced by phosphate starvation in Nicotiana alata (Dodds, 1996). It is believed that overexpression of an S-like RNase gene, such as the RSH1 gene, in Z. mays will result in a plant that is more resistant to phosphate starvation.

The uptake of phosphate at low concentrations poses an additional problem for plants. Phosphate rarely exceeds 10 μM in the soil and it diffuses very slowly, therefore requiring the plant to have specialized transporters. Other mechanisms for transporting phosphate across membranes between intracellular compartments are necesssary, because the concentration of phosphate is 1000-fold higher in these compartments than in the external solution. Higher plants generally store phosphate in the vacuole, where under non-limiting phosphorous conditions 85% to 95% of cellular phosphate is stored. However, the maintenance of a minimal level of phosphate in the cytosol is critical for normal metabolism, as phosphate concentration becomes a rate-limiting step in oxidative phosphorylation or the production of ATP. Additionally, phosphate is a key metabolite in the Calvin cycle, the sucrose synthesis pathway and a key part of adenylates and nucleotides. Cytoplasmic phosphate is maintained at a constant level (homeostasis) at the expense of vacuolar phosphate. Phosphate transport across the tonoplast is activated in response to lowered phosphate concentration and cytoplasmic alkalization (Chrispeels, 1999).

Phosphate is transported across the tonoplast and taken up by plant roots actively (requiring membrane H+-ATPase) by high-affinity transport proteins at low phosphate concentrations and low-affinity transporters when concentrations are high. The low-affinity transporters are expressed constitutively whereas the high-affinity transporters are induced under phosphate starvation conditions (Schachtman, 1998).

In addition to a possible role in phosphate scavenging, apyrase has been demonstrated to play a role in regulating the glycosylation state and activity of proteins in the Golgi apparatus (Gao et al., 1999; Zhong et al., 2000). This suggests that overexpression of apyrase proteins may have other effects on plant growth and development.

Phosphate transport has been reported to be augmented by apyrase when additional phosphate is supplied in the form of ATP. Apyrase scavenges phosphate from xATP by hydrolyzing and mobilizing the gamma-phosphate without promoting the transport of the purine or the ribose. Most apyrase are plasma membrane proteins but studies with the pea (Pisum sativum) gene, psNTP9, indicate that it is also extracellular. It is believed that apyrase may function to release inorganic phosphate from ATP present in the soil and mobilize it for plant uptake (Thomas, 1999). It is expected that overexpression of psNTP9 in Z. mays will result in an increased amount of soil phosphate from organic sources made available for plant uptake, thereby increasing plant vigor.

High affinity phosphate transporter genes (APT1 and APT2 (Smith 1997) and AtPT4 (Lu, 1997)) were isolated from Arabidopsis. Transporter genes have also been isolated from tomato (Liu, 1998) and potato (Leggewie, 1997). Sequence similarity between these plant transporter genes is high. It has been demonstrated that plants increase their capacity for phosphate transport across both the plasma membrane and the tonoplast during phosphate starvation by the increased expression of the transporter genes. Overexpression of tomato high-affinity transporter genes in tobacco resulted in higher levels of phosphate uptake during phosphate starvation (Mitsukawa, 1997). It is expected that overexpression of APT1, APT2 and AtPT4 in Z. mays will result in a plant with more vigorous growth during times of phosphate stress.

A low-affinity transporter gene, Pht2;1, has been cloned from Arabidopsis. The gene is predominantly expressed in green tissue and the amount of protein stays constant regardless of phosphate concentration. Its primary function appears to be phosphate loading of the leaf (Daram, 1999). It is believed that overexpression of Pht2;1 in Z. mays may result in higher cytosolic and mitochondrial levels of phosphate, thereby producing a more vigorous corn plant.

Phosphate starvation induces the expression of novel genes in the roots. One such gene was isolated from Medicago truncatula, MT4 (Burleigh, 1997). The MT4 gene shares sequence identity with the tomato gene TPSI1 (Liu, 1997) and the Arabidopsis AT4 gene. The proposed function-of these genes is to assist in phosphate acquisition during times of phosphate stress. It is believed that overexpression of the AT4 gene in Z. mays will result in corn plants more tolerant to low availability of phosphate in the soil.

Nucleic acid and polypeptide sequences for use in generation of transgenic crop plants having improved properties as described in the above strategy are provided in Table 1.

31. Phytochrome

Increased planting density has significantly contributed to gains in corn yield (Russell, 1991; Duvick, 1984). However, grain yield is observed to decrease once an optimal planting density is exceeded. The decrease in grain yield may be due to a reduction in the total amount of energy available to each plant but may also be due to phytochrome-mediated shade avoidance responses.

Manipulation of the shade avoidance response may allow corn plants to be grown at higher planting density and thus increase overall corn yield. Phytochrome has also been reported to regulate flowering time in many species including sorghum, barley and rice (Childs et al., 1991; Hanumappa et al., 1999; Izawa et al., 2000). This suggests that modulation of phytochrome response pathways may also be used to alter flowering time in maize.

The shade avoidance response is well studied in dicots (Neff et al., 2000; Morelli and Ruberti 2000). This response typically involves an elongation of the internode and a strengthening of apical dominance. Light enriched in far red due to absorption of red light by chlorophyll is thought to be perceived by both phyB and phyA (Reed et al., 1993; Casal, 1996; Shinomura et al., 2000) with some contributions by phyE and phyD in A. thaliana (Devlin et al., 1998; Devlin et al., 1999). Signals generated by the activated phytochrome proteins are relayed through a variety of different proteins including FAR1 (Hudson et al., 1999), NDPK2 (Tanaka et al., 1998; Choi et al., 1999; Pan et al., 2000), PIF3 (Ni et al., 1999; Halliday et al., 1999b), SPA1 (Hoecker et al., 1998), PKS1 (Fankhauser et al., 1999), PAT1 (Bolle et al., 2000), and AtHB-2 (Steindler et al., 1999). Several different models have been proposed to explain these complex signaling pathways, but insufficient information is available to build comprehensive pathway models.

In contrast to the dicots, much less is known about shade avoidance responses in monocots. Most of the available information comes from the study of a sorghum line deficient in phyB. The ma3 sorghum mutant lacks phyB and exhibits a constitutive shade avoidance phenotype similar to phyB mutant dicots. The plants have elongated shoots, low cholorophyll content and flower early (Childs et al., 1991). They also have altered gibberillic acid (GA) and ethylene production (Lee et al., 1998; Finlayson et al., 1999). Barley and rice deficient in phyB or heme oxidase, respectively, have also been described as early flowering, but have not been further characterized (Hanumappa et al., 1999; Izawa et al., 2000). In contrast to tobacco, where overexpression of phyA has been shown to repress the shade avoidance response and increase leaf yield (Robson et al., 1996), overexpression of phyA in rice did not result in dramatic phenotypic changes (Clough, 1995).

Overexpression of phyA has been reported to confer a short, dark green phenotype in many dicots. This has been associated with an increase in leaf yield in transgenic tobacco (Robson et al., 1996), antagonism of the shade avoidance response in potatoes (Heyer et al., 1995) and alteration of flowering time in A. thaliana (Bagnall et al., 1995). PhyA induced dwarfing is associated with a reduction in GA levels and can be repressed by the addition of exogenous GA (Jordan, 1995).

Overexpression of phyA in corn is expected to produce shorter, darker corn plants that may produce more ears per plant due to a reduction of apical dominance and GA levels. However, overexpression of oat phyA in rice did not result in dramatic phenotypic changes (Clough, 1995). PhyA has also been demonstrated to play a positive role in the shade avoidance response (Casal, 1996; Shinomura et al., 2000). Taken together with the lack of phenotype reported for rice, it may also be desirable to repress phyA in corn plants. Amino-terminal truncation of a rice phyA gene has been reported to inactivate endogenous phyA in tobacco (Emmler et al., 1995). Similar truncations can be used to inactivate phyA in corn.

Transgenic plants overexpressing phyB are reported to have reduced hypocotyl length and increased chlorophyll content. In transgenic potato, overexpression of phyB has been reported to increase total plant photosynthesis, reduced the rate of senescence and increased tuber yield (Thiele et al., 1999). PhyB overexpression has also been reported to alter flowering time in A. thaliana (Bagnall et al., 1995).

Overexpression of phyB in corn is expected to produce shorter, darker corn plants that may be higher yielding under high-density conditions due to a repression of the shade avoidance response or increased photosynthetic capacity. Reduction of phyB activity by the creation of a dominant negative amino-terminal truncation is expected to reduce corn flowering time.

Overexpression of phyC in A. thaliana and tobacco has been reported to increased leaf expansion rates (Qin et al., 1997; Halliday et al., 1997). This suggests that overexpression of phyC in corn might increase overall plant performance by rapidly increasing the amount of available photosynthetically active leaf area.

Heme oxidase is a plastid-localized enzyme that catalyzes the oxidative conversion of heme to biliverdin IX, the first committed step in phytochromobilin biosynthesis. A. thaliana and rice heme oxidase mutants, hy1 and se5, are devoid of active phytochrome (Muramoto et al., 1999; Davis et al., 1999; Izawa et al., 2000). The effects of these mutants have been mimicked by localized depletion of phytochrome activity by expression of mammalian biliverdin IX reductase, which degrades the phytochrome chromophore (Montgomery et al., 1999).

Knockout of the corn heme oxidases or degradation of the chromophore or intermediates in its metabolism would be expected to impair phytochrome-mediated responses in corn. This could lead to either a shift in flowering time or a reduction in the shade avoidance response.

The only reported biliverdin IX reductases are human and rat. However, a sequence with low, but significant homology, to biliverdin IX reductases has been reported in the Synechocystis genomic sequence (GenBank Accession No. S74645). Overexpression of this protein should result in a phytochrome deficient phenotype in maize.

A. thaliana spa1 mutants are hypersensitive to light signals perceived by phyA, suggesting that spa1 acts as a negative regulator of phyA signaling (Hoecker et al., 1998, 1999). SPA1 contains an N-terminal domain with a protein kinase motif and a C-terminal domain with at least 5 WD40 repeats. Overexpression of spa1 is expected to suppress phyA signaling and act to repress the shade avoidance response or delay flowering.

Nucleoside diphosphate kinases (NDPKs) are responsible for the exchange of gamma-phosphate residues between di- and triphosphonucleosides. They are known to play important roles in cellular signaling processes, lipid metabolism and carbohydrate metabolism. Humans have at least six nucleoside diphosphate kinases (nm23-H1 through nm23-H6). These proteins have been shown to play important roles in differentiation, metastasis and apoptosis. In many cases NDPK activity is modulated through interactions with other proteins. Likewise, interaction with NDPK has also been reported to modulate the activity of a number of proteins. NDPK has recently been shown to bind to and become activated by the Pr form of phytochrome (Choi et al, 1999). This observation, coupled with the observation that NPDK becomes rapidly phosphorylated upon exposure of etiolated seedlings to red light (Tanaka et al., 1998) and the inhibition of phytochrome induced cell elongation in rice plants with reduced levels of NDP kinase (Pan et al., 2000), suggests that nucleotide diphosphate kinases are direct mediators of light signals perceived by phytochrome.

Overexpression of NDP kinases in plants can be expected to induce many different phenotypes. However, it is possible the expression of either a native or an activated version of the protein may enhance phyB signaling and suppress the shade avoidance response.

A far red impaired response (FAR1) mutant was isolated in a screen for A. thaliana mutants which failed to respond to continuous far red light (Hudson et al 1999). As phyA alone is believed to mediate responses to continuous far red light in Arabidopsis (Dehesh et al., 1993), the genes identified in this screen are predicted to be impaired in phyA signalling. The far1 locus is said to encode a nuclear localized protein of unknown function. The sequence of the predicted FAR1 protein is closely related to many transposase sequences including maize jittery and mutator transposases. This information, coupled with the observation of a simple frame shift in the reported FAR1 mutant suggests that the gene identified as FAR1 may not be correct or may act in a manner which is unlikely to be reproducible in other species.

Overexpression of FAR1 is expected to simulate signals perceived during high fluence far red irradiation. This could mimic the shade avoidance response or delay flowering. Alternatively, deletion of these genes might block such signals and inhibit the shade avoidance response or hasten flowering.

Phytochrome A signal transduction 1 (PAT1) was identified in a screen similar to that used to isolate far1. A. thaliana seedlings from a T-DNA tagged population were screened for a failure to respond to continuous far red light (Bolle et al., 2000). The encoded protein is a novel member of the GRAS protein family. Other GRAS family members include scarecrow, gibberellin insensitive, repressor of gal in A. thaliana and d8 in maize (Pysh et al., 1999) and are involved in GA signal transduction and lateral patterning. The T-DNA insertion in PAT1 leads to the production of a truncated protein that may act in a dominant negative fashion.

Overexpression of full length PAT1 is expected to increase responsiveness to far red light and potentially hasten flowering. In contrast, overexpression of a truncated PAT1 orthologue or homologue would be expected to reduce sensitivity to far red light and potentially dampen the shade avoidance response. Disruption of the gene would be expected to have a similar phenotype.

Phytochrome kinase substrate 1 (PKS1) is a soluble protein of unknown function. It was identified in a yeast two-hybrid screen and found to interact with the C-terminal domain of phyA and phyB. PKS1 is phosphorylated by phyA and phyB in a light dependent manner. No effects were observed in transformants expressing anti-sense constructs of PKS1. However transformants overexpressing PKS1 were found to have elongated hypocotyls when grown in white light or red light. Because sensitivity to far red light was maintained, has been suggested that PKS1 can act as a negative regulator of phyB signaling (Fankhauser et al., 1999). Overexpression of PKS1 or homologous genes is expected to affect phyB signaling and thereby accelerate flowering time and alter the shade avoidance response. A single PKS-like sequence has been identified in the sequence of rice BAC 60A14 (accession AC021893).

Phytochrome interacting factor 3 (PIF3) is a b-HLH protein that was identified in a yeast two-hybrid screen for proteins able to interact with the C-terminal domain of phyA and phyB. Overexpression of PIF3 marginally increases sensitivity to continuous red and far red light as measured by hypocotyl extension rates (Ni et al., 1998). PIF3 is allelic to photocurrent1 (poc1), a T-DNA induced overexpression mutant that displays a phenotype similar to the PIF3 transgenic plants (Halliday et al., 1999). Overexpression of PIF3, or its corn homologues, is expected to increase sensitivity to red or far red light and potentially mimic a phyA or phyB overexpression phenotype. Supression of PIF3 expression, or its corn homologues, is expected to reduce sensitivity to red or far red light and potentially reduce the shade avoidance response or alter flowering time.

The PAS domain of PIF3 has been shown to interact directly with phyB (Zhu et al., 2000), suggesting that other PAS domain proteins may also interact with phytochrome and modulate it's responses. Overexpression of PAS domain proteins is expected to alter phytochrome responses and improve crop performance.

Expression levels of ATHB-2, a homeobox-leucine zipper class transcription factor, have been show to respond to the red/far red light ratio in which the plant is grown (Carabelli et al., 1993). A. thaliana plants overexpressing the protein show reduced cotyledon and hypocotyl expansion in a manner such as that of plants grown in environments enriched in far red light, thereby suggesting that ATHB-2 may act to mediate the shade avoidance response.

Disruption of corn ATHB-2 is expected to reduce the shade avoidance response and allow corn to better tolerate higher planting densities.

HY5 is a bZIP type transcription factor that has been shown to mediate high-irradiance light activation of gene expression was observed in both photosynthetic and nonphotosynthetic tissues (Chattopadhyay et al., 1998). HY5 interacts directly with COP1 (Ang et al., 1998) and is regulated by phosphorylation which alters its stability (Hardtke et al., 2000; Osterlund et al., 2000).

Overexpression of HY5 is expected to mimic high light environments and repress some aspects of the shade avoidance response.

Nucleic acid and polypeptide sequences for use in generation of transgenic crop plants having improved properties as described in the above strategy are provided in Table 1.

32. TOR Pathway

The TOR (Target of Rapamycin) signaling pathway in yeast plays a central role in regulating the transcription of genes involved in nitrogen assimilation, translation of genes involved in the cell cycle, ribosome biogenesis and amino acid permease stability in response to nutrients (especially nitrogen). Two P1 kinase homologs, TOR1 and TOR2, are the key components in the pathway. Functional homologs have been identified in human and C. elegans.

Through a database search, 10 P1 kinase homologs in Arabidopsis were identified. Most of them are potential homologs of P13,4 kinase. One of them (GenBank Accession Number AAF76442) is 52% similar overall to yeast TOR1. It is likely that this gene is the functional homolog of yeast TOR1.

The TOR signaling pathway is involved in nitrogen sensing in yeast. Since homologs of TOR kinase have been identified in Arabidopsis, it is likely that a similar pathway exists in higher plants. An Arabidopsis homolog of TAP42 (another component in the TOR pathway), TAP46, was identified using a yeast two hybrid system. This further supports the hypothesis that a TOR-like pathway may exist in plants.

The discovery that plants may have a TOR pathway is significant. It indicates that yeast, C. elegans, humans and plants may share the same pathway for nutrient sensing and couple the nutrient availability with cell division, translation, transcription, protein degradation as well as many morphological changes. Furthermore, the key components in the TOR pathway including TOR kinase and TAP46 are potential targets for improvement of nitrogen assimilation and stress resistance in crops.

FAP1 is a putative FKBP12 ligand related to Drosophilia shuttle craft and human FN-X1 containing at least 7 zinc finger-NF—X1 domains (Kunz et al., 2000). Interaction of FAP1 with FKBP12 is blocked by rapamycin. This class of zinc finger proteins has been postulated to play a role in protein ubquitination (Lorick et al., 1999).

FAP48 and FIT37 are FKBP12 interacting proteins that have been identified in human and A. thaliana (Chambraud et al., 1996; Faure et al., 1998). Like FAP1, interaction with FKBP12 is blocked by rapamycin. Sequence identity between FAP48 and FIT37 is low, but the function is conserved.

TAP46 and TAP42 are A. thaliana and yeast proteins that interact with PP2c in a TOR dependent manner (Di Como and Arndt. 1996; Harris et al., 1999). TAP42 is an essential protein and it's interaction with protein phosphatases is associated with cell growth (Jiang and Broach, 1999). Sequence identity between TAP46 and TAP42 is low, but the function is conserved.

eIF4E -4E-BP1 (also known as PHAS-1) is a key element in a regulatory system that governs translation initiation by controlling the availability of eIF4E1. One of the first steps in initiation involves binding of eIF4E to the m7GpppN cap, which is found at the end of almost all eukaryotic mRNAs. eIF4E also binds to eIF4G, a scaffold protein that organizes several other important initiation factors, including eIF3, which links the complex to the 40 S ribosomal subunit (Gingras et al., 1999). Nonphosphorylated PHAS-I binds tightly to eIF4E, preventing its association with eIF4G. When phosphorylated in the appropriate sites PHAS-I dissociates, allowing eIF4E to engage eIF4G to form the complex that facilitates initiation. PHAS-1 may be phosphorylated by TOR in vivo (Mothe-Satney et al., 2000).

Overexpression of eIF4E may mimic some of the effects of TOR activation by increasing levels of the free protein. Consistent with this idea, overexpression has been shown to transform mammalian cells and the protein is overexpressed in many human cancers (Zimmer et al., 2000; Haydon et al., 2000, Li et al., 1997).

The yeast 14-3-3 proteins BMH1 and BMH2 act as multicopy suppressors of the growth-inhibitory phenotype caused by rapamycin in budding yeast (Bertram et al., 1998). This may be due to a direct interaction between the proteins and TOR (Mori et al., 2000) or an indirect effect on proteins downstream of TOR (Cotelle et al., 2000).

Overexpression of BMH1 or BMH2 may mimic some of the effects of TOR activation either by enhancing signaling or protecting proteins that are normally degraded upon nutrient starvation.

It is expected that expression of genes relating to the TOR pathway will enhance nitrogen assimilation, chance yield by altering the cell cycle, or enhance the amino acid composition of a plant. Nucleic acid and polypeptide sequences for use in generation of transgenic crop plants having improved properties as described in the above strategy are provided in Table 1.

33. Plant Hemoglobins

Hemoglobins are ubiquitous oxygen-binding heme proteins that consist of a globular apoprotein and a iron-binding heme group (cofactor) which is associated with this apoprotein (either covalently or noncovalently) to form hemoglobin. They are found in prokaryotes, fungi, plants and animals and are distinguished from other oxygen-binding heme proteins by their sequence similarity. Plant and animal hemoglobins diverged from a common ancestor approximately 1.4 billion years ago (Brown et al., 1984).

In legume nodules, hemoglobins (commonly called leghemoglobins) are present in high concentrations and function to maintain a high level of oxygen supply to the mitochondria. This is necessary to produce the high levels of ATP required for nitrogen fixation while at the same time ensuring that the oxygen is sequestered away from nitrogenase, which is very sensitive to inhibition by oxygen. In plants that do not form symbiotic relationships with nitrogen fixing bacteria, hemoglobins are expressed during periods of low oxygen availability or high energy demand (Duff et al., 1998)

In previous studies, transgenic tobacco plants expressing functionally different hemoglobins have been produced (Dieryck eet al., 1997; Holmberg et al., 1997; Bulow et al., 1999; Barata et al., 2000). It has been demonstrated that bacterial hemoglobin promoted enhanced growth and germination and altered metabolite production in transgenic tobacco plants whereas the expression of functional human hemoglobin had no observable phenotypic effects (Dieryck et al., 1997). Hemoglobin therefore has the proven potential to improve seed germination, plant growth and ultimately, yield.

While tobacco plants overexpressing Vitreoscilla hemoglobin show a definite phenotypic effect (Holmberg et al., 1997), tobacco plants overexpressing soybean leghemoglobin in their chloroplasts did not significantly differ from wild-type (Barata et al, 2000). Since soybean leghemoglobin was used instead of Vilreoscilla hemoglobin in this study (Barata et al., 2000), it is unknown whether the lack of effect was due to the different binding capacity of the two hemoglobins or to chloroplast localization. Since human hemoglobin, also, did not give enhanced characteristics to tobacco when targeted to the chloroplasts (Dieryck et al., 1997) it is possible that the chloroplast is not the best location in the cell for hemoglobin. In order for hemoglobin to be effective in the chloroplast much higher concentrations would be necessary. One of the most important properties of hemoglobins is the affinity to which they bind oxygen. The bacterial hemoglobin has an affinity for oxygen that is over 100 times lower than that of soybean leghemoglobin. Therefore, it is possible that the binding affinity for oxygen is one of the major factors affecting the efficacy of hemoglobin in transgenic plants.

There are several theories on the function of hemoglobin in plants and several hypotheses on how hemoglobin might be used in a transgenic approach to improve plant metabolism. Since plant hemoglobins have a wide range of oxygen affinity, it is probable that they serve a number of different functions in plants. Hemoglobin may bind oxygen and prevent oxidative damage to the chloroplasts or other cellular compartments. Hemoglobin may also act as an oxygen sensor or as an oxygen carrier possibly to transport oxygen to the mitochondria. Recent results suggest that hemoglobin may function in the cytoplasm under conditions when the energy needs of the plant exceed the oxygen supply to somehow recycle reductant and keep glycolysis performing at maximum efficiency (Sowa et al., 1998; Duff et al., 1997). This would imply that hemoglobins would function at levels of oxygen below the affinity of the mitochondria for oxygen and would imply a low K_{D} for oxygen. This is believed to be the case for the nonsymbiotic hemoglobins. Another theory suggests that hemoglobin may function to prevent oxidative damage in the chloroplasts (Barata et al, 2000).

It is quite likely that due to the immense differences in oxygen affinity between barley and Vitreoscilla hemoglobin that their mode of action may be different and that recombinant (foreign) hemoglobin may act differently than the endogenous form. For example, the exogenous recombinant hemoglobin may increase oxygen available for respiration while the endogenous native hemoglobins may have a more specialized function. The localization of hemoglobin is also important in determining cellular function

Several different hemoglobins with different oxygen affinities are targeted to the chloroplast, mitochondria and cytosol of maize plants. The rice actin promoter is used to constitutively direct hemoglobin expression, but, seed, root and flooding inducible promoters are also used to specifically target hemoglobin to the tissue or enviromental stress conditions where it might be most effective.

It is expected that expression of plant hemoglobins will enhanced growth and seed germination.

Nucleic acid and polypeptide sequences for use in generation of transgenic crop plants having improved properties as described in the above strategy are provided in Table 1.

34. Polycomb Proteins for Apomixis

Controlled apomixis, the asexual reproduction of a plant through the seed, would greatly reduce production costs by allowing the “fixation” of heterosis. Mutations in A. thaliana have identified a set of genes that appear to repress embryo and endosperm development in the absence of fertilization (Pruess, 1999). Alteration of the activity of related genes in corn may lead to the induction of apomixis or also changes in grain quality by alteration of the ratio of the embryo to the endosperm.

Three genes have been identified in A. thaliana which allow partial embryo or endosperm development without fertilization: FIE1, FIS2 and Medea (fis1, f644 and emb173). FIE is 40% identical to polycomb group genes that encode a WD domain, including extra sex combs (esc) from Drosophila and embryonic ectoderm development (Eed) from mammals (Ohad et al., 1999). MEDEA contains a SET domain with 55% identity to another member of the polycomb group, enhancer of zeste (Ez)) (Grossniklaus et al. 1998). FIS2 encodes a protein predicted to contain a zinc finger and nuclear localization signal, suggesting that it is involved in transcriptional control (Luo et al. 1999). In vitro and in vivo binding assays indicate that the Drosophila esc and Ez proteins interact. Thus, the products of the Arabidopsis FIE and MEDEA genes are predicted to interact directly and to perform a similar role (Preuss, 1999).

FIE1, fertilization independent endosperm, is an Arabidopsis homolog of the WD motif-containing Polycomb proteins from Drosophila and mammals (Ohad et al., 1999). These proteins function as repressors of homeotic genes. A female gametophyte with a loss-of-function allele of FIE1 undergoes replication of the central cell nucleus and initiates endosperm development without fertilization. When fertilization is prevented in Arabidopsis plants heterozygous for the FIE1 mutant allele, siliques elongate and contain seedlike structures composed of an endosperm surrounded by a seed coat. However, no embryo development is observed (Ohad et al., 1996). FIE1 may act to suppress transcription of genes for central cell nuclear replication in the female gametophyte until fertilization occurs.

The FIS1 gene is similar to a gene designated MEDEA, a recently described Arabidposis gene related to the Polycomb-group gene Ez from Drosophila (Grossniklaus, 1998). Comparison of the sequences between FIS1 and MEDEA indicates that whereas all of the characteristic domains of Ez-related proteins are eliminated by the N-terminal stop codon in the FIS1 allele, all domains except the SET domain remain intact in the C-terminal disruption of the MEDEA mutant allele. The FIS1, F664 and EMB173 mutations all result in a similar phenotype—excessive proliferation of the endosperm and an early defect in embryo development. In contrast, MEDEA mutations appear to have the opposite effect, resulting in excessive proliferation of the embryo (Grossniklaus et al. 1998). However, this may be a gain-of-function phenotype because it is found only with alleles that eliminate the C-terminal SET domain. Early stop codons in the MEDEA coding sequence (FIS1, F644, and EMB173) instead cause the same phenotype as FIE or FIS2, with excessive endosperm proliferation, even in the absence of fertilization (Kiyosue et al. 1999; Luo et al. 1999). MEDEA is an imprinted gene that displays parent-of-origin-dependent monoallelic expression specifically in the endosperm which suggests that the embryo abortion observed in mutant seeds is due, at least in part, to a defect in endosperm function (Kinoshita et al., 1999).

In the FIS1, FIS2, and FIS3 (FIE) mutants of Arabidopsis, the phase 1 ovule proceeds without pollination to phases analogous to phases 2 and 3 of wild type seed development. The seed-like structures have free nuclear endosperm and an embryo (FIS1 and FIS2), free nuclear endosperm without an embryo (FIS1, FIS2, and FIS3), or seed-like structures with cellularized endosperm (FIS1 and FIS2). Even with pollination, the FIS seed develops only to an embryo-arrested heart/torpedo stage and does not become a mature seed with a U-shaped embryo. The conversion of embryo-arrested seed to mature seed may be blocked by gene Z that is not inactivated by pollination. Mutations in the hypothetical Y gene would convert the FIS seeds into embryo-arrested seeds without pollination, and additional mutation of the Z gene would give rise to normal seeds.

The predicted polypeptide sequence of FIS2 shows a putative C2H2 zinc-finger motif within the first 35 residues of the protein and 3 putative bipartite nuclear localization signals distributed between residues 267-540 (Lou et al., 1999). Like other zinc finger proteins, FIS2 has a high serine content (12.9%). The A and B repeats produce a distinctive signature when the FIS2 protein product is compared with itself by dot matrix analysis. The consensus sequences for A and B repeats are H-V-N-D-D-N-V-S-S-P-P-[R/K]-A-H-S-S-K-K and L-T-T-T-Q-P-A-I-A-E-S-S-E-P-K-V. Following the consensus sequence described above, half of the A repeats have a T-S-D-I sequence and the other half have N-E-S-T. T he central part of the A repeat has a consensus SPP[R/K] with similarity to the motif [T/S]PXX (X is usually a basic amino acid) that has been shown to bind in the minor groove of DNA.

FIS2 expression was not detected in Arabidopsis shoots, leaves, bolting stem, flower buds or siliques after Northern blot analysis using 20 μg of total RNA for each tissue sample. Similarly, RNAse protection assays did not generate a protected fragment, suggesting that FIS2 is expressed at low levels. The only confirmed expression of the gene is in late silique RNA where a FIS2 cDNA was isolated from the library at a frequency of 1:1 00,000.

Ovule development in mutant ovules from a FIS2/fis2 heterozygous Arabidopsis plant without pollination is equivalent to the development of pi/pi ovules 3 days after pollination, and cellularized endosperm cells occasionally are accompanied by an embryo-like structure at the micropylar end (Lou et al., 1999).

Polycomb group proteins form a complex that regulates gene expression through epigenetic silencing (Pirrotta, 1998). Their roles are best understood in Drosophila. After early patterns of homeotic gene expression are established, the polycomb proteins contribute to the maintenance of those patterns by silencing homeotic genes in appropriate regions. A complex of polycomb proteins interacts with DNA sequences (polycomb response elements, or PREs) that are scattered throughout the genome (Pirrotta, 1998). These interactions can silence the expression of genes that contain PREs as well as the expression of nearby genes. It is unclear if polycomb proteins directly interact with promoters and enhancers or if instead they remodel chromatin into less accessible forms. In any case, the absence of methylation in the Drosophila genome indicates that polycomb-mediated gene silencing, at least in flies, acts through methylation-independent mechanisms (Pruess et al., 1999).

At least six polycomb proteins are known in A. thaliana. Aside from MEDEA and FIE only one other polycomb protein has an associated function in A. thaliana. The CURLY LEAF gene of Arabidopsis is necessary for stable repression of a floral homeotic gene and encodes a protein with homology to the product of the polycomb-group gene Enhancer of zeste (Goodrich et al., 1997).

Overexpression of full length polycomb proteins or potentially dominant negative derivatives of them is expected to alter corn development and potentially lead to the development of apomixis or grain of altered composition due to alterations in embryo and endosperm size. Loss of function mutants can be expected to lead to similar results.

Nucleic acid and polypeptide sequences for use in generation of transgenic crop plants having improved properties as described in the above strategy are provided in Table 1.

35. Retinoblastoma (Rb) Like Genes

The retinoblastoma gene was one of the first tumor suppressor genes to be identified in mammals. The Rb tumor suppressor gene functions as a negative regulator of cell proliferation and normally acts to inhibit unregulated cell division in mammalian cells. Unlike mammalian cells, little is known how plants regulate G1 progression (Ach et al 1997). Several cDNA sequences were isolated from maize that encode Rb-related proteins and have been shown to be highly expressed in the shoot apex (Ach et al. 1997). The Rb-like proteins have been shown to interact with type D-Cyclins (Ach et al. 1997), a family of proteins that regulate the activity of cyclin dependent kinases (CDK) (Riou-Khamlichi et al. 1999) and control progression through the cell cycle (Cooper, 1995). It is suggested that G1 regulation in plant cells is controlled by mechanisms similar to the ones found in mammalian cells (Ach et al. 1997). The presence of CDKs and cyclins suggest that the mechanism for regulation has been conserved in eukaryotic evolution (Ach et al. 1997). Recent evidence suggests that G1 control involves the Rb-related proteins. Rb has also been shown to interact with other proteins such as the wheat dwarf virus (WDV) C1 gene (Xie et al. 1995). This protein contains the Rb binding motif LeuXCysXGlu that permits oncoproteins to inactivate Rb (Xie et al. 1995). Expression of WDV could serve as an additional way to inactivate the Rb gene either constitutively (rice actin) or at specific times or regions, e.g., seed specific expression using the GS1-2 (pedicel specific glutamine synthase-1) or bet1 (basal transfer layer specific) promoters. Anti-sense expression of the Rb-related proteins constituitively or at specific times or in specific tissues may allow for increased cell division rates, resulting in bigger plants, higher yields, increased turgor and a higher degree of seed set in times of water stress.

Nucleic acid and polypeptide sequences for use in generation of transgenic crop plants having improved properties as described in the above strategy are provided in Table 1.

36. Root Mass

Water availability is a key factor in determining plant yield. Water flux throughout the plant is primarily controlled by influx to the root, transport, photosynthetic rate and transpiration. Roots are usually the site of the highest resistance for the movement of water through the soil-plant-atmosphere flow (Kramer and Boyer, 1995). It is believed that alteration of root mass will positively influence maize growth and yield under water limiting conditions.

A number of studies have associated root mass and yield, or root mass and drought tolerance. Replicated, controlled field trials of corn in which three week old seminal roots were severed showed a 9 percent loss in grain yield (Kisselbach, 1949). This study indicated that early seminal root formation, not a significant part of the total root system, could have a significant affect on final yield. In addition, the size and structure of the sorghum root system has been shown to be associated with drought tolerant lines. The root mass of a stress tolerant sorghum line was not affected by water stress whereas a 30% reduction in root mass was detected in a water stress sensitive line (Cruz, et al., 1992). In soybean, Hudak (1995) showed in replicated trials that a drought tolerant variety had increased root mass, root volume, and relative surface area as compared to another commonly grown similar cultivar. All these studies associate, but do not directly demonstrate, increased root mass as a factor contributing to increased yield or drought stress resistance.

One method to alter the root mass and the root influx of water is through the regulation of aquaporin proteins. Aquaporins are highly selective water channel proteins that have an important role in water transport across cell membranes and have been identified in both animal and plant cells. They are characterized as Membrane Intrinsic Proteins (MIPs) and span the membrane six times (Preston, 1992). The transmembrane domains form a narrow pore that is thought to exclude everything but water (Steudle and Henzler, 1995). The plant MIPs are proposed to accelerate the influx of water into cells to maintain turgor pressure under low water conditions (Hollenback and Dietz, 1995). Classes of these proteins have been identified in Arabidopsis which are localized to the plasmalemma (PIP) (Kaldenhoff et al 1995) or to the vacuole (TIP). The function of plant PIPs and TIPs has been demonstrated as water channel proteins by expression of these mRNAs in Xenopus oocytes (Maurel et al., 1993).

To determine effects of altered aquaporins on plant morphology, Kaldenhoff et al.(1998), produced transgenic Arabidopsis plants expressing an antisense construct of PIP1b driven by the 35S promoter. The results indicated that the expression of both the PIP1a and PIP1b genes were reduced; leaf protoplasts had reduced water permeability and swelling rates in hypotonic solution. Overall water uptake of the antisense plants was not affected. The root mass to leaf ratio was increased five fold in the antisense plants. The root xylem pressure was the same as in control plants, even after osmotic change. These data suggest that the plant compensated for a reduction in the PIP1a aquaporin by increasing root surface thereby maintaining a normal level of water transport and xylem pressure.

Antisense expression of PIPb is expected to result in an increase in the fresh weight mass ratio of root to shoot. R1 transgenic and sibling nontransgenic control plants are grown under well watered and water limited conditions. The fresh weight of root mass and aerial parts is measured in seedlings from early and mid vegetative growth plants. In addition, the stomatal transpiration rate and turgor pressure of the leaves of transgenic and control plants are measured. Larger root mass phenotype is observed and turgor is measured at flowering stage under water stress and non-stressed conditions. In addition, a yield trial is performed.

cDNAs that are expressed in maize line B73 root tissue, but not in kernel tissue, were isolated by differential Southern hybridization. The root positive/kernel negative cDNAs were further analyzed by Southern and Northern analysis. One cDNA, designated RS 81cDNA, has 88% amino acid homology to maize aquaporin TIPs (GenBank accession AF057183), and high homology to other plant TIPs. The RS81 gene sequence encodes a protein of 249 amino acid residues and a predicted molecular weight of 24,895.9 Daltons. The protein possesses 131 hydrophobic residues and a plot of the hydrophobicity pattern predicts that RS81 has 6 hydrophobic domains, in keeping with its role as a transmembrane protein that spans the membrane six times.

The rs81 cDNA was fully sequenced and used to search GenBank. The rs81 cDNA sequence encodes a protein with 88% amino acid homology to maize aquaporin TIPS (GenBank Accession AF057183). The CDNA sequences have 81% identity to a Orysa saliva root EST (dbj(D23748), 68% nucleotide identity to a Hordeum vulgare mRNA encoding a gamma-TIP-like protein (emb(X80266) and 72% amino acid identity to a Nicotiana tabacum root specific tonoplast protein ( sp(P30571).

A second root specific cDNA that has high homology to plant PIPs was isolated and was designated RS288,. This gene is also expressed in root tissue as detected by northern blots. RS288 has 84% homology to Oryza sativa aquaporin PIP2a (GenBank accession AF062393), 82% homology to Arabidopsis PIP2a (GenBank accession X75883) and 84% homology to Arabidopsis PIP2b (GenBank accession X75884) and 82% homology to a Glycine max drought induced water channel protein (GenBank accession UZ7347). The RS288 gene sequence encodes a protein of 288 amino acid residues and a predicted molecular weight of 30,346.19 Da. The protein posseses 133 hydrophobic residues and a plot of the hydrophobicity pattern predicts that RS288 has 6 hydrophobic domains, in keeping with its role as a transmembrane protein that spans the membrane six times.

It is expected that alteration of expression of aquaporin or aquaporin-like proteins in a plant will lead to increased root mass and therefore an increased efficiency of water utilization.

A transcription factor, designated G9, causes phenotypic changes in Arabidopsis when overexpressed, e.g., more gorwth in roots grown on Murashige and Skoog (1962, hereinafter called MS) medium and hairy roots on medium containing methyl jasmonate. G9/RAV2/RAP2.8 (Okamuro et al., 1997) (Kagaya et al., 1999) belongs to a small subgroup within the AP2/EREBP family of transcription factors, whose distinguishing characteristic is that its members contain a second DNA-binding domain, in addition to the conserved AP2 domain, that is related to the B3 domain of VP1/ABI3 (Kagaya et al., 1999). It has been shown that the two DNA-binding domains of RAV1 (another member of this subgroup of proteins) can separately recognize each of two motifs that constitute a bipartite binding sequence and together cooperatively enhance its DNA-binding affinity and specificity (Kagaya et al., 1999). The similarity between G9 and all other AP2/EREBP proteins, from Arabidopsis and from other plant species, does not appear to be significant outside of the conserved AP2 and B3 domains. G9 appears to be constitutively expressed in Arabidopsis. However, overexpression of G9 causes phenotypic changes in the roots, i.e., more growth in roots grown on MS medium, and hairy roots on media containing methyl jasmonate. It is expected that overexpression of G9 in a plant will cause phenotypic changes in roots and enhance the water use efficiency of the plant.

Nucleic acid and polypeptide sequences for use in generation of transgenic crop plants having improved properties as described in the above strategy are provided in Table 1.

37. Short Vegetative Phase

In maize, a major quantitative trait locus (QTL) affecting early flowering, namely vegetative to generative transitional 1 (Vgt1), also called Maturity factor 1 (Mf1), has been mapped at about 5 cM (between UMC89a and EC2-11, PG7) in bin 8.05 of chromosome 8L. This QTL originated from Gaspe Flint and in a homozygous state enables corn plants to flower 7-10 days earlier than normal plants. The QTL exhibits a partial dominant effect in heterozygotes. It was believed that the early flowering of Vgt1 plants is due to the very early timing of tassel initiation rather a fast rate of growth, and Vgt1 may act in pathway to repress the transition from vegetative to floral development.

A repressor of the floral transition, named Short Vegetative Phase (SVP) was recently cloned from Arabidopsis (Hartmann, et al. 2000). It represents a new member of the MADS-box gene family of transcription factors. Homozygous SVP mutant plants flower significantly earlier than wildtype plants and a partial dominant effect is observed in heterozygotes. It is believed that SVP functions as a repressor of flower initiation. Based on similarity in function and flowering phenotype, it is believed that the orthologue of SVP in corn is Vgt1.

As Vgt1 corn plants flower 7-10 days earlier than normal corn plants, the identification of the Vgt1 gene in corn provides several potential applications, such as: transforming Gaspe Flint Vgt1 gene(s) into elite corn lines to produce early flowering elite corn; introgressing Gaspe flint Vgt1 gene(s) into elite corn lines by backcrossing using the molecular markers for the Vgt1 gene(s); and genetic engineering early maturity crops (corn, rice, wheat, and etc.) of high yield.

Nucleic acid and polypeptide sequences for use in generation of transgenic crop plants having improved properties as described in the above strategy are provided in Table 1.

38. Lodging Resistance

The Arabidopsis STURDY mutant identified by T-DNA tagging displays an increased cell number phenotype in the stems of mutant plants and therefore, is more lodging resistant than wild-type. In addition STURDY seeds weigh 30% more than wild-type seeds. The STURDY gene was cloned and encodes a patatin-like protein. Transgenic studies in Arabidopsis have confirmed that the STURDY phenotype is caused by the overexpression of STURDY. It is believed that crop lodging resistance and seed weight (yield) may be improved by expressing STURDY in the stems and seeds of crop plants. Furthermore, it is expected that the increase in seed weight will change the relative oil or protein content of the seeds and result in a higher grain quality. The detailed isolation and characterization of the STURDY mutant and STURDY gene are summarized in Huang et al., 2000. Patatin-like proteins from N. tabacum have been shown to have phospholipase A2 activity (Dhondt et al., 2000).

Nucleic acid and polypeptide sequences for use in generation of transgenic crop plants having improved properties as described in the above strategy are provided in Table 1.

39. Virus Resistance

Maize is subject to a large number of diseases caused by RNA viruses. In the United States, Maize Dwarf Mosaic Virus (MDMV) and Maize Chlorotic Dwarf Virus are major viral pathogens (MCDV). Outside of the United States, Maize Streak Virus (MSV) and Maize Rough Dwarf Virus (MRDV) are major pathogens found in Africa (MSV) and Europe, South America and Asia (MRDV). Infection with any of these viruses is associated with heavy yield reductions (McGee 1998).

Yeast contains a set of genes, dubbed superkiller genes (SKI genes), involved in the degradation of non-polyadenylated RNA (Jacobs, 1998; Benard et al., 1999; van Hoof et al., 2000; Searfoss and Wickner 2000). Mutations in these genes result in an increase in the copy number of L-A and M viruses with a resulting increase in the production of viral encoded toxins that gave the SKI mutations their name (Toh et al., 1978, Toh and Wickner, 1980). However, overexpression of at least one of these proteins, SKI7, results in a increase in the rate of degradation of non-polyadenylated viral RNA and curing of the M virus (Benard et al., 1999).

Increasing the rate of breakdown of non-polyadenylated RNA in plants may result in increased resistance to plant viruses that produce such RNA. Three of the major corn viruses MDMV (a potyvirus, ss linear RNA genome), MCDV (a waikavirus, ss linear RNA genome) and MRDV (a fijivirus, ds linear RNA genome) produce non-polyadenylated transcripts, suggesting that overexpression of genes involved in RNA breakdown may protect corn against these viral pathogens. Enhanced degradation of viral RNAs may also result in broad-spectrum resistance to plant RNA viruses.

Mutations in SKI2, SKI3, SKI4, SKT6, SKI7, and SKI8 all reduce the copy number of the double stranded RNA viruses L-A and M (Wickner, 1996). SKI2, SKI3 and SKI8 exist as a complex in vivo and are thought to be located in the nucleus (Brown et al., 2000). Of the three proteins in the complex, SKI2 is a helicase, SKI8 is a WD repeat containing protein (Matsumoto et al., 1993) and SKI3 contains 11 TRP repeats and is nuclear localized (Rhee et al., 1989), suggesting that the SKI2/SKI3/SKI8 complex is located in the nucleus. SKI6 is a 3′ to 5′ exonucleases related to RNAse P14 and can be replaced by its A. thaliana orthologue AtRrp411 (Chekanova et al., 2000). SKI7 is a GTPase belonging to the EF-Tu family. The SKI proteins are thought to be either components or regulators of the RNA degrading exosome (Mitchell and Tollervey, 2000).

Corn lines overexpressing SKI proteins or their orthologues are tested for resistance to ds RNA viruses such as Maize Rough Dwarf Virus and Mal de Rio Cuarto Virus as well as ss RNA viruses such as Maize Dwarf Mosaic Virus and Maize Chlorotic Dwarf Virus. Other types and classes of viruses are also screened. It is expected that plants overexpressing SKI genes or their orthologues will be virus resistance.

Nucleic acid and polypeptide sequences for use in generation of transgenic crop plants having improved properties as described in the above strategy are provided in Table 1.

40. Yield Associated Genes

Genes are selected that are transcriptionally regulated in a manner that correlates with high yield. The expression of the selected genes is modified in corn by overexpression, antisense or sense supression, tissue specific expression or development stage specific expression.

The 40S Ribosomal protein S4 (LIB3245-104-P1-N1-D11) is an RNA-binding ribosomal protein that plays a role in translational fidelity (Vincent and Liebman, 1992; Synetos et al., 1996; Dahlgren et al., 2000). Elevated levels of S4 RNA levels are associated with growth in tobacco mesophyll protoplasts (Marty et al., 1993) and in benign prostatic hyperplasia (Vaarala et al., 2000). Little information is available on the effects of S4 overexpression.

Chloroplastic fructose-1,6-bisphosphatase (LIB3245-466-P2-K1-G8 plays an important role in the Benson-Calvin cycle. The enzyme is regulated by thioredoxin in response to the redox potential of the chloroplast (Hirasawa et al., 1999) and may be a key factor in determining sensitivity to chilling induced photooxidative damage (Hutchison et al., 2000). Little information is available on the effects of altering expression of the chloroplastic form of this enzyme.

TBP-1 (TAT binding protein 1 or subunit 4 of 26S proteasome; is a AAA ATPase that forms part of the 19 S regulatory complex (RC) of the 26 S proteasome. Human and plant versions of TBP-1 have been shown to complement yeast TBP1 mutants (Schnall et al., 1994; Fu et al., 1999).

UBC2-1 is very similar to AtUBC2-1 (also called ubiquitin-conjugating enzyme 15) (Bartling et al., 1993) and has protein sequence motifs in common with ubiquitin conjugating enzymes as determined using PFAM. Ubiquitin-conjugating enzymes act in conjunction with E1 (ubiquitin-activators) and E3 (the ubiquitin-protein ligases) proteins to target proteins for degradation. Components of the ubiquitination system have been shown to directly interact with the 19 S regulatory complex of the 26 S proteasome (Tongaonkar et al., 1999; Xie and Varshavsky, 2000). A yeast ubiquitin-conjugating enzyme, UBC8, has also been shown to be responsible for turnover of fructose-1,6-bisphosphate phosphatase in response to a shift to glucose-containing media (Schule et al., 2000). However, ubiquitin is not found in the chloroplast of higher plants (Beers et al., 1992) and the significance of this is unknown.

RRP5 (LIB3245-141-P2-N1-C11) was identified in yeast to be an essential protein required for the production of 18S and 5.8S RNAs (Venema and Tollervey, 1996).

The CBP-like gene encodes a protein that is similar to the A. thaliana protein YUP8H12R.38. This protein is in turn related to the CREB binding protein of C. elegans. It is predicted to contain two TAZ and a single ZZ type zinc fingers (Ponting et al., 1996) and a PHD domain. This suggests that encodes a transcriptional co-activator similar to CBP or p300.

Nucleic acid and polypeptide sequences for use in generation of transgenic crop plants having improved properties as described in the above strategy are provided in Table 1.

41. Wax Biosynthesis

Epicuticular waxes on the leaf surface serve several functions, including resistance to water loss, insects and disease (McWhorter 1993). Wax production has been shown to increase in drought stressed plants (McWhorter 1993). Increased wax levels also make the plant more tolerant to high temperature stress (McWhorter 1993). Several factors condition the relation between epicuticular wax levels (EWL) and water use efficiency (Premachandra et al. 1994). First, the chemical composition and physical structure of waxes may influence the permeability of the cuticle and epicuticle to water. Second, the occlusion of stomatal pores by wax and presence of wax inside the stomatal antechamber may modify conductance and transpiration. Third, the cuticle of guard cells may be more highly permeable than other epidermal cells and wax deposition may reduce permeability. Finally, leaf reflectance in the visible and near-infared wavelengths may affect the balance between water vapor efflux and CO2 assimilation.

The overexpression of a gene encoding a protein involved in wax biosynthesis using a strong promoter may increase the water use efficiency of a plant, making it less susceptible to drought. Several genes involved with wax production have been reported: G11 (GenBank Accession U37428), G12 (GenBank Accession X88778), G18 (GenBank Accession U89509), G115 (GenBank Accession AF001012), Cer1 (GenBank Accession AF143746), Cer2 (GenBank Accession U40849), Cer3 (GenBank Accession X95962) and Cut1 (GenBank Accession AF129511).

Expression of the glossy loci in maize affects wax biosynthesis. Mutations at the G11 locus of maize quantitatively and qualitatively affect deposition of cuticular waxes on the surface of seedlings (Hansen et al. 1997). The G12 locus is required for the formation of the epicuticular wax layer of young plants (Tacke et al. 1995). A mutation in the G18 locus reduces the level and alters the composition of seedling cuticular waxes (Xu et al. 1997). The G18 protein is thought to function as a reductase during fatty acid elongation in the cuticular wax biosynthesis pathway (Xu et al. 1997). G115 loss of function mutants have altered transition from juvenile to adult growth by conditioning the abbreviated expression of juvenile epidermal traits and the coordinate precocious expression of adult epidermal characteristics (Moose et al. 1994). G115 shows sequence homology to the Arabidopsis floral and regulatory genes APETALA2 and AINTEGUMENTA (Moose et al. 1996). These findings suggest that overexpression of these specific glossy genes may result in increased wax production on the leaf cuticle. Antisense transformation of G115 may induce the plant to begin the adult phase prematurely.

In Arabidopsis the Eceriferum (CER) genes affect different steps of the wax biosynthesis pathway (Aarts et al 1995). CER1 encodes a novel protein involved in the conversion of long chain aldehydes to alkanes, an important step in wax biosynthesis (Aarts et al. 1995). G11 and CER1 show significant sequence homology. CER2 contains sequence similarity to maize gene G12. CER3 encodes one of 21 gene products involved in wax production (Hannoufa et al 1996). This gene however shows no homology to any other known protein, yet the second exxon of CER3 has a RRX12KK nuclear localization sequence (Hannoufa et al 1996).

In Arabidopsis, suppression of CUT1 gene expression results in wax-less stems and siliques as well as male sterility (Millar et al 1999). In CUT1 suppressed plants the C24 chain-length wax components predominate, suggesting that CUT1 is necessary for elongation of C24 long-chain fatty acids (Millar et al. 1999). CUT1 does not map to any other known Eceriferum loci suggesting it is a novel wax biosynthesis gene. The fact that these genes are linked so tightly to wax biosynthesis suggests that the overexpression of these genes may result in increased wax biosynthesis.

The overexpression of wax biosynthesis genes, as well as anti-sense expression of G115 may increase water use efficiency and make the plants less susceptible to drought. Increased wax production could also lower the plants susceptibility to insect damage, disease and high temperature stress.

Increased epicuticular wax levels can be measured in several ways. First, the increased epicuticular wax can be scored visually under a dissecting scope. Second, the wax level can be measured by removing it with chloroform, allowing the chloroform to evaporate and then measuring wax remaining (McWhorter et al. 1993). The wax weights are related to the total area of leaves or leaf sections extracted (McWhorter et al. 1993). Last, the effects of increased epicuticular wax can be determined in the greenhouse or field as it relates to water use efficiency.

Mendel Biotechnology has identified an A. thaliana transcription factor (G975) that leads to increased accumulation of epicuticular wax (WO 01/36597). Overexpression of this gene and it's homologues is tested as a means of increasing wax content in maize, improving water use efficiency in the plant and increasing yield. G975 is a member of the AP2/EREBP family (EREBP subfamily) of transcription factors. G975 is expressed in flowers and, at lower levels, in shoots, leaves, and siliques. GC-FID and GC-MS analyses of leaves from G975 overexpressing plants have shown that the levels of C29, C31, and C33 alkanes are substantially increased (up to 10-fold) compared to control plants. A number of additional compounds of similar molecular weight, presumably also wax components, accumulate to significantly higher levels in G975 overexpressing plants. Although total amounts of wax in G975 overexpressing plants have not yet been measured, C29 alkanes constitute close to 50% of the wax content in wild-type plants (Millar et al., 1998), suggesting that a major increase in total wax content occurs in these transgenic plants. However, the transgenic plants have an almost normal phenotype (although small morphological differences are detected in leaf appearance), indicating that overexpression of G975 is not deleterious to the plant. It is noteworthy that overexpression of G975 does not cause the dramatic alterations in plant morphology that have been reported for Arabidopsis plants in which the FATTY ACID ELONGATION1 gene was overexpressed (Millar et al., 1998). It is believed that G975 will specifically regulate the expression of genes involved in wax metabolism. One Arabidopsis AP2 gene that is significantly more closely related to G975 than the rest of the members of the AP2/EREBP family was identified. It is believed that this gene, G1387, will have a function (and therefore a use) related to that of G975.

Nucleic acid and polypeptide sequences for use in generation of transgenic crop plants having improved properties as described in the above strategy are provided in Table 1.

42. Alteration of Ear Development

Corn yield is ultimately a product of resources in the soil and energy from sunlight. At lower population density, individual plants perform very well, but the efficiency of resource utilization is low and some sunlight is wasted. At higher population density, soil resources and sunlight utilization is increased. As a result, the biomass produced per acre is higher at high population density. However, corn plants usually favor male flower development when growth conditions are limiting. At high population density, plants grow taller to compete for sunlight and to make sure the tassel is exposed in the open air. This growth behavior restricts ear development and limits grain yield. This invention relates to novel methods of modifying the growth behavior of corn plants by changing the expression level of a gene or genes such that ear development is promoted.

Transcript profiling experiments have shown that the gene represented by EST 700456686H1 (ZMSE001) is expressed at least 2 fold higher in normal developing ears as compared to in ears of similar size but with arrested development. A Blast search against Monsanto rice genomic sequence indicates that a homolog of this EST is located on rice BAC clone OJ990528_{—}08, which is on chromosome 1 at location 48.5 centimorgan. Several rice yield component QTLs, including 1000 grain weight and tiller number, have been found in this region of chromosome 1. Based on the above data, the gene represented by EST 700456686H1 is believed to be essential for corn ear development. Overexpression of this gene may lead to larger sink and higher yield, while suppression of expression of this gene may result in aborted ear development. A full length clone containing the ZMS001 gene, zmflb73245g08_FLI, was identified and the sequence provided herein.

Nucleic acid and polypeptide sequences for use in generation of transgenic crop plants having improved properties as described in the above strategy are provided in Table 1.

43. Carbonic Anhydrase

Some algae have a unique carbon dioxide concentrating mechanism by which they concentrate bicarbonate in their chloroplasts and cytosol. The CO₂ concentrating mechanism involves the accumulation of bicarbonate and conversion of the accumulated bicarbonate to CO₂ near the location of ribulose bisphosphate carboxylase (RUBISCO) in the chloroplast. Bicarbonate transporters may also be involved in transporting bicarbonate between the various compartments in algal cells.

In higher plants RUBISCO, the enzyme which catalyses the carbon fixation step in photosynthesis, utilizes CO₂ rather than bicarbonate. In C₃ and C₄ plants CO₂ availability is a determining factor in the efficiency of photosynthesis. In C₄ plants such as maize and sorghum one factor in determining the quantum efficiency of photosynthesis may be CO₂ leakage from the bundle sheath cell chloroplast (Furbank 1998). It has been estimated that between 10 and 30% of the CO₂ released in the bundle sheath cells leaks back to the mesophyll cell creating an inherent deficiency in C₄ photosynthesis.

Although C₄ plants have lower rates of photorespiration than C₃ plants, RUBISCO is a relatively inefficient enzyme in C4 plants. The level of CO₂ in vivo in the vicinity of RUBISCO is estimated to be about equal to the Kₘ(CO₂) of RUBISCO or substantially lower than the saturating level of CO₂ for the Vₘₐₓ in C4 plants (Sharkey 1998). Therefore, while oxygen is readily available for C₄ plants, increases in the CO₂ level are expected to increase the velocity of carbon fixation. Increasing the level of CO₂ in the vicinity of RUBISCO is one strategy to improve the quantum efficiency of photosynthesis in maize.

Carbonic anhydrase catalyses the interconversion of CO₂ and bicarbonate and is used by many aquatic organisms to concentrate CO₂ for photosynthesis. Consequently, the overexpression of specific forms of carbonic anhydrase in specific subcellular compartments may increase the in vivo level of CO₂ in maize and therefore the activity of RUBISCO.

There are a number of forms of carbonic anhydrase that occur in nature. In higher plants carbonic anhydrases have been detected in the cytosol, chloroplast stroma, and chloroplast thylakoid membrane. The most important carbonic anhydrase for photosynthesis may be the chloroplast thylakoid carbonic anhydrase. This type of carbonic anhydrase identified in microalgae such as Chlamydomonas and Chlorella and in higher plants such as maize and pea (reviewed in Stemler, 1997). Under alkaline conditions, the Kₘ (bicarbonate) of thylakoid carbonic anhydrase is 10 mM, whereas the Km of the stromal carbonic anhydrase is 40 mM. Therefore, under alkaline conditions created during photosynthesis in the chloroplast stroma, the thylakoid carbonic anhydrase probably plays a bigger role than the stromal carbonic anhydrase in catalyzing the dehydration of bicarbonate and in the maintenance of high CO₂ concentration near RUBSICO (Ignatova et al., 1998).

No plant thylakoid carbonic anhydrase genes have been cloned to date. However, a thylakoid carbonic anhydrase has been reported in Chlamnydomonas reinhardtii that is associated exclusively with the thylakoid membrane and is localized in the thylakoid lumen (Karlsson et al., 1998). It complements the phenotype of cia-3, a mutant that lacks an internal carbonic anhydrase and requires elevated levels of CO₂ for growth. The gene, cah3, encoding this unique thylakoid-type carbonic anhydrase has been cloned (GenBank accession # 408710). Plant stromal carbonic anhydrases are of the beta type, while mammalian carbonic anhydrases are of the alpha type. Alpha carbonic anhydrases are not only kinetically better (their Kₘ is half that of the plant carbonic anhydrases), but they are also less dependent on pH for activity as compared to the beta type carbonic anhydrases.

The thylakoid carbonic anhydrase isolated from Chlamydomonas is localized in the thylakoid lumen. This has led to the proposal of a carbon dioxide concentrating mechanism that involves the thylakoid lumen. According to this mechanism, stromal bicarbonate enters the thylakoid lumen via the pH gradient across the thylakoid membrane created by photosynthetic electron transport. The accumulated bicarbonate is dehydrated to CO₂ by the thylakoid carbonic anhydrase located in the lumen. Carbon dioxide then diffuses out from the lumen into the stroma where it is fixed by RUBISCO (Raven, 1997; Park et al., 1999). Expression of the alpha-carbonic anhydrase from Chlamydomonas is expected to increase bundle sheath CO₂ levels in maize.

A second way to improve photosynthetic efficiency is to accumulate bicarbonate in the chloroplast. Cyanobacteria and microalgae accumulate bicarbonate as a means to prevent the loss of CO₂ through diffusion. Accumulation of bicarbonate in the chloroplast of C₄ plants is expected to fulfill the same objective if a means to convert the bicarbonate to CO₂ in the thylakoids is available. This may be accomplished by the endogenous carbonic anhydrase or alternatively if the level of endogenous carbonic anhydrase is not high enough, by overexpressing both carbonic anhydrase and the bicarbonate transporter.

Bicarbonate accumulation in the chloroplast may be achieved by expressing genes encoding bicarbonate transporters in the plant that facilitate the accumulation of bicarbonate in cyanobacteria and microalgae. In cyanobacteria, bicarbonate transporters are located in the plasma membrane. Therefore, to utilize them for bicarbonate accumulation in the chloroplast, it will be necessary to target them to the chloroplast membrane. Two bicarbonate transporters have been cloned from Synechococcus. A high affinity Na⁺ dependent bicarbonate transporter has been shown to be encoded by the cmpABCD gene cluster (Omata et al., 1999, GenBank Accession numbers D26358 and M32999). The expression of this operon is induced under low CO₂ conditions. Bicarbonate transport activity of this transporter has been demonstrated and confirmed in Synechococcus (Omata et al, 1999). A second putative bicarbonate transporter, IctB, has been cloned from Synechococcus (Bonfil et al., 1998). This gene was identified and cloned from high CO₂ requiring mutants of Synechococcus that are impaired in bicarbonate transport (GenBank Accession number U62616).

Targeting of carbonic anhydrases and bicarbonate transporters to subcellular locations is desirable. For stromal localization, the RUBISCO small subunit transit peptide (RbcS TP) will be used, whereas for lumenal localization, the psbQ transit peptide (psbQ TP; Gaur et al, 1999) will be used. The Chlamydomonas thylakoid localized carbonic anhydrase has a targeting sequence and therefore, it will not be necessary to operably link a transit peptide to this gene. Similarly, the bicarbonate transporter from Chlamydomonas (LIP-36G1) has a targeting sequence. It will be desirable to include a transit peptide with the microbial (Anabaena) carbonic anhydrase to target it to the chloroplast lumen. It is proposed to target the bicarbonate transporter from Synechocystis (IctB) to both the chloroplast lumen and the chloroplast stroma since it is uncertain where it may provide the most benefit.

Improvements in photosynthesis may be achieved by utilizing either a thylakoid carbonic anhydrase or a bicarbonate transporter, each overexpressed independently. Alternatively, the two strategies may be combined and the two components could be expressed simultaneously in the same plant. This strategy will be the most useful in preventing CO₂ diffusion from the cytoplasm and in supplying CO₂ to RUBISCO. The bicarbonate transporter will allow accumulation of bicarbonate in the chloroplast. The accumulated bicarbonate is then converted to CO₂ by the thylakoid carbonic anhydrase, thus mimicking the CO₂ concentrating mechanism utilized by cyanobacteria and microalgae. Plants overexpressing the carbonic anhydrase will be crossed with plants overexpressing the transporters to produce plants overexpressing both genes simultaneously. Alternatively, both carbonic anhydrase and bicarbonate transporter genes may be introduced simulataneously into the plant using transformation methods known in the art.

Nucleic acid and polypeptide sequences for use in generation of transgenic crop plants having improved properties as described in the above strategy are provided in Table 1.

44. F-Box Proteins

F-box containing proteins have been shown to play important roles in plant development, including floral development, circadian clock, response of the plant growth regulators auxin and jasmonic acid (del Pozo and Estelle, 2000) and responses to environmental stresses. Proteins containing this conserved 40-50 amino acid domain are thought to act as adapters that link target proteins to ubiquitin ligase complexes called SCFs (named after their main components, Skp I, Cullin, and an F-box protein). Different F-box containing proteins have been found to associate with SCF and this interaction appears to mediate the specificity of the complexes' ubiquitin ligase activity (Craig and Tyers, 1999). Overexpression of F-box containing proteins has been shown to produce a number of potentially useful phenotypes in plants and other organisms, suggesting that members of this protein family represent a source of genes that may be used to alter or enhance plant performance and potentially increase plant yield or disease resistances when overexpressed. A number of plant F-box protein genes with functional associations are shown below.

F-Box Protein Genes

| Gene | Phenotype | Organism | Reference |
|---|---|---|---|
| VirF | Decreased virulence in | A. tumefaciens | Schrammeijer et al., |
| | mutant | | 2001 |
| FKF1 | Late flowering, clock | A. thaliana | Nelson et al., 2000 |
| | decoupling in mutant | | |
| Zeitlupe/LKP1 | Late flowering, clock | A. thaliana | Somers et al., 2000; |
| | decoupling in mutant | | Kiyosue and Wada, |
| | | | 2000 |
| UFO/Fimbriata/ | Meristem identity | A. thaliana/A. | Samach et al., 1999; |
| Stamina pistilloida | | majus/P. sativum | Wilkinson et al., 2000; |
| | | | Taylor et al., 2001 |
| TIR1 | Reduced auxin response | A. thaliana | Gray et al., 1999; |
| | in mutant | | Ruegger et al., 1998 |
| COI1 | Mutants fail to respond | A. thaliana | Xie et al., 1998 |
| | to jasmonate | | |

Constitutive overexpression of plant F-box containing genes has been reported to produce a number of phenotypes, suggesting that members of this gene family may provide a source of genes capable of modifying plant growth and development. For example, overexpression of either Zeitlupe/LKP1 or FKF1 with a constitutive promoter is reported to produce plants with elongated hypocotyls and petioles and a late flowering phenotype under long days (Nelson et al., 2000; Kiyosue and Wada, 2000). Overexpression of UFO has been reported to induce ectopic expression of AP3 and alterations in floral architecture (Lee et al., 1997). Overexpression of TIR1 has been reported to produce effects similar to those seen when auxin levels are raised, i.e., growth of the primary root was inhibited, root tips became agravitropic, lateral root development was promoted and increased proliferation of root hairs was seen at the primary root tip (Gray et al., 1999).

SCFs function as ubiquitin-ligase complexes and have been shown to trigger the degradation of a wide range of proteins. The F-box is a protein motif of approximately 50 amino acids that functions as a site of protein-protein interaction. The F-box motif links the F-box protein to other components of the SCF complex by binding to the core SCF component Skp I (Schulman et al., 2000).

F-box proteins have more recently been discovered to function in non-SCF protein complexes in a variety of cellular functions. There are 11 F-box proteins in budding yeast, 326 predicted in Caenorhabditis elegans, 22 in Drosophila, and at least 38 in humans. F-box proteins often include additional carboxy-terminal motifs capable of protein-protein interaction; the most common secondary motifs in yeast and human F-box proteins are WD repeats and leucine-rich repeats, both of which have been found to bind phosphorylated substrates to the SCF complex. The majority of F-box proteins have other associated motifs, and the functions of most of these proteins have not yet been defined. Arabidopsis has some 250 proteins that are predicted to contain F-box motifs. To date functions have been assigned to only five of these proteins.

Several criteria may be used to select genes to express in transgenic plants. Monocot homologues of the Arabidopsis F-box proteins associated with phenotypes (Zeitlulpe, FKF1, UFO, TIR1 and COI1) are selected. Corn and soy genes with F-box domains are selected. Proteins where F-box domains are found in conjunction with other functional domains (tubby, WD repeat) are selected for expression in plants.

Tubby domains are predicted to be DNA binding domains (Boggon et al., 1999) and have been identified in the mouse tubby gene and the human retinitis pigmentosa gene. Several clones of monocot genes encoding tubby-like transcription factors with and without F-box domains have been identified. Two of the maize clones appear to be full length. One clone, fC-zmro700093745, encodes a protein with an F-box followed by two tubby domains and the second clone, fC-zmle700440044a, encodes a protein predicted to have two tubby domains.

Nucleic acid and polypeptide sequences for use in generation of transgenic crop plants having improved properties as described in the above strategy are provided in Table 1.

45. HMG CoA Synthase

Mevalonic acid is the precursor of many isoprenoid derivatives, such as sterols, ubiquinones, growth regulators, brassinosteroids and terpenes that are necessary for normal growth, development and defense against pathogens. 3-hydroxy-3-methylglutaryl-coenzyme A synthase (HMGS), an enzyme in the mevalonate biosynthetic pathway, converts acetyl CoA and acetoacetyl CoA to HMGCoA. While there are many studies on the next enzyme in the pathway, HMG CoA reductase, HMGS is less characterized in plants.

In Brassica juncea, a multigene family encodes HMGS. HMGS1 is expressed in all plant parts and exhibits developmental regulation in flower, seed and seedling with highest expression during early development. ABA, mannitol and dehydration down-regulate HMGS expression in Brassica juncea and arrest seedling growth. Methyl jasmonate, salicylic acid and wounding induced HMGS expression suggesting its role in defense response. The B. juncea HMGS is related to HMGS from Arabidopsis thaliana, Pinus sylvestris, Homo sapiens, Blatella germanica and Schizosaccharomyces pombe. Characterisation and regulation of the HMGS gene in cereals such as maize, rice and wheat may elucidate the various components of the pathway in cereals.

Overexpression of HMGS may lead to increased accumulation of specific defense metabolites that could improve defense response (both abiotic and biotic) in maize, rice or wheat.

HMG CoA synthase has also been isolated from yeast. Yeast and soy fC-gmst700790793 are tested for their ability to improve corn performance by increasing disease resistance, or improving growth characteristics of the plant.

Nucleic acid and polypeptide sequences for use in generation of transgenic crop plants having improved properties as described in the above strategy are provided in Table 1.

46. Oxidative Stress

Over the course of a growing season a plant may be exposed to extremes of cold, heat and light (Shaw, 1988). Each of these stresses leads to the production of free radicals and can damage the plant to the extent that overall performance and grain yield are reduced. By protecting plants from the damaging effects of oxidative stress it will be possible to maintain the plant in a highly productive state and increase average grain yields.

Much of the damage associated with oxidative stress appears to be associated with the chloroplast. Cold temperatures, high light intensity and drought stress can all lead to the build up of excess reducing equivalents within photosynthetic reaction centers and the generation of singlet oxygen by photosystem I, photosystem II (Bowyer and Leegood, 1997) and possibly the Rieske Fe—S protein of the cytochrome b6/f complex. The highly reactive and short lived singlet oxygen is believed to inactivate photosystem II D1 protein and reduce oxygen to produce superoxide.

Experiments in which the ratio of electron transport to CO₂ assimilation has been measured have been used to provide support for the above model in corn and sorghum. In maize leaves at normal growth temperatures, the relationship between photosynthetic electron transport and CO₂ assimilation is highly conserved over a wide range of light intensities and CO₂ concentrations (Genty et al., 1989). The majority of the reductants generated by electron transport are consumed by CO₂ assimilation and the effect of other sinks, such as nitrogen metabolism, O₂ reduction via photorespiration, and the Mehler reaction, are minimal (Edwards and Baker, 1993). However, when maize leaves are grown at low temperatures the ratio of electron transport to CO₂ assimilation increases (Fryer et al., 1995, 1998; Massacci et al., 1995). A similar increase in the ratio of electron transport to CO₂ assimilation was also observed when sorghum leaves were drought stressed (Massacci et al., 1996). Therefore, electron sinks, other than CO₂ and O₂, may develop during periods of environmental stresses, thereby imposing restrictions on photosynthetic carbon metabolism.

Plants have developed an system of defenses to cope with the toxic compounds and damage associated with oxidative stress. Superoxide dismutase is the primary mechanism of defense against superoxide generated from singlet oxygen. This enzyme converts superoxide into hydrogen peroxide. Copper/Zinc (Cu/Zn) and iron (Fe) containing superoxide dismutases are found in the chloroplast stroma (Jackson et al., 1978). Overexpression of Fe superoxide dismutase (Van Camp, 1997; Thomas et al., 1999; Van Breusegem et al., 1999; McKersie et al., 2000) and copper/zinc (Gupta et al., 1993) has been reported to protect a wide range of plants from a variety of stresses. Both Fe and Cu/Zn SODs are found in the chloroplast stroma and appear to protect the plant from superoxide in stroma. There have also been reports of Fe SOD being more intimately associated with photosystem II (Navari-Izzo et al., 1999). Manganese (Nm) SOD is thought to be located in the mitochondria of higher plants (Kliebenstein et al., 1998). However, Mn SOD is associated with the thylakoids in cyanobacteria and appears to provide protection against singlet oxygen generated in the thylakoids (Thomas et al., 1998). Overexpression of Mn SOD in higher plants has been reported to protect plants from oxidative stress (Bowler et al., 1991; Zhu and Scandalios, 1992; McKersie et al., 1999). Germin has recently been identified as an extracellular MnSOD (Carter and Thronburg, 2000; Woo et al., 2000).

Carotenoids have also been suggested to play a primary role in the protection of photosynthetic organisms against oxidative stress. They are able to quench ³Chl and ¹O₂ and these functions were demonstrated in vitro in PSII complexes (Telfer et al., 1994). Under excess light, there is a rapid change in the carotenoid composition of the light harvesting complexes. The diepoxide xanthophyll violaxanthin (V) is rapidly and reversibly converted via the intermediate antheraxanthin (A) to the epoxide-free zeaxanthin (Z) under the action of the enzyme V deepoxidase (Bugos et al, 1998). Zeaxanthin appears to protect Arabidopsis thylakoid membranes from lipid peroxidation. However, it is not clear if this effect involves direct quenching of singlet oxygen or another mechanism (Havaux and Niyogi; 1999). Increases in antheraxanthan and zeaxanthin have been seen in drought stressed wheat (Loggini et al, 1999) and it is expected that increasing the zeaxanthin content of thylakoid membranes may protect plants from oxidative stress.

Hydrogen peroxide generated by superoxide dismutase is toxic to the chloroplast and must be rapidly removed. This is achieved by the activity of ascorbate-glutathione cycle peroxidase which reduces hydrogen peroxide to water and oxidizes ascorbate to form monodehydroascorbate radicals. Monodehydroascorbate radicals are then reduced to ascorbate by one of three different pathways in the chloroplast: 1) reduction by monodehydroascorbate reductase with the concomitant oxidation of NADPH; 2) reduction by ferredoxin; or 3) spontaneous disproportionation to form ascorbate and dehydroascorbate. Dehydroascorbate produced by disproportionation is reduced to ascorbate by reduced glutathione in a reaction catalyzed by dehydroascorbate reductase. Finally reduced glutathione is regenerated by glutathione reductase.

The specializations associated with C4 metabolism in corn pose some unique restraints on the ascorbate-glutathione cycle. A model for the maize ascorbate-glutathione cycle was proposed by Kingston-Smith and Foyer (2000). A key observation that this model attempts to explain is the heightened sensitivity of bundle sheath cells to oxidative damage as compared to mesophyll cells. Bundle sheath cells are deficient in photosystem II and the reducing equivalents generated by it. Photosystem I (PSI), reducing molecular oxygen to superoxide (O), is equally distributed between the mesophyll and bundle sheath cells. The superoxide dismutase (SOD) and ascorbate peroxidase (APX) are predominantly located in the bundle sheath cells. Reduced ascorbate (AA) is -oxidized by the action of APX to monodehydroascorbate (MDHA) which is regenerated either by disproportionation to AA and dehydroascorbate (DHA) or by the routes of enzymatic and non-enzymatic reduction available in the bundle sheath cells (Doulis et al., 1997). DHA and oxidized glutathione (GSSG) cannot be reduced in the bundle sheath cells and are transported to the mesophyll cells where the enzymes dehydroascorbate reductase (DHAR) and glutathione reductase (GR) are localized and reducing power is available. AA and reduced glutathione (GSH) are returned to the bundle sheath cells for continued antioxidant defense. Under optimal conditions intercellular transport allows adequate cycling of reduced and oxidized forms of antioxidants. Stress situations lead to extensive carbonyl formation in bundle sheath proteins, but not on mesophyll proteins. Insufficient transport capacity in between bundle sheath and mesophyll cells leads to protein damage.

The model of Kingston-Smith and Foyer predicts that transport of ascorbate or dehydroascorbate is limiting under stress conditions in the bundle sheath of maize. A putative ascorbate/dehydroascorbate transporter has been identified in plants (Horemans et al., 1998), but not cloned. However, vitamin C transporters have been identified in mammalian systems (Daruwala et al., 1999). BLAST searching with the human genes identifies a family of Arabidopsis permeases that may also function as vitamin C transporters. A yeast glutathione transporter has also been recently identified (Bourbouloux et al, 2000). Overexpression of this gene may alter redox balance between different cell types and potentially increase the ability of bundle sheath cells to tolerate oxidative stress.

In addition to the pathways described above, a number of genes have been shown to increase tolerance to oxidative stress by means that are less well understood. Some of these genes are listed below.

Genes for Improving Tolerance to Oxidative Stress

| Gene | Accession | Phenotype | Reference |
|---|---|---|---|
| Superoxide detoxification | | | |
| FeSOD | P21276 | Maize, alfalfa, | Van Camp, 1997; Van |
| | | tobacco and | Breusegem et al., 1999; |
| | | Synechocystis | Thomas et al., 1999; |
| | | resistance | McKersie et al., 2000 |
| Cu/Zn SOD | | Tobacco resistance | Gupta et al., 1993 |
| Mn SOD | | Yeast, tobacco and | Bowler et al., 1991; Zhu |
| | | alfalfa resistance | and Scandalios, 1992; |
| | | | McKersie et al., 1999 |
| Hydrogen peroxide | | | |
| detoxification | | | |
| Catalase | | Tobacco resistance | Shikanai et al., 1998 |
| Other Mechanisms | | | |
| peroxisomal ascorbate | AAF23294.1 | Tobacco resistance | Wang et al., 1999 |
| peroxidase | | | |
| peroxidase | | Arabidopsis resistance | Ezaki et al., 2000 |
| Glutathione S-transferase | | Arabidopsis resistance | Ezaki et al., 2000 |
| Ferritin* | | Tobacco resistance | Deak et al., 1999 |
| GDP-dissociation inhibitor | | Arabidopsis resistance | Ezaki et al., 2000 |
| Blue copper binding protein | | Arabidopsis resistance | Ezaki et al., 2000 |
| NADPH oxidoreductase | CAA89838.1 | Yeast resistance | Babiychuk et al., 1995; |
| | | | Mano et al., 2000 |
| serine acetyltransferase | | Tobacco resistance | Blaszczyk et al., 1999 |
| 1Cys-peroxiredoxin | | Tobacco resistance | Lee et al., 2000 |
| CEF (sec24) | | Yeast resistance | Belles-Boix et al., 2000a |
| CEO1 | AAC98011.1 | Yeast resistance | Belles-Boix et al., |
| | | | 2000b |
| HSP21 | | <<plant>> resistance | Harndahl et al., 1999 |
| Glyoxalase I* | | Tobacco resistance | Veena et al., 1999 |
| PorA/B | | Arabidopsis seedling | Sperling et al., 1997 |
| | | resistance | |
| 2-cys peroxiredoxin | AAF02131.1 | Antisenese sensitivity | Baier and Dietz, 1999 |
| Thioredoxin m and x | | Yeast resistance | Issakidis-Bourguet et |
| | | | al., 2001 |
| Annexin | AAC49472.1 | Yeast resistance | Gidrol et al., 1996 |

Many of the genes are likely to play a role in maintaining the redox driven ascorbate-glutathione detoxification cycle (i.e. peroxidase, GST, blue copper protein, thioredoxin, ferritin) or in damage repair (i.e. 1Cys-peroxiredoxin). Vesicular trafficking appears to play a role in resistance to oxidative stress as is evidenced by improved stress tolerance displayed by tobacco and yeast strains overexpressing GDI (Zarsky et al., 1997; Ezaki et al., 2000) and CEF (Belles-Boix et al., 2000a). In other cases the mechanism of action is less clear (i.e. CEO, annexin). Nucleic acid and polypeptide sequences for use in generation of transgenic crop plants having improved properties as described in the above strategy are provided in Table 1.

47. ATP Synthesis

ATP synthase (FoF1-ATPase) is found in the plasma membrane of bacteria and in the thylakoid membrane of chloroplasts and the cristae membrane of mitochondria of higher plants. ATP synthase catalyzes the production of ATP from ADP and inorganic phosphate. ATP provides energy for cell metabolism by converting energy from a transmembrane electrochemical gradient of protons into the phosphoric acid anhydride bond of ATP. FoF1-ATPase consists of a membrane embedded portion (Fo) which contains the proton translocation machinery, and the soluble F1 portion which contains the nucleotide binding site on which ATP is synthesized. When F1 is detached from Fo and decoupled from the proton-motive driving force, it catalyses ATP hydrolysis and released energy. ATP synthases consists of ten different subunits that total more than 20 polypeptide chains (Girvin, 1998; Junge, 1997; Kaim, 1999).

ATP synthases, also referred to as F-ATPases, are structurally and evolutionarily related to the H+-ATPases located in the plasma membrane of higher plants. This relationship is evident both in their overall structure and the sequence of the membrane spanning proton pump subunits and extrinsic nucleotide binding subunits. However, F-ATPases and H+-ATPases differ in basic function. H+-ATPase couples ATP hydrolysis to proton transport across the membrane generating an electrochemical gradient which is used to drive the secondary transport of various ions and metabolites by carriers and channels (Baunsgaard, 1996; Sze, 1999). H+-ATPase is also thought to play a role in the regulation of cytosolic pH (Morsomme, 1996; Pichon, 1994) and control cell expansion by cell wall acidification (Cosgrove, 1997). It is believed that H+-ATPase is regulated by hormones, light, phytotoxins and environmental stresses (Oufattole, 2000; Michelet, 1995; Wu, 1998; Young, 1998). It possibly acts as an intermediate in certain signal transduction pathways (Michelet, 1995; Palmgren, 1998).

Aminophospholipid ATPase, a member of a new sub-family of P-type ATPases, was suggested to transport lipid-like molecules across the membrane bilayer conferring cold tolerance (Gomes, 2000). Plants gain cold tolerance when membrane phospholipids remain fluid at lower temperatures. An increased concentration of phosphathidylcholine in the membrane's outer leaflet and an increased concentration of anionic phospholipids in the inner leaflet has been associated with distinct lipid fluidity (Tavernier, 1995). Transmembrane redistribution of aminophospholipids in response to cold has also been reported in poikilothermic animals (Miranda, 1996). An asymmetric distribution of phospholipids could also influence the plant's sensitivity to cold indirectly by interfering with signal transduction pathways. It is known that lipids activate a calcium-dependent protein kinase (Szczegielnial, 2000).

The F1 sector and Fo sector of ATP synthase were cloned and sequenced from the purple nonsulfur photosynthetic bacterium Rhodobacter capsulans. ATP synthase F1 extrinsic membrane sector consists of five different subunits (α,β,γ, δ, ε) connected by a thin stalk to the second intrinsic membrane sector (Borghese, 1998). The Fo sector also consists of five different subunits: atpI; atpB; atpE; atpX and atpF (Borghese, 1997). The gene structure of ATP synthase has been studied for a number of organisms (reviewed in Boyer, 1997). The atpHAGDC (F1) operon and atpIBEXF (Fo) operon of R. capsulans represents the complete gene sequence of F1Fo-ATPases. Overexpression of ATP synthase may result in corn plants with higher ATP concentrations and therefore, plants which are capable of more vigorous growth.

H+-ATPases are encoded by a gene family of about ten genes that are differentially expressed depending on cell type, developmental stage and environmental factors (Oufattole, 2000). cDNA clones belonging to two subfamilies have been isolated. Differences between H+-ATPase isoforms have been studied using heterologous expression of Arabidopsis and Nicotiana plumbaginifolia H+-ATPase genes in yeast (Axelsen, 1999; Houlne, 1994; Morsomme, 1998; Palmgren,1994; Piotrowski, 1998; Regenberg, 1995). The data shows that H+-ATPases are not functionally identical. For instance, certain H+-ATPase isoforms might be specialized in different transport functions such as mineral acquisition, phloem loading and stomatal opening (Bouche-Pillon, 1994; DeWitt, 1995; DeWitt. 1991). In N. plumbaginifolia, nine pma (plasma membrane H+ATPase) genes have been identified. Subfamilies I (pma1, pma2, pma3) and II (pma4) represent the major genes expressed. Several transgenic pma4-cosuppressed (reduced expression) plants were produced in tobacco. H+-ATPase activity was reduced by 40-50% as compared to wild type in these plants. Phenotypes were reduced growth, delayed flowering, shorter anther filaments, male sterility, and tip chlorosis of the first mature leaves. Sugar translocation between source and sink tissues via apoplastic phloem loading has been postulated in several species based on the identification of H+/sucrose symporters (Bouche-Pillon, 1994; DeWitt, 1995). PMA4 is expressed in various cell types, but highly expressed in phloem tissue. The apical tissue of transformants with reduced PMA4 expression due to cosuppression showed 29 to 71% less sugar content for sucrose, glucose and fructose, indicating a reduction in translocation from source to sink organs. PMA4 is also expressed in guard cells. In the cosuppressed lines, the guard cells were shriveled and many stomata closed as compared to open wild type stomata (Zhao, 2000).

Overexpression of PMA4 is expected to result in corn plants that are more vigorous due to greater ion transport across membranes. Also, it is expected to result in plants that are more tolerant to drought stress due to reduced embryo abortion caused by low sucrose transport to sink tissue. Mineral uptake in nutrient poor soils may also augmented.

CAP1 was cloned from maize roots and shares sequence identity with ER-type Ca2+-ATPases. CAP1 is novel in that it possesses a C-terminal CaM-binding domain characteristic of PM-type pumps. The CAP1 protein was functionally characterized using a yeast expression system. Yeast mutants defective in Ca2+-ATPases were transformed with CAP1 and calcium transport was restored. Structural changes in CaM and the CAP protein, monitored using CD spectra, were typical of CaM target complexes. Other studies have indicated that CaM regulated Ca2+ pumping activity in maize is predominantly distributed in the ER or the tonoplast. CAP1 transcripts in maize roots were induced during the early hours of anoxia. This appeared to indicate a feedback regulation of cytosolic calcium levels during stress stimulated by calmodulin (Subbaiah, 2000). Calmodulin stimulated Ca2-ATPases have been cloned in soybean (Chung, 2000) and Arabidopsis (Geisler, 2000) with similar stress tolerant findings. Overexpression of CAP1 is expected to increase stress tolerance in a plant.

P-type ATPases are also known. They function by alternately adding and removing a high energy phosphate to an asparate residue to effect the transport of a metal ion. This asparate is contained in a conserved sequence of amino acids typical to the P-type ATPase family (Halleck, 1998). CA2+-ATPases are P-type ATPases. Ca2+ concentration in plant cells is correlated with a variety of external signals such as touch, temperature extremes, ABA, auxin, red light, fungal attack, salinity, drought, anoxia, oxidative stress and hypoxia (reviewed in Bush, 1995). Cellular responses are activated by increased calcium levels which trigger signal transduction pathways, regulation of enzyme activity, ion channel activity and gene expression (Sanders,1999). Ca2+-ATPases are believed to be a major Ca2+ transporter for the endoplasmic reticulum (ER), Golgi apparatus, vacuole, plastid inner membrane and plasma membrane (Liang, 1997;Sanders, 1999). Two distinct types of Ca2+-ATPase pumps have been identified, types IIA and type IIB. Type IIA include ER-type CA2+-ATPases and do not appear to be activated by calmodulin. Type IIB Ca2+ pumps include the plasma membrane type and are stimulated by binding calmodulin (Harper, 1998).

A novel P-type ATPase has been cloned in Arabidopsis belonging to the gene family of aminophosholipid ATPases represented by ALA1 to ALA11. Hydrophobicity analysis of the ALA1 protein indicated 10 transmembrane helices that were very similar to the other ALA proteins. The biochemical function of ALA1 was performed using a lipid internalization assay in yeast expressing the ALA homolog DRS2. The physiological function of ALA1 was tested by expressing ALA1 in a drs2 mutant strain of yeast. ALA1 restored cold tolerance to the mutant. ALA1 antisense plants were generated in Arabidopsis. When these plants were grown at chilling temperatures, antisense lines had smaller rosettes, smaller stems and fewer siliques than wild type (Gomes, 2000). Overexpression of ALA1 in corn is expeccted to result in a plant with increased cold tolerance.

Nucleic acid and polypeptide sequences for use in generation of transgenic crop plants having improved properties as described in the above strategy are provided in Table 1.

48. ATP/ADP Transporters

Plant cells synthesize most ATP using oxidative phosphorylation which occurs in the mitochondrial matrix. Mitochondria import ADP from the cytosol and export ATP in exchange. Adenylates (ATP/ADP) cannot penetrate biomembranes due to their size and negative charge. Therefore, this exchange is mediated by an ATP/ADP transporter protein (AAC) located in the mitochondrial inner membrane (Nelson et al, 1993). AAC is a member of the mitochondrial carrier family comprising a large group of transporters with six transmembrane domains that share the same evolutionary ancestor (Aquila et al, 1987). This carrier contains an obligatory one-for-one ATP/ADP exchange mechanism (Schunemann et al, 1993). Yeast has been used to study the function and structure of AAC (Heidkamper et al, 1996; Muller et al, 1996; Muller et al, 1997; Nelson et al, 1993). Overexpression of AAC in corn should alter cytosolic levels of ATP to meet the plant's energy requirements for increased vigor.

A second adenylate transporter present in the inner membrane of plant plastids has an opposite transport direction. The plastidic ATP/ADP transporter protein (AATP) imports cytosolic ATP in exchange for ADP (Kampfenkel et al, 1995; Neuhaus et al, 1997). These plastidic transport proteins belong to the twelve transmembrane domain family of solute transporters. Plastidic transporters contain an obligatory one-for-one ATP/ADP exchange mechanism but do not share sequence homology with the mitochondrial carrier and are insensitive to AAC inhibitors (reviewed in Winkler and Neuhaus, 1999). Plastidic ATP/ADP transporter genes have been studied in several organisms including spinach, pea and Escherichia coli (Tjaden et al, 1998(a); Tjaden et al. 1998(b)). Novel plastidic transporters have been cloned in Arabidopsis (Mohlmann et al, 1998; Neuhaus et al, 1997). The main function of the plastidic transporter is to supply the plastid's stroma with cytosolic ATP required for anabolic reactions. Starch and fatty acid synthesis catalyzed by ADPglucose pyrophosphorylase and acetyl-CoA carboxylase are strictly dependent on this exogenous supply of ATP (Emes and Nuehaus, 1998).

Tjaden et al. (1998) reported that ATP/ADP transporter activity may represent a rate-limiting function in plant development. The Arabidopsis thaliana cDNA coding for AATP1 was transformed into potato tubers. Transgenic plants expressing the AATP1 gene in a sense orientation showed a substantial increase in the yield of starch. Plants expression the AATP1 gene in an antisense orientation showed a lower level of AATP expression and tubers were smaller in size than wild-type. The reported results correlated with increased ATP uptake into amyloplasts of plants expressing the gene in a sense orientation and lower uptake of ATP in plants epxressing the gene in an antisense. It is expected that overexpression of an ATP/ADP transporter in maize will result in an increase in yield.

Nucleic acid and polypeptide sequences for use in generation of transgenic crop plants having improved properties as described in the above strategy are provided in Table 1.

49. AAA-type ATPases

The plastid is the principle site of fatty acid and essential amino acid biosynthesis in a plant cell. Proteins involved in plastid membrane biogenesis and plastid division may have profound impacts on fatty acid and/or essential amino acid biosynthesis, particularly in grains. These proteins will also likely have an impact on photosynthesis, when expressed in photosynthetically active tissues. Proteins required for plastid division and membrane biogenesis may also affect the size and/or the number of plastids.

AAA, which is an acronym for ATPases Associated to a variety of cellular Activities, belongs to the Walker superfamily of ATPases. Two typical Walker ATPase motifs, named Walker box-A and box B, have been identified in all known AAA ATPase proteins. In addition to these two motifs, AAA-like proteins have a second highly conserved region called the second region of homology (SRH). SRH distinguishes the AAA family from other ATPases. The sequence encompassing Walker box A, box B, and SRH, typically 200-250 amino acids, is termed the AAA cassette. AAA-like proteins usually contain one (type I) or two (type II) copies of the AAA cassette. The number of copies does not appear to define any specific function.

No common function of the AAA-like protein family has been defined. Although AAA proteins are considered as ATPases, the ATPase activity is low, and the proteins have a low sensitivity to a number of ATPase inhibitors. AAA proteins are involved in various cellular functions, including cell division, uncomplexed protein degradation, vesicle-mediated protein transport, peroxisome biogenesis, and plastid division. Chaperone-like activities of some members have also been reported.

AAA homologues were identified in maize by similarity searching using the red alga chloroplast AAA gene (FtsHcp; Itoh et al, 1999) as a query. A number of sequences were identified with high homology to FtsHcp. Among these sequences, one has two copies of the AAA cassette, while the rest show one copy. Based on sequence similarity with known AAA-type genes, two families of proteins were identified, chloroplast and nuclear encoded. Furthermore, two families were identified based on the number of AAA cassettes, i.e., sequences containing one or two AAA cassettes. The sequence that shows highest homology to a AAA type protein is investigated for effects on oil and amino acid contents in grain.

More than 200 members of the AAA family have been identified from bacteria, yeast, animals and plants. No AAA proteins have been reported in maize, but two ESTs from Zea mays were disclosed by Beyer (1997) as AAA-type proteins. Mutations in YME1, a AAA member from Saccharomyces cerevisiae, cause several morphological and functional defects in mitochondria (Thorsness et al. 1993). Furthermore, the chloroplast- and nuclear-encoded red alga FtsH-like genes are suggested to involved in organelle division (Itoh et al. 1999). Overexpression of FtsZ, a plastid division gene, results in the alteration of the size and number of plastids (U.S. Pat. No. 5,981,836).

It is expected that expression of AAA-type proteins in maize will affect fatty acid and/or amino acid composition of the seed. It is desirable to express AAA-type proteins constituitively or specifically in the developing endosperm or embryo. Furthermore, targeting of the protein to the plastid may be desirable.

Nucleic acid and polypeptide sequences for use in generation of transgenic crop plants having improved properties as described in the above strategy are provided in Table 1.

50. Plant Architecture

Breeding strategies that select for altered morphological traits such as reduced plant stature (Peng et al., 999) or stem diameter and canopy structure (Gifford and Evans, 1981) have been used to improve yield in wheat, rice and sugarcane. Historically, this approach has also been successful in corn (Doebley and Wang, 1997). However, recent attempts at selection based on morphology alone have not resulted in improved yield in corn plants (Hageman and Lambert, 1988). In spite of this, analysis of the growth of corn plants and modeling studies suggest that corn yields may be improved via changes in canopy architecture (Duncan, 1971; Mock and Pearce, 1975). This knowledge, coupled with recent improvements in the understanding of leaf development (Tsiantis et al., 1999; Byrne et al., 2000), suggests that transgenic strategies to aimed at altering corn leaf size or shape may result in increases in yield due to improved light capture.

Genes affecting leaf morphology have been identified in maize, rice and Arabidopsis. Nearly all of these genes are transcription factors that may act by altering balances of plant growth regulators. The pleiotropic effects observed when genes affecting leaf morphology are overexpressed in transgenic plants or dominant mutations occur in the endogenous gene suggests that expression of one or more of these genes with a constitutive promoter will identify genes that contribute to plant architecture. Improvements in agronomic performance, such as yield, may require more localized expression in the plant, such as tissue specific or developmentally time expression.

Arabidopsis transcription factors that alter leaf morphology were identified. These genes may provide a means of engineering leaf or floral architecture to alter crop yield. Genes that are expected to effect plant architecture are described below.

Genes Regulating or Altering Leaf Development

| Gene | Effect | Family | Reference |
|---|---|---|---|
| Rough Sheath2/ | Repressor of knox | Myb family | Tsiantis et al., 1999; |
| Asymmetric leaves1 | genes | | Timmermans et al., 1999 Byrne |
| | | | et al., 2000 |
| Knotted1 | Compound | homeobox | Hareven et al., 1996; |
| | leaves/ectopic | | Nishimura et al., 2000 |
| | veination | | |
| Gnarley1 | Sheath/floral | homeobox | Foster et al., 1999a, b |
| | morphology | | |
| Liguleless3 | blade-to-sheath | homeobox | Muehlbauer et al., 1999 |
| | transformation | | |
| OSH | Ectopic leaves/altered | homeobox | Sentoku et al., 2000 |
| | phylotaxy | | |
| Liguleless1 | Ligule/auricle | Squamosoa | Moreno et al., 1997 |
| | formation and | promoter | |
| | leaf/sheath boundary | binding | |
| Liguleless2 | Ligule/auricle | bZIP | Walsh and Freeling, 1999 |
| | development/phase | | |
| | transition | | |
| Rough sheath 1 | unregulated cell | homeobox | Schneeberger et al., 1995 |
| | division and | | |
| | expansion | | |
| G1411 | Reduced apical | AP2 | |
| | dominance | | |
| G1449 | Larger leaf | AUX_IAA | |
| G1496 | Larger leaf | HLH | |
| G1073 | Larger plants, serrated | AT-Hook | |
| | leaves | | |
| G1635 | Reduced apical | myb | |
| | dominance | | |
| G559 | Reduced apical | bZIP | |
| | dominance | | |
| G865 | Reduced apical | AP2 | |
| | dominance | | |

Nucleic acid and polypeptide sequences for use in generation of transgenic crop plants having improved properties as described in the above strategy are provided in Table 1.

51. Phytosiderophores

Soil pH has a major effect on nutrient availability to plants. Metal ions, such as iron, have a limited solubility in high pH soils and, therefore, limited availability of metal ions to a plant may be limiting to plant growth and development in high pH soils. In particular, although iron may be abundant in high pH soils, chlorosis may develop as a result of limited iron availability to the plant.

Graminaceous plants have developed a mechanism for extracting iron from the soil.

Mugineic acid phtyosiderophores (MAs) are secreted from the roots of graminaceous plants and chelate iron in the soil, thereby increasing its bioavailability. Chelated Fe (III)-phytosiderophore complexes are taken up by the root through the action of a transporter in the plasma membrane.

Two enzymes were identified that are induced in response to iron deficiency and are believed to be involved in synthesis of MAs, nicotianamine synthase (NAS) and nicotianamine aminotransferase (NAAT) (Takashi et al., 1999). Secretion of MAs is increased under low iron availability conditions and the level of MAs is believed to correlate with the tolerance of a plant to low availability of iron. It is expected that altering NAAT or NAS activity through overexpression of a transgene will increase the tolerance of a plant to low iron availability through increased production of MAs. Expression of a barley NAAT encoding gene in rice increased plant tolerance to low bioavailability of iron (Takahashi et al., 2001)

A maize gene, ys1 (yellow stripe 1), encoding a transporter of Fe(III)-siderophores has been identified (Curie et al, 2001). It is expected that modified expression of this gene in maize will result in increased iron uptake and plant performance.

Nucleic acid and polypeptide sequences for use in generation of transgenic crop plants having improved properties as described in the above strategy are provided in Table 1. Expression of these genes is expected to increase bioavailability of iron and agronomic performance in plants, especially under conditions of growth in high pH soils.

52. Carbohydrate Transporters

Photosynthesis in mature plant leaves produces reduced carbon that is distributed to non-photosynthetic tissue and organs. “Sources” are organs such as the mature leaves that export the carbon, primarily in the forms of sugars, and “sinks” are organs such as roots and flowers that import sugars for growth and development. The phloem is a highly specialized tissue that evolved to transport sugars.

Long distance sugar transport via the phloem is a major determinant of crop yield. Plasmodesmata serve an especially important role in the phloem. Most plant cells are connected with neighboring cells by channels or plasmodesmata that allow small solutes to cross between cells (Mezitt and Lucas, 1996). Very little is known about plasmodesmata at the molecular level. Microscopic observations have determined that during phloem development, specialized pairs of cells called companion cells (CC) and sieve elements (SE) are formed from a common parent cell and they remain tightly connected by plasmodesmata (Overall and Blackman, 1996). As the plant matures, plasmodesmata between sieve elements widen and form sieve plates (Sjolund, 1997). This creates a living tube through which the phloem sap moves (Balachandran et al, 1997; Oparka et al, 1994;).

The most abundant compound in the phloem sap for most plants is sucrose (Delrot, 1989). There are two mechanisms by which sucrose is loaded/unloaded in the phloem. In symplastic loading, plasmodesmata act as molecular bridges that allow sucrose to move across the cell wall without being transported across a membrane. This transport is passive, down a mass pressure flow gradient. Alternatively, in apoplastic loading, sucrose travels through the extracellular cell wall space from leaf mesophyll cells to the sieve elements. This apoplastic mechanism requires sucrose transporter proteins in the plasma membranes of the sieve elements/companion cells to move it into the phloem (reviewed in Patrick, 1997). These transporters must move the sucrose against a concentration gradient by using the proton motive force created by H+-ATPases (Gahrtz et al, 1994). Once loaded in the phloem, the sucrose is transported osmotically down a pressure flow gradient. The uptake of sucrose into the sieve element is believed to increase the hydrostatic pressure difference between the ends of the phloem conduits so as to drive the mass flow movement of the phloem sap. At the sink tissue, sucrose is unloaded actively into the apoplast and passively down an osmotic gradient for symplastic unloading. Osmolytes such as potassium and amino acids contribute to the driving force of sap flow. The simultaneous withdrawal of osmolytes and water at the sink tissue further increases hydrostatic pressure differences (Delrot, 1989; Galutz et al, 1994; Giaquinta, 1983; Kuhn et al, 1997; vanBel, 1993; Ward et al, 1998).

Membrane sucrose transport genes have been studied widely (Boorer et al, 1996; Burkle et al, 1998; Gahrtz et al, 1996; Harrington et al, 1997; Hirose et al, 1997; Weise et al, 2000). To clone the plant genes, a yeast strain was generated that was capable of metabolizing internal sucrose due to plant sucrose activity but was unable to hydrolyze extracellular sucrose (Riesmeier et al, 1992). Genes encoding sucrose transporters (SUTs) from spinach and potato were cloned by functional complementation in yeast (Riesmeier et al, 1992, 1993). Several SUT proteins were classified as proton symporters because they were energy dependent and sensitive to protonophores (Matsukura et al, 2000; Shakya and Sturm, 1998; Stadler et al, 1998). Similarly, Arabidopsis SUC1 and SUC2 genes encode sucrose transporters of the proton symporters type (Sauer and Stolz, 1994; Stadler and Sauer, 1996; Truernit and Sauer, 1995; Zhou et al, 1997). The SUT and SUC type transporter genes encode highly hydrophobic proteins with 12 membrane spanning domains. In situ hybridization studies demonstrated that the tobacco SUT1 and Arabidopsis SUC2 encoded proteins are present in the plasma membrane of the companion cell/sieve element complexes (reviewed in Ward, 1998). The SUT1 gene encoding a sucrose transporter was cloned from Nicotiana tabacum and was shown to be involved in phloem loading. Transgenic tobacco plants expressing an antisense SUT1 gene had five-to ten-fold higher leaf sucrose and starch contents than wild type plants, suggesting a reduction in sucrose transport. Plants expressing the antisense SUT1 gene had crinkled leaves, increased anthocyanin and retarded growth rates. Soluble carbohydrates accumulated which led to a reduced rate of photosynthesis. There was a reduction in the development of the roots and delayed or impaired flowering. Efflux measurements of sucrose from the antisense plants showed a strong reduction in phloem transport (Burkle et at, 1998). A similar accumulation of carbohydrates occurred in potato leaves that were cold girdled to block phloem loading (Lemoine et al, 1996).

Sucrose is unloaded symplasticly via plasmodesmata and it is unloaded into the apoplast of sink tissue. Apoplastic sucrose is imported directly into the sink tissue by SUT/SUC genes or hydrolyzed by an apoplastic invertase into hexoses, i.e., glucose and fructose (Roitsch et al, 1995). The first plant monosaccharide transporter gene HUP1 was cloned from Chlorella kessleri by screening autotrophic versus heterotrophic cells after the addition of hexoses to the medium (Sauer and Tanner, 1989). The hexose carrier function in the plasma membrane of Chenopodium suspension cells was probed by transmembrane exchange diffusion experiments (Gogarten and Bentrup, 1989). The yeast hexose transporters (HXTs) are uniporters (Ozcan and Johnston, 1995) and HUP1 is a symporter (Sauer et al, 1990a; Aoshima et al, 1993). The two genes are homologous and encode proteins with a 12 membrane-spanning domain. Functions were demonstrated by heterologous expression of plant genes in yeast and higher plant hexose transporters were cloned by heterologous hybridization (Sauer et al, 1990b). Additional H+-hexose symporters have been studied (Tubbe and Buckhout, 1992; Weber et al, 1997). The monosaccharide transporter gene family in Arabidopsis is represented by STP (Aoshima et al, 1993; Boorer et al., 1994). The expression patterns of the hexose transporter genes suggest that they function to uptake hexoses into sink tissue (Sauer and Stadler, 1993).

Since sugar transporter proteins have been proven essential for transporting sugar into developing sink tissue, overexpressed levels of these genes in corn is expected to produce a more vigorous and higher yielding plant.

Nucleic acid and polypeptide sequences for use in generation of transgenic crop plants having improved properties as described in the above strategy are provided in Table 1.

53. Carbon Assimilation

Atmospheric carbon fixation (photosynthesis-dark reactions) by plants, algae and photosynthetic bacteria represents the source of energy in these organisms. The Calvin cycle, located in the stroma of the chloroplast, is the primary pathway of carbon assimilation in higher plants. Carbon assimilates either leave the cycle as substrates for sucrose or starch biosynthesis or continue through the cycle to regenerate the carbon acceptor molecule, ribulose-1,5-bisphosphate. Sedoheptulose-1,7-bisphosphatase (SBPase) is an enzyme that catalyzes an essentially irreversible reaction in the branch region where Calvin cycle intermediates can be diverted to sucrose and starch biosynthesis, and therefore may be essential in regulating carbon partitioning between the regeneration phase of the Calvin cycle and sucrose and starch biosynthesis.

SBPase is found only in the chloroplast of higher plants, where it dephosphorylates sedoheptulose-1,7-bisphosphate (SBP) to form sedoheptulose-7-phosphate and inorganic phosphate. This enzyme is specific for sedoheptulose-1,7-bisphosphate and is inhibited by its products as well as glycerate (Schimkat et al., 1990) and fructose-2,6-bisphosphate (Cadet and Meunier, 1988b). Light, a reducing agent, and Mg²⁺ are required for activity (Woodrow, 1982; Cadet and Meunier, 1988a). The enzyme is a homodimer with a subunit molecular mass of 35-38 kDa (Nishizawa and Buchanan, 1981; Cadet and Meunier, 1988c). It has been shown that the removal of more than 80% of the enzymatic activity of SBPase in tobacco plants using antisense expression of an SBPase gene resulted in chlorosis, reduced growth rates, and reduced carbon assimilate levels (Harrison et al, 1998). Reduction in the quantum efficiency of photosystem II was also observed, which resulted in a reduction in the carbohydrate content of leaves. Analysis of carbohydrate content showed that less starch was accumulated, but sucrose levels were maintained. These results indicate that SBPase is a potential rate-limiting step in carbohydrate metabolism.

Various sedoheptulose 1,7-bisphosphatases have been characterized biochemically, and the corresponding mRNAs (cDNA) have been cloned from an alga (GenBank accession number X74418; Hahn and Kuck, 1994) and some higher plants such as Triticum aestivum (GenBank accession number X65540; Raines et al., 1992), Spinaceae oleracea (GenBank accession number L76556; Martin et al., 1996) and Arabidopsis thaliana (GenBank accession number S74719; Willingham et al. 1994). It is expected that overexpression of a nucleic acid sequence encoding SBPase in a transgenic plant will improve carbon assimilation, export and storag; increased photosynthetic capacity, and extended photosynthetic ability.

SBPase is regulated by redox activity catalyzed by the thioredoxin/ferredoxin system. In the dark wheat SBPase is inactive due to disulfide bond formation between csyteine residue 52 and cysteine residue 57 (numbers correspond to mature wheat SBPase, Raines et al. 1999). In the light, thioredoxin reduces the disulfide linkage resulting in an active enzyme. It is expected that modifying the cysteine residues through mutagenesis will prevent formation of the disulfide bond and therefore prevent inactivation of the protein by oxidation. Site-directed mutagenesis to change cysteine residue 110 and cysteine residue 115 was performed on Chlorella SBPase in order to permanently deregulate the enzyme. It is expected that overexpression of a deregulated form of SBPase will produce a more beneficial effect on metabolism than overexpressing the wild type enzyme.

Fructose 1,6 bisphosphate phosphatase (FBPase) has cytosolic and plastidic isoforms. The plastidic form, involved in the Calvin Cycle in higher plants, catalyzes an essential reversible reaction and has therefore been considered as a possible rate limiting reaction for photosynthesis. FBPase, like SBPase, is regulated by redox regulated by light (Chiadmi et al, 1999). It has been shown that the reduction of chloroplastic FBPase activity by antisense expression of an FBPase gene produced a marked reduction in photosynthetic rate and the size and number of starch granules in the leaves of transgenic potato. Thus FBPase is a potential rate-limiting step in photosynthesis.

Bisphosphatase genes from the facultative chemoautotroph Ralstonia eutropha (Alcaligenes eutrophus) and from the algae Synechococcus lepoliensis encode proteins with a dual activity, FBPase and SBPase, in the Calvin cycle (Yoo and Bowien, 1995; Gerbling et al., 1986). A dual function SBPase/FBPase will be used to test the efficacy of enhancing both phosphatase activities of the Calvin Cycle.

The Chlorella SBPase and Ralstonia SBPase/FBPase were cloned (WO 00/70062). Chlorella SBPase was deregulated by site directed mutagenesis of csyteine residue 110 and cysteine residue 115 and been shown to deregulate SBPase overexpressed in a transient protoplast system.

Nucleic acid and polypeptide sequences for use in generation of transgenic crop plants having improved properties as described in the above strategy are provided in Table 1.

54. Flavonoids

Flavonoids are a group of compounds, derived from the phenylpropanoid and acetyl CoA-malonyl CoA pathways. They are ubiquitous in higher plants and present a remarkable structural and functional diversity. Anthocyanins are the glycosylated end products of the pathway that contributes to most of the bright red-purple-violet pigmentation in flowers and fruits of higher plants. Extensive research of the flavonoid pathway in maize, Petunia, and Antirrhinum serve as a knowledge base for the biochemistry, genetics and molecular biology of the pathway for other plant species (Coe et al., 1988, Dooner et al., 1991, De Vlaming et al., 1984, Martin et al., 1991).

The condensation of p-coumaryl CoA (from the phenylpropanoid pathway) with three molecules of malonyl CoA (produced by the condensation of acetyl CoA and CO₂ catalyzed by acetyl CoA carboxylase), catalyzed by the enzyme chalcone synthase, results in the production of the first flavonoid intermediate, a 5,7,4′-trihydroxychalcone (or chalco-naringenin). Modifications such as hydroxylation, methylation, methoxylation, oxidation/reduction, glycosylation and acylation result in structural variants of flavonoids, including chalcones, aurones, flavones, flavanones, flavonols, dihydrochalcones, flavan 3,4-diols, proanthocyanidins, catechins, biflavonoids, isoflavonoids and anthocyanins.

Flavonoids are induced under a variety of abiotic and biotic stresses, such as light, cold, and pathogen attack. There is increasing evidence on the stress responsive nature of the flavonoid pathway in higher plants with emphasis on response to UV irradiation and pathogen attack. The accumulation of flavonoids appears to be one of the very first metabolic responses to a variety of stress situations in plants. It is expected that expression of transgenes relating to flavonoid biosynthesis will enhance abiotic and biotic stress resistance in plants.

Among dicots, the flavonoid biosynthetic pathway has been elucidated in soybean and Arabidopsis. The flavonoid pathway in Arabidopsis was determined based on studies of UV sensitive and altered seed pigmentation mutants. Eleven loci were identified for flavonoid biosynthesis in Arabidopsis (Shirley et al., 1995). Chalcone synthase (CHS), chalcone isomerase (CHI), flavonoid 3′ hydroxylase (F3H), and flavonol synthase (FLS), are encoded by genes expressed early in Arabidopsis development, whereas dihydroflavonol 4-reductase (DFR) and leucoanthocyanidin dioxygenase (LDOX) are encoded by genes expressed late in Arabidopsis development (Pelletier et al., 1999). Several other loci controlling anthocyanin synthesis have also been identified (Albert et al, 1997, Borevitz et al., 2000, Devic et al., 1999).

In comparison to dicots, there is little information from monocots (in particular cereals) on the role of the flavonoid pathway in stress response. Accumulation of flavones and isovitexin glycosides was demonstrated in rye epidermis (Tevini et al., 1991, Reuber et al., 1996). Furthermore, UV-B induction in rye leads to a significant increase in flavonoids in the epidermal layer (50% of the normal whole leaf levels). Rice seedlings (cv. Purpleputtu) exposed to UV-B showed hyper accumulation of the anthocyanins, cyanidin and peonidin glycosides (Reddy et al., 1994). An increase in isoorientin glycoside derivatives was found in an UV tolerant rice genotype with no such changes in the sensitive line (Markham et al., 1998).

Cereals accumulate flavonoids upon infection by a range of pathogens. The 3-deoxyanthocyanidins, apigeninidin, luteolinidin and apigeninidin acyl esters were shown to be produced in Sorghum infected with Colletotrichum graminicola (Nicholson et al., 1987, Snyder and Nicholson, 1990, Hipskind et al., 1990, 1996). Studies on the early events during infection of Sorghum indicate the accumulation of these flavonoids as inclusions in cells under attack which finally coalesce and surround the infection site. The resistant and susceptible genotypes of Sorghum were crossed to determine the genetic basis of phytoalexin response. It was observed that all of the resistant progeny plants accumulated the 3-deoxyanthocyanidins to higher levels than the susceptible progeny (Tenkuano et al., 1993). The mold resistant species of Sorghum showed increased accumulation of flavan 4-ols. The probable use of flavan 4-ol content as a marker to select resistant genotypes in field has been suggested (Jambunathan et al., 1990). Flavonoid aglycones are known to be more toxic to pathogens than their glycoside derivatives. Naringenin and several flavonoids were shown to be toxic to rice pathogens (Padmavati et al., 1997).

The accumulation of anthocyanins in maize upon infection with Helminthosporium maydis was reported by Heim et al. (1993). In resistant plants anthocyanins accumulate in a restricted zone around the lesions. The concentration of these pigments was higher in the resistant than in the susceptible plants. Sakuranetin, a methyl ether of naringenin, was detected in rice leaves under blast infection (Kodama et al., 1992, Grayer et al., 1995) and UV irradiation in rice. The content of sakuranetin in the blast-resistant cultivar was much higher than in a susceptible cultivar (Dillon et al., 1997).

A hydroxylated product of naringenin, eriodictyol, retards the growth of the corn earworm, Helicoverpa zea, a major insect pest of maize and other crops (Elliger et al., 1980a). Maysin (a C-glycosylflavone), produced in maize silks, deters growth of the corn earworm (Elliger, 1980b). Host-plant resistance to corn earworm by antibiosis is caused by the presence of maysin, apimaysin and methoxymaysin and related compounds in maize silks. The p1 locus encodes a myb-like transcription factor that regulates the synthesis of C-glycosylflavones, phlobaphenes and 3-deoxyanthocyanins. The flavones are oxidized to quinones, which bind amino acids, thereby causing a reduction in available amino acids and inhibition of larval growth. QTLs controlling maysin and apimaysin concentrations have been identified and their association with corn earworm resistance has been shown (Byrne et al., 1996, McMullen et al., 1998).

A C-glycosylflavonoid from rice, isovitexin, abundant in young leaves of barley and rice hull, was shown to have antioxidant properties (Ramaratnam et al, 1989, Shibamoto et al., 1994).

The introduction of anthocyanin regulatory gene constructs R (regulatory gene) and C1 (Colored-1) from maize into the glabrous mutant, ttg of Arabidopsis led to the restoration of anthocyanin and trichome production (Lloyd et al., 1992). The use of the R gene as a visible marker for transformation has been demonstrated in maize (Ludwig et al., 1990).

The isoflavonoid pathway is almost exclusively found in the Leguminosae. The key enzyme that directs the phenylpropanoid pathway intermediates from flavonoids to isoflavonoids is the cytochrome P450 monooxygenase, isoflavone synthase (IFS). The isoflavonoid compounds are well known for their antimicrobial and antioxidant properties. This pathway is absent in cereals. Engineering cereal crops for production of isoflavonoids could be a useful strategy to improve stress response (Padmavati and Reddy, 1999). Recently, the production of isoflavones genistein and daidzein in Arabidopsis and maize was demonstrated by the introduction of soybean IFS (Yu et al., 2000). This indicates that the flavonoid pathway in cereals such as rice, maize and wheat can be extended to produce isoflavonoids.

The Arabidopsis genes ttg1 and ttg2 have been demonstrated to play a role in regulation of flavonoid accumulation (de Vetten et al., 1997; Walker et al., 1999). Similar to strategies involving overexpression of C1, B, R, P or related transcription factors, overexpression of ttg1, ttg2 or their homologues may result in increased accumulation of flavonoids and potentially enhanced plant performance or pathogen resistance.

Nucleic acid and polypeptide sequences for use in generation of transgenic crop plants having improved properties as described in the above strategy are provided in Table 1.

55. Growth Regulating Factor 1

Gibberillic acid (GA) plays a major role in regulating stem, hypocotyl, petiole, and internode elongation. In addition GA play a role in germination, flowering and male fertility. A gene was identified in deep water rice (Growth Regulating Factor 1, GRF1, GeneBank Accession No. AF201895), which increases rapidly in rice intemodes in response to GA and submergence (van der Knapp et al., 2000). Arabidopsis plants over expressing the rice GRF1 gene demonstrated reduced elongation of inflorescence stems, reduced apical dominance, female sterility, reduced male fertility, delayed germination, and delayed flowering (van der Knaap, 2000).

Maize and rice orthologues of GRF1 are disclosed herein. It is expected that expression of GRF1 and GRF1-like genes in a crop plant such as maize will decrease plant height when plants are grown at a high population density, thereby increasing yield.

Nucleic acid and polypeptide sequences for use in generation of transgenic crop plants having improved properties as described in the above strategy are provided in Table 1.

It is expected that one or more polynucleotide or polypeptide sequences of the present invention will contribute to crop improvement, preferably in maize, in accordance with the strategies disclosed herein and listed in Table 1. The particular genes and proteins that will confer a desirable phenotype in a crop plant are disclosed in Table 1, wherein each polynucleotide and polypeptide sequence is associated with a strategy for crop improvement. It is further expected that one may over express any of the genes of the present invention or suppress the expression of any one of the genes. Methods for gene suppression, including antisense, cosuppression and RNAi technology are known to those of skill in the art. Furthermore, one may desire to expression one or more of the genes of the present invention under control of a promoter which is tissue specific, developmental timing specific, constituitive or inducible. Suitable promoters for use in the present invention are known to those of skill in the art or disclosed herein. Furthermore, it is expected that one may desire to express the sequences disclosed herein in various subcellular locations, such as nuclei, plastids, including chloroplasts and amyloplasts, mitochondria or other subcellular locations. It addition one may desire to target proteins extracellularly. Extracellular targeting may involve targeting to the endoplasmic reticulum of the cell in the process of movement to the extracellular space. Targeting to subcellular and extracellular locations will involve the use of expression elements such as targeting peptides, e.g, nuclear or chloroplast targeting peptides or other sign peptides such as those that direct proteins to the endoplasmic reticulum and extracellular spaces. A variety of targeting or signal peptides and their uses in plants are known to those of skill in the art or are disclosed herein.

Of particular interest in the present invention is maize seed characterized by an enhanced phenotype as compared to its parental maize line. Such maize seed is obtainable by observing transformed plants for the above specified traits or for serendipitously imparted phenotypes resulting from the introduction of a transgenic DNA construct into a non-predetermined location in the genomic DNA of tissue from a parental maize line. The transgenic DNA construct is introduced into the genome in sufficient quantity to produce transgenic cells which can be cultured into plants of transgenic maize having an enhanced phenotype as compared to the parental maize line. Such transgenic maize cells are cultured into transgenic plants which produce progeny transgenic seed. Preferably, the screening program is designed to evaluate multiple events of a plurality of distinct transgenic DNA constructs, e.g. from 2 to 20 or more transgenic events of each of from 2 to 20 or more transgenic DNA constructs, e.g. at least 50 or more or up to 100 or more transgenic DNA constructs. Although the design of a transgenic DNA construct can be based on a rational expectation of a phenotype modification, the method of the invention requires observation of an unexpected, yet desired enhanced phenotype. A useful population for screening for unexpected enhanced phenotypes may comprise 40 or more unique transgenic plants, e.g. at least 100 transgenic plants or even up to 1000 or more unique transgenic plants. Even larger populations can be provided by crossing transgenic plants with other plant lines to provide hybrid populations of transgenic plants, such populations can comprises tens of thousands of transgenic plants for screening.

In methods of this invention transgenic plants and seeds are evaluated for desired phenotypes allowing the selection of seeds. Methods of this invention can be practiced with an optional repeating of a cycle of germinating transgenic seed, growing subsequent generation plants from said transgenic seed, observing phenotypes of said subsequent generation plants and collecting seeds from subsequent generation plants having a desirable enhanced phenotype.

It is contemplated that transgenic maize seed of this invention characterized by an enhanced phenotype will result from use of not only the heterologous DNA listed in Table 1 but also homolgs, orthologs and/or paralogs of such heterologous DNA or similar DNA which has been artificially modified to avoid or minimize an undesired effect but yet still produce the originally observed enhanced phenotype associated with the heterologous DNA listed in Table 1. Thus, heterologous DNA for use in this invention comprises not only DNA coding for a protein of a polypeptide listed in Table 1, e.g. with an amino acid sequence of SEQ ID NO: 369 to SEQ ID NO: 738, but also DNA coding for a protein with an amino acid sequence which is at least 60% identical, e.g. at least 65%, 70% or 75% identical, in some cases more preferably at least 80%, 85%, 90% or 95% identical, to a sequence of SEQ ID NO: 369 to SEQ ID NO: 739. In another aspect of this invention the transgenic maize with an enhanced trait is provided by using heterologous DNA with a nucleic acid sequence of SEQ ID NO: 1 to SEQ ID NO: 368 or a homologous DNA coding for a protein of similar function but with a nucleic acid sequence which is at least 70% identical, e.g. at least 75%, 80%, 85%, 90% or 95% identical, to a sequence of SEQ ID NO: I to SEQ ID NO: 368. Sequence identity is determined over a sequence of substantially the full length of a sequence listed in Table 1.

E. Methods for Plant Transformation

Suitable methods for plant transformation for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, and U.S. Pat. No. 5,464,765, each specifically incorporated herein by reference in their entirety), by Agrobacterium-mediated transformation (U.S. Pat. No. 5,591,616 and U.S. Pat. No. 5,563,055; each specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in their entirety), etc. Through the application of techniques such as these, maize cells as well as those of virtually any other plant species may be stably transformed, and these cells developed into transgenic plants. In certain embodiments, acceleration methods are preferred and include, for example, microprojectile bombardment and the like.

1. Electroporation

Where one wishes to introduce DNA by means of electroporation, it is contemplated that the method of Krzyzek et al. (U.S. Pat. No. 5,384,253, incorporated herein by reference in its entirety) will be particularly advantageous. In this method, certain cell wall-degrading enzymes, such as pectin-degrading enzymes, are employed to render the target recipient cells more susceptible to transformation by electroporation than untreated cells. Alternatively, recipient cells are made more susceptible to transformation by mechanical wounding.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; D'Halluin et al, 1992), wheat (Zhou et al., 1993), and soybean (Christou et al., 1987).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in PCT Publication WO 92/17598 (specifically incorporated herein by reference). Other examples of species for which protoplast transformation has been described include barley (Lazerri, 1995), sorghum (Battraw and Hall, 1991), maize (Bhattacharjee et al., 1997), wheat (He et al., 1994) and tomato (Tsukada, 1989).

2. Microprojectile Bombardment

One method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Publication WO 95/06128; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System (BioRad, Hercules, Calif.), which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species. Examples of species for which have been transformed by microprojectile bombardment include monocot species such as maize (PCT Publication WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al, 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (Van Eck et al. 1995), and legumes in general (U.S. Pat. No. 5,563,055, specifically incorporated herein by reference in its entirety).

3. Agrobacterium-Mediated Transformation

Agrobacterium-mediated transfer is a preferred system that is widely applicable for introducing genes into plant. The use of Agrobacterium-mediated plant integrating vectors to introduce DNA into plant cells is well known in the art. See, for example, the methods described by Fraley et al. (1985), Rogers et al. (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al, 1997; Zhang et al., 1997; U.S. Pat. No. 5,591,616, specifically incorporated herein by reference in its entirety), wheat (McCormac et al., 1998), barley (Tingay et al., 1997; McCormac et al., 1998), and maize (Ishida et al., 1996; U.S. Pat. No. 5,981,840).

Modern Agrobacterium transformation vectors are capable of replication in E. coli as well as Agrobacterium, allowing for convenient manipulations as described (Klee et al, 1985). Moreover, recent technological advances in vectors for Agrobacterium-mediated gene transfer have improved the arrangement of genes and restriction sites in the vectors to facilitate the construction of vectors capable of expressing various polypeptide encoding genes. The vectors described (Rogers et al., 1987) have convenient multi-linker regions flanked by a promoter and a polyadenylation site for direct expression of inserted polypeptide encoding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes which cause tumorigenesis or rhizogenesis, respectively, which are used as the vectors and contain the genes of interest that are subsequently introduced into plants. Preferred strains would include but are not limited to Agrobacterium tumefaciens strain C58, a nopaline-type strain that is used to mediate the transfer of DNA into a plant cell, octopine-type strains such as LBA4404 or succinamopine-type strains e.g., EHA101 or EHA105. The use of these strains for plant transformation has been reported and the methods are familiar to those of skill in the art.

Those of skill in the art are aware of the typical steps in the plant transformation process. The Agrobacterium can be prepared either by inoculating a liquid such as Luria Burtani (LB) media directly from a glycerol stock or streaking the Agrobacterium onto a solidified media from a glycerol stock, allowing the bacteria to grow under the appropriate selective conditions, generally from about 26° C.-30° C., more preferably about 28° C., and taking a single colony from the plate and inoculating a liquid culture medium containing the selective agents. Alternatively a loopful or slurry of Agrobacterium can be taken from the plate and resuspended in liquid and used for inoculation. Those of skill in the art are familiar with procedures for growth and suitable culture conditions for Agrobacterium as well as subsequent inoculation procedures. The density of the Agrobacterium culture used for inoculation and the ratio of Agrobacterium cells to explant can vary from one system to the next, and therefore optimization of these parameters for any transformation method is expected.

Typically, an Agrobacterium culture is inoculated from a streaked plate or glycerol stock and is grown overnight, and the bacterial cells are washed and resuspended in a culture medium suitable for inoculation of the explant. Suitable inoculation media for the present invention include, but are not limited ½ MSPL (2.2 g/L GIBCO (Carlsbad, Calif.) MS salts, 2 mg/L glycine, 0.5 g/L niacin, 0.5 g/L L-pyridoxine-HCl, 0.1 mg/L thiamine, 115 g/L L-proline, 26 g/L D-glucose, 68.5 g/L sucrose, pH 5.4) or ½ MS VI (2.2 g/L GIBCO (Carlsbad, Calif.) MS salts, 2 mg/L glycine, 0.5 g/L niacin, 0.5 g/L L-pyridoxine-HCl, 0.1 mg/L thiamine, 115 g/L L-proline, 10 g/L D-glucose, and 10 g/L sucrose, pH 5.4). The inoculation media may be supplemented with a growth inhibiting agent (PCT Publication WO 01/09302). The range and concentration of the growth inhibition agent can vary and depends of the agent and plant system. Growth inhibiting agents including, but not limited to, silver nitrate, silver thiosulfate, or carbenicillin are the preferred growth inhibition agents. The growth inhibiting agent is added in the amount necessary to achieve the desired effect. Silver nitrate is preferably used in the inoculation media at a concentration of about 1 μM (micromolar) to 1 mM (millimolar), more preferably 5 μM-100 μM. The concentration of carbenicillin used in the inoculation media is about 5 mg/L to 100 mg/L, more preferably about 50 mg/L. A compound which induces Agrobacterium virulence genes such as acetosyringone can also be added to the inoculation medium.

In a preferred embodiment, the Agrobacterium used for inoculation are pre-induced in a medium such as a buffered media with appropriate salts containing acetosyringone, a carbohydrate, and selective antibiotics. In a preferred embodiment, the Agrobacterium cultures used for transformation are pre-induced by culturing at about 28° C. in AB-glucose minimal medium (Chilton et al., 1974; Lichtenstein and Draper, 1986) supplemented with acetosyringone at about 200 μM and glucose at about 2%. The concentration of selective antibiotics for Agrobacterium in the pre-induction medium is about half the concentation normally used in selection. The density of the Agrobacterium cells used is about 10⁷-10¹⁰ cfu/ml of Agrobacterium. More preferably, the density of Agrobacterium cells used is about 5×10⁸−4×10⁹ cfu/me. Prior to inoculation the Agrobacterium can be washed in a suitable media such as ½ MS.

The next stage of the transformation process is the inoculation. In this stage the explants and Agrobacterium cell suspensions are mixed together. The mixture of Agrobacterium and explant(s) can also occur prior to or after a wounding step. By wounding as used herein is meant any method to disrupt the plant cells thereby allowing the Agrobacterium to interact with the plant cells. Those of skill in the art are aware of the numerous methods for wounding. These methods would include, but are not limited to, particle bombardment of plant tissues, sonicating, vacuum infiltrating, shearing, piercing, poking, cutting, or tearing plant tissues with a scalpel, needle or other device. The duration and condition of the inoculation and Agrobacterium cell density will vary depending on the plant transformation system. The inoculation is generally performed at a temperature of about 15° C.-30° C., preferably 23° C.-28° C. from less than one minute to about 3 hours. The inoculation can also be done using a vacuum infiltration system.

After inoculation, any excess Agrobacterium suspension can be removed and the Agrobacterium and target plant material are co-cultured. The co-culture refers to the time post-inoculation and prior to transfer to a delay or selection medium. Any number of plant tissue culture media can be used for the co-culture step. For the present invention, a reduced salt media such as half-strength MS-based co-culture media is used and the media lacks complex media additives including but not limited to undefined additives such as casein hydolysate, and B5 vitamins and organic additives. Plant tissues after inoculation with Agrobacterium can be cultured in a liquid media. More preferably, plant tissues after inoculation with Agrobacterium are cultured on a semi-solid culture medium solidified with a gelling agent such as agarose, more preferably a low EEO agarose. The co-culture duration is from about one hour to 72 hours, preferably less than 36 hours, more preferably about 6 hours to 35 hours. The co-culture media can contain one or more Agrobacterium growth inhibiting agent(s) or combination of growth inhibiting agents such as silver nitrate, silver thiosulfate, or carbenicillin. The concentration of silver nitrate or silver thiosulfate is preferably about 1 μM to 1 mM, more preferably about 5 μM to 100 μM, even more preferably about 10 μM to 50 μM, most preferably about 20 μM. The concentration of carbenicillin in the co-culture medium is preferably about 5 mg/L to 100 mg/L more preferably 10 mg/L to 50 mg/L, even more preferably about 50 mg/L. The co-culture is typically performed for about one to three days more preferably for less than 24 hours at a temperature of about 18° C.-30° C., more preferably about 23° C.-25° C. The co-culture can be performed in the light or in light-limiting conditions. Preferably, the co-culture is performed in light-limiting conditions. By light-limiting conditions as used herein is meant any conditions which limit light during the co-culture period including but not limited to covering a culture dish containing the plant/Agrobacterium mixture with a cloth, foil , or placing the culture dishes in a black bag, or placing the cultured cells in a dark room. Lighting conditions can be optimized for each plant system as is known to those of skill in the art.

After co-culture with Agrobacterium, the explants can be placed directly onto selective media. The explants can be sub-cultured onto selective media in successive steps or stages. For example, the first selective media can contain a low amount of selective agent, and the next sub-culture can contain a higher concentration of selective agent or vice versa. The explants can also be placed directly on a fixed concentration of selective agent. Alternatively, after co-culture with Agrobacterium, the explants can be placed on media without the selective agent. Those of skill in the art are aware of the numerous modifications in selective regimes, media, and growth conditions that can be varied depending on the plant system and the selective agent. In the preferred embodiment, after incubation on non-selective media containing the antibiotics to inhibit Agrobacterium growth without selective agents, the explants are cultured on selective growth media. Typical selective agents include but are not limited to antibiotics such as geneticin (G418), kanamycin, paromomycin, herbicides such as glyphosate or phosephinothericine, or other growth inhibitory compounds such as amino acid analogues, e.g., 5 methyltryptophan. Additional appropriate media components can be added to the selection or delay medium to inhibit Agrobacterium growth. Such media components can include, but are not limited to antibiotics such as carbenicillin or cefotaxime.

After the co-culture step, and preferably before the explants are placed on selective or delay media, cells can be analyzed for efficiency of DNA delivery by a transient assay that can be used to detect the presence of one or more gene(s) contained on the transformation vector, including, but not limited to a screenable marker gene such as the gene that codes for β-glucuronidase (GUS). The total number of blue spots (indicating GUS expression) for a selected number of explants is used as a positive correlation of DNA transfer efficiency. The efficiency of T-DNA delivery and the effect of various culture condition manipulations on T-DNA delivery can be tested in transient analyses as described. A reduction in the T-DNA transfer process can result in a decrease in copy number and complexity of integration as complex integration patterns can originate from co-integration of separate T-DNAs (DeNeve et al., 1997). The effect of culture conditions of the target tissue can be tested by transient analyses and more preferably, in stably transformed plants. Any number of methods are suitable for plant analyses, including but not limited to, histochemical assays, biological assays, and molecular analyses.

After effecting delivery of exogenous DNA to recipient cells, the next steps generally concern identifying the transformed cells for further culturing and plant regeneration. As mentioned herein, in order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

4. Other Transformation Methods

Transformation of plant protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Publication WO 95/06128, specifically incorporated herein by reference in its entirety; Thompson, 1995) and rice (Nagatani, 1997).

F. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin, G418 and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One example of a herbicide which is useful for selection of transformed cell lines in the practice of the invention is the broad spectrum herbicide glyphosate. Glyphosate inhibits the action of the enzyme EPSPS, which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, PCT Publication WO 97/04103. The best characterized mutant EPSPS gene conferring glyphosate resistance comprises amino acid changes at residues 102 and 106, although it is anticipated that other mutations will also be useful (PCT Publication WO 97/04103). Furthermore, a naturally occurring glyphosate resistant EPSPS may be used, e.g., the CP4 gene isolated from Agrobacterium encodes a glyphosate resistant EPSPS (U.S. Pat. No. 5,627,061).

To use the bar-bialaphos or the EPSPS-glyphosate selective systems, tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is believed that ranges of 0.1-50 mg/l bialaphos or 0.1-50 mM glyphosate will find utility in the practice of the invention. Bialaphos and glyphosate are provided as examples of agents suitable for selection of transformants, but the technique of this invention is not limited to them.

Another herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al, 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

It further is contemplated that the herbicide dalapon, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. Pat. No. 5,780,708).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468 and U.S. Pat. No. 6,118,047.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells that are expressing luciferase and manipulate cells expressing in real time. Another screenable marker which may be used in a similar fashion is the gene coding for green fluorescent protein (GFP) or a gene coding for other fluorescing proteins such as DsRed® (Clontech, Palo Alto, Calif.).

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause I 00% inhibition followed by screening of growing tissue for expression of a screenable marker gene such as luciferase or GFP would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene (WO 99/60129).

G. Regeneration and Seed Production

Cells that survive the exposure to the selective agent, or cells that have been scored positive in a screening assay, may be cultured in media that supports regeneration of plants. In an exemplary embodiment, MS and N6 (Chu et al., 1975) media may be modified by including further substances such as growth regulators. Preferred growth regulators for plant regeneration include cytokins such as 6-benzylamino pierine, zeahin or the like, and abscisic acid. Media improvement in these and like ways has been found to facilitate the growth of cells at specific developmental stages. Tissue may be maintained on a basic media with auxin type growth regulators until sufficient tissue is available to begin plant regeneration efforts, or following repeated rounds of manual selection, until the morphology of the tissue is suitable for regeneration, then transferred to media conducive to maturation of embryoids. Cultures are transferred every 1-4 weeks, preferably every 2-3 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets were transferred to soiless plant growth mix, and hardened off, e.g., in an environmentally controlled chamber at about 85% relative humidity, 600 ppm CO₂, and 25-250 microeinsteins m⁻²s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous expressible gene. Note however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

H. Characterization

To confirm the presence of the exogenous DNA or “transgene(s)” in the regenerating plants, a variety of assays may be performed. Such assays include, for example, “molecular biological” assays, such as Southern and Northern blotting and PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

1. DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR). Using this technique discreet fragments of DNA are amplified and detected by gel electrophoresis. This type of analysis permits one to determine whether a gene is present in a stable transformant, but does not necessarily prove integration of the introduced gene into the host cell genome. Typically, DNA has been integrated into the genome of all transformants that demonstrate the presence of the gene through PCR analysis. In addition, it is not possible using PCR techniques to determine whether transformants have exogenous genes introduced into different sites in the genome, i.e., whether transformants are of independent origin. Using PCR techniques it is possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition, it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

It is contemplated that using the techniques of dot or slot blot hybridization, which are modifications of Southern hybridization techniques, one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene. Whereas DNA analysis techniques may be conducted using DNA isolated from any part of a plant, RNA will only be expressed in particular cells or tissue types and hence it will be necessary to prepare RNA for analysis from these tissues. PCR techniques, referred to as RT-PCR, also may be used for detection and quantitation of RNA produced from introduced genes. In this application of PCR it is first necessary to reverse transcribe RNA into DNA, using enzymes such as reverse transcriptase, and then through the use of conventional PCR techniques amplify the DNA. In most instances PC techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species. It is further contemplated that TAQMAN® technology (Applied Biosystems, Foster City, Calif.) may be used to quantitate both DNA and RNA in a transgenic cell.

2. Gene Expression

While Southern blotting and PCR may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following an increase in fluorescence as anthranilate is produced, to name two.

Very frequently the expression of a gene product is determined by evaluating the phenotypic results of its expression. These assays also may take many forms, including but not limited to, analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

I. Event Specific Transgene Assays

Southern blotting, PCR and RT-PCR techniques can be used to identify the presence or absence of a given transgene but, depending upon experimental design, may not specifically and uniquely identify identical or related transgene constructs located at different insertion points within the recipient genome. To more precisely characterize the presence of transgenic material in a transformed plant, one skilled in the art could identify the point of insertion of the transgene and, using the sequence of the recipient genome flanking the transgene, develop an assay that specifically and uniquely identifies a particular insertion event. Many methods can be used to determine the point of insertion such as, but not limited to, Genome Walker™ technology (CLONTECH, Palo Alto, Calif.), Vectorette™ technology (Sigma, St. Louis, Mo.), restriction site oligonucleotide PCR (Sarkar et al., 1993; Weber et al., 1998), uneven PCR (Chen and Wu, 1997) and generation of genomic DNA clones containing the transgene of interest in a vector such as, but not limited to, lambda phage.

Once the sequence of the genomic DNA directly adjacent to the transgenic insert on either or both sides has been determined, one skilled in the art can develop an assay to specifically and uniquely identify the insertion event. For example, two oligonucleotide primers can be designed, one wholly contained within the transgene and one wholly contained within the flanking sequence, which can be used together with the PCR technique to generate a PCR product unique to the inserted transgene. In one embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the transgene. In another embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the genomic sequence adjacent to the insertion site. Confirmation of the PCR reaction may be monitored by, but not limited to, size analysis on gel electrophoresis, sequence analysis, hybridization of the PCR product to a specific radiolabeled DNA or RNA probe or to a molecular beacon (Tyagi and Kramer, 1996), or use of the primers in conjugation with a TAQMAN™ probe and technology (Applied Biosystems, Foster City, Calif.)

J. Site Specific Integration or Excision of Transgenes

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. This random insertion of introduced DNA into the genome of host cells can be detrimental to the cell if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence.

Site-specific integration can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. Thus random integration of transgenes is more common in plants. To maintain control over the copy number and the location of the inserted DNA, randomly inserted DNA sequences can be removed. One manner of removing these random insertions is to utilize a site-specific recombinase system (U.S. Pat. No. 5,527,695).

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast (Golic and Lindquist, 1989), the Gin recombinase of phage Mu (Maeser et al, 1991), the Pin recombinase of E. coli (Enomoto et al., 1983), and the R/RS system of the pSR1 plasmid (Araki et al., 1992). The bacteriophage P1 Cre/lox and the yeast FLP/FRT systems constitute two particularly useful systems for site specific integration or excision of transgenes. In these systems; a recombinase (Cre or FLP) will interact specifically with its respective site-specific recombination sequence (lox or FRT, respectively) to invert or excise the intervening sequences. The sequence for each of these two systems is relatively short (34 bp for lox and 47 bp for FRT) and therefore, convenient for use with transformation vectors.

The FLP/FRT recombinase system has been demonstrated to function efficiently in plant cells. Experiments on the performance of the FLP/FRT system in both maize and rice protoplasts indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage PI, recombination between lox sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, 1987). Cre was expressed from an inducible yeast GAL1 promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

K. Deletion of Sequences Located Within the Transgenic Insert

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

It is known that homologous recombination results in genetic rearrangements of transgenes in plants. Repeated DNA sequences have been shown to lead to deletion of a flanked sequence in various dicot species, e.g. Arabidopsis thaliana (Swoboda et al., 1994; Jelesko et al., 1999), Brassica napus (Gal et al., 1991; Swoboda et al, 1993) and Nicotiana tabacum (Peterhans et al, 1990; Zubko et al., 2000). One of the most widely held models for homologous recombination is the double-strand break repair (DSBR) model (Szostak et al., 1983).

Deletion of sequences by homologous recombination relies upon directly repeated DNA sequences positioned about the region to be excised in which the repeated DNA sequences direct excision utilizing native cellular recombination mechanisms. The first fertile transgenic plants are crossed to produce either hybrid or inbred progeny plants, and from those progeny plants, one or more second fertile transgenic plants are selected which contain a second DNA sequence that has been altered by recombination, preferably resulting in the deletion of the ancillary sequence. The first fertile plant can be either hemizygous or homozygous for the DNA sequence containing the directly repeated DNA which will drive the recombination event.

The directly repeated sequences are located 5′ and 3′ to the target sequence in the transgene. As a result of the recombination event, the transgene target sequence may be deleted, amplified or otherwise modified within the plant genome. In the preferred embodiment, a deletion of the target sequence flanked by the directly repeated sequence will result.

Alternatively, directly repeated DNA sequence mediated alterations of transgene insertions may be produced in somatic cells. Preferably, recombination occurs in a cultured cell, e.g., callus, and may be selected based on deletion of a negative selectable marker gene, e.g., the periA gene isolated from Burkholderia caryolphilli which encodes a phosphonate ester hydrolase enzyme that catalyzes the hydrolysis of glyceryl glyphosate to the toxic compound glyphosate (U.S. Pat. No. 5,254,801).

L. Methods of Evaluating Phenotype

Expression, and in some cases suppression, of the various genes embodied by heterologous DNA used in the present invention leads to improved phenotypes in transformed plants. Phenotypic data is collected during the transformation process in callus as well as during plant regeneration, as well as in plant tissue. Phenotypic data can also be collected in transformed callus relating to the morphological appearance as well as growth of the callus, e.g., shooty, rooty, starchy, mucoid, non-embryogenic, increased growth rate, decreased growth rate, dead. It is expected that one of skill in the art may recognize other phenotypic characteristics in transformed callus and plants and select transformed plants having enhanced traits with minimal drag on other key traits, e.g. yield. Phenotypic data is also collected during the process of plant regeneration as well as in regenerated plants transferred to soil. It is expected that one of skill in the art may recognize other phenotypic characteristics in transformed plants.

Although a wide variety of phenotypes are monitored during the process of plant breeding and testing in both inbred and hybrid plants. For example, in R0 plants (plants directly regenerated from callus) and R1 plants (the direct progeny of R0 plants), plant characteristic phenotypes and plant seed characteristic phenotypes can be observed. In R2 and R3 plants, days to pollen shed, days to silking, and plant type can be observed. Metabolite profiling of R2 plants can be conducted. A variety of phenotypes can also be assayed in hybrids of transgenic maize of this invention. For example, yield, moisture, test weight, nutritional composition, chlorophyll content, leaf temperature, stand, seedling vigor, plant height, leaf number, tillering, brace roots, stay green, stalk lodging, root lodging, plant health, barreness/prolificacy, green snap, pest resistance (including diseases, viruses and insects) and metabolic profiles can be recorded. In addition, phenotypic characteristics of grain harvested from hybrids will be recorded, including number of kernels per row on the ear, number of rows of kernels on the ear, kernel abortion, kernel weight, kernel size, kernel density and physical grain quality. Furthermore, characteristics such as photosynthesis, leaf area, husk structure, kernel dry down rate and internode length may be measured in hybrids or inbreds. It is expected that transcriptional profiling may be performed on transgenic plants expressing genes of the present invention.

In a further embodiment of the method of the invention, the transformation and selection steps may be followed by conventional plant improvement techniques thus leading to seeds having an even further improvement in the enhanced phenotype. In still another embodiment the seeds of the invention may be subjected to one or more further transformation treatments.

The maize plants with enhanced phenotype may be used in breeding programs for the development of elite maize lines or hybrids, which programs are aimed at the production of varieties meeting the requirements of farming practice regarding yield, disease resistance and other agronomically important traits in major maize growing areas in the world. Seeds resulting from these programs may be used in the growing of commercial maize crops.

To confirm hybrid yield in transgenic plants expressing genes of the present invention, it may be desirable that hybrids be tested over multiple years at multiple locations in a geographical location where maize is conventionally grown, e.g. in Iowa, Illinois or other locations in the Midwestern United States, under “normal” field conditions as well as under stress conditions, e.g. under drought or population density stress. One of skill in the art knows how to design a yield trial such that a statistically significant yield difference can be detected between two hybrids at the desired rate of precision.

M. Breeding Plants of the Invention

In addition to direct transformation of a particular plant genotype with a construct prepared according to the current invention, transgenic plants may be made by crossing a plant having a construct of the invention to a second plant lacking the construct. For example, a selected coding region operably linked to a promoter can be introduced into a particular plant variety by crossing, without the need for ever directly transforming a plant of that given variety. Therefore, the current invention not only encompasses a plant directly regenerated from cells which have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention.

Backcrossing can be used to improve a starting plant. Backcrossing transfers a specific desirable trait from one source to an inbred or other plant that lacks that trait. This can be accomplished, for example, by first crossing a superior inbred (A) (recurrent parent) to a donor inbred (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred, but are like the superior parent for most or almost all other genes. The last backcross generation would be selfed to give progeny which are pure breeding for the gene(s) being transferred, i.e. one or more transformation events.

Therefore, through a series a breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants which are true breeding for one or more transgenes. By crossing different inbred plants, one may produce a large number of different hybrids with different combinations of transgenes. In this way, plants may be produced which have the desirable agronomic properties frequently associated with hybrids (“hybrid vigor”), as well as the desirable characteristics imparted by one or more transgene(s).

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

It is desirable to introgress the genes of the present invention into maize hybrids for characterization of the phenotype conferred by each gene in a transformed plant. The host genotype into which the transgene was introduced, preferably LH59, is an elite inbred and therefore only limited breeding is necessary in order to produce high yielding maize hybrids. The transformed plant, regenerated from callus is crossed, to the same genotype, e.g., LH59. The progeny are self pollinated twice and plants homozygous for the transgene are identified. Homozygous transgenic plants are crossed to a testcross parent in order to produce hybrids. The test cross parent is an inbred belonging to a heterotic group which is different from that of the transgenic parent and for which it is known that high yielding hybrids can be generated, for example hybrids are produced from crosses of LH59 to either LH195 or LH200.

N. Methods of Evaluating Phenotype

Expression of the genes of the present invention leads to various phenotypes as disclosed herein in transformed cells and plants. Phenotypic data is collected during the transformation process in callus as well as during plant regeneration, as well as in plants and progeny. Phenotypic data is collected in transformed callus relating to the morphological appearance as well as growth of the callus, e.g., shooty, rooty, starchy, mucoid, non-embryogenic, increased growth rate, decreased growth rate, dead. It is expected that one of skill in the art may recognize other phenotypic characteristics in transformed callus.

Phenotypic data is also collected during the process of plant regeneration as well as in -regenerated plants transferred to soil. Phentoypic data includes characteristics such as normal plants, bushy plants, narrow leaves, striped leaves, knotted phenotype [REF}, chlorosis, albino, anthocyanin production, buggy whipped (a phenomenon known to the art in which the most recently emerged leaves are elongated and wrap around each other), or altered tassels, ears or roots. It is expected that one of skill in the art may recognize other phenotypic characteristics in transformed plants.

A wide variety of phenotypes are monitored during the process of plant breeding and testing in both inbred and hybrid plants. For example, in R0 and R1 plants (plants directly regenerated from callus and the direct progeny of those plants), plant type (general morphological characteristics such as those described above for plantlets) and nutritional composition of grain produced by the plants are recorded. Nutritional composition analysis may include amino acid composition, amount of protein, starch and oil, characteristics of protein, starch and oil, fiber, ash, mineral content may all be measured. It is expected that one of skill in the art may include analyses of other components of the grain. In R2 and R3 plants, days to pollen shed, days to silking, and plant type are observed. Furthermore, metabolite profiling of R2 plants is conducted. Using methods available to those of skill in the art, 50 to 100 or more metabolites may be analyzed in a plant, thereby establishing a metabolic fingerprint of the plant. In addition in R3 plants, leaf extension rate is measured under field conditions. A variety of phenotypes will be assayed in hybrids comprising a transgene of the present invention. For example, yield, moisture, test weight, nutritional composition, chlorophyll content, leaf temperature, stand, seedling vigor, plant height, leaf number, tillering, brace roots, stay green, stalk lodging, root lodging, plant health, barreness/prolificacy, green snap, pest resistance (including diseases, viruses and insects) and metabolic profiles will be recorded. In addition, phenotypic characteristics of grain harvested from hybrids will be recorded, including number of kernels per row on the ear, number of rows of kernels on the ear, kernel abortion, kernel weight, kernel size, kernel density and physical grain quality. Furthermore, characteristics such as photosynthesis, leaf area, husk structure, kernel dry down rate and internode length may be measured in hybrids or inbreds. It is expected that transcriptional profiling may be performed on transgenic plants expressing genes of the present invention.

In order to determine hybrid yield in transgenic plants expressing genes of the present invention, it is recognized that hybrids must be tested at multiple locations in a geographical location where maize is conventionally grown, e.g., Iowa, Illinois or other locations in the Midwestern United States. It is expected that more than one year of yield testing is desirable in order to identify transgenes which contribute to improvement of a maize hybrid. Therefore, transgenic hybrids will be evaluated in a first year at a sufficient number of locations to identify at least an approximately 10% yield difference from a non-transgenic hybrid counterpart. A second year of yield tests is conducted at sufficient locations and with suffucient repetitions to be able to idenify a 4% yield difference between two hybrids. Furthermore, in the second year of yield tests, hybrids will be evaluated under normal field conditions as well as under stress conditions, e.g., under conditions of water or population density stress. One of skill in the art knows how to design a yield trial such that a statistically significant yield difference can be detected between two hybrids at the desired rate of precision.

O. Additional Definitions

As used herein genetic transformation: means a process of introducing a DNA sequence or construct (e.g., a vector or expression cassette) into a cell or protoplast in which that exogenous DNA is incorporated into a chromosome or is capable of autonomous replication.

As used herein, exogenous gene means a gene which is not normally present in a given host genome in the exogenous gene's present form. In this respect, the gene itself may be native to the host genome, however, the exogenous gene will comprise the native gene altered by the addition or deletion of one or more different regulatory elements.

As used herein, expression means the combination of intracellular processes, including transcription and translation undergone by a coding DNA molecule such as a structural gene to produce a polypeptide.

As used herein, expression cassette means a chimeric DNA molecule which is designed for introduction into a host genome by genetic transformation. Preferred expression cassettes will comprise all of the genetic elements necessary to direct the expression of a selected gene.

As used herein, expression vector means a vector comprising at least one expression cassette.

As used herein, obtaining, when used in conjunction with a transgenic plant cell or transgenic plant, means either transforming a non-transgenic plant cell or plant to create the transgenic plant cell or plant, or planting transgenic plant seed to produce the transgenic plant cell or plant.

As used herein, progeny means any subsequent generation, including the seeds and plants therefrom, which is derived from a particular parental plant or set of parental plants.

As used herein, promoter means a recognition site on a DNA sequence or group of DNA sequences that provides an expression control element for a structural gene and to which RNA polymerase specifically binds and initiates RNA synthesis (transcription) of that gene.

As used herein, R₀ transgenic plant means a plant which has been directly transformed with a selected DNA or has been regenerated from a cell or cell cluster which has been transformed with a selected DNA.

As used herein, regeneration means the process of growing a plant from a plant cell (e.g, plant protoplast, callus or explant).

As used herein, selected DNA means, a DNA segment which one desires to introduce into a plant genome by genetic transformation.

As used herein, selected gene means a gene which one desires to have expressed in a transgenic plant, plant cell or plant part. A selected gene may be native or foreign to a host genome, but where the selected gene is present in the host genome, will include one or more regulatory or functional elements which differ from native copies of the gene.

As used herein, transformation construct means a chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

As used herein, transformed cell means a cell the DNA complement of which has been altered by the introduction of an exogenous DNA molecule into that cell.

As used herein, transgene means a segment of DNA which has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

As used herein, transgenic plant means a plant or progeny plant of any subsequent generation derived therefrom, wherein the DNA of the plant or progeny thereof contains an introduced exogenous DNA segment not originally present in a non-transgenic plant of the same strain. The transgenic plant may additionally contain sequences which are native to the plant being transformed, but wherein the “exogenous” gene has been altered in order to alter the level or pattern of expression of the gene.

As used herein, transit peptide means a polypeptide sequence which is capable of directing a polypeptide to a particular organelle or other location within a cell.

As used herein, vector means a DNA molecule capable of replication in a host cell and/or to which another DNA segment can be operatively linked so as to bring about replication of the attached segment. A plasmid is an exemplary vector.

As used herein, purified refers to a nucleic acid molecule or polypeptide separated from substantially all other molecules normally associated with it in its native state. More preferably, a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The terms “isolated and purified” and “substantially purified” are not intended to encompass molecules present in their native state.

The molecules and organisms of the invention may also be recombinant. As used herein, the term recombinant describes (a) nucleic acid molecules that are constructed or modified outside of cells and that can replicate or function in a living cell, (b) molecules that result from the transcription, replication or translation of recombinant nucleic acid molecules, or (c) organisms that contain recombinant nucleic acid molecules or are modified using recombinant nucleic acid molecules.

It is understood that the molecules of the invention may be labeled with reagents that facilitate detection of the agent (e.g., fluorescent labels (Prober, et al., Science 238:336-340 (1987); Albarella et al., EP 144914, chemical labels (Sheldon et al., U.S. Pat. No. 4,582,789; Albarella et al., U.S. Pat. No. 4,563,417, modified bases (Miyoshi et al., EP 119448.

As used herein reduced protein activity and enhanced protein activity in a recombinant cell or organism is determined by reference to a wild type cell or organism and can be determined by direct or indirect measurement. Direct measurement of protein activity might include an analytical assay for the protein, per se, or enzymatic product of protein activity. Indirect assay might include measurement of a property affected by the protein. Enhanced protein activity can be achieved by linking a constitutive promoter to the gene encoding the protein. Reduced protein activity can be achieved by a variety of mechanisms including antisense, mutation or knockout. Antisense RNA will reduce the level of protein expressed and the activity will be reduced as compared to wild type expression levels. A mutation in the gene coding for a protein may not decrease the protein expression, but instead interfere with the protein's function to cause reduced protein activity. A knockout can be achieved by homologous recombination with less than the whole gene.

### EXAMPLES

Using the methods disclosed herein any one of the coding sequences listed in SEQ ID NO: 1-982 is inserted into a plant expression vector, i.e., operably linked to a promoter and other regulatory elements as described previously herein. Genes are inserted in the sense or antisense orientation as appropriate to confer the expected phenotype. A constituitive promoter, such as the rice actin1 promoter is preferably linked to the coding sequence. However, in certain instances use of a tissue specific promoter, e.g., seed specific, may be desirable.

The methods described below are illustrative of methods to express a DNA sequence of interest in a transgenic plant. However, those of skill in the art will know other methods of achieving similar results.

### Example 1

Construction of the Destination Vector

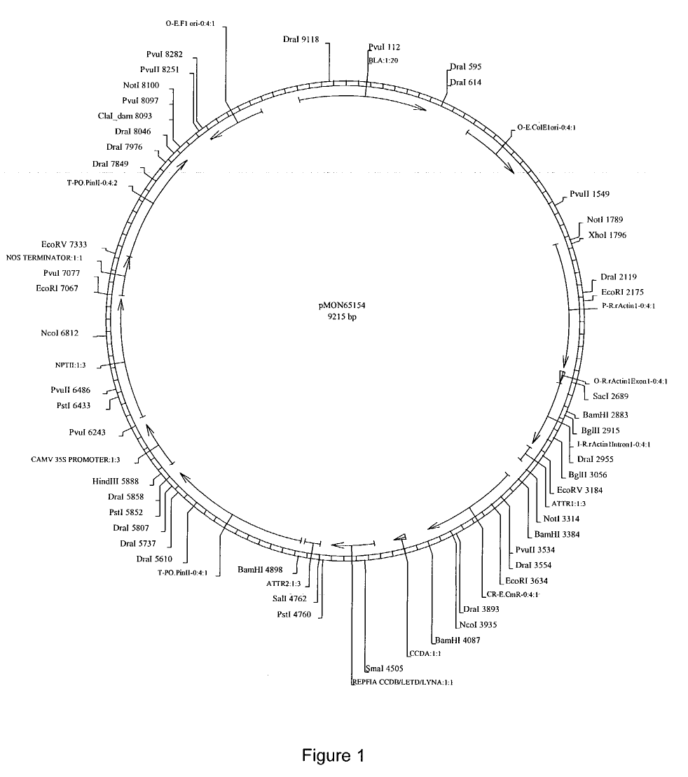

A GATEWAY™ Destination (Invitrogen Life Technologies, Carlsbad, Calif.) plant expression vector was constructed (pMON65154, FIG. 1) using methods known to those of skill in the art. The elements of the expression vector are summarized in 7. The backbone of the plasmid pMON65154 comprising the bacterial replication functions and an ampicillin resistance gene expressed in E. coli were derived from the plasmid pSK-. The plant expression elements in pMON64154 are available to those of skill in the art and references are provided for each element in Table 2. All references in Table 2 to location refer to base pair coordinates for each element on the plasmid-map disclosed in FIG. 1. Generally, pMON65 154 comprises a selectable marker expression cassettte comprising a Cauliflower Mosaic Virus 35S promoter operablly linked to a gene encoding neomycin phosphotransferase II (nptII). The 3′ region of the selectable marker expression cassette comprises the 3′ region of the Agrobacterium tumefaciense nopaline synthase gene (nos) followed 3′ by the 3′ region of the potato proteinase inhibitor II (pinII) gene. The plasmid pMON 65154 further comprises a plant expression cassette into which a gene of interest may be inserted using GATEWAY™ cloning methods. The GATEWAY™ cloning cassette is flanked 5′ by a rice actin 1 promoter, exon and intron and flanked 3′ by the 3′ region of the potato pinII gene. Using GATEWAY™ methods, the cloning cassette was replaced by a gene of interest. The vector pMON65154 and derivaties thereof comprising a gene of interest, were particularly useful in methods of plant transformation via direct DNA delivery, such as microprjectile bombardment. One of skill in the art could construct an expression vector with similar features using methods known in the art. Furthermore, one of skill in the art would appreciate that other promoters and 3′ regions would be useful for expression of a gene of interest and other selectable markers may be used.

TABLE 2
Elements of Plasmid pMON65154

| CASSETTE | FUNCTION | ELEMENT | LOCATION | REFERENCE |
|---|---|---|---|---|
| Plant gene of | Promoter | Rice actin 1 | 1796-2638 | Wang et al., 1992 |
| interest | | | | |
| expression | | | | |
| | Enhancer | Rice actin 1 | 2639-3170 | Wang et al., 1992 |
| | | exon 1, intron | | |
| | | 1 | | |
| GATEWAY ™ | Recombination | AttR1 | 3188-3312 | GATEWAY ™ Cloning |
| cloning | | | | Technology Instruction |
| | | | | Manual (Invitrogen |
| | | | | Life Technologies, |
| | | | | Carlsbad, CA) |
| | Bacterial | CmR gene | 3421-4080 | GATEWAY ™ Cloning |
| | chloramphenical | | | Technology Instruction |
| | resistance gene | | | Manual (Invitrogen |
| | | | | Life Technologies, |
| | | | | Carlsbad, CA) |
| | Bacterial negative | ccdA, ccdB | 4200-4727 | GATEWAY ™ Cloning |
| | selectable markers | genes | | Technology Instruction |
| | | | | Manual (Invitrogen |
| | | | | Life Technologies, |
| | | | | Carlsbad, CA) |
| | GATEWAY ™ | attR2 | 4768-4892 | GATEWAY ™ Cloning |
| | recombination site | | | Technology Instruction |
| | | | | Manual (Invitrogen |
| | | | | Life Technologies, |
| | | | | Carlsbad, CA) |
| Plant gene of | 3′ region | Potato pinII | 4907-5846 | An et al., 1989 |
| interest | | | | |
| expression | | | | |
| cassette | | | | |
| Plant selectable | Promoter | Cauliflower | 5895-6218 | Odell et al., 1985 |
| marker gene | | Mosaic Virus | | |
| expression | | 35S | | |
| cassette | | | | |
| | Selectable marker | nptII | 6252-7046 | Beck et al., 1982 |
| | gene | | | |
| | 3′ region | nos | 7072-7327 | Bevan et al., 1983 |
| | 3′ region | pinII | 7339-8085 | An et al., 1989 |
| Maintenance in | Origin of | ColE1 | 858-1267 | Oka et al, 1979 |
| E. coli | replication | | | |
| Maintenance in | Origin of | F1 | 8273-3673 | Ravetch et al., 1977 |
| E. coli | replication | | | |
| Maintenance in | Ampicillin | Bla | 8909-551 | Heffron et al., 1979 |
| E. coli | resistance | | | |

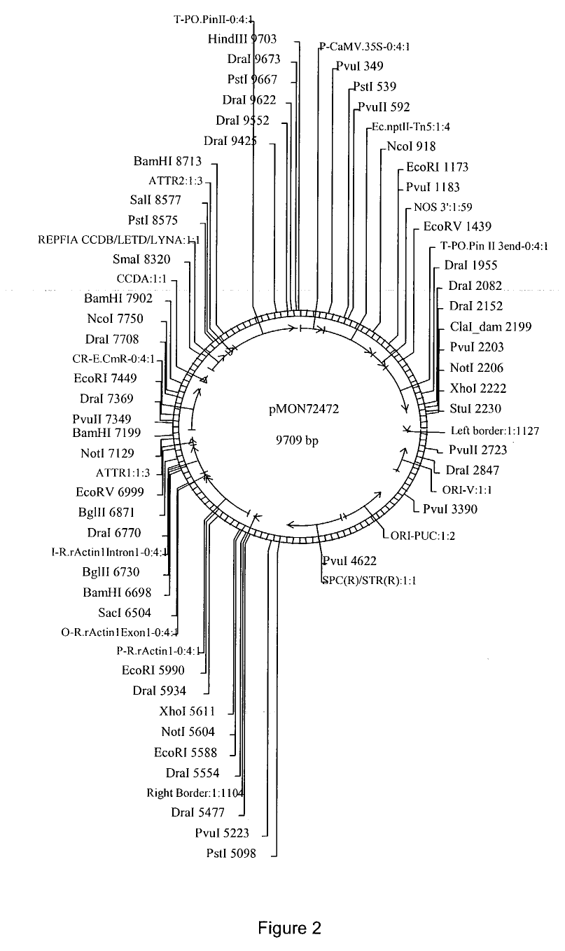

A separate plasmid vector (pMON72472, FIG. 2) was constructed for use in Agrobacterium mediated methods of plant transformation. The plasmid pRG76 comprises the gene of interest plant expression, GATEWAY™ cloning, and plant selectable marker expression cassettes present in pMON65154. In addition left and right T-DNA border sequences from Agrobacterium were added to the plasmid. The right border sequence is located 5′ to the rice actin 1 promoter and the left border sequence is located 3′ to the pinII 3′ sequence situated 3′ to the nptII gene. Furthermore the pSK-backbone of pMON65164 was replaced by a plasmid backbone to facilitate replication of the plasmid in both E. coli and Agrobacterium tumefaciens. The backbone comprises an oriV wide host range origin of DNA replication functional in Agrobacterium, the rop sequence, a pBR322 origin of DNA replication functional in E. coli and a spectinomycin/stretptomycin resistance gene for selection for the presence of the plasmid in both E. coli and Agrobacterium.

The elements present in plasmid vector pRG81 are described in Table 3.

TABLE 3
Genetic Elements of Plasmid Vector pRG81

| CASSETTE | FUNCTION | ELEMENT | LOCATION | REFERENCE |
|---|---|---|---|---|
| Plant gene of | Promoter | Rice actin 1 | 5610-6452 | Wang et al., 1992 |
| interest | | | | |
| expression | | | | |
| | Enhancer | Rice actin 1 | 6453-6984 | Wang et al., 1992 |
| | | exon 1, intron 1 | | |
| GATEWAY ™ | Recombination | AttR1 | 7002-7126 | GATEWAY ™ Cloning |
| cloning | | | | Technology Instruction |
| | | | | Manual (Invitrogen |
| | | | | Life Technologies, |
| | | | | Carlsbad, CA) |
| | Bacterial | CmR gene | 7235-7894 | GATEWAY ™ Cloning |
| | chloramphenical | | | Technology Instruction |
| | resistance gene | | | Manual (Invitrogen |
| | | | | Life Technologies, |
| | | | | Carlsbad, CA) |
| | Bacterial negative | ccdA, ccdB | 8014-8541 | GATEWAY ™ Cloning |
| | selectable | genes | | Technology Instruction |
| | markers | | | Manual (Invitrogen |
| | | | | Life Technologies, |
| | | | | Carlsbad, CA) |
| | GATEWAY ™ | attR2 | 8582-8706 | GATEWAY ™ Cloning |
| | recombination | | | Technology Instruction |
| | site | | | Manual (Invitrogen |
| | | | | Life Technologies, |
| | | | | Carlsbad, CA) |
| Plant gene of | 3′ region | Potato pinII | 8721-9660 | An et al., 1989 |
| interest | | | | |
| expression | | | | |
| cassette | | | | |
| Plant selectable | Promoter | Cauliflower | 1-324 | Odell et al., 1985 |
| marker gene | | Mosaic Virus | | |
| expression | | 35S | | |
| cassette | | | | |
| | Selectable marker | nptII | 358-1152 | Beck et al., 1982 |
| | gene | | | |
| | 3′ region | nos | 1178-1433 | Bevan et al., 1983 |
| | 3′ region | pinII | 1445-2191 | An et al., 1989 |
| Agrobacterium | DNA transfer | Left border | 2493-2516 | Zambryski et al., 1982; |
| mediated | | | | GenBank Accession |
| transformation | | | | AJ237588 |
| Maintenance of | Origin of | Ori-V | 2755-3147 | Honda et al., 1988 |
| plasmid in E. | replication | | | |
| coli or | | | | |
| Agrobacterium | | | | |
| Maintenance of | Origin of | ColE1 | 3545-4199 | Oka et al., 1972 |
| plasmid in E. | replication | | | |
| coli | | | | |
| Maintenance of | Spectinomycin/sts | Spc/Str | 4242-5030 | Fling et al., 1985 |
| plasmid in E. | treptomycin | | | |
| coli or | resistance | | | |
| Agrobacterium | | | | |
| Agrobacterium | DNA transfer | Right border | 5514-5538 | Zambryski et al., 1982; |
| mediated | | | | GenBank Accession |
| transformation | | | | AJ237588 |

### Example 2

Isolation of Coding Sequences

Coding sequences were amplified by PCR prior to insertion in a GATEWAY™ Destination plant expression vector such as pMON65154. All coding sequences were available as either a cloned full length sequence or as DNA sequence information which allowed amplification of the desired sequence from a cDNA library. Primers for PCR amplification were designed at or near the start and stop codons of the coding sequence, in order to eliminate most of the 5′ and 3′ untranslated regions. PCR products were tailed with attB1 and attB2 sequences in order to allow cloning by recombination into GATEWAY™ vectors (Invitrogen Life Technologies, Carlsbad, Calif.).

Two methods were used to produce attB flanked PCR amplified sequences of interest. Both methods are described in detail in the GATEWAY™ Cloning Technology Instruction Manual (Invitrogen Life Technologies, Carlsbad, Calif.). In the first method, a single primer set comprising attB and template specific sequences was used. The primer sequences are as follows:

attB1 forward primer:
5′ GGG CAC TTT GTA CAA GAA AGC TGG GTN template specific sequence 3′
attB2 reverse primer
5′ GGGG CAC TTT GTA CAA GAA AGC TGG GTN template specific sequence 3′

Examples of various PCR primers sets for use in amplification of attB flanked PCR amplifications using a single primer set are disclosed herein as SEQ ID NO: X-Y. Following amplification of attB flanked desired sequences, the PCR product was purified by [INSERT METHOD] and used to produce a GATEWAY™ Entry Vector as described in Example 3 herein.

Alternatively, attB adapter PCR was used to prepare attB flanked PCR products. attB1 adapter PCR uses two sets of primers, i.e., gene specific primers and primers to install the attB sequences. Desired DNA sequence primers were designed which included 12 base pairs of the attB1 or attB2 sequences at the 5′ end. The primers that were used were as follows:

attB1 gene specific forward primer
5′ CCTGCAGGACCATG forward gene specific primer 3′
attB2 gene specific reverse primer
5′ CCTGCAGGCTCGAGCTA reverse gene specific primer

3′

The second set of primers were attB adapter primers with the following sequences:

attB1 adapter forward primer
5′ GGGGACAAGTTTGTACAAAAAAGCAGGCTCCTGCAGGACCATG 3′
attB2 adapter reverse primer
5′ GGGGACCACTTTGTACAAGAAAGCTGGGTCCCTGCAGGCTCGAGCTA

3′.

attB1 and attB2 flanked sequences were amplified by PCR according to the methods described by Invitrogen Life Technologies (Carlsbad, Calif.). attB flanked PCR products were purified and recovered from a gel as described above. Examples of gene specific primers of the current invention are disclosed in SEQ ID NO: X-Y.

In some instances, attB flanked sequences were recovered from PCR, but could not be inserted into the Donor Vector using GATEWAY™ technology. Conventional cloning methods using ligases were used to insert a DNA sequence into an Entry Vector (Invitrogen Life Technologies, Carlsbad, Calif.) when GATEWAY™ recombination into the Donor Vector failed. The choice of Entry Vector depended on the compatibility of restriction endonuclease sites in the Entry Vector and desired insert sequence. The Entry Vector was digested with a selected restriction endonuclease to remove the ccdB gene, dephosphorylated and gel purified. The selected restriction endonuclease depended on the Entry Vector used and the sequence of the desired insert sequence. For example, the ccdB gene was removed from pENTR11 using EcoR1 or other combinations of restriction endonucleases such as EcoRV, and XmaI or NcoI and XhoI. Other restriction nucleases could be used with other Entry Vectors for use in the GATEWAY™ process. To use restriction endonuclease digested Entry Vectors, it was necessary to be able to produce compatible sticky ends on the desired PCR product. Sticky ends could be produced by a number of methods known to those of skill in the art, such as restriction endonuclease digestion, adapter ligation or addition of restriction sites during PCR.

In some instances, it was not possible to produce compatible sticky ends on a PCR fragment and an Entry Vector. Alternatively, compatible sticky ends could be produced directed by restriction enzyme digestion of a cDNA clone. It was possible, however, to blunt end ligate PCR fragments into an Entry Vector. Using this method, the Entry Vector was cut with a restriction endonuclease to remove the ccdB gene. A gel purified linear Entry Vector was made blunt ended with T4 DNA polymerase. One of skill in the art is aware of other methods of making blunt ended DNA molecules, such as the use of Klenow DNA polymerase. The PCR product was made blunt ended and preferably dephosphorylated by incubation with T4 DNA polymerase, or another suitable polymerase, T4 polynucleotide kinase and a phosphatase enzyme. The Entry Vector and PCR product were blunt end ligated using methods known in the art. Ligation products were transformed into E. coli and plasmids from individual colonies analyzed for presence of the insert DNA and the desired orientation relative to the attL sites in the Entry Vector. Clones with the attL1 sequence next to the amino end of the open reading frame were selected.

Preferably, the TA method of cloning PCR products (Marchuk et al., 1991) was used when attB flanked PCR products could not be inserted into a plasmid using GATEWAY™ methods. The TA method takes advantage of Taq polymerase terminal transferase activity. An Entry Vector was cut with a restriction endonuclease and made blunt ended using the methods described herein. The blunt ended linear Entry Vector was incubated with dTTP and Taq polymerase resulting in the addition of a single thymidine residue at the 3′ end of each DNA strand. Since Taq polymerase has a strong preference for dATP, PCR products are most often produced with a single adenosine added to the 3′ end. Therefore,the Entry Vector and PCR product have complimentary single base 3′ overhangs. Following ligation under conditions known to those of skill in the art, plasmids were transformed into E. coli. Plasmids were isolated from individual colonies and analyzed to identify plasmids with the desired insert in the correct orientation. Alternatively, PCR products, tailed with attB sites were TA cloned into a commercial TA cloning vector, such as pGEM-T EASY (Promega Corporation, Madison, Wis).

All PCR amplification products were sequenced prior to introduction into a plant. PCR inserts in Destination expression vectors produced by GATEWAY™ methods were sequenced to confirm that the inserted sequenced encoded the expected amino acid sequence. If Entry Vectors were produced using ligation methods, the inserted sequence was sequenced in the Entry Vector prior to production of the Destination expression vector using GATEWAY™ technology. Point mutations which did not affect the amino acid coding sequence, i.e., silent mutations, were accepted.

### Example 3

Construction of Expression Vectors

GATEWAY™ cloning methods (Invitrogen Life Technologies, Carlsbad, Calif.) were used to construct expression vectors for use in maize transformation. The GATEWAY™ methods are fully described in the GATEWAY™ Cloning Technology Instruction Manual (Invitrogen Life Technologies, Carlsbad, Calif.). Use of the GATEWAY™ system facilitates high throughput cloning of coding sequences into a plant expression vector. Gene sequences flanked by attB1 and attB2 sequences were produced by PCR as described in Example 2. Depending on which recombination sequence, attB1 and attB2, was placed 5′ and 3′ to the coding sequence, sense or antisense expression vectors were produced. A plant expression vector, pMON65 154, into which any coding sequence could be inserted in a sense or antisense orientation was constructed as described in Example 1 and was used as a destination vector in the GATEWAY™ cloning process.

Two alternative processes were used for inserting a PCR amplified coding sequence into a plant expression vector. In the first method, a PCR product comprising the coding sequence of interest flanked by attB1 and attB2 sequences at the 5′ and 3′ ends was incubated with the donor vector (pDONR201™, Invitrogen Life Technologies, Carlsbad, Calif.) in the presence of BP CLONASE™. GATEWAY™ entry clones were produced from this reaction and transformed into E. coli. Plasmid DNA was isolated from entry clones. Inserted coding sequences could be sequenced from entry vectors in order to confirm the fidelity of PCR amplification. Plasmid DNA, isolated from entry clone E. coli colonies, was incubated with linearized destination vector, preferably pMON65 154, in the presence of LR CLONASE™ to produce plant expression vectors comprising the coding sequence of interest. DNA from the LR CLONASE™ reaction was transformed into E. coli. Plasmid DNA from destination expression vectors was isolated and sequenced in order to determine correct orientation and sequence of the plant expression vector.

In the second method of generating plant expression vectors, a PCR product flanked by attB1 and attB2 sequences was incubated with a donor vector (pDONR201™, Invitrogen Life Technologies, Carlsbad, Calif.), and BP CLONASE™ as described above. Following incubation, an aliquot of the reaction mix was further incubated with linearized destination vector and LR CLONASE™. The resultant DNA was transformed into E. Coli and plant expression vectors containing the coding sequence of interest selected using PCR or Southern blot analysis techniques known in the art. Both methods of producting plant expression vectors comprising a coding sequence of interest were described by Invitrogen Life Technologies (GATEWAY™ Cloning Technology Instruction Manual).

Alternatively, Entry Vectors were produced using restriction endonucleases and ligases. Entry Vectors are available from Invitrogen Life Technlogies (Carlsbad, Calif.). Each entry vector, e.g., pENTR1A, pENTR2B, pENTR3C, pENTR4, and pENTR11, has unique cloning and expression features. pENTR11 was preferably used in the practice of the present invention. Those of skill in the art will recognize the usefulness of the other Entry Vectors. Before using restriction endonucleases and ligases to insert desired sequences into one of the Entry Vectors, it was necessary to restriction digest the Entry Vector on each side of the ccdB gene. A number of different combinations of restriction endonucleases were used depending on the restriction sites present on the DNA sequence to be inserted into the Entry Vector. Preferably the Entry Vector was dephosphorylated and gel purified after restriction digestion. The desired DNA sequence was inserted into the Entry Vector using conventional methods of molecular biology known to those of skill in the art. TA cloning (U.S. Pat. No. 5,827,657) is a preferable method of cloning PCR fragments into an Entry Vector.

Vectors (designated as pMON and a 5 digit number) and coding sequences contained there that were produced using the GATEWAY™ cloning methods are listed in Table 4 (Example 9). It is expected that the majority of the coding sequences of the present invention may be cloned into a plant expression vectors using the methods derscribed herein.

### Example 4

Preparation of Recipient Cells

LH59 plants were grown in the greenhouse. Ears were harvested from plants when the embryos were 1.5 to 2.0 mm in length, usually 10 to 15 days after pollination, and most frequently 11 to 12 days after pollination. Ears were surface sterilized by spraying or soaking the ears in 80% ethanol, followed by air drying. Alternatively, ears were surface sterilized by immersion in 50% CLOROX™ containing 10% SDS for 20 minutes, followed by three rinses with sterile water.

Immature embryos were isolated from individual kernels using methods known to those of skill in the art. Immature embryos were cultured on medium 211 (N6 salts, 2% sucrose, 1 mg/L 2,4-D, 0.5 mg/L niacin, 1.0 mg/L thiamine-HCl, 0.91 g/L L-asparagine, 100 mg/L myo-inositiol, 0.5 g/L MES, 100 mg/L casein hydrolysate, 1.6 g/L MgCl₂, 0.69 g/L L-proline, 2 g/L GELGRO™, pH 5.8) containing 16.9 mg/L AgNO₃, (designated medium 211V) for 3-6 days, preferably 3-4 days prior to microprojectile bombardment.

### Example 5

Transformation of Maize Immature Embryos Using A. tumefaciens

Methods of Agrobacterium mediated transformation of maize cells and other monocots are known (Hiei et al., 1997; U.S. Pat. No. 5,591,616; U.S. Pat. No. 5,981,840; published EP patent application EP 0 672 752). Although various strains of Agrobacterium may be used (see references above), strain ABI is used preferably by the present inventors. The ABI strain of Agrobacterium is derived from strain A208, a C58 nopaline type strain, from which the Ti plasmid was eliminated by culture at 37° C., and further containing the modified Ti plasmid pMP90RK (Koncz and Schell, 1986). An Agrobacterium tumefaciens binary vector system (An et al., 1998) is preferably used to transform maize. Alternative cointegrating Ti plasmid vectors have been described (Rogers et al., 1988) and could be used to transform maize. A binary vector comprising one or more genes of interest may be introduced into a disarmed Agrobacterium strain using electroporation (Wen-jun and Forde, 1989) or triparental mating (Ditta et al., 1980). A binary vector may contain a selectable marker gene, a screenable marker gene and/or one or more genes that confer a desirable phenotypic trait on the transformed plant.

Prior to co-culture of maize cells, Agrobacterium cells may be grown at 28° C. in LB (DIFCO) liquid medium comprising appropriate antibiotics to select for maintenance of the modified Ti plasmid and binary vector. For example, ABI/pMON30113, may be grown in LB medium containing 50 ug/ml kanamycin to select for maintenance of the pMP90RK modified Ti plasmid and 100 ug/ml spectinomycin to select for maintenance of the binary vector pMON30113. It will be obvious to one of skill in the art to use appropriate selection agents to maintain plasmids in the host Agrobacterium strain. Prior to inoculation of maize cells, Agrobacterium cells are grown overnight at room temperature in AB medium (Chilton et al, 1974) comprising appropriate antibiotics for plasmid maintenance and 200 uM acetosyringone. Immediately prior to inoculation of maize cells, Agrobacterium are preferably pelleted by centrifugation, washed in ½ MSVI medium (2.2 g/L GIBCO (Carlsbad, Calif.) MS salts, 2 mg/L glycine, 0.5 g/L niacin, 0.5 g/L L-pyridoxine-HCl, 0.1 mg/L thiamine, 115 g/L L-proline, 10 g/L D-glucose, and 10 g/L sucrose, pH 5.4) containing 200 uM acetosyringone, and resuspended at 0.1 to 1.0×10⁹ cells/ml in ½ MSPL medium (2.2 g/L GIBCO (Carlsbad, Calif.) MS salts, 2 mg/L glycine, 0.5 g/L niacin, 0.5 g/L L-pyridoxine-HCl, 0.1 mg/L thiamine, 115 g/L L-proline, 26 g/L D-glucose, 68.5 g/L sucrose, pH 5.4) containing 200 uM acetosyringone. One of skill in the art may substitute other media for ½ MSVI or ½ MSPL.

Immature maize embryos are isolated as described previously. Embryos are inoculated with Agrobacterium 0-7 days after excision, preferably immediately after excision. Alternatively, immature embryos may be cultured for more than 7 days. For example, embryogenic callus may be initiated as described above and co-cultured with Agrobacterium. Preferably, immature maize embryos are excised, immersed in an Agrobacterium suspension in ½ MSPL medium prepared as described above and incubated at room temperature with Agrobacterium for 5-20 minutes.

Following inoculation embryos are transferred to one-half strength MS medium (Murashige and Skoog, 1962) containing 3.0 mg/L 2,4-dichlorophenyoxyacetic acid (2,4-D), 1% D-glucose, 2% sucrose, 0.115 g/L L-proline, 0.5 mg/L thiamine-HCl, 200 uM acetosyringone, and 20 uM silver nitrate or silver thiosulfate. Immature embryos are co-cultured with Agrobacterium for 1 to 3 days at 23° C. in the dark. One of skill in the art may substitute other media for the described media.

Co-cultured embryos are transferred to medium 15AA (462 mg/L (NH4)SO4, 400 mg/L KH2PO4, 186 mg/L MgSO4-7H20, 166 mg7L CaCl2-2H20, 10 mg/L MnSO4-H2O, 3 mg/L H3B03, 2 mg/L ZnSO4-7H20, 0.25 mg/L NaMoO4-2H20, 0.025 mg/L CuSO4-5H20, 0.025 mg/L CoCl2-6H20, 0.75 mg/L KI, 2.83 g/L KNO3, 0.2 mg/L niacin, 0.1 mg/L thiamine-HCl, 0.2 mg/L pyridoxine-HCl, 0.1 mg/L D-biotin, 0.1 mg/L choline chloride, 0.1 mg/L calcium pantothenate, 0.05 mg/L folic acid, 0.05 mg/L p-aminobenzoic acid, 0.05 mg/L riboflavin, 0.015 mg/L vitamin B12, 0.5 g/L casamino acids, 33.5 mg/L Na2EDTA, 1.38 g/L L-proline, 20 g/L sucrose, 10 g/L D-glucose), or MS medium containing 1.5 mg/L 2,4-D, 500 mg/L carbenicillin, 3% sucrose, 1.38 g/L L-proline and 20 uM silver nitrate or silver thiosulfate and cultured for 0 to 8 days in the dark at 27° C. without selection. Culture media used for selection of transformants and regeneration of plants preferably contains 500 mg/L carbenicillin. One of skill in the art may substitute other antibiotics that control growth of Agrobacterium. Other culture media that support cell culture may be used alternatively. In the absence of a delay of selection (0 day culture), selection may be initiated on 25 mg/L paromomycin. Selection medium may comprise medium 211 (described above) or a variant of medium 211 in which N6 salts are replaced by MS salts. After two weeks, embryogenic callus are transferred to culture medium containing 100 mg/L paromomycin and subcultured at about two week intervals. When selection is delayed following co-culture, embryos are initially cultured on medium containing 50 mg/L paromomycin followed by subsequent culture of embryogenic callus on medium containing 100-200 mg/L paromomycin. One of skill in the art will culture tissue on concentrations of paromomycin which inhibit growth of cells lacking the selectable marker gene, but a concentration on which transformed callus will proliferate. Alternatively, one may use other selectable markers to identify transformed cells. It is believed that initial culture on 25 to 50 mg/L paromocyin for about two weeks, followed by culture on 50-200 mg/L paromoycin will result in recovery of transformed callus. Transformants are recovered 6 to 8 weeks after initiation of selection. Plants are regenerated from transformed embryogenic callus as described above for transformants recovered following microprojectile bombardment.

### Example 6

Agrobacterium Mediated Transformation of Maize Callus

This example describes methods for transformation of maize callus using Agrobacterium. The method is exemplified using an nptII selectable marker gene and paromomycin selective agent. One of skill in the art will be aware of other selectable marker and selective agent combinations that could be used alternatively.

Callus was initiated from immature embryos using methods known to those of skill in the art. For example, 1.5 mm to 2.0 mm immature embryos were excised from developing maize seed of a genotype such as LH59 and cultured with the embryonic axis side down on medium 211V (described in Example 4 above), usually for 8-21 days after excision. Alternatively, established an established callus culture may be initiated and maintained by methods known to those of skill in the art.

Agrobacterium was prepared for inoculation of plant tissue according to the methods described in Example 5. Fifty to 100 pieces of callus was transferred to a 60 mm×20 mm petri dish containing about 15 ml of Agrobacterium suspension at 0.1 to 1.0×10⁹ cfu/ml. A piece of callus was usually all of the callus produced by an immature embryo in up to 21 days of culture or a piece of established callus of 2 mm to 8 mm in diameter. Callus was incubated for about 30 minutes at room temperature with the Agrobacterium suspension, followed by removal of the liquid by aspiration.

About 50 μL of sterile distilled water was added to a Whatman #1 filter paper in a 60 mm×20 mm petri dish. After 1-5 minutes, 15 to 20 pieces of callus were transferred to each filter paper and the plate sealed with PARAFILM®, for example. The callus and Agrobacterium were co-cultured for about 3 days at 23° C. in the dark.

Calli were transferred from filter paper to medium 211 with 20 μM silver nitrate and 500 mg/L carbenicillin and cultured in the dark at 27° C. to 28° C. for 2-5 days, preferably 3 days. Selection was initiated by transferring callus to medium 211 containing 20 μM silver nitrate, 500 mg/L carbenicillin and 25 mg/L paromomycin. After 2 weeks culture in the dark at 27° C. to 28° C., callus was transferred to medium 211 with 20 μM silver nitrate, 500 mg/L carbenicillin and 50 mg/L paromomycin (medium 211QRG). Callus was subcultured after two weeks to fresh medium 211 QRG and further cultured for two weeks in the dark at 27° C. to 28° C. Callus was then transferred to medium 211 with 20 μM silver nitrate, 500 mg/L carbenicillin and 75 mg/L paromomycin. After 2-3 weeks culture in the dark at 27° C. to 28° C., paromomycin resistant callus was identified. One of skill in the art would recognize that times between subcultures of callus are approximate and one may be able to accelerate the selection process by transferring tissue at more frequent intervals, e.g., weekly rather than biweekly.

Plants were regenerated from transformed callus, transferred to soil and grown in the greenhouse as described in Example 9. Following Agrobacterium mediated transformation, medium 217 (see Example 9) further contained 500 mg/L carbenicillin and medium 127T (see Example 9) further contained 250 mg/L carbenicillin. Transformed maize plants comprising genes of the present invention that were produced using Agrobacterium mediated transformation are summarized in Table 4 (Example 9).

### Example 7

Methods of Microprojectile Bombardment

Approximately four hours prior to microprojectile bombardment, immature embryos were transferred to medium 211SV (medium 211V with the addition of sucrose to 12%). Twenty five immature embryos were preferably placed in a 60×15 mm petri dish, arranged in a 5×5 grid with the coleoptilar end of the scutellum pressed slightly into the culture medium at a 20 degree angle. Tissue was maintained in the dark prior to bombardment.

Prior to microprojectile bombardment, a suspension of gold particles was prepared onto which the desired DNA was precipitated. Ten milligrams of 0.6 μm gold particles (BioRad) were suspended in 50 μL buffer (150 mM NaCl, 10 mM Tris-HCl, pH 8.0). Twenty five μL of a 2.4 nM solution of the desired DNA was added to the suspension of gold particles and gently vortexed for about five seconds. Seventy five μL of 0.1M spermidine was added and the solution vortexed gently for about 5 seconds. Seventy five μL of a 25% solution of polyethylene glycol (3000-4000 molecular weight, American Type Culture Collection) was added and the solution was gently vortexed for five seconds. Seventy five μL of 2.5 M CaCl₂ was added and the solution vortexed for five seconds. Following the addition of CaCl₂, the solution was incubated at room temperature for 10 to 15 minutes. The suspension was subsequently centrifuged for 20 seconds at 12,000 rpm (Sorval MC-12V centrifuge) and the supernatant discarded. The gold particle/DNA pellet was washed twice with 100% ethanol and resuspended in 10 mL 100% ethanol. The gold particle/DNA preparation was stored at −20° C. for up to two weeks.

DNA was introduced into maize cells using the electric discharge particle acceleration gene delivery device (U.S. Pat. No. 5,015,580). The gold particle/DNA suspension was coated on Mylar sheets (Du Pont Mylar polyester film type SMMC2, aluminum coated on one side, over coated with PVDC co-polymer on both sides, cut to 18 mm square) by dispersion of 310 to 320 μL of the gold particle/DNA suspension on a sheet. After the gold particle suspension settled for one to three minutes, excess ethanol was removed and the sheets were air dried. Microprojectile bombardment of maize tissue was conducted as described in U.S. Pat. No. 5,015,580. AC voltage may be varied in the electric discharge particle delivery device. For microprojectile bombardment of LH59 pre-cultured immature embryos, 35% to 45% of maximum voltage was preferably used. Following microproj ectile bombardment, tissue was cultured in the dark at 27° C.

### Example 8

Selection of Transformed Cells

Transformants were selected on culture medium comprising paromomycin, based on expression of a transgenic neomycin phosphotransferase II (nptII) gene. Twenty four hours after DNA delivery, tissue was transferred to 211V medium containing 25 mg/L paromomycin (medium 211HV). After three weeks incubation in the dark at 27° C., tissue was transferred to medium 211 containing 50 mg/L paromomycin (medium 211G). Tissue was transferred to medium 211 containing 75 mg/L paromomycin (medium 211XX) after three weeks. Transformants were isolated following 9 weeks of selection. Table 4 (Example 9) dislcoses results of transformant experiments using the methods of microprojectile bombardment disclosed herein.

### Example 9

Regeneration of Fertile Transgenic Plants

Fertile transgenic plants were produced from transformed maize cells. Transformed callus was transferred to medium 217 (N6 salts, 1 mg/L thiamine-HCl, 0.5 mg/L niacin, 3.52 mg/L benzylaminopurine, 0.91 mg/L L-asparagine monohydrate, 100 mg/L myo-inositol, 0.5 g/L MES, 1.6 g/L MgCl₂-6H₂O, 100 mg/L casein hydrolysate, 0.69 g/L L-proline, 20 g/L sucrose, 2 g/L GELGRO™, pH 5.8) for five to seven days in the dark at 27° C. Somatic embryos mature and shoot regeneration began on medium 217. Tissue was transferred to medium 127T (MS salts, 0.65 mg/L niacin, 0.125 mg/L pyridoxine-HCl, 0.125 mg/L thiamine-HCl, 0.125 mg/L Ca pantothenate, 150 mg/L L-asparagine, 100 mg/L myo-inositol, 10 g/L glucose, 20 g/L L-maltose, 100 mg/L paromomycin, 5.5 g PHYTAGAR™, pH 5.8) for shoot development. Tissue on medium 127T was cultured in the light at 400-600 lux at 26° C. Plantlets are transferred to soil, preferable 3 inch pots, about four to 6 weeks after transfer to 127T medium when the plantlets are about 3 inches tall and have roots. Plants were maintained for two weeks in a growth chamber at 26° C., followed by two weeks on a mist bench in a greenhouse before transplanting to 5 gallon pots for greenhouse growth. Plants were grown in the greenhouse to maturity and reciprocal pollinations were made with the inbred LH59. Seed was collected from plants and used for further breeding activities. Table 4 (below) summarizes transgenic plants containing the genes of the present invention that have been transferred to soil. The occurrence of pollinations is further noted in Table 4 . In addition, the production of seed from fertile transgenic maize plants comprising the genes of the present invention are summarized in Table 4.

TABLE 4

| | | | | Events With | | Events |
|---|---|---|---|---|---|---|
| | pMON Vector | | Events | Plants | Events | Producing |
| PHE_ID | Number | Transformation Technique | Created | Regenerated | Pollinated | Seed |
| PHE0000006 | PMON68861 | Microprojectile bombardment | 203 | 68 | 18 | 17 |
| PHE0000009 | PMON67803 | Microprojectile bombardment | 182 | 30 | 15 | 6 |
| PHE0000010 | PMON67800 | Microprojectile bombardment | 131 | 68 | 35 | 27 |
| PHE0000012 | PMON67806 | Microprojectile bombardment | 112 | 36 | 12 | 7 |
| PHE0000012 | PMON67808 | Microprojectile bombardment | 441 | 88 | 35 | 26 |
| PHE0000013 | PMON69451 | Microprojectile bombardment | 234 | 88 | 25 | 19 |
| PHE0000013 | PMON69452 | Microprojectile bombardment | 486 | 119 | 45 | 33 |
| PHE0000016 | PMON67750 | Microprojectile bombardment | 125 | 11 | 7 | 6 |
| PHE0000017 | PMON68850 | Microprojectile bombardment | 140 | 26 | 16 | 14 |
| PHE0000022 | PMON67826 | Microprojectile bombardment | 111 | 43 | 16 | 12 |
| PHE0000024 | PMON68354 | Microprojectile bombardment | 318 | 134 | 78 | 68 |
| PHE0000024 | PMON68354 | Microprojectile bombardment | 318 | 134 | 78 | 68 |
| PHE0000025 | PMON68396 | Agrobacterium | 36 | | | |
| PHE0000025 | PMON68396 | Agrobacterium | 36 | | | |
| PHE0000034 | PMON67805 | Microprojectile bombardment | 158 | 53 | 25 | 22 |
| PHE0000038 | PMON68383 | Microprojectile bombardment | 78 | 19 | 3 | 2 |
| PHE0000039 | PMON67807 | Microprojectile bombardment | 120 | 25 | 8 | 7 |
| PHE0000040 | PMON67801 | Microprojectile bombardment | 42 | 17 | 11 | 8 |
| PHE0000040 | PMON77889 | Microprojectile bombardment | 118 | 7 | | |
| PHE0000051 | PMON68859 | Microprojectile bombardment | 50 | 26 | 16 | 15 |
| PHE0000052 | PMON67813 | Microprojectile bombardment | 168 | 62 | 37 | 27 |
| PHE0000055 | PMON68355 | Microprojectile bombardment | 242 | 36 | 19 | 15 |
| PHE0000056 | PMON68364 | Microprojectile bombardment | 501 | 94 | 26 | 19 |
| PHE0000057 | PMON68350 | Microprojectile bombardment | 128 | 52 | 20 | 11 |
| PHE0000058 | PMON68351 | Microprojectile bombardment | 89 | 45 | 21 | 17 |
| PHE0000059 | PMON68370 | Microprojectile bombardment | 120 | 24 | 7 | 5 |
| PHE0000060 | PMON68356 | Microprojectile bombardment | 76 | 22 | 13 | 11 |
| PHE0000064 | PMON67804 | Microprojectile bombardment | 138 | 33 | 12 | 10 |
| PHE0000067 | PMON67816 | Microprojectile bombardment | 151 | 66 | 24 | 19 |
| PHE0000068 | PMON67824 | Microprojectile bombardment | 6 | 4 | 2 | 1 |
| PHE0000069 | PMON67821 | Microprojectile bombardment | 102 | 22 | 10 | 8 |
| PHE0000070 | PMON67825 | Microprojectile bombardment | 288 | 94 | 45 | 33 |
| PHE0000072 | PMON67828 | Microprojectile bombardment | 117 | 29 | 7 | 5 |
| PHE0000073 | PMON68357 | Microprojectile bombardment | 127 | 44 | 35 | 27 |
| PHE0000074 | PMON68352 | Microprojectile bombardment | 127 | 30 | 9 | 7 |
| PHE0000076 | PMON68851 | Microprojectile bombardment | 167 | 31 | 11 | 6 |
| PHE0000077 | PMON67827 | Microprojectile bombardment | 166 | 40 | 11 | 10 |
| PHE0000077 | PMON75301 | Agrobacterium | 9 | | | |
| PHE0000078 | PMON69471 | Microprojectile bombardment | 135 | 31 | 11 | 11 |
| PHE0000078 | PMON77877 | Microprojectile bombardment | 28 | 9 | | |
| PHE0000079 | PMON67752 | Microprojectile bombardment | 219 | 44 | 26 | 25 |
| PHE0000086 | PMON67812 | Microprojectile bombardment | 192 | 60 | 25 | 20 |
| PHE0000091 | PMON68358 | Microprojectile bombardment | 119 | 36 | 8 | 5 |
| PHE0000092 | PMON68359 | Microprojectile bombardment | 99 | 34 | 23 | 12 |
| PHE0000098 | PMON73168 | Microprojectile bombardment | 49 | 20 | 2 | 2 |
| PHE0000102 | PMON67815 | Microprojectile bombardment | 145 | 19 | 10 | 4 |
| PHE0000104 | PMON68608 | Microprojectile bombardment | 108 | 30 | 22 | 18 |
| PHE0000106 | PMON69457 | Microprojectile bombardment | 33 | 22 | 5 | 4 |
| PHE0000108 | PMON67849 | Microprojectile bombardment | 71 | 24 | 11 | 7 |
| PHE0000114 | PMON68361 | Microprojectile bombardment | 92 | 18 | 13 | 12 |
| PHE0000115 | PMON68362 | Microprojectile bombardment | 56 | 2 | | |
| PHE0000116 | PMON68367 | Microprojectile bombardment | 165 | 55 | 18 | 14 |
| PHE0000117 | PMON68368 | Microprojectile bombardment | 100 | 35 | 9 | 4 |
| PHE0000118 | PMON67811 | Microprojectile bombardment | 39 | 19 | 16 | 11 |
| PHE0000119 | PMON68363 | Microprojectile bombardment | 99 | 46 | 17 | 11 |
| PHE0000120 | PMON68853 | Microprojectile bombardment | 141 | 40 | 12 | 9 |
| PHE0000121 | PMON68854 | Microprojectile bombardment | 270 | 55 | 14 | 12 |
| PHE0000122 | PMON74402 | Microprojectile bombardment | 261 | 24 | 2 | 2 |
| PHE0000123 | PMON68855 | Microprojectile bombardment | 61 | 28 | 17 | 11 |
| PHE0000124 | PMON68856 | Microprojectile bombardment | 161 | 34 | 13 | 12 |
| PHE0000125 | PMON68369 | Microprojectile bombardment | 135 | 77 | 46 | 44 |
| PHE0000126 | PMON69458 | Microprojectile bombardment | 112 | 29 | 14 | 12 |
| PHE0000127 | PMON68887 | Microprojectile bombardment | 55 | 35 | 26 | 24 |
| PHE0000133 | PMON68860 | Microprojectile bombardment | 256 | 91 | 25 | 19 |
| PHE0000152 | PMON77899 | Microprojectile bombardment | 94 | 2 | | |
| PHE0000153 | PMON67817 | Microprojectile bombardment | 187 | 69 | 27 | 13 |
| PHE0000154 | PMON67818 | Microprojectile bombardment | 143 | 32 | 24 | 22 |
| PHE0000158 | PMON73169 | Microprojectile bombardment | 138 | 41 | 23 | 17 |
| PHE0000160 | PMON75485 | Microprojectile bombardment | 50 | 22 | 8 | 2 |
| PHE0000162 | PMON75488 | Microprojectile bombardment | 79 | 25 | 12 | 3 |
| PHE0000164 | PMON73170 | Microprojectile bombardment | 60 | 25 | 9 | 6 |
| PHE0000168 | PMON68857 | Microprojectile bombardment | 66 | 34 | 23 | 19 |
| PHE0000173 | PMON73171 | Microprojectile bombardment | 104 | 36 | 10 | 7 |
| PHE0000174 | PMON72465 | Microprojectile bombardment | | | | |
| PHE0000176 | PMON68388 | Microprojectile bombardment | 55 | 24 | 13 | 11 |
| PHE0000177 | PMON68881 | Microprojectile bombardment | 39 | 23 | 14 | 14 |
| PHE0000178 | PMON73166 | Microprojectile bombardment | 42 | 13 | 6 | 4 |
| PHE0000179 | PMON69467 | Microprojectile bombardment | 47 | 11 | 5 | 3 |
| PHE0000181 | PMON76326 | Microprojectile bombardment | 103 | 9 | | |
| PHE0000182 | PMON74420 | Microprojectile bombardment | 164 | 27 | 3 | 3 |
| PHE0000185 | PMON69468 | Microprojectile bombardment | 53 | 27 | 9 | 9 |
| PHE0000186 | PMON69460 | Microprojectile bombardment | 52 | 31 | 7 | 3 |
| PHE0000188 | PMON73167 | Microprojectile bombardment | 168 | 43 | 12 | 8 |
| PHE0000192 | PMON68394 | Agrobacterium | 81 | 2 | 2 | 2 |
| PHE0000193 | PMON68889 | Microprojectile bombardment | 78 | 21 | 8 | 7 |
| PHE0000219 | PMON68865 | Microprojectile bombardment | 52 | 24 | 7 | 7 |
| PHE0000220 | PMON74434 | Microprojectile bombardment | 43 | 28 | 21 | 19 |
| PHE0000223 | PMON69478 | Microprojectile bombardment | 126 | 38 | 28 | 24 |
| PHE0000227 | PMON68376 | Microprojectile bombardment | 119 | 40 | 11 | 6 |
| PHE0000231 | PMON72498 | Microprojectile bombardment | 102 | 29 | 20 | 19 |
| PHE0000232 | PMON68895 | Microprojectile bombardment | 129 | 35 | 9 | 7 |
| PHE0000234 | PMON73159 | Microprojectile bombardment | 48 | | | |
| PHE0000235 | PMON73161 | Microprojectile bombardment | 25 | 10 | 4 | 3 |
| PHE0000237 | PMON68891 | Microprojectile bombardment | 45 | 22 | 9 | 6 |
| PHE0000238 | PMON69466 | Microprojectile bombardment | 88 | 37 | 13 | 12 |
| PHE0000239 | PMON72466 | Microprojectile bombardment | 98 | 29 | 15 | 13 |
| PHE0000240 | PMON72468 | Microprojectile bombardment | 78 | 20 | 10 | 8 |
| PHE0000242 | PMON72470 | Microprojectile bombardment | 134 | 28 | 11 | 9 |
| PHE0000243 | PMON72467 | Microprojectile bombardment | 54 | 29 | 11 | 6 |
| PHE0000244 | PMON68372 | Microprojectile bombardment | 117 | 32 | 11 | 7 |
| PHE0000245 | PMON68373 | Microprojectile bombardment | 27 | 26 | 20 | 17 |
| PHE0000246 | PMON68374 | Microprojectile bombardment | 112 | 54 | 12 | 9 |
| PHE0000247 | PMON68375 | Microprojectile bombardment | 150 | 42 | 16 | 8 |
| PHE0000249 | PMON74422 | Microprojectile bombardment | 52 | 20 | 9 | 7 |
| PHE0000252 | PMON74407 | Microprojectile bombardment | 52 | 28 | 15 | 8 |
| PHE0000254 | PMON73172 | Microprojectile bombardment | 109 | 29 | 10 | 8 |
| PHE0000255 | PMON72459 | Microprojectile bombardment | 73 | 31 | 12 | 9 |
| PHE0000256 | PMON75302 | Agrobacterium | 161 | 37 | 1 | 1 |
| PHE0000258 | PMON68371 | Microprojectile bombardment | 113 | 40 | 8 | 5 |
| PHE0000259 | PMON74404 | Microprojectile bombardment | 113 | 26 | 18 | 15 |
| PHE0000260 | PMON75487 | Microprojectile bombardment | 45 | 14 | 9 | 1 |
| PHE0000262 | PMON68892 | Microprojectile bombardment | 141 | 11 | 2 | 2 |
| PHE0000263 | PMON74412 | Microprojectile bombardment | 95 | 27 | 13 | 8 |
| PHE0000264 | PMON68866 | Microprojectile bombardment | 128 | 38 | 12 | 11 |
| PHE0000265 | PMON69469 | Microprojectile bombardment | 73 | 8 | 4 | 3 |
| PHE0000266 | PMON69470 | Microprojectile bombardment | 47 | 19 | 9 | 8 |
| PHE0000267 | PMON68867 | Microprojectile bombardment | 82 | 39 | 16 | 15 |
| PHE0000273 | PMON74423 | Microprojectile bombardment | 20 | 3 | | |
| PHE0000276 | PMON68868 | Microprojectile bombardment | 97 | 65 | 6 | 5 |
| PHE0000277 | PMON68890 | Microprojectile bombardment | 107 | 17 | 5 | 4 |
| PHE0000278 | PMON68886 | Microprojectile bombardment | 88 | 31 | 14 | 9 |
| PHE0000279 | PMON68896 | Microprojectile bombardment | 127 | 44 | 10 | 7 |
| PHE0000280 | PMON72451 | Microprojectile bombardment | 79 | 27 | 8 | 6 |
| PHE0000281 | PMON72452 | Microprojectile bombardment | 44 | 3 | 1 | 1 |
| PHE0000283 | PMON69472 | Microprojectile bombardment | 118 | 17 | 4 | 3 |
| PHE0000284 | PMON72453 | Microprojectile bombardment | | | | |
| PHE0000286 | PMON72454 | Microprojectile bombardment | | | | |
| PHE0000287 | PMON68898 | Microprojectile bombardment | 61 | 34 | 15 | 9 |
| PHE0000291 | PMON72455 | Microprojectile bombardment | 34 | 26 | 8 | 6 |
| PHE0000292 | PMON68888 | Microprojectile bombardment | 57 | 28 | 14 | 7 |
| PHE0000294 | PMON68897 | Microprojectile bombardment | 79 | 27 | 5 | 4 |
| PHE0000295 | PMON68894 | Microprojectile bombardment | 83 | 37 | 13 | 12 |
| PHE0000296 | PMON68893 | Microprojectile bombardment | 60 | 5 | 2 | 2 |
| PHE0000297 | PMON68899 | Microprojectile bombardment | 49 | 19 | 5 | 5 |
| PHE0000298 | PMON68874 | Microprojectile bombardment | 5 | | | |
| PHE0000299 | PMON68875 | Microprojectile bombardment | 64 | 16 | 7 | 7 |
| PHE0000300 | PMON68876 | Microprojectile bombardment | 106 | 30 | 8 | 5 |
| PHE0000301 | PMON68877 | Microprojectile bombardment | 127 | 31 | 14 | 11 |
| PHE0000302 | PMON68878 | Microprojectile bombardment | 119 | 35 | 5 | 1 |
| PHE0000303 | PMON68879 | Microprojectile bombardment | 4 | | | |
| PHE0000304 | PMON68873 | Microprojectile bombardment | 89 | 42 | 19 | 11 |
| PHE0000305 | PMON68880 | Microprojectile bombardment | 71 | 32 | 11 | 7 |
| PHE0000306 | PMON68882 | Microprojectile bombardment | 88 | 35 | 7 | 6 |
| PHE0000307 | PMON68883 | Microprojectile bombardment | 82 | 26 | 6 | 4 |
| PHE0000308 | PMON68884 | Microprojectile bombardment | 153 | 48 | 16 | 7 |
| PHE0000309 | PMON68885 | Microprojectile bombardment | 51 | 16 | 8 | 5 |
| PHE0000310 | PMON68377 | Microprojectile bombardment | 60 | 22 | 7 | 5 |
| PHE0000311 | PMON72458 | Microprojectile bombardment | 74 | 35 | 15 | 11 |
| PHE0000312 | PMON72456 | Microprojectile bombardment | 67 | 24 | 12 | 9 |
| PHE0000313 | PMON68378 | Microprojectile bombardment | 79 | 23 | 7 | 7 |
| PHE0000314 | PMON68379 | Microprojectile bombardment | 82 | 11 | 6 | 5 |
| PHE0000315 | PMON68381 | Microprojectile bombardment | 165 | 39 | 22 | 19 |
| PHE0000316 | PMON68382 | Microprojectile bombardment | 188 | 30 | 16 | 9 |
| PHE0000317 | PMON68380 | Microprojectile bombardment | 142 | 50 | 14 | 12 |
| PHE0000322 | PMON74403 | Microprojectile bombardment | 110 | 24 | 7 | 7 |
| PHE0000323 | PMON68400 | Agrobacterium | 167 | 27 | 1 | |
| PHE0000324 | PMON73162 | Microprojectile bombardment | 46 | 21 | 11 | 11 |
| PHE0000325 | PMON68384 | Microprojectile bombardment | 64 | 24 | 10 | 8 |
| PHE0000326 | PMON72463 | Microprojectile bombardment | 50 | 31 | 11 | 9 |
| PHE0000327 | PMON69481 | Microprojectile bombardment | 68 | 32 | 16 | 14 |
| PHE0000328 | PMON74416 | Microprojectile bombardment | 134 | 22 | 14 | 14 |
| PHE0000330 | PMON73164 | Microprojectile bombardment | 121 | 25 | 9 | 8 |
| PHE0000332 | PMON68385 | Microprojectile bombardment | 29 | 10 | 3 | 2 |
| PHE0000333 | PMON75470 | Microprojectile bombardment | 52 | 8 | 4 | 2 |
| PHE0000334 | PMON68395 | Agrobacterium | 328 | 68 | 4 | 4 |
| PHE0000335 | PMON74413 | Microprojectile bombardment | 164 | 24 | 12 | 7 |
| PHE0000336 | PMON74414 | Microprojectile bombardment | 177 | 41 | 25 | 22 |
| PHE0000338 | PMON68628 | Microprojectile bombardment | 149 | 32 | 25 | 22 |
| PHE0000339 | PMON68627 | Microprojectile bombardment | 21 | 7 | | |
| PHE0000339 | PMON75490 | Microprojectile bombardment | 60 | 4 | 1 | |
| PHE0000340 | PMON68629 | Microprojectile bombardment | 116 | 25 | 14 | 13 |
| PHE0000341 | PMON68397 | Agrobacterium | 14 | | | |
| PHE0000344 | PMON73163 | Microprojectile bombardment | 54 | 23 | 9 | 8 |
| PHE0000345 | PMON74411 | Microprojectile bombardment | 158 | 30 | 23 | 18 |
| PHE0000346 | PMON73165 | Microprojectile bombardment | 180 | 32 | 10 | 7 |
| PHE0000347 | PMON68386 | Microprojectile bombardment | 190 | 33 | 22 | 17 |
| PHE0000348 | PMON68387 | Microprojectile bombardment | 79 | 33 | 22 | 17 |
| PHE0000349 | PMON68389 | Microprojectile bombardment | 102 | 34 | 15 | 11 |
| PHE0000350 | PMON74410 | Microprojectile bombardment | 114 | 25 | 15 | 14 |
| PHE0000352 | PMON74409 | Microprojectile bombardment | 66 | 28 | 16 | 14 |
| PHE0000353 | PMON73160 | Microprojectile bombardment | 132 | 54 | 12 | 9 |
| PHE0000356 | PMON72464 | Microprojectile bombardment | 144 | 40 | 10 | 8 |
| PHE0000357 | PMON69474 | Microprojectile bombardment | 98 | 20 | 4 | 4 |
| PHE0000358 | PMON69475 | Microprojectile bombardment | 44 | 16 | 8 | 7 |
| PHE0000359 | PMON69476 | Microprojectile bombardment | 7 | 1 | 1 | 1 |
| PHE0000372 | PMON72460 | Microprojectile bombardment | 88 | 28 | 14 | 14 |
| PHE0000382 | PMON74401 | Microprojectile bombardment | 40 | 27 | 10 | 9 |
| PHE0000386 | PMON67834 | Microprojectile bombardment | 145 | 39 | 29 | 26 |
| PHE0000390 | PMON67836 | Microprojectile bombardment | 20 | 11 | 4 | 4 |
| PHE0000391 | PMON67835 | Microprojectile bombardment | 39 | 15 | 9 | 5 |
| PHE0000392 | PMON76335 | Microprojectile bombardment | 50 | 20 | | |
| PHE0000395 | PMON67840 | Microprojectile bombardment | 71 | 27 | 15 | 12 |
| PHE0000396 | PMON67838 | Microprojectile bombardment | 58 | 29 | 16 | 14 |
| PHE0000397 | PMON67839 | Microprojectile bombardment | 99 | 24 | 5 | 4 |
| PHE0000398 | PMON72488 | Microprojectile bombardment | 224 | 5 | 2 | 2 |
| PHE0000399 | PMON72485 | Microprojectile bombardment | | | | |
| PHE0000400 | PMON72486 | Microprojectile bombardment | 5 | | | |
| PHE0000401 | PMON67837 | Microprojectile bombardment | 53 | 18 | 13 | 13 |
| PHE0000402 | PMON67833 | Microprojectile bombardment | 52 | 13 | 5 | 3 |
| PHE0000403 | PMON67831 | Microprojectile bombardment | 39 | 18 | 6 | 6 |
| PHE0000404 | PMON67832 | Microprojectile bombardment | 19 | 9 | 1 | 1 |
| PHE0000411 | PMON68614 | Agrobacterium | 4 | | | |
| PHE0000412 | PMON67843 | Microprojectile bombardment | 42 | 30 | 19 | 10 |
| PHE0000413 | PMON67844 | Microprojectile bombardment | 124 | 30 | 16 | 13 |
| PHE0000414 | PMON67845 | Microprojectile bombardment | 80 | 28 | 14 | 9 |
| PHE0000415 | PMON67846 | Microprojectile bombardment | 105 | 25 | 15 | 11 |
| PHE0000416 | PMON67847 | Microprojectile bombardment | 30 | 11 | 4 | 4 |
| PHE0000418 | PMON69497 | Microprojectile bombardment | 144 | 26 | 18 | 16 |
| PHE0000419 | PMON67848 | Microprojectile bombardment | 143 | 26 | 21 | 16 |
| PHE0000420 | PMON74415 | Microprojectile bombardment | 85 | 28 | 10 | 8 |
| PHE0000423 | PMON72497 | Microprojectile bombardment | 77 | 22 | 16 | 8 |
| PHE0000425 | PMON72495 | Microprojectile bombardment | 84 | 19 | 3 | 3 |
| PHE0000426 | PMON74408 | Microprojectile bombardment | 48 | 21 | 17 | 14 |
| PHE0000428 | PMON74417 | Microprojectile bombardment | 80 | 29 | 14 | 14 |
| PHE0000429 | PMON74418 | Microprojectile bombardment | 97 | 20 | 8 | 6 |
| PHE0000433 | PMON74424 | Microprojectile bombardment | 27 | 4 | 1 | 1 |
| PHE0000434 | PMON74419 | Microprojectile bombardment | 60 | 7 | 3 | 3 |
| PHE0000435 | PMON75499 | Microprojectile bombardment | 64 | 4 | | |
| PHE0000437 | PMON68630 | Microprojectile bombardment | 144 | 31 | 17 | 15 |
| PHE0000439 | PMON74425 | Microprojectile bombardment | 7 | | | |
| PHE0000440 | PMON72473 | Microprojectile bombardment | 97 | 22 | 13 | 11 |
| PHE0000441 | PMON72474 | Microprojectile bombardment | 130 | 35 | 20 | 12 |
| PHE0000445 | PMON74426 | Microprojectile bombardment | | | | |
| PHE0000451 | PMON72475 | Microprojectile bombardment | 133 | | | |
| PHE0000452 | PMON72476 | Microprojectile bombardment | 110 | 12 | 8 | 6 |
| PHE0000454 | PMON72477 | Microprojectile bombardment | 130 | 25 | 12 | 9 |
| PHE0000469 | PMON68636 | Microprojectile bombardment | 80 | 26 | 17 | 16 |
| PHE0000471 | PMON73772 | Microprojectile bombardment | 74 | 31 | 8 | |
| PHE0000473 | PMON75471 | Microprojectile bombardment | 21 | 7 | 3 | 2 |
| PHE0000485 | PMON69498 | Microprojectile bombardment | 56 | 24 | 15 | 15 |
| PHE0000486 | PMON69496 | Microprojectile bombardment | 63 | 25 | 7 | 6 |

### Example 10

Isolation of Nucleic Acids from Plants

Nucleic acids were isolated from leaf tissue of R0 plants, collected and flash frozen in a 96 well collection box, 0 to 2 weeks after plantlets were transferred to soil. Approximately 100 milligrams of tissue was collected from each plant and stored at −80° C. until analysis.

DNA and RNA were isolated from a single tissue sample using the Qiagen RNeasy 96™ kit (Qiagen Inc., Valencia, Calif.) with modifications. One hundred milligrams of frozen tissue was homogenized in 700 μL RNeasy™ RTL buffer (Qiagen Inc., Valencia, Calif.) using a Bead Beater™ (Biospec Products, Bartlesville, Okla.). Samples were centrifuged at 3200 rpm for 15 minutes and all of the supernatant transferred the wells of a Promega WIZARD™ clearing plate (Promega Corporation, Madison, Wis.). The sample solutions were clarified by vacuum filtration through the clearing plate. The cleared supernatant was used for nucleic acid extractions.

For DNA extractions, 70 μL of the cleared sample was transferred to a v-well PCR plate, covered with adhesive foil, and heated to 95° C. for 8 minutes. The samples were incubated at 0° C. for five minutes, followed by centrifugation for 3 minutes to remove insoluble materials. A Sephadex G-50 gel filtration box (Edge Biosystems, Gaithersburg, Mo.) was conditioned for 2 min at 2000 rpm. Forty μL of the heat-treated supernatant was loaded into each well and the box centrifuged for two minutes at 2500 rpm. An additional 20 μL of TE buffer was added to the column effluent and the sample plate was stored at −20° C. until analysis.

For RNA extractions, five hundred microliters of cleared solution was transfer to a clean 96 well sample box. Two hundred and fifty microliters of 100% ethanol was added to each sample and the sample was thoroughly mixed. All of the approximately seven hundred and fifty microliters of solution was then loaded into the wells of a Qiagen RNeasy™ binding plate in a Promega WIZARD™ filtation unit. Five hundred microliters of RW1 buffer (Qiagen Inc., Valencia, Calif.) was added to each well and the buffer removed by vacuum filtration. Eighty microliters of RNAase free DNAase (Qiagen Inc., Valencia, Calif.) was added to each well, incubated at room temperature for 15 minutes the DNAase solution drawn through the wells by vacuum filteration. An additional five hundred microliters RW1 buffer (Qiagen Inc., Valencia, Calif.) was added to the wells and the buffer removed by vacuum filtration. The sample was further washed by vacuum filtration with 500 μL RPE buffer 2× (Qiagen, Valencia, Calif.). The extraction plate was placed on a microtiter plate and centrifuged for three minutes at 3000 rpm to remove any residual RPE buffer solution in the filter. Eighty microliters of RNA grade water (DNAse free) was added to each well, followed by incubation at room temperature for two minutes. The extraction plate and microtiter plate were centrifuged for three minutes at 3000 rpm and the RNA preparation stored frozen in the collection plate at −80° C.

### Example 11

Assays for Copy Number

Copy number of transgenes in R0 plants was determined using TAQMAN® methods. The pMON65154 and pRG76 GATEWAY™ destination vectors were constructed with a sequence derived from the 3′ region of the potato pinII gene which could be used to assay copy number of transgene insertions. The pinII forward and reverse primers were as follows:

| | | |
|---|---|---|
| Forward primer | 5′ ccccaccctgcaatgtga 3′ | (SEQ ID NO:737) |
| Reverse primer | 5′ tgtgcatccttttatttcatacattaattaa 3′ | (SEQ ID NO:738) |

The pinII TAQMAN ® probe sequence was
5′ cctagacttgtccatcttctggattggcca 3′

The probe was labelled at the 5′ end with the fluorescent dye FAM (6-carboyxfluorescein) and the quencher dye TAMRA (6-carboxy-N,N,N′,N′-tetramethylrhodamine) was attached via a linker to the 3′ end of the probe. The TAQMAN® probe was obtained from Applied Biosystems (Foster City, Calif.). SAT, a single copy maize gene was used as an internal control in TAQMAN® copy number assays. The SAT primers were as follows:

| | |
|---|---|
| Forward primer | |
| 5 ′gcctgccgcagaccaa 3′ | (SEQ ID NO:739) |
| Reverse primer | |
| 5′ atgcagagctcagcttcatc 3′ | (SEQ ID NO:740) |

The SAT TAQMAN ® probe sequence was

| | |
|---|---|
| 5′ tccagtacgtgcagtccctcctcc 3′ | (SEQ ID NO:741) |

the probe was labelled at its 5′ end with the fluorescent dye VIC™ (Applied Biosystems, Foster City, Calif.) and the quencher dye TAMRA at is 3′end.

TAQMAN® PCR was performed according to the manufacturer's instructions (Applied Biosystems, Foster City, Calif.). Five to 100 nanograms DNA was used in each assay. PCR amplification and TAQMAN® probe detection were performed in 1× TAQMAN® Universal PCR Master Mix (Applied Biosystems, Foster City, Calif.) which contains AmpliTaq Gold® DNA polymerase, AmpErase® UNG, dNTPs with dUTP, Passive Reference 1, and optimized buffer. Eight hundred nM each forward and reverse pinII primers and 150 nM pinII TAQMAN® probe were used in the TAQMAN® assay. 200 nM each Sat forward and reverse primers and 150 nM Sat TAQMAN® probe were used in the TAQMAN® copy number assay. TAQMAN® PCR was carried out for 2 minutes at 50° C., 10 minutes at 95° C, followed by 40 cycles of 15 seconds at 95° C. and one minute at 60° C. Real time TAQMAN® probe fluorescence was measured using an ABI Prism 7700 Sequence Detection System or ABI7900HT Sequence Detection System (Applied Biosystems, Foster City, Calif.). C_{T} values were calculated according to the TAQMAN® EZ RT-PCR kit instruction manual (Applied Biosystems, Foster City, Calif.). The ΔΔ C_{T} value was calculated as C_{T} (internal control gene (Sat))—C_{T} (transgene)—C_{T} (internal control gene (Sat) in nontransgenic plant). The copy number was assigned as follows according to the following.

| Copy Number | Criteria |
|---|---|
| 1 | −0.5 < ^{ΔΔC}_{T} < 0.50 |
| 2 | 0.5 < ^{ΔΔC}_{T} < 1.50 |
| >2 | ^{ΔΔC}_{T} > 1.50 |

Plants comprising genes of the present invention were anyalyzed by TAQMAN® methods for copy number. Copy number in plants containing various genes of the present invention is summarized in Table 5 below.

TABLE 5

| | | Number of | Number 0 | 1-2 Copy | Number of Plants |
|---|---|---|---|---|---|
| PHE_ID | pMON No | Plants Assayed | Copy Plants | Plants | Expressing |
| PHE0000006 | PMON68861 | 45 | 2 | 16 | 12 |
| PHE0000009 | PMON67803 | 17 | 2 | 7 | 7 |
| PHE0000010 | PMON67800 | 41 | 1 | 28 | 23 |
| PHE0000012 | PMON67808 | 37 | 5 | 23 | 26 |
| PHE0000012 | PMON67806 | 20 | 5 | 10 | 11 |
| PHE0000013 | PMON69452 | 53 | 12 | 24 | 30 |
| PHE0000013 | PMON69451 | 38 | 3 | 25 | 18 |
| PHE0000016 | PMON67750 | 8 | 7 | 1 | 2 |
| PHE0000017 | PMON68850 | 20 | 5 | 11 | 10 |
| PHE0000022 | PMON67826 | 26 | 3 | 12 | 12 |
| PHE0000024 | PMON68354 | 51 | 11 | 35 | 8 |
| PHE0000024 | PMON68354 | 51 | 11 | 35 | 8 |
| PHE0000034 | PMON67805 | 30 | 4 | 11 | 13 |
| PHE0000038 | PMON68383 | 11 | 4 | 3 | 2 |
| PHE0000039 | PMON67807 | 22 | 6 | 10 | 7 |
| PHE0000040 | PMON67801 | 13 | | 5 | 9 |
| PHE0000051 | PMON68859 | 20 | | 13 | 15 |
| PHE0000052 | PMON67813 | 45 | 8 | 20 | 23 |
| PHE0000055 | PMON68355 | 33 | 3 | 23 | 28 |
| PHE0000056 | PMON68364 | 31 | 5 | 18 | 16 |
| PHE0000057 | PMON68350 | 33 | 3 | 14 | 19 |
| PHE0000058 | PMON68351 | 27 | 5 | 11 | 15 |
| PHE0000059 | PMON68370 | 20 | 2 | 10 | 17 |
| PHE0000060 | PMON68356 | 18 | 2 | 15 | 13 |
| PHE0000064 | PMON67804 | 9 | | 4 | 7 |
| PHE0000067 | PMON67816 | 36 | 4 | 21 | 24 |
| PHE0000068 | PMON67824 | 2 | | 2 | |
| PHE0000069 | PMON67821 | 17 | 6 | 5 | 7 |
| PHE0000070 | PMON67825 | 49 | 3 | 21 | 28 |
| PHE0000072 | PMON67828 | 19 | 8 | 9 | 2 |
| PHE0000073 | PMON68357 | 23 | 3 | 16 | 15 |
| PHE0000074 | PMON68352 | 17 | 5 | 8 | 7 |
| PHE0000076 | PMON68851 | 18 | | 13 | 5 |
| PHE0000077 | PMON67827 | 25 | 3 | 10 | 13 |
| PHE0000079 | PMON67752 | 33 | 7 | 14 | 23 |
| PHE0000086 | PMON67812 | 36 | 3 | 16 | 24 |
| PHE0000091 | PMON68358 | 12 | 1 | 9 | 7 |
| PHE0000092 | PMON68359 | 17 | 1 | 12 | 8 |
| PHE0000098 | PMON73168 | 16 | 4 | 7 | 7 |
| PHE0000102 | PMON67815 | 12 | 1 | 8 | 2 |
| PHE0000104 | PMON68608 | 24 | 1 | 11 | 22 |
| PHE0000106 | PMON69457 | 16 | 1 | 7 | 12 |
| PHE0000108 | PMON67849 | 18 | 1 | 11 | 16 |
| PHE0000114 | PMON68361 | 12 | 4 | 6 | 6 |
| PHE0000115 | PMON68362 | 2 | 2 | | |
| PHE0000116 | PMON68367 | 30 | 3 | 15 | 9 |
| PHE0000117 | PMON68368 | 21 | 7 | 10 | 12 |
| PHE0000118 | PMON67811 | 13 | 2 | 2 | 5 |
| PHE0000119 | PMON68363 | 21 | 3 | 16 | 5 |
| PHE0000120 | PMON68853 | 18 | 2 | 12 | 10 |
| PHE0000121 | PMON68854 | 26 | 3 | 12 | 25 |
| PHE0000122 | PMON74402 | 17 | 5 | 11 | 10 |
| PHE0000123 | PMON68855 | 21 | 4 | 16 | 12 |
| PHE0000124 | PMON68856 | 24 | | 17 | 20 |
| PHE0000125 | PMON68369 | 63 | 8 | 24 | 39 |
| PHE0000126 | PMON69458 | 21 | 4 | 13 | 14 |
| PHE0000127 | PMON68887 | 26 | 2 | 13 | 19 |
| PHE0000133 | PMON68860 | 63 | 6 | 33 | 37 |
| PHE0000153 | PMON67817 | 44 | 5 | 25 | 28 |
| PHE0000154 | PMON67818 | 29 | 3 | 21 | 21 |
| PHE0000158 | PMON73169 | 24 | 3 | 5 | 12 |
| PHE0000160 | PMON75485 | 1 | | 1 | 1 |
| PHE0000162 | PMON75488 | 7 | 2 | 4 | 6 |
| PHE0000164 | PMON73170 | 23 | 5 | 6 | 13 |
| PHE0000168 | PMON68857 | 25 | 1 | 13 | 18 |
| PHE0000173 | PMON73171 | 22 | 6 | 6 | 10 |
| PHE0000176 | PMON68388 | 22 | 4 | 10 | 18 |
| PHE0000177 | PMON68881 | 15 | 6 | 6 | 7 |
| PHE0000178 | PMON73166 | 10 | 1 | 9 | 4 |
| PHE0000179 | PMON69467 | 9 | 4 | 5 | 4 |
| PHE0000182 | PMON74420 | 13 | 2 | 6 | 6 |
| PHE0000185 | PMON69468 | 20 | 10 | 7 | 3 |
| PHE0000186 | PMON69460 | 22 | 6 | 12 | 15 |
| PHE0000188 | PMON73167 | 37 | 7 | 23 | 22 |
| PHE0000192 | PMON68394 | 2 | | 2 | 2 |
| PHE0000193 | PMON68889 | 12 | 1 | 8 | 10 |
| PHE0000219 | PMON68865 | 15 | 6 | 7 | 8 |
| PHE0000220 | PMON74434 | 19 | | 12 | 19 |
| PHE0000223 | PMON69478 | 34 | 4 | 7 | 6 |
| PHE0000227 | PMON68376 | 34 | 3 | 14 | 19 |
| PHE0000231 | PMON72498 | 25 | 2 | 10 | 22 |
| PHE0000232 | PMON68895 | 17 | 4 | 7 | 13 |
| PHE0000235 | PMON73161 | 9 | 1 | 6 | 4 |
| PHE0000237 | PMON68891 | 22 | 6 | 11 | 18 |
| PHE0000238 | PMON69466 | 26 | 8 | 13 | 9 |
| PHE0000239 | PMON72466 | 29 | 5 | 17 | 21 |
| PHE0000240 | PMON72468 | 16 | 4 | 7 | 11 |
| PHE0000242 | PMON72470 | 22 | 6 | 8 | 10 |
| PHE0000243 | PMON72467 | 19 | 2 | 11 | 17 |
| PHE0000244 | PMON68372 | 31 | 5 | 20 | 13 |
| PHE0000245 | PMON68373 | 22 | 3 | 10 | 19 |
| PHE0000246 | PMON68374 | 45 | 8 | 22 | 17 |
| PHE0000247 | PMON68375 | 28 | 5 | 11 | 15 |
| PHE0000249 | PMON74422 | 18 | 7 | 7 | 11 |
| PHE0000252 | PMON74407 | 28 | 6 | 16 | 18 |
| PHE0000254 | PMON73172 | 16 | 5 | 10 | 2 |
| PHE0000255 | PMON72459 | 27 | 3 | 20 | 22 |
| PHE0000256 | PMON75302 | 1 | 1 | | 1 |
| PHE0000258 | PMON68371 | 34 | 3 | 15 | 20 |
| PHE0000259 | PMON74404 | 24 | 1 | 12 | 19 |
| PHE0000262 | PMON68892 | 8 | 5 | 1 | 3 |
| PHE0000263 | PMON74412 | 19 | | 17 | 17 |
| PHE0000264 | PMON68866 | 21 | 4 | 15 | 5 |
| PHE0000265 | PMON69469 | 4 | 3 | 1 | 2 |
| PHE0000266 | PMON69470 | 16 | 8 | 5 | 4 |
| PHE0000267 | PMON68867 | 22 | 5 | 14 | 7 |
| PHE0000273 | PMON74423 | 2 | 1 | 1 | 1 |
| PHE0000276 | PMON68868 | 35 | 4 | 15 | 17 |
| PHE0000277 | PMON68890 | 16 | 1 | 11 | 8 |
| PHE0000278 | PMON68886 | 24 | | 17 | 18 |
| PHE0000279 | PMON68896 | 35 | 3 | 19 | 24 |
| PHE0000280 | PMON72451 | 22 | 10 | 7 | 14 |
| PHE0000281 | PMON72452 | 1 | | 1 | 1 |
| PHE0000283 | PMON69472 | 14 | | 3 | 5 |
| PHE0000287 | PMON68898 | 29 | 6 | 16 | 19 |
| PHE0000291 | PMON72455 | 24 | 3 | 11 | 15 |
| PHE0000292 | PMON68888 | 26 | 3 | 20 | 20 |
| PHE0000294 | PMON68897 | 14 | 3 | 6 | 9 |
| PHE0000295 | PMON68894 | 33 | 5 | 12 | 16 |
| PHE0000296 | PMON68893 | 5 | 1 | 1 | 2 |
| PHE0000297 | PMON68899 | 16 | 1 | 9 | 11 |
| PHE0000299 | PMON68875 | 8 | | 6 | 7 |
| PHE0000300 | PMON68876 | 18 | 6 | 8 | 10 |
| PHE0000301 | PMON68877 | 24 | 2 | 17 | 16 |
| PHE0000302 | PMON68878 | 25 | 6 | 12 | 2 |
| PHE0000304 | PMON68873 | 34 | 7 | 21 | 19 |
| PHE0000305 | PMON68880 | 27 | 9 | 11 | 10 |
| PHE0000306 | PMON68882 | 29 | 6 | 15 | 22 |
| PHE0000307 | PMON68883 | 19 | 5 | 12 | 15 |
| PHE0000308 | PMON68884 | 34 | 5 | 18 | 20 |
| PHE0000309 | PMON68885 | 11 | 2 | 8 | 9 |
| PHE0000310 | PMON68377 | 22 | 6 | 8 | 10 |
| PHE0000311 | PMON72458 | 25 | 3 | 13 | 16 |
| PHE0000312 | PMON72456 | 21 | 4 | 7 | 16 |
| PHE0000313 | PMON68378 | 14 | | 9 | 5 |
| PHE0000314 | PMON68379 | 7 | | 3 | 7 |
| PHE0000315 | PMON68381 | 36 | 7 | 15 | 30 |
| PHE0000316 | PMON68382 | 21 | 3 | 8 | 16 |
| PHE0000317 | PMON68380 | 40 | 7 | 7 | 24 |
| PHE0000322 | PMON74403 | 21 | 3 | 8 | 16 |
| PHE0000324 | PMON73162 | 14 | 2 | 9 | 12 |
| PHE0000325 | PMON68384 | 19 | 1 | 7 | 16 |
| PHE0000326 | PMON72463 | 27 | 3 | 12 | 12 |
| PHE0000327 | PMON69481 | 24 | 3 | 17 | 22 |
| PHE0000328 | PMON74416 | 15 | 3 | 11 | 10 |
| PHE0000330 | PMON73164 | 23 | 4 | 9 | 15 |
| PHE0000332 | PMON68385 | 7 | 1 | 6 | 6 |
| PHE0000333 | PMON75470 | 1 | | 1 | 1 |
| PHE0000334 | PMON68395 | 5 | 1 | | 5 |
| PHE0000335 | PMON74413 | 15 | 3 | 6 | 13 |
| PHE0000336 | PMON74414 | 34 | 5 | 20 | 27 |
| PHE0000338 | PMON68628 | 26 | 6 | 9 | 14 |
| PHE0000339 | PMON68627 | 5 | | 4 | 2 |
| PHE0000340 | PMON68629 | 22 | 2 | 9 | 16 |
| PHE0000344 | PMON73163 | 20 | 1 | 13 | 18 |
| PHE0000345 | PMON74411 | 28 | 2 | 8 | 23 |
| PHE0000346 | PMON73165 | 26 | 3 | 6 | 15 |
| PHE0000347 | PMON68386 | 32 | 4 | 20 | 29 |
| PHE0000348 | PMON68387 | 27 | 2 | 21 | 20 |
| PHE0000349 | PMON68389 | 29 | 3 | 16 | 19 |
| PHE0000350 | PMON74410 | 18 | 1 | 17 | 13 |
| PHE0000352 | PMON74409 | 24 | 5 | 13 | 21 |
| PHE0000353 | PMON73160 | 38 | 10 | 21 | 24 |
| PHE0000356 | PMON72464 | 29 | 6 | 12 | 16 |
| PHE0000357 | PMON69474 | 9 | | 4 | 4 |
| PHE0000358 | PMON69475 | 14 | 1 | 12 | 2 |
| PHE0000359 | PMON69476 | 1 | | | 1 |
| PHE0000372 | PMON72460 | 24 | 3 | 13 | 20 |
| PHE0000382 | PMON74401 | 20 | | 13 | 18 |
| PHE0000386 | PMON67834 | 32 | 2 | 5 | 2 |
| PHE0000390 | PMON67836 | 10 | 1 | 4 | 8 |
| PHE0000391 | PMON67835 | 7 | 2 | 4 | 3 |
| PHE0000395 | PMON67840 | 18 | 2 | 9 | 15 |
| PHE0000396 | PMON67838 | 22 | 4 | 8 | 16 |
| PHE0000397 | PMON67839 | 22 | 7 | 11 | 13 |
| PHE0000398 | PMON72488 | 4 | 1 | 1 | 2 |
| PHE0000401 | PMON67837 | 13 | | 7 | 11 |
| PHE0000402 | PMON67833 | 5 | 1 | 3 | 3 |
| PHE0000403 | PMON67831 | 11 | 1 | 4 | 9 |
| PHE0000404 | PMON67832 | 6 | | 5 | 1 |
| PHE0000412 | PMON67843 | 27 | 2 | 17 | 21 |
| PHE0000413 | PMON67844 | 23 | 3 | 6 | 17 |
| PHE0000414 | PMON67845 | 16 | 4 | 9 | 10 |
| PHE0000415 | PMON67846 | 22 | 2 | 15 | 17 |
| PHE0000416 | PMON67847 | 9 | 2 | 4 | 6 |
| PHE0000418 | PMON69497 | 19 | 2 | 7 | 15 |
| PHE0000419 | PMON67848 | 24 | 4 | 2 | 5 |
| PHE0000420 | PMON74415 | 18 | 2 | 8 | 16 |
| PHE0000423 | PMON72497 | 19 | 2 | 12 | 19 |
| PHE0000425 | PMON72495 | 6 | | 4 | 5 |
| PHE0000426 | PMON74408 | 15 | | 8 | 14 |
| PHE0000428 | PMON74417 | 15 | 2 | 8 | 12 |
| PHE0000429 | PMON74418 | 15 | 2 | 4 | 13 |
| PHE0000433 | PMON74424 | 3 | 2 | 1 | 1 |
| PHE0000434 | PMON74419 | 6 | 1 | 3 | 4 |
| PHE0000437 | PMON68630 | 23 | 2 | 8 | 19 |
| PHE0000440 | PMON72473 | 18 | 2 | 10 | 15 |
| PHE0000441 | PMON72474 | 29 | 5 | 13 | 23 |
| PHE0000452 | PMON72476 | 12 | 3 | 3 | 10 |
| PHE0000454 | PMON72477 | 17 | 2 | 9 | 15 |
| PHE0000469 | PMON68636 | 20 | | 3 | 16 |
| PHE0000473 | PMON75471 | 1 | | 1 | 1 |
| PHE0000485 | PMON69498 | 19 | 3 | 9 | 17 |
| PHE0000486 | PMON69496 | 14 | 1 | 8 | 8 |

### Example 12

Assays for Gene Expression

Expression of a transgene of the present invention was assayed by TAQMAN® RT-PCR using the TAQMAN® EZ RT-PCR kit from Applied Biosystems (Foster City, Calif.). RNA expression was assyed relative to expression in a transgenic standard, a transgenic maize event designated DBT418, comprising a B. thuringiensis cryIA(c) gene operably linked to a pinII 3′ untranslated region. The DBT418 event expresses the cryIA(c) gene at a level which confers commercial levels of resistance to lepdiopteran insects such as Europeani Corn Borer and was commercially sold by DEKALB Genetics Corporation under the brand name DEKALBt®. The pMON65154 and pRG76 GATEWAY™ destination vectors were constructed with a sequence derived from the 3′ region of the potato pinII gene which could be used to assay transgene transcript levels for any coding sequence inserted into the Destination Vector. The pinII primers and probe described in Example 11 were used for TAQMAN® RT-PCR. Ubiquitin fusion protein (UBI) RNA was used as an internal control in all TAQMAN® RT-PCR assays. The UBI primers used were as follows:

| | |
|---|---|
| Forward primer | |
| 5′ cgtctacaatcagaaggcgtaatc 3′ | (SEQ ID NO:743) |
| Reverse primer | |
| 5′ ccaacaggtgaatgcttgatagg 3′ | (SEQ ID NO:744) |

The sequence of the UBI TAQMAN ® probe was

| | |
|---|---|
| 5′ catgcgccgctttgcttc 3′ | (SEQ ID NO:745) |

The UBI TAQMAN® probe was labelled at its 5′ end with the fluorescent dye VIC™ (Applied Biosystems, Foster City, Calif.) and the quencher dye TAMRA at is 3′ end Reverse transcription, PCR amplification and TAQMAN® probing were performed according to the one step procedure described in the TAQMAN® EZ RT-PCR kit (Applied Biosystems, Foster City, Calif.). Five to 100 nanograms total RNA was used in each assay. In vitro transcribed control RNA from the DBT418 event was included as a control on every plate and run over a concentration range from 0.01 picograms to 10 picograms. Total RNA from DBT418 leaf and from the non-transgenic inbred LH59 were run as positive and negative controls respectively. RT-PCR was performed in TAQMAN® EZ Buffer (50 mM Bicine, 115 mM potassium acetate, 0.01 mM EDTA, 60 mM Passive Reference 1, 8% glycerol, pH 8.2, Applied Biosystems, Foster City, Calif.) containing 3 mM manganese acetate, 300 μM each dATP, dCTP, dGTP, and dUTP, 100 units rTth™ (Applied Biosystems, Foster City, Calif.) DNA polymerase, and 25 units AmpErase UNG (Applied Biosytems, Foster City, Calif.). RT-PCR was carred out as follows: 2 minutes at 50° C, 30 minutes at 60° C., 5 minutes at 95° C., followed by 40 cycles of 20 seconds at 95° C. and 1 minute at 60° C. 400 nM each forward and reverse primers were used for amplification of the pinII sequence and 200 nM TAQMAN® pinII probe used for detection. UBI RNA was amplified using 400 nM each forward and reverse primers and 200 nM UBI TAQMAN® probe was used for detection. TAQMAN® fluorescence was measured using an ABI Prism 7700 Sequence Detection System or ABI7900HT Sequence Detection System (Applied Biosystems, Foster City, Calif.). The C_{T} and ΔΔC_{T} values were calculated as described in Example 11. Expression of transgenes of the present invention was quantitated relative to transgene expression in DBT418 and reported as a ratio of transgene expression to DBT418 expression, i.e., 2^{-(ΔΔC}^{_{T}}⁾ (transgene)/2^{-(ΔΔC}^{_{T}}⁾ (DBT418). Expression of various genes of the present invention in maize is summarized in Table 5.

### Example 13

Phenotype Evaluation

For each plant representing a transgenic event, F1 seed was planted in a field producing plants which were assayed for phenotype and for the selectable kanamycin resistant marker. Each of the plants were self pollinated to produce F2 seed. Seed from nptII-positive plants, e.g. a few ears from each transgenic event, was planted and grown to produce F2 plants which were assayed for phenotype and kanamycin resistance. Kanamycin-resistant F2 plants were self pollinated to produce F3 seed. F3 seed was screened for complete resistance to kanamycin indicating a homozygous transgene. Seeds from homozygous F3 ears were planted in the field to produce F3 plants which were self pollinated to produce P4 seed. F3 plants were also crossed to tester inbred lines to produce F1 hybrid transgenic seed. Phenotypes such as yield are determined from F1 hybrid transgenic seed; other phenotypes can be determined from either F1 hybrid transgenic lines or F1, F2, F3 or F4 inbred transgenic lines.

A variety of transgenic plants were grown in field conditions allowing observation of multiple events of the unexpected phenotypes listed in Table 6 below. See also Applicants' copending PCT patent application entitled “Transgenic Maize with Enhanced Phenotype” filed Dec. 4, 2002, the entirety of which application is incorporated herein by reference thereto.

TABLE 6

| Gene | | No. of | | Observed tissue | |
|---|---|---|---|---|---|
| Seq ID | Gene Name | events | Event | type | Observation results |
| 241 | corn SVP-like | 6 | ZM_M17107 | Tassel, central axis | Elongated |
| 241 | corn SVP-like | 6 | ZM_M17109 | Tassel, central axis | Elongated |
| 241 | corn SVP-like | 6 | ZM_M17113 | Tassel, central axis | elongated |
| 241 | corn SVP-like | 6 | ZM_M17114 | Tassel, central axis | elongated |
| 241 | corn SVP-like | 6 | ZM_M18338 | Tassel, floret, anther | no extrusion |
| 241 | corn SVP-like | 6 | ZM_M18350 | Stem, internode | increased length |
| 66 | Receiver domain (ARR2-like) | 5 | ZM_M15362 | Tassel, floret, anther | no extrusion |
| 66 | Receiver domain (ARR2-like) | 5 | ZM_M15363 | Tassel, floret, anther | no extrusion |
| 66 | Receiver domain (ARR2-like) | 5 | ZM_M15364 | Tassel, floret, anther | no extrusion |
| 66 | Receiver domain (ARR2-like) | 5 | ZM_M15365 | Tassel, floret, anther | no extrusion |
| 66 | Receiver domain (ARR2-like) | 5 | ZM_M16223 | Tassel, floret, anther | no extrusion |
| 68 | Receiver domain (TOC1-like) 3 | 4 | ZM_M12575 | Stem, internode | decreased length |
| 68 | Receiver domain (TOC1-like) 3 | 4 | ZM_M12576 | Stem, internode | decreased length |
| 68 | Receiver domain (TOC1-like) 3 | 4 | ZM_M12583 | Leaf, blade | interveinal chlorosis |
| 68 | Receiver domain (TOC1-like) 3 | 4 | ZM_M13665 | Stem, internode | Other - see notes |
| 71 | Receiver domain (RR3-like) 6 | 4 | ZM_M24601 | Tassel, floret, anther | no extrusion |
| 71 | Receiver domain (RR3-like) 6 | 4 | ZM_M24603 | Tassel, floret, anther | no extrusion |
| 71 | Receiver domain (RR3-like) 6 | 4 | ZM_M24623 | Tassel, floret, anther | no extrusion |
| 71 | Receiver domain (RR3-like) 6 | 4 | ZM_M24624 | Tassel, floret, anther | no extrusion |
| 197 | corn HY5-like | 2 | ZM_M16307 | Stem, internode | decreased length |
| 197 | corn HY5-like | 2 | ZM_M17106 | Stem, internode | decreased length |
| 165 | RNAse S | 2 | ZM_M24554 | Stem, internode | thin |
| 165 | RNAse S | 2 | ZM_M24557 | Leaf, complete | light green |
| 119 | soy HSF | 2 | ZM_M20417 | Stem, internode | decreased length |
| 119 | soy HSF | 2 | ZM_M20418 | Tassel, floret, anther | no extrusion |
| 14 | cytochrome P450 | 2 | ZM_M23446 | Leaf, blade | early senescence |
| 14 | cytochrome P450 | 2 | ZM_M23450 | Leaf, blade | early senescence |
| 3 | sorghum proline permease | 2 | ZM_M18655 | Stem, internode | decreased length |
| 3 | sorghum proline permease | 2 | ZM_M19446 | Leaf, complete | light green |
| 4 | rice AA transporter | 2 | ZM_M18625 | Plant, complete | delayed growth |
| 4 | rice AA transporter | 2 | ZM_M18626 | Tassel, floret | feminized-silk/seed |
| | | | | | formation |
| 203 | helix-loop-helix protein (PIF3 | 2 | ZM_M18452 | Tassel, central axis | shortened, increased |
| | | | | | girth |
| 203 | helix-loop-helix protein (PIF3 | 2 | ZM_M20549 | Tassel, central axis | shortened, increased |
| | | | | | girth |
| 349 | soy myb transcription factor | 7 | ZM_M24248 | Stem, internode | increased length |
| 349 | soy myb transcription factor | 7 | ZM_M24251 | Stem, internode | increased length |
| 349 | soy myb transcription factor | 7 | ZM_M24349 | Stem, internode | increased length |
| 349 | soy myb transcription factor | 7 | ZM_M24350 | Stem, internode | increased length |
| 349 | soy myb transcription factor | 7 | ZM_M24808 | Stem, internode | increased length |
| 349 | soy myb transcription factor | 7 | ZM_M24810 | Stem, internode | increased length |
| 349 | soy myb transcription factor | 7 | ZM_M24811 | Stem, internode | increased length |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

U.S. Pat. No. 4,535,060. Inhibition resistant 5-enolpyruvyl-3-phosphoshikimate synthetase, production and use. Inventor(s): Comai, Luca.

U.S. Pat. No. 4,563,417. Nucleic acid hybridization assay employing antibodies to intercalation complexes. Inventor(s): Albarella, James P, Anderson, Leslie H D.

U.S. Pat. No. 4,582,789. N-substituted-4,9-dioxa-1,12-dodecanediamines useful for preparing nucleic acid intercalators. Inventor(s): Sheldon III, Edward Lewis, Levenson, Corey Howard, Mullis Kary Banks, Rapoport, Henry.

U.S. Pat. No. 4,959,317. Site-specific recombination of DNA in eukaryotic cells. Inventor(s):Sauer, Brian L.

U.S. Pat. No. 5,106,739. CaMv 355 enhanced mannopine synthase promoter and method for using same. Inventor(s): Comai, Luca, Moran, Paul M.

U.S. Pat. No. 5,254,801. Heterologous dominant conditional lethal gene which is a phosphonate monoester hydrolase and use thereof in plants. Inventor(s): Dotson, Stanton B, Kishore, Ganesh M.

U.S. Pat. No. 5,302,523. Transformation of plant cells. Inventor(s): Coffee, Ronald, Dunwell, James M.

U.S. Pat. No. 5,384,253. Genetic transformation of maize cells by electroporation of cells pretreated with pectin degrading enzymes. Inventor(s): Krzyzek, Richard A, Laursen, Cheryl, R M, Anderson, Paul C.

U.S. Pat. No. 5,464,765. Transformation of plant cells. Inventor(s): Coffee, Ronald, Dunwell, James M.

U.S. Pat. No. 550,318. Methods and compositions for the production of stably transformed, fertile monocot plants and cells thereof Inventor(s): Adams, Thomas R, Chambers, Sheryl A, Daines, Richard J, Gordon-Kamm, W J, Kausch, Albert P, Lemaux, Peggy G, Mackey, Catherine J, Mangano, Mary L, O'Brien, James V, Rice, Thomas B, Spencer, T Michael, Start, William G, Willetts, Nancy G.

U.S. Pat. No. 5,508,184. Process for transforming plant protoplast. Inventor(s): Negrutiu, loan, Potrykus, Ingo.

U.S. Pat. No. 5,508,468. Fertile transgenic corn plants. Inventor(s): Lundquist, Ronald C, Walters, David A, Kirihara, Julie A.

U.S. Pat. No. 5,510,471. Chimeric gene for the transformation of plants. Inventor(s): Lebrun, Michel, Leroux, Bernard, Sailland, Alain.

U.S. Pat. No. 5,527,695. Controlled modification of eukaryotic genomes. Inventor(s): Hodges, Thomas K, Lyznik, Leszed A.

U.S. Pat. No. 5,538,877. Method for preparing fertile transgenic corn plants. Inventor(s): Lundquist, Ronald C, Walters, David A.

U.S. Pat. No. 5,538,880. Method for preparing fertile transgenic corn plants. Inventor(s): Lundquist, Ronald C, Walters, David A.

U.S. Pat. No. 5,545,818. Expression of Bacillus thuringiensis cry proteins in plant plastids. Inventor(s): McBride, Kevin E, Maliga, Pal.

U.S. Pat. No. 5,550,318. Methods and compositions for the production of stably transformed, fertile monocot plants and cells thereof. Inventor(s): Chambers, Sheryl A, Gordon-Karmm, William J, Spencer T Michael, Rice, Thomas B, Adams Thomas R, Kausch, Albert P, LeMaux, Peggy G, Mangano, Mary L, O'Brien, James V, Start, William G, Daines, Richard J, Mackey, Catherine J, Willetts, Nancy G.

U.S. Pat. No. 5,563,055. Method of Agrobacterium-mediated transformation of cultured soybean cells. Inventor(s): Townsend, Jeffrey A, Thomas, Laurie A.

U.S. Pat. No. 5,591,616. Method of transforming monocotyledon. Inventor(s): Hiei, Yukou Japan Tobacco Inc P, Komari, Toshihiko Japan Tobacco.

U.S. Pat. No. 5,605,793. Methods for in vitro recombination. Inventor(s): Stemmer, Villem P C.

U.S. Pat. No. 5,627,061. Glyphosate-tolerant 5-enolpyruvylshikimate-3-phosphate synthases. Inventor(s): Barry, Gerard F, Kishore, Ganesh M, Padgette, Stephen R, Stallings, William C.

U.S. Pat. No. 5,658,772. Site-specific recombination of DNA in plant cells. Inventor(s): Hsu, Francis Chuoh, Russell, Sandra, Hoff, Sauer, Brian, Lee, Odell, Joan Tellefsen, Shen, Jennie Bih-Jien.

U.S. Pat. No. 5,728,925. Chimaeric gene coding for a transit peptide and a heterologous polypeptide. Inventor(s): Herrera-Estrella, Luis, Van Den Broeck, Buido, Van Montagu, Mark, Schell, Jeff, Schreier, Peter, Bohnert, Hans J, Kausch, Albert P, Timko, Michael P, Cashmore, Anthony R.

U.S. Pat. No. 5,780,708. Fertile transgenic corn plants. Inventor(s): Walters, David A, Lundquist, Ronald C.

U.S. Pat. No. 5,811,238. Methods for generating polynucleotides having desired characteristics by iterative selection and recombination. Inventor(s): Crameri, Andreas, Stemmer, Willem P C.

U.S. Pat. No. 5,830,721. DNA mutagenesis by random fragmentation and reassembly. Crameri, Andreas, Stemmer, Willem P C.

U.S. Pat. No. 5,837,458. Methods and compositions for cellular and metabolic engineering. Minshull, Jeremy, Stemmer, Willem P C.

U.S. Pat. No. 593,950. Production of enzymes having desired activities by mutagenesis. Inventor(s): Short, Jay M.

U.S. Pat. No. 5,965,408. Method of DNA reassembly by interrupting synthesis. Inventor(s): Short, Jay M.

U.S. Pat. No. 5,981,836: Plant plastid division genes. Inventor(s): Osteryoung; Katherine W, Reno, N V.

U.S. Pat. No. 5,981,840. Methods for agrobacterium-mediated transformation. Inventor(s): Cai, Tishu, Zhao, Zuo-Yu, Gu, Weining, Pierce, Dorothy A.

U.S. Pat. No. 6,118,047. Anthranilate synthase gene and method of use thereof for conferring tryptophan overproduction. Girffor, Matthew C, Chomet, Paul S, Kriz, Alan L, Anderson, Paul C.

U.S. Pat. No. 6,194,636. Maize RS324 promoter and methods for use thereof. McElroy, David, Laccetti, Lucille B, Orozco Jr, Emil M.

U.S. Pat. No. 6,207,879: Maize RS81 promoter and methods for use thereof. Inventor(s): McElroy, David, Orozco, Jr, Emil M, Laccetti, Lucille B.

WO 8402913. Chimeric genes suitable for expression in plant cells. Inventor(s): Rogers, Stephen G, Fraley, Robert T.

WO 9506128. Fertile, transgenic maize plants and methods for their production. Inventor(s): Gordon-Kamm, William J, Dams, Thomas R., Orr, Peter M, Anderson, Paul C, Kausch, Albert P, Daines, Richard J, Mackey, Catherine J, Orozoco, Emil M Jr, Stephens, Michael A.

WO 95/30005. Genes regulating the response of Zea mays to water deficit. Inventor(s): Chomet, Paul, Dellaporta, Stephen L., Orr, Peter, Krueger, Roger W., Lowe, Brenda A.

WO 9704103. Mutated 5-enol pyruvylshikimate-3-phosphate synthase, gene coding for said protein and transformed plants containing said gene. Inventor(s): Lubrun, Michel, Sailland, Alain, Freyssinet, Georges.

WO 9741228. Use of the green fluorescent protein as a screenable marker for plant transformation. Inventor(s): Gordon-Kamm, William, Pierce, Dortothy A, Bidney, Dennis, Millerm Mike, Ross, Margit, Wang, Lijuan, Bowen, Benjamin, Sandahl, Gary, Scelonge, Christopher.

WO 9831837. Evolution of whole cells and organisms by recursive sequency recombination. Inventor(s): Delcardayre, Stephen B, Minshull, Jeremy, Ness, Jon E, Patten, Phillip, Tobin, Mathew, B, Stemmer, Willem P C.

WO 9842728. Recombination of polynucleotide sequences using random or defined primers. Inventor(s): Shao, Zhixin, Zhao, Huimin, Aftholter, Joseph A, Arnold, Frances H, Giver, Lorraine J.

WO 9842851: Plants with modified growth. Inventor(s): Murray, James Augustus Henry.

WO 9846775. Oxidative stress resistance gene. Inventor(s): Kiraly, Zoltan, Sass, Laszlo, Balazs, Bame, Deak, Maria, Dudits, Denes, Toeroek, Karolyne.

WO 9958659. Methods and compositions for expression of transgenes in plants. Inventor(s): Kriz, Alan L, Voyles, Dale A, Luethy, Michael H.

WO 9960129. Methods and compositions for transgene identification. Inventor(s): Spencer, T Michael, Kriz, Alan L.

WO 9965927. Method for producing polynucleotides with desired properties. Inventor(s): Krebber, Claus M.

WO 0017364. Cyclin polynucleotides, polypeptides and uses thereof. Inventor(s): Gordon-Kamm, W J, Gregory, Carolyn A, Hoerster, George J, Tao, Yumin, Lowe, Keith S, McElver, John A.

WO 0000597: Nonsymbiotic plant hemoglobins to maintain cell energy status. Inventor(s): Phillip, G, Duff S M G, Xianzhou, N, Hill, R, Dumin, D, Sowa, A.

WO 0070062. Expression of sedoheptulose 1,7-bisphosphatase in transgenic plants. Inventor(s): Staub, R L, Miller P W.

WO 0109302. A novel Agrobacterium-mediated plant transformation method. Inventor(s): Armstrong, Charles L, Rout, Jyoti R.

WO 0126459. Flowering Time Modification. Inventor(s): Ratcliff, O, Heard, J, Samaha, R, Creelman, R, Keddie, J, Jiang, C, Reuber, L, Riechmann, J L.

WO 0215675. Genes for Modifying Plant Traits IV. Inventor(s):Pilgrim, M, Creelman, R, Dubell, A J, Heard, J, Jiang, C, Keddie, J, Adam, L, Ratcliff, O, Reuber, J L, Riechmann, J L, Yu, G L, Pineda, O.

Aarts, M G, Keijzer, C J, Stiekema, W J, Pereira, A. Molecular characterization of the CER1 gene of arabidopsis involved in epicuticular wax biosynthesis and pollen fertility. Plant Cell. December 1995; 7 (12):2115-27.

Abarca, D, Madueno, F. Martinez-Zapater, J M, Salinas, J. 1999 Dimerization of Arabidopsis 14-3-3 proteins: structural requirements within the N-terminal domain and effect of calcium. FEBS Lett. 462:377-82.

Ach, R., Durfee, T, Miller, A, Taranto, P, Hanley, L, Zambryski, P and Gruissem, W 1997. RRB1 and RRB2 encode maize retinoblastoma-related proteins that interact with a plant D-type cyclin and geminivirus replication protein. Molecular and Cellular Biology; 17(9):5077-5086.

Albanis, D, Mariconti, L, Ricagno, S, Pitto, L, Moroni, C, Kristin, H and Cella, R 2000 DcE2F a functional plant E2F-like transcriptional activator from Daucus carota. The Journal of Biological Chemistry;275(25): 19258-19267.

Albert, H, Dale, E C, Lee, E, Ow, D W. Site-specific integration of DNA into wild-type and mutant lox sites placed in the plant genome. Plant Journal 1995;7(4):649-659.

Albert, S, Delseny, M, Devic, M 1997 BANYULS, a novel negative regulator of flavonoid biosynthesis in the Arabidopsis seed coat. Plant J;11(2):289-299.

Albury, M S, Dudley, P, Watts, F Z, Moore, A L. 1996. Targeting the plant alternative oxidase protein to Schizosaccharomyces pombe mitochondria confers cyanide-insensitive respiration. J. Biol. Chem;271(29): 17062-17066.

Albury, M S, Affourtit, C, Moore, A L. 1998. A highly conserved glutamante residue (Glu-270) is essential for plant alternative oxidase activity. J.Biol Chem;273:301-305.

Alia, Hayashi H, Mustardy, L, Deshnium, P, Ida, M, Murata, N. Transformation of Arabidopsis thaliana with the coda gene for choline oxidase; accumulation of glycine betaine and enhanced tolerance to salt and cold stress. Plant J July 1977;12(1):133-142.

Alia, Hayashi H, Sakamoto, A, and Murata, N. Enhancement of the tolerance of Arabidopsis to high temperatures by genetic engineering of the synthesis of glycine betaine. Plant J October 1998; 16(2):155-161.

Allen, R. Dissection of Oxidative Stress Tolerance Using Transgenic Plants. Plant Physiology 1995; 107:1049-1054.

Allende, M L, Amsterdam, A, Becker, T, Kawakami, K, Gaiano, N, Hopkins, N. 1996 Genes Dev December 15;10(24):3141-55.

Allison, L A. 2000 The role of sigma factors in plastid transcription. Biochimie;82(6-7):537-548.

Allison, L A, North, M T, Neville, L A. 1995 Differential binding of oocyte-type and somatic-type 5S rRNA to TFIIIA and ribosomal protein L5 in Xenopus oocytes: specialization for storage versus mobilization. Dev Biol;168:284-295.

Allison, L A, Romaniuk, P J, Bakken, A H. 1991 RNA-protein interactions of stored 5S RNA with TFIIIA and ribosomal protein L5 during Xenopus oogenesis. Dev Biol;144:129-144.

Alonso, J M, Ecker, J R. 2001 The ethylene pathway: a paradigm for plant hormone signaling and interaction. Sci STKE2001: RE1.

An et al., The Plant Cell, Vol. 1, 115-122, January 1989

Andersen, P, Kaasen, I, Styrvold, O, Boulnois, G, Strom, A. 1988 Molecular cloning, physical mapping and expression of the bet gene governing the osmoregulatory choline-glycine betaine pathway of Escerichia coli. Journal of General Microbiology; 134:1737-1746.

Andersen, J M et. al. 1988 P-700 content and polypeptide profile of chlorophyll-protein complexes of spinach and barley thylakoids. Biochim.Biophys.Acta;591 :113-126.

Ang, L H, Chattopadhyay, S, Wei, N, Oyama, T, Okada, K, Batschauer, A, Deng, X W. 1998 Molecular interaction between COP1 and HY5 defines a regulatory switch for light control of Arabidopsis development. Mol Cell;1:213-222.

Ansaldi, R, Chaboud, A, Dumas, C. 2000 Multiple S gene family members including natural antisense transcripts are differentially expressed during development of maize flowers. J Biol Chem;275:24146-24155.

Aoki, H, Dekany, K, Adams, S L and Ganoza, M C. 1997 The gene encoding the elongation factor P protein is essential for viability and is required for protein synthesis. J. Biol. Chem;272 :32254-32259.

Aoshima, H, Yamada, M, Sauer, N, Komor, E, and Schobert, C. 1993 Heterologous expression the proton/hexose cotransporter from Chlorella in Xenopus oocytes and its characterization with respect to sugar specificity, pH and membrane potential. J. Plant Physiol; 141 :293-297.

Apse, M. Aharon, G., Snedden, W. and Blumwald E. 1999 Salt tolerance conferred by overexpression of a vacuolar Na+/H+ antiport in Arabidopsis. Science;285: 1256-1258.

Aquila, H, Link, T A. Klingenberg, M. 1987 Solute carriers involved in energy transfer of mitochondria form a homologous protein family. FEBS Lett;212:1-9.

Aramayo, R, Timberlake, W E. 1990 Sequence and molecular structure of the Aspergillus nidulans yA (laccase I) gene. Nucleic Acids Res;18(11):3415.

Aravind, L, Makarova, K S, Koonin, E V. 2000 SURVEY AND SUMMARY: holliday junction resolvases and related nucleases: identification of new families, phyletic distribution and evolutionary trajectories. Nucleic Acids Res;28:3417-3432.

Arazi, T, Baum, G, Snedden, W A, Shelp, B J, Fromm, H. 1995 Molecular and biochemical analysis of calmodulin interactions with the calmodulin-binding domain of plant glutamate decarboxylase. Plant Physiol;108:551-561.

Arteca, R. 1995. Brassinosteroids. In Plant Hormones Physiology. Biochemistry and Molecular Biology, ed P J Davies, pp206-13. Dordrecht: Kluwer, 2^{nd} ed.

Artus, N N, Uemura, M, Steponkus, P L, Gilmour, S J, Lin, C, Thomashow, M F. 1996 Constitutive expression of the cold-regulated Arabidopsis thaliana COR15a gene affects both chloroplast and protoplast freezing tolerance. Proc Natl Acad Sci USA.;93: 13404-13409.

Ashikari, M, Wu, J, Yano, M, Sasaki, T, Yoshimura, A. 1999 Rice gibberellin-insensitive dwarf mutant gene Dwarf 1 encodes the alpha-subunit of GTP-binding protein. Proc Natl Acad Sci USA; 96:10284-10289.

Athwal, G S, Lombardo, C R, Huber, J L, Masters, S C, Fu, H, Huber, S C. 2000 Modulation of 14-3-3 protein interactions with target polypeptides by physical and metabolic effectors. Plant Cell Physiol;41 :523-533.

Aurisano, N, Bertani, A, Reggiani, R. 1995 Anaerobic accumulation of 4-aminobutyrate in rice seedlings; causes and significance. Phytochemistry;38:1147-1150.

Axelsen, K B, Venema, K, Jahn, T, Baunsgaard, L, and Palmgren, M G. 1999 Molecular dissection of the C-terminal regulatory domain of the plant plasma membrane H⁺-ATPase AHA2: Mapping of residues that when altered give rise to an activated enzyme. Biochemistry, June 1;38(22):7227-7234.

Babakov, A V, Chelysheva, V V, Klychnikov, O I, Zorinyanz, S E, Trofimova, M S, De Boer, A H. 2000 Involvement of 14-3-3 proteins in the osmotic regulation of H+-ATPase in plant plasma membranes. Planta;21 :446-448.

Babcock, D, Herringron, J, Goodwin, P, BaePank, Y, Hille, B. 1997 Mitochondrial participation in the intracellular Ca2+ network. J. Cell Biol;136:833-834.

Babiychuk, E, Kushnir, S, Belles-Boix, E, Van Montagu, M, Inze, D. 1995 Arabidopsis thaliana NADPH oxidoreductase homologs confer tolerance of yeasts toward the thiol-oxidizing drug diamide. J Biol Chem;270:26224-26231.

Bach, T J, Benveniste, P. 1997. Cloning of cDNAs or genes encoding enzymes of sterol biosynthesis from plants and other eukaryotes: Heterologous expression and complementation analysis of mutations for functional characterization. Prog. Lipid Res;36:197-226.

Bagnall, D J, King, R W, Whitelam, G C, Boylan, M T, Wagner, D, Quail, P H. 1995 Flowering responses to altered expression of phytochrome in mutants and transgenic lines of Arabidopsis thaliana (L.) Heynh. Plant Physiol; 108:1495-1503.

Baier, M, Dietz, K J. 1999 Protective function of chloroplast 2-cysteine peroxiredoxin in photosynthesis. Evidence from transgenic Arabidopsis. Plant Physiol;119:1407-1414.

Bajguz, A, Czerpak, R. 1996. Effect of brassinosteroids on growth and proton extrusion in the alga Chlorella vulgaris. J. Plant Growth Regul;15:153-156.

Balachandran, S, Xiang, Y, Schobert, C, Thompson, G A, and Lucas, W J. 1997 Phloem sap proteins from Cucurbita maxima and Ricinus communis have the capacity to traffic cell to cell through plasmodesmata. Proc. Natl. Acad. Sci. USA;94:14150-14155.

Balzi, E, Wang, M, Leterme, S, Dyck, L, Goffeau, A. 1994 PDR5, a novel yeast multidrug resistance conferring transporter controlled by the transcription regulator PDR1. J.Biol.Chem;269:2206-2214.

Barata, R M, Chaparro, A, Chabregas, S M, Gonzalez, R, Labate, C A, Azevedo, R A, Sarath, G, Lea, P J, Silva-Filho, M C. 2000 Targeting of the soybean leghemoglobin to tobacco chloroplasts: effects on aerobic metabolism in transgenic plants. Plant Sci;155(2):193-202.

Bariola, P A, Howard, C J, Taylor, C B, Verburg, M T, Jaglan, V D, Green, P J. 1994 The Arabidopsis ribonuclease gene RNS1 is tightly controlled in response to phosphate limitation. Plant J;6:673-685.

Barroso, J B, Corpas, F J, Carreras, A, Sandalio, L M, Valderrama, R, Palma, J M, Lupianez, J A, del Rio, L A. 1999 Localization of nitric-oxide synthase in plant peroxisomes. J Biol Chem;274:36729-36733.

Bartig, D, Lemkemeier, K, Frank, J, Lottspeich, F, and Klink, F. 1992 The archaebacterial hypusine-containing protein. Structural features suggest common ancestry with eukaryotic translation initiation factor 5A. Eur. J. Biochem;204:751-758.

Bartling, D, Rehling, P, Weiler, E W. 1993 Functional expression and molecular characterization of AtUBC2-1, a novel ubiquitin-conjugating enzyme (E2) from Arabidopsis thaliana. Plant Mol Biol;23:387-396.

Bates, P W, Vierstra, R D. 1999 UPL1 and 2, two 405 kDa ubiquitin-protein ligases from Arabidopsis thaliana related to the HECT-domain protein family. Plant J;20:183-195.

Baudat, F, Nicolas, A. 1997 Clustering of meiotic double-strand breaks on yeast chromosome III. Proc Natl Acad Sci USA;94:5213-5218.

Baum, G, Chen, Y, Arazi, T, Takatsuji, H, Fromm H. 1993 A plant glutamate decarboxylase containing a calmodulin binding domain. Cloning, sequence, and functional analysis. J Biol Chem;268:19610-19617.

Baum, G, Lev-Yadun, S, Fridmann, Y, Arazi, T, Katsnelson, H, Zik, M, Fromm, H. 1996 Calmodulin binding to glutamate decarboxylase is required for regulation of glutamate and GABA metabolism and normal development in plants. EMBO J;15:2988-2996.

Baunsgaard, L, Venema, K, Axelsen, K B, Villalba, J M, Welling, A, Wollenweber, B, and Palmgren, M G. 1996 Modified plant plasma membrane H⁺-ATPase with improved transport coupling efficiency identified by mutant selection in yeast. Plant J;10:451-458.

Beck et al., Gene, 19 (1982), 327-336

Becraft, P W, Stinard, P S, McCarty, D R. 1996 CRINKLY4: A TNFR-like receptor kinase involved in maize epidermal differentiation. Science;273:1406-1409.

Beers, E P, Moreno, T N, Callis, J. 1992 Subcellular localization of ubiquitin and ubiquitinated proteins in Arabidopsis thaliana. J Biol Chem;267:15432-15439.

Beligni, M V, Lamattina, L. 2000 Nitric oxide stimulates seed germination and de-etiolation, and inhibits hypocotyl elongation, three light-inducible responses in plants. Planta;210:215-221.

Bell, E, Creelman, R A, Mullet, J E. 1995 A chloroplast lipoxygenase is required for wound-induced jasmonic acid accumulation in Arabidopsis. Proc. Natl. Acad. Sci. USA;92:8675-8679.

Belles-Boix, E, Babiychuk, E, Montagu, M V, Inze, D, Kushnir, S. 2000a CEF, a sec24 homologue of arabidopsis thaliana, enhances the survival of yeast under oxidative stress conditions. J Exp Bot;51:1761-1762.

Belles-Boix, E, Babiychuk, E, Van Montagu, M, Inze, D, Kushnir, S. 2000b CEO 1, a new protein from arabidopsis thaliana, protects yeast against oxidative damage. FEBS Lett;482:19-24.

Benard, L, Carroll, K, Valle, R C P, Masison, D C, Wickner, R B. 1999 The ski7 antiviral protein is an EF 1-alpha homolog that blocks expression of non-Poly(A) mRNA in Saccharomyces cerevisiae. J Virol;73:2893-2900.

Benfey, P N, Chua, N -H. Regulated genes in transgenic plants. Science 1989;244:174-181.

Benne, R, Brown-Luedi, M L, Hershey, J W. 1978 Purification and characterization of protein synthesis initiation factors eIF-1, eIF-4C, eIF-4D, and eIF-5 from rabbit reticulocytes. J Biol Chem;253 :3070-3077.

Bensen, R J, Johal, G S, Crane, V C, Tossberg, J T, Schanable, P S, Meeley, R B, Briggs, S P. Cloning and characterization of the maize An1 gene. Plant Cell January 1995;7(1):75-84.

Benveniste, P. 1986 Sterol biosynthesis. Annu. Rev. Plant Physiol;37:275-308.

Berberich, T, Sano, H, Kusano, T. 1999 Involvement of a MAP kinase, ZmMPK5, in senescence and recovery from low-temperature stress in maize. Mol Gen Genet. 262:534-42.

Berberich, T, Kusano, T. Cycloheximide induces a subset of low temperature-inducible genes in maize. Mol Gen Genet 1997;254:275-283.

Berberich, T, Sano, H, and Kusano, T. Involvement of a MAP kinase, ZmMPK5, in senescence and recovery from low-temperature stress in maize. Mol. Gen. Genet. 1999;262:534-542.

Bertin, P. Somaclonal variation and chilling tolerance improvement in rice: changes in fatty acid composition. Plant Growth Regulation January 1998; 24(1):31-41.

Bertolotti, A, Ron, D. 2001 Alterations in an IRE1-RNA complex in the mammalian unfolded protein response. J Cell Sci. 114(Pt 17):3207-3212.

Bertram, P G, Zeng, C, Thorson, J, Shaw, A S, Zheng, X F. 1998 The 14-3-3 proteins positively regulate rapamycin-sensitive signaling. Curr Biol; 8:1259-1267.

Bevan, M, Barnes, W M, Chilton, M D. Structure and transcription of the nopaline synthase gene region of T-DNA. Nucleic Acids Res January 25, 1983; 11(2):369-385.

Beyer, A. Sequence analysis of the AAA protein family. Protein Sci October 1997; 6(10):2043-2058.

Biegen, C, Weiler, E W. 1998 Structure and regulation of OPR1 and OPR2: two closely related genes encoding 12-oxophytodienoic acid-10,11-reductases from Arabidopsis thaliana. Planta; 208:155-165.

Blaszczyk, A, Brodzik, R, Sirko, A. 1999 Increased resistance to oxidative stress in transgenic tobacco plants overexpressing bacterial serine acetyltransferase. Plant J; 20:237-243.

Bleecker, A B, Esch, J J, Hall, A E, Rodriguez, F I, Binder B M. 1998 The ethylene-receptor family from Arabidopsis: structure and function. Philos Trans R Soc Lond B Biol Sci 353:1405-1412.

Bloch, K E. 1983. Sterol structure and membrane function. Crit. Rev. Biochem;14:47-91.

Blumwald, E. and Gelli, A. 1997. A secondary inorganic ion transport in plant vacuoles. Advanced Botany Research. 25:401-407

Boggon, T J, Shan, W S, Santagata, S, Myers, S C, Shapiro, L. 1999 Implication of tubby proteins as transcription factors by structure-based functional analysis. Science;286:2119-2125.

Bohlmann, J, Meyer-Gauen, G, Croteau, R. 1998 Plant terpenoid synthases: molecular biology and phylogenetic analysis. Proc Natl Acad Sci USA;95:4126-4133.

Bolarin, M, Santa-Cruz, A, Cayuela, E, Perez-Alfocea, F 1995 Short-term solute changes in leaves and roots of cultivated and wild tomato seedlings under salinity. J. Plant Physiol;147: 463-468.

Bolle, C, Koncz, C, Chua, N H. 2000 PAT1, a new member of the GRAS family, is involved in phytochrome A signal transduction. Genes Dev; 14:1269-1278.

Bolognese, C. and McGraw, P. The isolation and characterization in yeast of a gene for Arabidopsis S-adenosylmethionine: phophoethanolomine N-methyltransferase. Plant Physiology 2000;124:1800-1813.

Bonello, J -F, Opsahl-Ferstak, H -G, Peres, P, Dumas, C, Rogowsky, P M. 2000 Esr genes show different levels of expression in the same region of maize endosperm. Gene;246:219-227.

Bonfil, D J, Ronen-Tarazi, M, Sultemeyer, D, Lieman-Hur-witz, J, Schatz, D, and Kaplan, A. 1998 A putative HC03-transporter in the cyanobacterium Synechococcus sp. Strain PCC 7942. FEBS Lett;430:236-240.

Boorer, K J, Loo, D D F, Frommer, W B, Wright, E M. 1996 Transport mechanism of the cloned potato H⁺/sucrose transporter StSUT1. J. Biol. Chem;271:25139-25144.

Boorer, K J, Loo, D D F, Wright, E M. 1994 Steady-state and presteady-state kinetics of the H⁺/hexose cotransporter (STP1) from Arabidopsis thaliana expressed in Xenopus oocytes. J. Biol. Chem;269:20417-20424.

Borevitz, J O, Xia, Y, Blount, J, Dixon, R A, Lamb, C. 2000 Activation tagging identifies a conserved MYB regulator of phenylpropanoid biosynthesis. Plant Cell;12:2383-2393.

Borghese, B, Crimi, M, Fava, L, Melandri, B A. 1998 The ATP Synthase atpHAGDC (Fi) Operon from Rhodobacter capsulatus. J Bacter; 180: 416-421.

Borghese, B, Turina, P, Lambertini, L, Melandri, B A. 1998 The atpBEXF operon coding for the Fo sector of the ATP synthase from the purple nonsulfur photosynthetic bacterium Rhodobacter capsulans. Arch Microbiol;170: 385-388.

Boscheinen, O, Lyck, R, Queitsch, C, Treuter, E, Zimarino, V, Scharf, K D. 1997 Heat stress transcription factors from tomato can functionally replace HSF1 in the yeast Saccharomyces cerevisiae. Mol Gen Genet;255:322-331.

Bouche-Pillon, S, Fleurat-Lessard, P, Fromont, J -C, Serrano, R, and Bonnemain, J -L. 1994 Immunolocalization of the plasma membrane H⁺-ATPase in minor veins of Vicia faba in relation to phloem loading. Plant Physiol;105:691-697.

Bourbouloux, A, Shahi, P, Chakladar, A, Delrot, S, Bachhawat, A K. 2000 Hgt1p, a high affinity glutathione transporter from the yeast Saccharomyces cerevisiae. J Biol Chem. May 5; 275(18):13259-13265.

Bowler, C, Slooten, L, Vandenbranden, S, De Rycke, R, Botterman, J, Sybesma, C, Van Montagu, M, Inze, D. 1991 Manganese superoxide dismutase can reduce cellular damage mediated by oxygen radicals in transgenic plants. EMBO J;10:1723-1732.

Bowyer, J R, Leegood, R C. 1997 Photosynthesis. In: Plant Biochemistry, Dey P M and Harborn J B eds, pg 49-110. Academic Press.

Boyer, P D. 1997 The ATP synthase—a splendid molecular machine. Annu Rev Biochem;66:717-749.

Bray, M, Driscoll, J, Huggins, J W. Treatment of lethal Ebola virus infection in mice with a single dose of an S-adenosyl-L-homocysteine hydrolase inhibitor. Antiviral Res. February 2000; 45(2):135-147.

Breitkruz, K E, Shelp, B J. 1995 Subcellular compartmentation of the 4-aminobutyrate shunt in protoplasts of developing soybean cotyledons. Plant Physiol;108:99-103.

Bressan, D A, Baxter, B K, Petrini, J H. 1999 The Mre11-Rad50-Xrs2 protein complex facilitates homologous recombination-based double-strand break repair in Saccharomyces cerevisiae. Mol Cell Biol;19:7681-7687.

Brewster, J L, de Valoir, T, Dwyer, N D, Winter, E, Gustin, M C. An Osmosensing Signal Transduction Pathway in Yeast. Science 1993;259:1760-1763.

Briat, J -F, Lobreaux, S, Grignon, N, Vansuyt, G. Review: Regulation of plant ferritin synthesis: how and why. Cellular and Molecular Life Sciences; 56 1999:155-166.

Brown, G G, Lee, J S, Brisson, N, Verma, D P. The evolution of a plant globin gene family. J Mol Evol 1984;21:19-32.

Brown, J T, Bai, X, Johnson, A W. 2000 The yeast antiviral proteins Ski2p, Ski3p, and Ski8p exist as a complex in vivo. RNA;6:449-457.

Bruce, W B, Quail, P H. 1990 Cis-acting elements involved in photoregulation of an oat phytochrome promoter in rice. Plant Cell;2:1081-1089.

Brzobohaty, B, Moore, I, Kristoffersen, P, Bako, L, Campos, N, Schell, J, Palme, K. 1993 Release of active cytokinin by a beta-glucosidase localized to the maize root meristem. Science;262:1051-1054.

Buchanan, B et al. Biochemistry and Molecular Biology of Plants. American Society of Plant Physiologists 2000:1189-1197.

Bugos, R C, Hieber, A D, Yamamoto, H Y. 1998 Xanthophyll cycle enzymes are members of the lipocalin family, the first identified from plants. J Biol Chem;273:15321-15324.

Bulow, L, Holmberg, N, Lilius, G, Bailey, J E. The metabolic effects of native and transgenic hemoglobins on plants. Trends Biotechnol. January 1999;17(1):21-24.

Bürkle, L, Hibberd, J M, Quick, W P, Kühn, C, Himer, B, and Frommer, W B. 1998 The H⁺-sucrose co-transporter NtSUT1 is essential for sugar export from tobacco leaves. Plant Physiol;118:59-68.

Burleigh, S H, Harrison, M J. 1999 The Down-Regulation of Mt4-Like Genes by Phosphate Fertilization Occurs Systemically and Involves Phosphate Translocation to the Shoots. Plant Physiol;119:241-248.

Buschges, R, Hollricher, K, Panstruga, R, Simons, G, Wolter, M, Frijters, A, van Daelen, R, van der Lee, T, Diergaarde, P, Groenendijk, J, Topsch, S, Vos, P, Salamini, F, Schulze-Lefert, P. 1997 The barley Mlo gene: a novel control element of plant pathogen resistance. Cell 1997;88:695-705.

Bush, D S. 1995 Calcium regulation in plant cells and its role in signaling. Annu Rev Plant Physiol Plant Mol Bio;46:95-122.

Byrne, M E, Barley, R, Curtis, M, Arroyo, J M, Dunham, M, Hudson, A, Martienssen, R A. 2000 Asymmetric leaves1 mediates leaf patterning and stem cell function in Arabidopsis. Nature;408:967-971.

Byrne, P F, McMullen, M D, Snook, M E, Musket, T A, Theuri, J M, Widstrom, N W. 1996 Quantitative trait loci and metabolic pathways: Genetic control of the concentration of maysin, a corn earworm resistance factor, in maize silks. Proc. Nat. Acad. Sci. USA;93:8820-8825.

Cabrillac, D, Cock, J M, Dumas, C, Gaude, T. 2001 The S-locus receptor kinase is inhibited by thioredoxins and activated by pollen coat proteins. Nature;410:220-223.

Cadet, F, Meunier, J C. pH and kinetic studies of chloroplast sedoheptulose-1,7-bisphosphatase from spinach (Spinacia oleracea). Biochem J Jul. 1, 1988;253(1):249-254.

Cadet, F, Meunier, J C. Spinach (Spinacia oleracea) chloroplast sedoheptulose-1,7-bisphosphatase. Activation and deactivation, and immunological relationship to fructose-1,6-bisphosphatase. Biochem J Jul. 1, 1988; 253(1):243-248.

Cadet, F, Meunier, J C, Ferte, N. Isolation and purification of chloroplastic spinach (Spinacia oleracea) sedoheptulose-1,7-bisphosphatase. Biochem J 1987;241:71-74.

Calderon-Urrea, A, Dellaporta, S L. 1999 Cell death and cell protection genes determine the fate of pistils in maize. Development; 126:435-441.

Caliebe, A, Grimm, R, Kaiser, G, Lubeck, J, Soll, J, Heins, L. 1997 The chloroplastic protein import machinery contains a Rieske-type iron-sulfur cluster and a mononuclear iron-binding protein. EMBO J;16:7342-7350.

Campbell, C E, Kircher, H W. 1980. Senita cactus: A plant with interrupted sterol biosynthetic pathways. Phytochemistry;19:2777-2779.

Canovas, D, Vargas, C, Kneip, S, Moron, M, Ventosa, A, Bremer, E, Nieto, J. Genes for the synthesis of the osmoprotectant glycine betaine from choline in the moderately halophobic bacterium Halomonas elongata DSM3043. Microbiolgy 2000;146:455-463.

Cao et al. 2001. Molecular cloning and characterization of a cDNA encoding soybean nodule IMPD dehydrogenase. BBA;1520:242-246.

Cao, H, Chen, S. 1995 Brassinosteroid-induced rice lamina joint inclination and its relation to indole-3-acetic acid and ethylene. Plant Growth Regul;16:189-196.

Carabelli, M, Sessa, G, Baima, S, Morelli, G, Ruberti, I. 1993 The Arabidopsis Athb-2 and -4 genes are strongly induced by far-red-rich light. Plant J;4:469-479.

Carels, N, Hatey, P, Jabbari, K, Bernardi, G. Compositional properties of homologous coding sequences from plants. J Mol Evol. January 1998;46(1);45-53.

Carlson, R E. 1990 Heat stress, plant-available soil moisture, and corn yields in Iowa: A short- and long-term view. J. Prod. Agric;3:293-297.

Carter, C, Thornburg, R W. 2000 Tobacco nectarin I. Purification and characterization as a germin-like, manganese superoxide dismutaste implicated in defense of floral reproductive tissues. J Biol Chem;275:36726-36733.

Cary, A J, Liu, W, Howell, S H. 1995 Cytokinin action is coupled to ethylene in its effects on the inhibition of root and hypocotyl elongation in Arabidopsis thaliana seedlings. Plant Physiol;107:1075-1082.

Casagrande, R, Stern, P, Diehn, M, Shamu, C, Osario, M, Zuniga, M, Brown, P O, Ploegh, H. 2000 Degradation of proteins from the ER of S. cerevisiae requires an intact unfolded protein response pathway. Mol Cell;5(4):729-735.

Casal, J J. 1996 Phytochrome A enhances the promotion of hypocotyl growth caused by reductions in levels of phytochrome B in its far-red-light-absorbing form in light-grown Arabidopsis thaliana. Plant Physiol;112:965-973.

Castleberry, R. M. 1979 Latente, a new type of drought tolerance?. Proc Annu Corn Sorghum Res Conf;34:46-56.

Chambraud, B, Radanyi, C, Camonis, J H, Shazand, K, Rajkowski, K, Baulieu, EE. 1996 FAP48, a new protein that forms specific complexes with both immunophilins FKBP59 and FKBP12. Prevention by the immunosuppressant drugs FK506 and rapamycin. J Biol Chem;271 :32923-32929.

Chan, S N, Harris, L, Bolt, E L, Whitby, M C, Lloyd, R G. 1997 Sequence specificity and biochemical characterization of the RusA Holliday junction resolvase of Escherichia coli. J Biol Chem 272:14873-14882.

Chapman, D. 1998 Phospholipase activity during plant growth and development and in response to environmental stress. Trends Plant Sci;3:419-426.

Chattopadhyay, S, Ang, L H, Puente, P, Deng, X W, Wei, N. 1998 Arabidopsis bZIP protein HY5 directly interacts with light-responsive promoters in mediating light control of gene expression. Plant Cell;10:673-683.

Cheikh, N and Jones, R J. 1994 Disruption of maize kernel growth and development by heat stress. Plant Physiol;106:45-51.

Chekanova, J A, Shaw, R J, Wills, M A, Belostotsky, D A. 2000 Poly(A) tail-dependent exonuclease AtRrp41p from arabidopsis thaliana rescues 5.8 S rRNA processing and mRNA decay defects of the yeast ski6 mutant and is found in an exosome-sized complex in plant and yeast cells. J Biol Chem;275:33158-33166.

Chen, Y F, Matsubayashi, Y, Sakagami, Y. 2000 Peptide growth factor phytosulfokine-alpha contributes to the pollen population effect. Planta;211:752-755.

Chen, F, Foolad, M R. 1997 Molecular organization of a gene in barley which encodes a protein similar to aspartic protease and its specific expression in nucellar cells during degeneration. Plant Mol Biol;35:821-831.

Chen, Q, Hung, F C, Fromm, L, Overbeek, P A. 2000 Induction of cell cycle entry and cell death in postmitotic lens fiber cells by over expression of E2F1 or E2F2. Invest Ophthalmol Vis Sci;41 :4223-4231.

Chen, X, Wu, R. Direct amplicification of unknown genes and fragments by Uneven polymerase chain reaction. Gene Feb. 7, 1997; 185(2):195-199.

Chen, Z P, Chen, K Y. 1997a Dramatic attenuation of hypusine formation on eukaryotic initiation factor 5A during senescence of IMR-90 human diploidfibroblasts. J Cell Physiol;170:248-254.

Chen, Z P, Chen, K Y. 1997b Marked elevation of hypusine formation activity on eukaryotic initiation factor 5A in v-HA-RAS transformed mouse NIH3T3 cells. Cancer Lett;115:235-241.

Chiadmi, M, Navaza, A, Miginiac-Maslow, M, Jacquot, J P, Cherfils, J. Redox signalling in the chloroplast: structure of oxidized pea fructose-1,6-bisphosphate phosphatase. EMBO J Dec. 1, 1999; 18(23):6809-6815.

Chiang, P K, Gordon, R K, Tal, J, Zeng, G C, Doctor, B P, Pardhasaradhi, K, McCann, P P. S-Adenosylmethionine and methylation. FASEB J 1996; 10:471-480.

Childs, K L, Pratt, L H, Morgan, P W. 1991 Genetic regulation of development in Sorghum bicolor. VI. The ma3 allele results in abnormal phytochrome physiology. Plant Physiol;108:345-351.

Chivasa, S, Berry, J O, Rees, T, Carr, J P. 1999 Changes in gene expression during development and thermogenesis in Arum. Aust. J. of Plant Physiol;26:391-399.

Cho, M. H., Shears, S. B., Boss, W. F. 1993 Changes in phosphatidylinositol metabolism in response to hyperosmotic stress in Daucus carota L. cells grown in suspension culture. Plant Physiol.103, 637-647.

Choe, S, Dilkes, B. Gregory, B D, Ross, A S, Yuan, H, Noguchi, T, Fujioka, S, Takatsuto, S, Tanaka, A, Yoshida, S. 1999a. Arabidopsis dwarfl is defective in the conversion of 24-methylenecholesterol to campesterol in brassinosteroid biosynthesis. Plant Physiol;119:897-907.

Choe, S, Noguchi T, Fujioka, S, Takatsuto S, Tissier C P, Gregory B D, Ross A S, Tanaka A, Yoshida S, Tax F E 1999b. The Arabidopsis dwf7/ste1 mutant is defective in the ⁷sterol C-5 desaturation step leading to brassinosteroid biosynthesis. Plant Cell; 11:207-221.

Choi, G, Yi, H, Lee, J, Kwon, Y K, Soh, M S, Shin, B, Luka, Z, Hahn, T R, Song, P S. 1999 Phytochrome signalling is mediated through nucleoside diphosphate kinase 2. Nature;401 :610-613.

Cholewa, E, Cholewinski, A J, Shelp, B J, Snedden, W A, Bown, A W. 1997 Cold stimulated—aminobutyric acid synthesis is mediated by an increase in cytosolic Ca²⁺, not by an increase in cytosolic H⁺. Can J Bot;75:375-382.

Chow, W S, Melis, A, Anderson, J M. Adjustments of photosystem stoichiometry in chloroplasts improve the quantum efficiency of photosynthesis. Proc. Natl.Acad.Sci. USA Oct. 1, 1990; 87(19):7502-7506.

Chrispeels, M J, Crawford, N M, Schroeder, J I. 1999. Proteins for Transport of Water and Mineral Nutrients across the Membranes of Plant Cells. Plant Cell;11:661-676.

Chu, C C., C C. Wang, C S. Sun, C. Hus, K C. Yin, C Y. Chu and F Y. Bi. 1975 Establishment of an efficient medium for anther culture of rice through comparative experiments on the nitrogen sources. Sci. Sin.18: 659-668.

Chung, W S, Lee, S H, Kim, D H, Kim, M C, Park, C Y, Park, H C., Lim, C O, Harper, J F, Cho, M J. 2000 Identification of a calmodulin-regulated soybean Ca+-ATPase (SCA1) that is located in the plasma membrane. Plant Cell;12:1393-1407.

Chung, Y Y, Kim, S R, Finkel, D, Yanofsky, M F, An, G. 1994 Early flowering and reduced apical dominance result from ectopic expression of a rice MADS box gene. Plant Mol Biol.26:657-65.

Cicek, M, Blanchard, D J, Bevan, D R, Esen, A. The aglycone specificity determining sites are different in DIMBOA-glucosidase (maize □-glucosidase) and dhurrinase (sorghum □-Glucosidase). J Biol Chem Jun. 30, 2000;275(26):20002-20011.

Clark, J K and Sheridan, W F. 1991 Isolation and characterization of 51 embryo-specific mutations of maize. Plant Cell;3:935-951.

Clark, M S. 1977 Plant Molecular Biology: A Laboratory Manual, Springer-Verlag.

Clarke, A, Desikan, R, Hurs,t R D, Hancock, J T, Neill, S J. 2000 NO way back: nitric oxide and programmed cell death in Arabidopsis thaliana suspension cultures. Plant J;24:667-677.

Close, T J, Kortt, A A, Chandler P M. 1989 A cDNA-based comparison of dehydration-induced proteins (dehydrins) in barley and corn. Plant Mol Biol;13:95-108.

Close, T J, Lammers, P J. 1993 An osmotic stress protein of cyanobacteria is immunologically related to plant dehydrins. Plant Physiol. March 1993;101(3):773-779.

Clough, R C, Casal, J J, Jordan, E T, Christou, P, Vierstra, R D. 1995 Expression of functional oat phytochrome A in transgenic rice. Plant Physiol;109:1039-1045.

Clouse, S D, Sasse, J M. 1998. Brassinosteroids: Essential regulators of plant growth and development. Annu. Rev. Plant Physiol. Plant Mol. Biol;49: 427-451.

Clouse, S D., Hall, A F, Langford, M, McMorris, T C, Baker, M E J. 1993. Physiological and molecular effects of brassinosterioids on Arabidopsis thaliana. J. Plant Growth Regul; 12:61-66.

Cockcroft, C E, den Boer, B G, Healy, J M, Murray, J A. 2000 Cyclin D control of growth rate in plants. Nature. 405:575-579.

Coe, Jr, E H, Neuffer, M G, Hoisington, D A. 1988) The genetics of corn. In “Corn and Corn Improvement 3^{rd.} ed.” (eds. Sprague G F and Dudley J W), p.81-258. American Society of Agronomy, Madison, Wis.

Coleman, S T, Fang, T K, Rovinsky, S A, Turano, F J, Moye-Rowley, W S. Expression of a glutamate decarboxylase homologue is required for normal oxidative stress tolerance in Saccharomyces cerevisiae. J Biol Chem Jan. 5, 2001;276(1):244-250.

Collart et al. 1996. Cloning and Characterization of the gene encoding IMP dehydrogenase from Arabidopsis thaliana. Gene. 174:217-220.

Colon-Carmona, A, Chen, D L, Yeh, K C, Abel, S. 2000 Aux/IAA proteins are phosphorylated by phytochrome in vitro. Plant Physiol.124:1728-38.

Condorelli, G, Roncarati, R, Ross, J Jr, Pisani, A, Stassi, G, Todaro, M, Trocha, S, Drusco, A, Gu, Y, Russo, M A, Frati, G, Jones, S P, Lefer, D J, Napoli, C, Croce, C M. 2001 Heart-targeted overexpression of caspase3 in mice increases infarct size and depresses cardiac function. Proc Natl Acad Sci USA;98:9977-9982.

Connell, P, Ballinger, C A, Jiang, J, Wu, Y, Thompson, L J, Hohfeld, J, Patterson, C. 2001 The co-chaperone CHIP regulates protein triage decisions mediated by heat-shock proteins. Nat Cell Biol;3 :93-96.

Cooper, G. 1995 Oncogenes, second edition. Jones and Bartlett publishers. Sudbury, Mass.

Cooper, H L, Park, M H, Folk, J E, Safer, B, Braverman, R. 1983 Identification of the hypusine-containing protein hy+ as translation initiation factor eIF-4D. Proc Natl Acad Sci USA; 80:1854-1857.

Cooper, P, Selman, I. 1974 An analysis of the effects of tobacco mosaic virus on growth and the changes in the free amino compounds in young tomato plants. Ann Bot;38:625-638.

Coruzzi, G M, Zhou, L. 2001 Carbon and nitrogen sensing and signaling in plants: emerging ‘matrix effects’. Curr Opin Plant Biol. June;4(3):247-253.

Cosgrove, D J. 1997 Relaxation in a high-stress environment: the molecular basis of extensible cell walls and cell enlargement. Plant Cell;9:1031-1041.

Cotelle, V, Meek, S E, Provan, F, Milne, F C, Morrice, N, MacKintosh, C. 2000 14-3-3s regulate global cleavage of their diverse binding partners in sugar-starved Arabidopsis cells. EMBO J;19:2869-2876.

Covic, L, Silva, N F, Lew, R R. Functional characterization of ARAKIN (ATMEKK1): a possible mediator in an osmotic stress pathway in higher plants. BBCA;1451, 1999:242-254.

Craig, K L, Tyers, M. 1999 The F-box: a new motif for ubiquitin dependent proteolysis in cell cycle regulation and signal transduction. Prog Biophys Mol Biol;72:299-328.

Crawford, L, Brown, A, Breitkreuz, K, Guinel, F. 1994 The synthesis of y-aminobutric acid in response to treatments reducing cytosolic pH. Plant Physiol;104: 865-871.

Crawford, N A, Droux, M, Kosower, N S, Buchanan, B B. 1989 Evidence for function of the ferredoxin/thioredoxin system in the reductive activation of target enzymes of isolated intact chloroplasts. Arch. Biochem. Biophys;271:223-239.

Creelman, R A, and Mullet, J E. 1997 Biosynthesis and action of jasmonates in plants. Annu. Rev. Plant Physiol. Plant Mol. Biol;48:355-381.

Creelman, R A., and Mullet, J E. 1995 Jasmonic acid distribution and action in plants: Regulation during development and response to biotic and abiotic stress. Proc. Natl. Acad. Sci. USA;92:4114-4119.

Crowley, J H, Tove, S, and Parks, L W. 1998. A calcium-dependent ergosterol mutant of Saccharomyces cerevisiae. Curr. Genet;34:93-99.

Cruz, R T, Jordan, W R, and Drew, M C. 1992. Structural changes and associated reduction of hydraulic conductance in roots of Sorghum bicolor L. following exposure to water deficit. Plant Phys;99:203-212.

Cruz-Hernandez, A, Gomez-Lim, M A. 1995. Alternative oxidase from mango (Mangifera indica L.) is differentially regulated during fruit ripening. Planta;197:569-576.

Curie, C, Panaviene, Z, Loulergue, C, Dellaporta, S L, Briat, J F, Walker, E L. 2001 Maize yellow stripel encodes a membrane protein directly involved in Fe(III) uptake. Nature;409:346-349.

Cutler, H, Yokota, T, Adam, G eds. 1991. Brassinosteroids Chemistry, Bioactivity, and Applications. Washington, DC: Am. Chem. Soc.

D'Halluin, K, Bonne, E, Bossut, M, Beuckeleer, M de, Leemans, J. Transgenic maize plants by tissue electroporation. The Plant Cell December 1992;4(12):1495-1505.

Dahlgren, A, Ryden-Aulin, M. 2000 A novel mutation in ribosomal protein S4 that affects the function of a mutated RF1. Biochimie;82:683-691.

Daniel, X, Lacomme, C, Morel, J B, Roby, D. 1999 A novel myb oncogene homologue in Arabidopsis thaliana related to hypersensitive cell death. Plant J;20:57-66.

Daram, P, Brunner, S, Rausch, C, Steiner, C, Amrhein, N, Bucher, M. 1999. Pht2; 1 Encodes a Low-Affinity PhosphateTransporter from Arabidopsis. Plant Cell;11:2153-2166.

Daruwala, R, Song, J, Koh, W S, Rumsey, S C, Levine, M. 1999 Cloning and functional characterization of the human sodium-dependent vitamin C transporters hSVCT1 and hSVCT2. FEBS Lett; 460:480-484.

Daub, M E 1999 Cercosporin, a photosensitizing toxin from Cercospora species. Phytopathology;72:370-374.

Davis, S J, Kurepa, J, Vierstra, R D. 1999 The Arabidopsis thaliana HY1 locus, required for phytochrome-chromophore biosynthesis, encodes a protein related to heme oxygenases. Proc Natl Acad Sci USA;96:6541-6546.

Day, D A, Dry, I B, Soole, K L, Wiskich, J T, Moore, A L. 1991 Regulation of alternative pathway activity in plant mitochondria: deviations from Q-pool behavior during oxidation of NADH and quinols. Plant Physiol;95:948-953.

Day, D A, Whelan, J, Millar, A H, Siedow, J N, Wiskich, J T. 1995 Regulation of alternative oxidase in plants and fungi. Aust. J. Plant Physiol;22:497-509.

Day, D A, Wiskich, J T. 1995 Regulation of alternative oxidase activity in higher plants. J. Bioenerg. Biomembr;27:379-385.

De Block, M, Botterman, J, Vandiwiele, M, Dockx, J, Thoen, C, Gossele, V, Movva, N R, Thompson, C, Van Montagu, M, Leemans, J. EMBO J 1987; 6:2513-2518; see also PCT Publication number WO 87/05629, published Sep. 24, 1987.

De Block, M., De Brouwer, D., Tenning, P. 1989. Transformation of Brassica napus and Brassica oleracea using Agrobacterium tumefaciens and the expression of the bar and neo genes in the transgenic tissue. Plant Physiology, 91: 694-701.

de Vetten N, Quattrocchio F, Mol J, Koes R. 1997 The an11 locus controlling flower pigmentation in petunia encodes a novel WD-repeat protein conserved in yeast, plants, and animals. Genes Dev;11:1422-1434.

de Vetten N C, Lu G, Feri R J. 1992 A maize protein associated with the G-box binding complex has homology to brain regulatory proteins. Plant Cell;4:1295-1307.

De Vlaming, P, Comu, A, Farcy, E, Gerats, A G M, Maizonnier, D, Wiering, H, Wijsman, H J W. 1984 Petunia hybrida 2. A short description of the action of 91 genes, their origin and their map location. Plant Mol. Rep;2:21-42.

Deak, M, Horvath, G V, Davletova, S, Torok, K, Sass, L, Vass, I, Barna, B, Kiraly, Z, Dudits, D. 1999 Plants ectopically expressing the iron-binding protein, ferritin, are tolerant to oxidative damage and pathogens. Nat Biotechnol; 17:192-196.

Dechend, R, Hirano, F, Lehmann, K, Heissmeyer, V, Ansieau, S, Wulczyn, F G, Scheidereit, C, Leutz, A. 1999 The Bcl-3 oncoprotein acts as a bridging factor between NF-kappaB/Rel and nuclear co-regulators. Oncogene;18:3316-3323.

Dehesh, K, Franci, C, Parks, B M, Seeley, K A, Short, T W, Tepperman J M, Quail P H. 1993 Arabidopsis HY8 locus encodes phytochrome A. Plant Cell;5:1081-1088.

Dehesh, K, Hung, H, Tepperman, J M, Quail, P H. 1992 GT-2: a transcription factor with twin autonomous DNA-binding domains of closely related but different target sequence specificity. EMBO J;11:4131-4144.

Dekeyser, R, Claes, B, Marichal, M, Van Montagu, M, Caplan, A. Evaluation of selectable markers for rice transformation. Plant Physiol 1989;90:217-223.

del Pozo, J C, Estelle, M. 2000 F-box proteins and protein degradation: an emerging theme in cellular regulation. Plant Mol Biol;44:123-128.

del Pozo, J C, Allona, I, Rubio, V, Leyva, A, de la Pena, A, Aragoncillo, C, Paz-Ares, J. A type 5 acid phosphatase gene from Arabidopsis thaliana is induced by phosphate starvation and by some other types of phosphate mobilising/oxidative stress conditions. Plant J September 1999; 19(5):579-589.

Della-Cioppa, G, Bauer, S C, Taylor, M T, Rochester, D E, Klein, B K, Shah, D M, Fraley R T, Kishore, G M. Targeting a herbicide-resistant enzyme from Escherichia coli to chloroplasts of higher plants. Bio/Technology 1987;5:579-584.

Dellaporta et al. In: Chromosome Structure and Function: Impact of new Concepts, 18^{th} Stadler Genetics Symposium, 11:263-282, 1988.

DeLong A, Calderon-Urrea A, Dellaporta S L. 1993 Sex determination gene TASSELSEED2 of maize encodes a short-chain alcohol dehydrogenase required for stage-specific floral organ abortion. Cell;74:757-768.

Delrot, S. 1989. Phloem loading. In Transport of Photoassimilates, D A Baker and J A Milburn, eds (London: Longman Scientific), pp. 167-205.

Deltour, R, Fransolet, S, Loppes, R. 1981 Inorganic phosphate accumulation and phosphatase activity in the nucleus of maize embryo root cells. J Cell Sci;47:77-89.

Deveraux, Q L, Reed, J C. 1999 IAP family proteins—suppressors of apoptosis. Genes Dev;13:239-252.

Devic, M, Guilleminot, J, Debeaujon, I, Bechtold, N, Bensaude, E, Koornneef, M, Pelletier, G, Delseny, M. 1999 The BANYULS gene encodes a DFR-like protein and is a marker of early seed coat development. Plant J;9(4):387-398.

Devlin, P F, Patel, S R, Whitelam, G C. 1998 Phytochrome E influences internode elongation and flowering time in Arabidopsis. Plant Cell;10:1479-1487.

Devlin, P F, Robson, P R, Patel, S R, Goosey, L, Sharrock, R A, Whitelam, G C. (1999) Phytochrome D acts in the shade-avoidance syndrome in Arabidopsis by controlling elongation growth and flowering time. Plant Physiol;119:909-915.

Devoto, A, Piffanelli, P, Nilsson, I, Wallin, E, Panstruga, R, von Heijne, G, Schulze-Lefert, P. 1999 Topology, subcellular localization, and sequence diversity of the Mlo family in plants. J Biol Chem. 1999;274:34993-35004.

DeWitt, N D, Harper, J F, and Sussman, M R. 1991 Evidence for a plasma membrane proton pump in phloem cells of higher plants. Plant J;1:121-128.

DeWitt, N D, Sussman, M R. 1995 Immunocytological localization of an epitope-tagged plasma membrane proton pump (H⁺-ATPase) in phloem companion cells. Plant Cell;7:2053-2067.

Dhaese, P, DeGreve, H, Gielen, J, Seurinch, J, Van Montagu, M, Schell, J. Identification of sequences involved in the polyadenylation of higher plant nuclear transcripts using Agrobacterium T-DNA genes as models. EMBO Journal 1983;2(3):419-426.

Dhondt, S, Geoffroy, P, Stelmach, B A, Legrand, M, Heitz, T. 2000 Soluble phospholipase A2 activity is induced before oxylipin accumulation in tobacco mosaic virus-infected tobacco leaves and is contributed by patatin-like enzymes. Plant J. August 2000;23(4):431-440.

Di Como, C J, Arndt, K T. 1996 Nutrients, via the Tor proteins, stimulate the association of Tap42 with type 2A phosphatases. Genes Dev; 10: 1904-1916.

Dickinson, C D. Slow-growth phenotype of transgenic tomato expressing apoplastic invertase. Plant Physiology February 1991;95(2):420-425.

Dickman, M B, Park, Y K, Oltersdorf, T, Li, W, Clemente, T, French, R. 2001 Abrogation of disease development in plants expressing animal antiapoptotic genes. Proc Natl Acad Sci USA; 98:6957-6962.

Dieryck, W, Pagnier, J. Poyart, C, Maarden, M C, Gruber, V, Bournat, P, Baudino, S, Merot, B. 1997 Human haemoglobin from transgenic tobacco. Nature;386(6620):29-30.

Dieterle, M, Zhou, Y C, Schafer, E, Funk, M, Kretsch, T. 2001 EID1, an F-box protein involved in phytochrome A-specific light signaling. Genes Dev;15:939-944.

Dietrich, R A, Richberg, M H, Schmidt, R, Dean, C, Dangl, J L. 1997 A novel zinc finger protein is encoded by the Arabidopsis LSD1 gene and functions as a negative regulator of plant cell death. Cell;88:685-694.

Dillon, V M, Overton, J, Grayer, R J, Harborne, J B. 1997 Differences in the phytoalexin response among rice cultivars of difference resistance to blast. Phytochem;44(4):599-603.

Dixit, R, Nasrallah, J B. 2001 Recognizing self in the self-incompatibility response. Plant Physiol;125:105-108.

Djajanegara, I, Holtzapffel, R, Finnegan, P M, Hoefnagel, M H, Berthold, D A, Wiskich, J T, Day, D A. 1999 A single amino acid change in the plant alternative oxidase alters the specificity of organic acid activation. FEBS Lett;454:220-224.

Dodds, P N, Clarke, A E, Newbigin, E. Molecularization of an S-like Rnase of Nicotiana alata that is induced by phosphate starvation. Plant Mol Bio. May 1996;31 :227-238.

Doebley, J, Wang, R L. Genetics and the evolution of plant form: an example from maize. Cold Spring Harb Symp Quant Biol. 1997;62:361-367.

Doerner, P, Jorgensen, J E, You, R, Steppuhn, J, Lamb, C. 1996 Control of root growth and development by cyclin expression. Nature Apr. 11, 1996;380(6574):520-523.

Doerner, P. 2000 Plant stem cells: The only constant thing is change. Curr Biol;10:R826-829.

Dooner, H K, Robbins, T P, Jorgenssen, R A. 1991 Genetic and developmental control of anthocyanin biosynthesis. Ann. Rev. Genet;25:173-200.

Doulis, A, Debian, N, Kingston-Smith, A, Foyer, C. 1997 Characterization of chilling sensitivity in maize: differential localization of antioxidants in maize leaves. Plant Physiol;114:1031-1037.

Dove, S. K., Cooke, F. T., Douglas, M. R., Sayers, L. G., Parker, P. J., Michell, R. H. 1997 Osmotic stress activates phosphatidylinositol-3,5-bisphosphate synthesis. Nature;390: 187-192.

Dresselhaus, T, Cordts, S, Lorz, H. 1999 A transcript encoding translation initiation factor eIF-5A is stored in unfertilized egg cells of maize. Plant Mol Biol;39:1063-1071.

Drobak, B. K. & Watkins, P. A. 2000 Inositol (1,4,5) trisphosphate production in plant cells: an early response to salinity and hyperosmotic stress. FEBS Lett;481: 240-244.

Duff, S M G, Guy, P A, Nie, X, Durnin, D C, Hill, R D. Haemoglobin expression in germinating barley. Seed Sci. Res. December 1998;8(4):431-436.

Duff, S M G, Wittenberg, J B, Hill, R D. Expression, purification, and properties of recombinant barley. J. Biol. Chem. Jul. 4, 1997;272(27):16746-16752.

Duina, A A, Kalton, H M, Gaber, R F. 1998 Requirement for Hsp9O and a CyP-40-type cyclophilin in negative regulation of the heat shock response. J Biol Chem;273:18974-18978.

Duncan, W G. 1971 Leaf angles, leaf area and canopy photosynthesis. Crop Sci;11:482-485.

Duangploy, S, Zuber, M S and Cumbie, B G. Inheritance of Multiple Aleurone Layering. MGCNL 50:90-91.

Dunn, M. A., Brown, K., Lightowlers, R., Hughes, M. A., A low-temperature responsive gene from barley encodes a protein with single-stranded nucleic acid-binding activity which is phosphorylated in vitro. Plant Mol. Biol. 1996, 30(5):947-959.

Dure, L. 1993 The Lea proteins of higher plants. Control of Plant Gene Expression, CRC Press, Inc. Boca Raton, Fla. Pages:325-335.

Dure, L. 1993 A repeating 11-mer amino acid motif and plant desiccation. Plant J;3:363-369.

Duvick, D N. 1984 Genetic contributions to yield gains of US. hybrid maize, 1930 to 1980. Genetic contributions to yield gains of five major crop plant. CSSA Special Publication Number 7 pg.15-49.

Easterling, D R, Meehl, G A, Parmesan, C, Changnon, S A, Karl, T R, Meams, L O. Climate extremes: observations, modeling, and impacts. Science Sep. 22, 2000;289(5487):2068-2074.

Edwards, D, Murray, J A H, Smith, A G. 1998 Multiple genes encoding the conserved CCAAT-box transcription factor complex are expressed in Arabidoipsis. Plant Physiol;117:1015-1022.

Edwards, G E, Baker, N R. 1993 Can CO2 assimilation in maize leaves be predicted accurately from chlorophyll fluorescence analysis? Photosynth Res;37:89-92.

Elliger, C A, Chan, B C, Waiss, A C. 1980a Flavonoids as larval growth inhibitors. Naturwissenschaften;67:358-360.

Elliger, C A, Chan, B G, Waiss, A C Jr, Ludlin, R E, Haddon, W F. 1980b C-Glycosylflavones from Zea mays that inhibit insect development. Phytochem;19(2):293-297.

Ellis, J G, Llewellyn D J, Walker, J C, Dennis, E S, Peacock, W J. The ocs element: a 16 base pair palindrome essential for activity of the octopine synthase enhancer. EMBO Journal 1987;6(11):3203-3208.

El-Metainy, A Y, Omar, A A. 1981 Biochemical genetics of Zea mays L. I. Isozymes and the linkage relationship between AP-1 and AP-2 loci. Biochem Genet;19:635-640.

Elthon, T E, Nickels, R L, McIntosh, L. 1989 Monoclonal antibodies to the alternative oxidase of higher plant mitochondria. Plant Physiol 89:1311-1317.

Emes, M J, Neuhaus, H E. 1998 Metabolism and transport in non-photosynthetic plastids. J. Exp. Bot;48:1995-2005.

Emi T, Kinoshita T, Shimazaki Ki K. 2001 Specific Binding of vf14-3-3a Isoform to the Plasma Membrane H(+)-ATPase in Response to Blue Light and Fusicoccin in Guard Cells of Broad Bean. Plant Physiol.125:1115-1125.

Emmler, K, Stockhaus, J, Chua, N H, Schafer, E. 1995 An amino-terminal deletion of rice phytochrome A results in a dominant negative suppression of tobacco phytochrome A activity in transgenic tobacco seedlings. Planta;197:103-110.

English, D. 1996 Phosphatidic acid: A lipid messenger involved in intracellular and extracellular signaling. Cell. Signaling;8:341-347.

English, D., Cui, Y., and Siddiqui, R. A. 1996 Messenger functions of phosphatidic acid. Chem. Phys. Lipids;80:117-132.

Escoubas, J M, Lomas, M, LaRoche, J, Falkowski, P G. Light intensity regulation of cab gene transcription is signaled by the redox state of the plastoquinone pool. Proc. Natl. Acad. Sci, USA; Oct. 24, 1995;92(22):10237-10241.

Espineda, C E, Linford, A S, Devine, D, Brusslan, J A. The AtCAO gene, encoding chlorophyll a oxygenase, is required for chlorophyll b synthesis in Arabidopsis thaliana. Proc. Natl. Acad. Sci, USA; Aug. 31, 1999;96(18):10507-10511.

Eulgem T, Rushton P J, Robatzek S, Somssich I E. 2000 The WRKY superfamily of plant transcription factors. Trends Plant Sci;5:199-206.

Eulgem, T et al 1999: Early nuclear events in plant defence signalling: rapid gene activation by WRKY transcription factors. EMBO.J, 18: 4689-4699.

Evans, M, Poethig, R. Gibberellins promote vegetative phase change and resproductive maturity in maize. Plant Physiology 1995; 108(2):475-487.

Ezaki, B, Gardner, R C, Ezaki, Y, Matsumoto, H. 2000 Expression of aluminum-induced genes in transgenic arabidopsis plants can ameliorate aluminum stress and/or oxidative stress. Plant Physiol;122:657-665.

Ezekiel, U R, Zassenhaus, H P. 1993 Localization of a cruciform cutting endonuclease to yeast mitochondria. Mol Gen Genet;240:414-418.

Faiss, M, Zalubilova, J, Strnad, M, Schmulling, T. 1997 Conditional transgenic expression of the ipt gene indicates a function for cytokinins in paracrine signaling in whole tobacco plants. Plant J;12:401-415.

Fan L, Zheng S, Wang X. 1997 Antisense suppression of phospholipase D alpha retards abscisic acid- and ethylene-promoted senescence of postharvest Arabidopsis leaves. Plant Cell;9:2183-2196.

Farese, R V, Jr, Herz, J. 1998 Cholesterol metabolism and embryogenesis. Trends Genet;3:115-120.

Farmer, E E, Weber, H, Vollenweider, S. 1998 Fatty acid signaling in Arabidopsis. Planta;206:167-174.

Farras, R, Ferrando, A, Jasik, J, Kleinow, T, Okresz, L, Tiburcio, A, Salchert, K, del Pozo, C, Schell, J, Koncz, C. 2001 SKP1-SnRK protein kinase interactions mediate proteasomal binding of a plant SCF ubiquitin ligase. EMBO J;20:2742-2756.

Faure, J D, Gingerich, D, Howell, S H. 1998 An Arabidopsis immunophilin, AtFKBP12, binds to AtFIP37 (FKBP interacting protein) in an interaction that is disrupted by FK506. Plant J;15:783-789.

Fennoy, S L, Bailey-Serres, J. Synonymous codon usage in Zea mays L. nuclear genes is varied by levels of C and G-ending codons. Nucleic Acids Res. Nov. 25, 1993;21(23):5294-5300.

Fernandez, D E, Heck, G R, Perry, S E, Patterson, S E, Bleecker, A B, Fang, S C. 2000 The embryo MADS domain factor AGL15 acts postembryonically. Inhibition of perianth senescence and abscission via constitutive expression. Plant Cell;12:183-198.

Fernandez-Larrea, J, and Stahl, U. 1996 Isolation and characterization of a laccase gene from Podospora anserina. Mol. Gen. Genet;252:539-551.

Finlayson, S A, Lee, I J, Mullet, J E, Morgan, P W. 1999 The mechanism of rhythmic ethylene production in sorghum. The role of phytochrome B and simulated shading. Plant Physiol;119:1083-1089.

Finnegan, P M, Whelan, J, Millar, A H, Zhang, Q, Smith, M K, Wiskich, J T, Day, D A. 1997 Differential expression of the multigene family encoding the soybean mitochondrial alternative oxidase. Plant Physiol;114:455-466.

Finnemann, J, Schjoerring, J K. 2000 Post-translational regulation of cytosolic glutamine synthetase by reversible phosphorylation and 14-3-3 protein interaction. Plant J;24:171-181.

Fling et al., Nucleic Acids Res., Oct. 11, 1985; 13(19): 7095-7106

Fobis-Loisy, I, Loridon, K, Lobreaux, S, Lebrun, M and Briat, J F. Structure and differential expression of two maize ferritin genes in response to iron and abscisic acid. European J. Biochem. Aug. 1, 1995;231(3),609-619.

Foissner, I, Wendehenne, D, Langebartels, C, Durner, J. 2000 Technical Advance: In vivo imaging of an elicitor-induced nitric oxide burst in tobacco. Plant J;23:817-824.

Forchhammer, K, Tandeau, de Marsac N. 1995 Phosphorylation of the PII protein (glnB gene product) in the cyanobacterium Synechococcus sp. strain PCC 7942: analysis of in vitro kinase activity. J Bacteriol;177(20):5812-5817.

Foster, T, Veit, B. Hake, S. 1999b Mosaic analysis of the dominant mutant, Gnarleyl-R, reveals distinct lateral and transverse signaling pathways during maize leaf development. Development; 126:305-313.

Foster, T, Yamaguchi, J, Wong, B C, Veit, B, Hake, S. 1999a Gnarleyl is a dominant mutation in the knox4 homeobox gene affecting cell shape and identity. Plant Cell;11:1239-1252.

Foyer, C H, Valadier, M -H, Migge, A, Becker, T W. 1998 Drought-induced effects on nitrate reductase activity and mRNA and on the coordination of nitrogen and carbon metabolism in maize leaves. Plant Physiol;117:283-292.

Frankel A, and Clarke S. 1999 RNase treatment of yeast and mammalian cell extracts affects in vitro substrate methylation by type I protein arginine N-methyltransferases. Biochem Biophys Res Commun. 259:391-400.

Frankel A, and Clarke S. 2000 PRMT3 is a distinct member of the protein arginine N-methyltransferase family J Biol Chem. 2000 275:32974-82.

Fraser A G, James C, Evan G I, Hengartner M O. 1999 Caenorhabditis elegans inhibitor of apoptosis protein (IAP) homologue BIR-1 plays a conserved role in cytokinesis. Curr Biol;9:292-301.

Frey, M, Stettner, C, Pare, P W, Schmelz, E A, Tumlinson, J H, Gierl, A. An herbivore elicitor activates the gene for indole emission in maize. Proc Natl Acad Sci USA Dec. 19, 2000; 97(26):14801-14806.

Friedrichsen, D M, Joazeiro, C A, Li, J, Hunter, T, Chory, J. 2000 Brassinosteroid-insensitive-1 is a ubiquitously expressed leucine-rich repeat receptor Serine/Threonine kinase. Plant Physiol; 123:1247-1256.

Fryer, M J, Oxborough, K, Martin, B, Ort, D R, Baker, N R. 1995 Factors associated with depression of photosynthetic quantum efficiency in maize at low growth temperature. Plant Physio;108:761-767.

Fu, H, Doelling, J H, Rubin, D M, Vierstra, R D. 1999 Structural and functional analysis of the six regulatory particle triple-A ATPase subunits from the Arabidopsis 26S proteasome. Plant J; 18:529-539.

Fujimoto, S Y, Ohta, M, Usui, A, Shinshi, H, Ohme-Takagim, M. Arabidopsis ethylene-responsive element binding factors act as transcriptional activators or repressors of GCC box-mediated gene expession. Plant Cell March 2000;12(3):393-404.

Fujisawa, Y, Kato, T, Ohki, S, Ishikawa, A, Kitano, H, Sasaki, T, Asahi, T, Iwasaki, Y. 1999 Suppression of the heterotrimeric G protein causes abnormal morphology, including dwarfism, in rice. Proc Natl Acad Sci USA;96:7575-7580.

Furini, A., Koncz, C., Salamini, F., and Bartels, D. 1997 High level transcription of a member of a repeated gene family confers dehydration tolerance to callus tissue of Craterostigma plantagineum. EMBO J: 16:3599-3608.

Gachotte, D, Meens R, and Benveniste, P. 1995. An Arabidopsis mutant deficient in sterol biosynthesis: Heterologous complementation by ERG3 encoding a 7-sterol-C-5-desaturase from yeast. Plant J;8:407-416.

Gagne, J, Shiu, S, Downes, B P, Vierstra, R D 2001 Genome analysis of the SCF and HECT E3 families in Arabidopsis thaliana. 12^{th} International Conference on Arabidopsis Research. Abstract 78.

Gahrtz, M, Schmelzer, E, Stolz, J, Sauer, N. 1996 Expression of the PmSUC1 sucrose carrier gene from Plantago major L. is induced during seed development. Plant J;9:93-100.

Gahrtz, M, Stolz, J, and Sauer, N. 1994 A phloem-specific sucrose H⁺ symporter from Plantago major L. supports the model of apoplastic phloem loading. Plant J;6:697-706.

Gallego, P P, Whotton, L, Picton, S, Grierson, D, Gray, J E. 1995 A role for glutamate decarboxylase during tomato ripening: the characterisation of a cDNA encoding a putative glutamate decarboxylase with a calmodulin-binding site. Plant Mol Biol;27:1143-1151.

Gan, S, Amasino, R M. (1995) Inhibition of leaf senescence by autoregulated production of cytokinin. Science;270: 1986-1988.

Gao, X D, Kaigorodov, V, Jigami, Y. 1999 YND1, a homologue of GDA1, encodes membrane-bound apyrase required for Golgi N- and O-glycosylation in Saccharomyces cerevisiae. J Biol Chem July 23;274(30):21450-21456.

Garay-Arroyo, A, Colmenero-Flores, J M, Garciarrubio, A, Covarrubias, A A. 2000 Highly hydrophilic proteins in prokaryotes and eukaryotes are common during conditions of water deficit. J Biol Chem;275:5668-5674.

Garay-Arroyo, A, Colmenero-Flores, J M, Garciarrubio, A, Covarrubias, A A. 2000 Highly hydrophilic proteins in prokaryotes and eukaryotes are common during conditions of water deficit. J Biol Chem;25;275:5668-5674.

Garlid, K D, Orosz, D E, Modriansky, M, Vassanelli, S, Jezek, P. On the mechanism of fatty acid-induced proton transport by mitochondrial uncoupling protein. J. Biol. Chem. Feb. 2, 1996; 271 (5):2615-2620.

Gasior, S L, Wong, A K, Kora, Y, Shinohara, A, Bishop, D K. 1998 Rad52 associates with RPA and functions with rad55 and rad57 to assemble meiotic recombination complexes. Genes Dev;12:2208-2221.

Gaur, G M, Kochlar, A, Maheshwar, S C, Tyagi, A K. 1999 Nucleotide sequence of psbQ gene for 16-kDa protein of oxygen evolving complex from Arabidopsis thaliana and regulation of expresssion. DNA Res;6:173-177.

Gausing, K. A barley gene (rsh1) encoding a ribonculease S-like homologue specifically expressed in young light-grown leaves. Planta March 2000;210(4):574-579.

Gaxiol, R., Rao, R., Sherman, A., Grisafi, P., Alper, S. and Fink, G. 1999 The Arabidopsis thaliana proton tranporters, AtNHX1 and Avp1, can function in cation detoxification in yeast. Pro. Nat. Acad;96:1480-1485.

Gaymard, F, Boucherez, J, Briat, J -F. Characterization of a ferritin mRNA from Arabidopsis thaliana accumulated in response to iron through an oxidative pathway independent of abscisic acid. Biochemical Journal 1996;318:67-73.

Geisler, M, Frnagne, N, Gomes, E, Martinola, E, Palmgren, M G. 2000 The ACA4 gene of Arabidopsis encodes a vacuolar membrane calcium pump that improves salt tolerance in yeast. Plant Physiol; 124:1814-1827.

Gelperin, D, Weigle, J, Nelson, K, Roseboom, P, Irie, K, Matsumoto, K, Lemmon, S. 1995 14-3-3 proteins: potential roles in vesicular transport and Ras signaling in Saccharomyces cerevisiae. Proc Natl Acad Sci USA;92: 11539-11543.

Gelvin, S. and Schilperoort, R. (1994) Plant Molecular Biology Manual (2nd ed.). Kluwer Academic, Dordrecht

Genty, B, Briantais, J -M, Baker, N R. 1989 The relationship between the quantum yield of photosynthetic electron transport and quenching of chlorophyll fluorescence. Biochim Biophys Acta;990:87-92.

Gerbling, K P, Steup, M, Latzko, E. Frusctose 1,6-bisphosphatase Form B from synechococcus leopoliensis Hydrolyzes both fructose and sedoheptulose bisphosphate. Plant Physiol 1986;80(3):716-720.

Gerday, C, Aittaleb, M, Bentahir, M, Chessa, J P, Claverie, P, Collins, T, D'Amico, S, Dumont, J, Garsoux, G, Georlette, D, Hoyoux, A, Lonhienne, T, Meuwis, M A, Feller, G. Cold-adapted enzymes: from fundamentals to biotechnology. Trends Biotechnol March 2000;18(3):103-107.

Giaquinta, R T. 1983 Phloem loading of sucrose. Annu. Rev. Plant Physiol;34:347-387.

Gidrol, X, Sabelli, P A, Fern, Y S, Kush, A K. 1996 Annexin-like protein from Arabidopsis thaliana rescues delta oxyR mutant of Escherichia coli from H2O2 stress. Proc Natl Acad Sci USA; 93:11268-11273.

Gierl, A, Frey, M. 1999 The hydroxamic acid pathway. Novartis Found Symp;223:150-157.

Gifford, R M, Evans, L T. 1981 Photosynthesis, carbon partitioning and yield. Annu. Rev. Plant Physiol;32:485-509.

Gillikin, J W, Zhang, F, Coleman, C E, Bass, H W, Larkins, B A, Boston, R S. 1997 A defective signal peptide tethers the floury-2 zein to the endoplasmic reticulum membrane. Plant Physiol;114(1):345-352.

Gillissen, B, Burkle L, Andre, B, Kuhn, C, Rentsch, D, Brandl, B, Frommer, W B. A new family of high-affinity transporters for adenine, cytosine, and purine derivatives in Arabidopsis. Plant Cell February 2000;12(2):291-300.

Gingras, A C, Raught, B, Sonenberg, N. 1999 eIF4 initiation factors: effectors of mRNA recruitment to ribosomes and regulators of translation. Annu Rev Biochem;68:913-963.

Gipp W F, Cline T R, Rogler J C. 1968 The metabolizable energy of opaque-2 and floury-2 corn for the chick. Poult Sci.47(6):2018-2021.

Girvin, M E, Rastogi, V K, Abildgaard, F, Markley, J L, Fillingame, R H. 1998 Solution structure of the transmembrane H+-transporting subunit c of the FlFo ATP synthase. Biochem;37:8817-8824.

Glover, J R, Lindquist, S. 1998 Hsp104, Hsp70, and Hsp40: a novel chaperone system that rescues previously aggregated proteins. Cell;94:73-82.

Goetz, M, Godt, D E, Roitsch, T. 2000. Tissue-specific induction of the mRNA for an extracellular invertase isoenzyme of tomato by brassinosteroids suggests a role for steroid hormones in assimilate partitioning. Plant J;22:515-522.

Gogarten, J P, Bentrup, F W. 1989 Substrate specificity of the hexose carrier in the plasma membrane of Chenopodium suspension cells probes by transmembrane exchange diffusion. Planta; 178:52-60.

Gomes, E, Jakobsen, M K, Axelsen, K B, Geisler, M, Palmgren, M G. 2000 Chilling tolerance in Arabidopsis involves ALA1, a member of a new family of putative aminophospholipid translocases. Plant Cell; 12:2441-2453.

Gomez-Gomez, L, Boller, T. 2000 FLS2: an LRR receptor-like kinase involved in the perception of the bacterial elicitor flagellin in Arabidopsis. Mol Cell;5:1003-1011.

Gonzalez-Barroso, M M, Fleury, C, Bouillaud, F, Nicholls, D G, Rial, E. 1998. The Uncoupling Protein UCP1 Does Not Increase the Proton Conductance of the Inner Mitochondrial Membrane by Functioning as a Fatty Acid Anion Transporter. J of Biol. Chem; 273:15528-15532.

Goodrich, J, Puangsomlee, P, Martin, M, Long, D, Meyerowitz, E M, Coupland, G. 1997 A Polycomb-group gene regulates homeotic gene expression in Arabidopsis. Nature;386:44-51.

Gordon, E D, Mora, R., Meredith, S C, Lee, C. and Lindquist, S. L. 1987 Eukaryotic initiation factor 4D, the hypusine-containing protein, is conserved among eukaryotes. J. Biol. Chem;262:16585-16589.

Gorschen, E. Dunaeva, M, Hause, B, Reeh, I, Wasternack, C, Parthier, B. 1997 Expression of the ribosome-inactivating protein JIP60 from barley in transgenic tobacco leads to an abnormal phenotype and alterations on the level of translation. Planta;202:470-478.

Gosti, Francoise, Beaudoin, Nathalie, Serizet, Carine, Webb, Alex A R, Vartanian, Nicloe and Giraudat, Jerome. ABI1 Protein Phosphatase 2C is a nagative regulator of Abscisic acid signaling. The Plant Cell; 11:1897-1909, 1999.

Gottlieb, E and Steitz, J A. 1989 Function of the mammalian La protein: evidence for its action in transcription termination by RNA polymerase III. EMBO J;8:851-861.

Gouvea, C M C P. 1997 NO-releasing substances that induce growth elongation in maize root segments. Plant growth regulation; 21:183-187.

Gray, J, Close, P S, Briggs, S P, Johal, G S. 1997 A novel suppressor of cell death in plants encoded by the Lls1 gene of maize. Cell; 89:25-31.

Gray, W M, del Pozo, J C, Walker, L, Hobbie, L, Risseeuw, E, Banks, T, Crosby, W L, Yang, M, Ma, H, Estelle, M. 1999 Identification of an SCF ubiquitin-ligase complex required for auxin response in Arabidopsis thaliana. Genes Dev; 13:1678-1691.

Grayer, R J, Harborne, J B, Overton, J, Dillon, V M. 1995 Flavonoid phytoalexins and resistance of rice plants against the rice blast fungus, Pyricularia oryzae. In: Flavonoids and Bioflavonoids, eds S. Antus, M. Gabor and K. Vetschera. Akademia Kiado,Budapest, pp. 119.

Green, B R, Durnford, D G. 1996 The chlorophyll-carotenoid proteins of oxygenic photosynthesis. Ann. Rev. Plant Physio. Plant Mol. Biol;47:685-714.

Grossniklaus, U, Vielle-Calzada, J P, Hoeppner, M A, Gagliano, W B. 1998 Maternal control of embryogenesis by MEDEA, a polycomb group gene in Arabidopsis. Science;280:446-450.

Guddat, U, Bakken, A H, Pieler, T. 1990 Protein-mediated nuclear export of RNA: 5S rRNA containing small RNPs in xenopus oocytes. Cell;60:619-628.

Gunawardena, A H, Pearce, D M, Jackson, M B, Hawes, C R, Evans, D E. 2001 Characterisation of programmed cell death during aerenchyma formation induced by ethylene or hypoxia in roots of maize (Zea mays L.). Planta;212:205-214.

Gupta, A S, Heinen, J L, Holaday, A S, Burke, J J, Allen, R D. 1993 Increased resistance to oxidative stress in transgenic plants that overexpress chloroplastic Cu/Zn superoxide dismutase. Proc Natl Acad Sci USA;90:1629-1633.

Hageman, R H, Lambert, R J. 1988 The use of physiological traits for corn improvement. In: Corn and corn improvement, 3^{rd} edition. Sprague G F and Dudley J W eds. Pg 431-461.

Hahn, D, Kuck, U. Necleotide sequence of a cDNA encoding the chloroplast sedpheptulose-1,7-bisphosphatase from chlamydomonas reinhardtii. Plant Physiol 1994;104(3):1101-1102.

Halleck, M S, Pradhan, D, Blackman, C, Berkes, C, Williamson, P, Schlegel, R A. 1998 Multiple members of a third subfamily of P-Type ATPases identified by genomic sequences and ESTs. Gen Res;8(4):354-361.

Halliday, K J, Hudson, M, Ni, M, Qin, M, Quail, P H. 1999b poc1: an Arabidopsis mutant perturbed in phytochrome signaling because of a T DNA insertion in the promoter of PIF3, a gene encoding a phytochrome-interacting bHLH protein. Proc Natl Acad Sci USA; 96:5832-5837.

Halliday, K J, Thomas, B, Whitelam, G C. 1997 Expression of heterologous phytochromes A, B or C in transgenic tobacco plants alters vegetative development and flowering time. Plant J;12:1079-1090.

Hamilton, C M, Frary, A, Lewis, C, Tanksley, S D. Stable transfer of intact high molecular weight DNA into plant chromosomes. Proc Natl Acad Sci USA Sep. 3, 1996;93(18):9975-9979.

Hanai, H, Matsuno, T, Yamamoto, M, Matsubayashi, Y, Kobayashi, T, Kamada, H, Sakagami, Y. 2000 A secreted peptide growth factor, phytosulfokine, acting as a stimulatory factor of carrot somatic embryo formation. Plant Cell Physiol;41 :27-32.

Hanai, H, Nakayama, D, Yang, H, Matsubayashi, Y, Hirota, Y, Sakagami, Y. 2000 Existence of a plant tyrosylprotein sulfotransferase: novel plant enzyme catalyzing tyrosine O-sulfation of preprophytosulfokine variants in vitro. FEBS Lett;470:97-101.

Hanauske-Abel, H M, Park, M H, Hanauske, A R, Popowicz, A M, Lalande, M, Folk, J E. 1994 Inhibition of the G1-S transition of the cell cycle by inhibitors of deoxyhypusine hydroxylation. Biochim Biophys Acta;1221 :115-124.

Hannoufa, A, Negruk, V, Eisner, G, Lemieux, B. The CER3 gene of Arabidopsis thaliana is expressed in leaves, stems, roots, flowers and apical meristems. Plant J. Sep. 1996;10(3):459-467. PMID: 8811860; UI: 96407843.

Hansen, G. 2000 Evidence for Agrobacterium-induced apoptosis in maize cells. Mol Plant Microbe Interact;13:649-657.

Hansen, C. H., Wittstock, U., Olsen, C. E., Hick, A. J., Pickett, J. A., and Halkier, B. A. 2001. Cytochrome P450 CYP79F1 from Arabidopsis catalyzes the conversion of dihomomethionine and trihomomethionine to the corresponding aldoximes in the biosynthesis of aliphatic glucosinolates. J. Biol. Chem;276:11078-11085.

Hansen, J D, Pyee, J, Xia, Y, Wen, T J, Robertson, D S, Kolattukudy, P E, Nikolau, B J and Schnable, P S. The glossyl locus of maize and an epidernis-specific cDNA from Kleinia odora define a class of receptor-like proteins required for the normal accumulation of cuticular waxes. Plant Physiol. 1997; 113:1091-1100.

Hanumappa, M, Pratt, L H, Cordonnier-Pratt, M M, Deitzer, G F. 1999 A photoperiod-insensitive barley line contains a light-labile phytochrome B. Plant Physiol;119:1033-1040.

Hara, K, Yagi, M, Kusano, T, Sano, H. 2000 Rapid systemic accumulation of transcripts encoding a tobacco WRKY transcription factor upon wounding. Mol Gen Genet;263 :30-37.

Haran, S, Logendra, S, Seskar, M, Bratanova, M, Raskin, I. 2000 Characterization of Arabidopsis Acid Phosphatase Promoter and Regulation of Acid Phosphatase Expression. Plant Physiol;124:615-626.

Hardtke, C S, Gohda, K, Osterlund, M T, Oyama, T, Okada, K, Deng, X W. 2000 HY5 stability and activity in arabidopsis is regulated by phosphorylation in its COP1 binding domain. EMBO J;19:4997-5006.

Hareven, D, Gutfinger, T, Parnis, A, Eshed, Y, Lifschitz, E. 1996 The making of a compound leaf: genetic manipulation of leaf architecture in tomato. Cell;184:735-744.

Hamdahl, U, Hall, R B, Osteryoung, K W, Vierling, E, Bormnan, J F, Sundby, C. 1999 The chloroplast small heat shock protein undergoes oxidation-dependent conformational changes and may protect plants from oxidative stress. Cell Stress Chaperones;4:129-138.

Harper, J F, Hong, B, Hwang, I, Guo, H Q, Stoddard, R, Huang, J F, Palmgren, MG, Sze, H. A novel calmodulin-regulated Ca2+-ATPase (ACA2) from Arabidopsis with an N-terminal authinhibitory domain. J Biol Chem Jan. 9, 1998; 273(2):1099-1106.

Harrington, G N, Franceschi, V R, Offler, C E, Patrick, J W, Harper, J F, Frommer, W B, Tegeder, M, and Hitz, W D. 1997 Cell specific expression of three genes involved in plasma membrane sucrose transport in developing Vicia faba seed. Protoplasma;197:160-173.

Harris, D M, Myrick, T L, Rundle, S J. 1999 The Arabidopsis homolog of yeast TAP42 and mammalian alpha4 binds to the catalytic subunit of protein phosphatase 2A and is induced by chilling. Plant Physiol;121 :609-617.

Harrison, Willingham, Lloyd, Raines. Reduced sedoheptulose-1,7-bisphosphatase levels in transgenic tobacco lead to decreased photosynthetic capacity and altered carbohydrate accumulation. Planta 1998; 204(1):27-36.

Hartmann, U, Hohmann, S, Nettesheim, K, Wisman, E, Saedler, H, Huijser, P. Molecular cloning of SVP: a negative regulator of the floral transition in Arabidopsis. Plant J February 2000; 21(4):351-360.

Hartwick, L. M., Llewellyn, D. J. and Dennis, E. S. 1997 The Arabidopsis thaliana genome has multiple divergent forms of phosphatidylinositol-specific phospholipase C. Gene;202:151-156.

Hasegawa, P M, Bressan, R A, Zhu, J-., Bohnert, H J. 2000 Plant cellular and molecular responses to high salinity. Annu. Rev. Plant Physiol. Plant Mol. Biol;51:463499.

Haseloff, J, Siemering, K R, Prasher, D C, Hodge, S. Removal of a cryptic intron and subcellular localization of green fluorescent protein are required to mark transgenic Arabidopsis plants brightly. Proc Natl Acad Sci USA Mar. 18, 1997; 94(6):2122-2127.

Haseloff, J. GFP variants for multispectral imaging of living cells. Methods Cell Biology, 1999;58;139-151.

Hashizume, R, Fukuda, M, Maeda, I, Nishikawa, H, Oyake, D, Yabuki, Y, Ogata, H, Ohta, T. 2001 The RING heterodimer BRCA1-BARD1 is a ubiquitin ligase inactivated by a breast cancer-derived mutation. J Biol Chem.276:14537-14540.

Hatakeyama, S, Yada, M, Matsumoto, M, Ishida, N, Nakayama, K I. 2001 U-Box proteins as a new family of ubiquitin-protein ligases. J Biol Chem. In press

Havaux, M, Niyogi, K K. 1999 The violaxanthin cycle protects plants from photooxidative damage by more than one mechanism. Proc Natl Acad Sci USA;96:8762-8767.

Haydon, M S, Googe, J D, Sorrells, D S, Ghali, G E, Li, B D. 2000 Progression of eIF4e gene amplification and overexpression in benign and malignant tumors of the head and neck. Cancer;88:2803-2810.

Hayes, et al. Nucleic Acid Hybridizaiton, A Practical Approach, IRL Press, Washington D.C., 1985.

He, C, Fong, S H, Yang, D, Wang, G L. 1999 BWMK1, a novel MAP kinase induced by fungal infection and mechanical wounding in rice. Mol Plant Microbe Interact;December;12(12):1064-1073.

He, Y, Tang, W, Swain, J D, Green, A L, Jack, T P, Gan, S. 2001 Networking senescence-regulating pathways by using arabidopsis enhancer trap lines. Plant Physiol;126:707-716.

He, Z, Zhu, Q, Dabi, T, Li, D, Weigel, D, Lamb, C. 2000 Transformation of rice with the Arabidopsis floral regulator LEAFY causes early heading. Transgenic Res;9:223-227.

He, Z, Wang, Z, Li J, Zhu, Q, Lamb, C, Ronald, P, Chory, J. 2000. Perception of Brassinosteroids by the Extracellular Domain of the Receptor Kinase BRI1. Science;288:2360-2363.

Heidkamper, D, Mtiller, V, Nelson, D R, Klingenberg, M. 1996 Probing the role of positive residues in the ADP/ATP carrier from yeast. The effect of six arginine mutations on transport and the four ATP versus ADP exchange modes. Biochem;35,16144-16152.

Heim, D, Nicholson, R L, Pascholati, S F, Hagerman, A E, Billet, W. 1993 Etiolated maize mesocotyls: A tool for investigating disease interactions. Phytopathol;73:424-428.

Helin, K, Wu, C L, Fattaey, A R, Lees, J A, Dynlacht, B D, Ngwu, C, Harlow, E. 1993 Heterodimerization of the transcription factors E2F-1 and DP-1 leads to cooperative trans-activation. Genes Dev;7:1850-1861.

Helliwell, S B, Losko, S, Kaiser, C A. 2001 Components of a ubiquitin ligase complex specify polyubiquitination and intracellular trafficking of the general amino acid permease. J Cell Biol;153:649-662.

Hershey, J W. Expression of initiation factor genes in mammalian cells. Biochimie. 1994; 76(9):847-852. Review.

Heyer, A G, Mozley, D, Landschutze, V, Thomas, B, Gatz, C. 1995 Function of phytochrome A in potato plants as revealed through the study oftransgenic plants. Plant Physiol;109:53-61.

Higgins, C F. ABC transporters: from micro-organisms to man. Annu.Rev.Cell Biol 1992;8:67-113.

Higo, K, Ugawa, Y, Iwamoto, M and Korenaga, T 1999 Plant cis-acting regulatory DNA elements (PLACE) database. cleic Acids Research;27(1):297-300.

Hinchee, M A, Connor-Ward, D V, Newell, C A, McDonnell, R E, Sato, S J, Casser, C S, Fischhoff, D A, Re, D B, Fraley R T, Horsch, R B. Production of transgenic soybean plants using Agrobacterium-mediated DNA transfer. Bio/Technology August 1988;6:915-922.

Hinderhofer, K, Zentgraf, U. 2001 Identification of a transcription factor specifically expressed at the onset of leaf senescence. Planta;213:469-473.

Hipskind, J, Wood, K, Nicholson, R L. 1996 Localized stimulation of anthocyanin accumulation and delineation of pathogen ingress in maize genetically resistant to Bipolaris maydis race O. Physiol. Mol Plant Pathol;49:247-256.

Hipskind, J D, Hanau, R, Leite, B, Nicholson, R L. 1990 Phytoalexin accumulation in Sorghum: Identification of an apigeninidin acyl ester. Physiol. Mol. Plant Pathol;36:381-396.

Hirasawa, M, Schurmann, P, Jacquot, J P, Manieri, W, Jacquot, P, Keryer, E, Hartman, F C, Knaff, D B. 1999 Oxidation-reduction properties of chloroplast thioredoxins, ferredoxin: thioredoxin reductase, and thioredoxin f-regulated enzymes. Biochemistry;38:5200-5205.

Hirayama, T, Ohto, C, Mizoguchi, T, and Shinozaki, K. 1995 A gene encoding a phosphatidylinositol-specific phospholipase C is induced by dehydration and salt-stress in Arabidopsis thaliana. Proc. Natl. Acad. Sci. USA;92:3903-3907.

Hirose, T, Imaizumi, N, Scofield, G N, Furbank, R T, and Ohsugi, R. 1997 cDNA cloning and tissue-specific expression of a gene for sucrose transporter from rice (Oryza sativa L.). Plant Cell Physiol;38:1389-1396.

Hirschman, J E, Jenness, D D. 1999 Dual lipid modification of the yeast ggamma subunit Ste18p determines membrane localization of Gbetaganmna. Mol Cell Biol;19:7705-7711.

Hiser, C, Kapranov, P, McIntosh, L. 1996 Genetic modification of respiratory capacity in potato. Plant Physiol;110:277-286.

Hoeberichts, F A, Orzaez, D, van der Plas, L H, Woltering, E J. 2001 Changes in gene expression during programmed cell death in tomato cell suspensions. Plant Mol Biol;45:641-54.

Hoecker, U, Tepperman, J M, Quail, P H. 1999 SPA1, a WD-repeat protein specific to phytochrome A signal transduction. Science;284:496-499.

Hoecker, U, Xu, Y, Quail, P H. 1998 SPA1: a new genetic locus involved in phytochrome A-specific signal transduction. Plant Cell;10:19-33.

Hoeijmakers, J H, Egly, J M, Vermeulen, W. 1996 NTFIIH: a key component in multiple DNA transactions. Curr Opin Genet Dev;6:26-33.

Holford, I C R. 1997 Soil phosphorus: its measurement, and its uptake by plants. Aust J Soil Res;35:227-239.

Hollenbach, B, and Dietz, K -J. 1995 Molecular cloning of emip, a member of the Major Intrinsic Protein (MIP) gene family, preferentially expressed in epidermal cells of barley leaves. Bot. Acta;108:425-431.

Holmberg, N, Lilius, G, Bailey, J E, Bulow, L. Transgenic tobacco expressing Vitreoscilla hemoglobin exhibits enhanced growth and altered metabolite production. Nature Biotech. March 1997; 15(3):244-247.

Holmstrom, K, Somersalo, S, Mandal, A, Palva, T, and Welin, B. Improved tolerance to salinity and low temperature in transgenic tobacco producing glycine betaine. Journal of Experimental Botany 2000; 51,343:177-185.

Honda et al., Gene, Sep. 7, 1988; 68(2) :221-8

Hong, N A, Kabra, N H, Hsieh, S N, Cado, D, Winoto, A. 1999 In vivo overexpression of Dad1, the defender against apoptotic death-1, enhances T cell proliferation but does not protect against apoptosis. J Immunol;163:1888-1893.

Hooley, R. Gibberellins: perception, transduction and responses. Plant Mol. Biology December 1994; 26(5):1529-1555.

Horemans, N. Asard, H, Caubergs, R J. 1998 Carrier mediated uptake of dehydroascorbate into higher plant plasma membrane vesicles shows trans-stimulation. FEBS Lett;421(1):41-44.

P. Horton, A. V. Ruban, and R. G. Walters 1996 Regulation of light harvesting in green plants. Ann. Rev. Plant Physio. Plant Mol. Biol 47:655-684.

Houlne, G, and Boutry, M. (1994) Identification of an Arabidopsis thaliana gene encoding a plasma membrane H⁺-ATPase whose expression is restricted to anther tissues. Plant J;5:311-317.

Hsieh, M H, Lam, H M, van de Loo, F J, Coruzzi, G. 1998 A PII-like protein in Arabidopsis: putative role in nitrogen sensing. Proc Natl Acad Sci USA;95(23):13965-13970.

Huang, C, Barker, S J, Langridge, P, Smith, F W, Graham, R D. 2000. Zinc Deficiency Up-Regulates Expression of High-Affinity PhosphateTransporter Genes in Both Phosphate-Sufficient and -Deficient Barley Roots. Plant Physiol;124:415-422.

Huang, J, Hirji, R. Adam, L, Rozwadowski, K L, Hammerlindl, J K, Keller, W A, Selvaraj, G. Genetic engineering of glycine betaine production toward enhancing stress tolerance in plants: metabolic limitations. Plant Physiol. March 2000; 122(3):747-756.

Huang, S, Cerny, R E, Bhat, D, Brown, S M. (2000) Cloning of an Arabidopsis patatin-like gene, STURDY, by activation T-DNA tagging. Plant Physiol February 2001;125:573-584.

Huang, S, Kerschbaum, H H, Engel, E, Hermann, A. 1997 Biochemical characterization and histochemical localization of nitric oxide synthase in the nervous system of the snail, Helix pomatia. J Neurochem;69:2516-2528.

Huberman et al., 1995. Regulation and role of IMPD in cell replication, malignant transformation, and differentiation. In: A. Sahota, M. Taylor (Eds), Purine and Pyrimidine metabolism in Man, Vol. 8, Plenum Press, New York, 1995, pp 741-746.

Hudak, C M. 1995 Vegetative growth analysis of a drought resistant soybean plant introduction. Crop Science;35:464-471.

Hudson, M, Ringli, C, Boylan, M T, Quail, P H. 1999 The FAR1 locus encodes a novel nuclear protein specific to phytochrome A signaling. Genes Dev;13:2017-2027.

Hueros, G, Varotto S, Salamini, F, Thompson, R D. (1995) Molecular characterization of BET1, a gene expressed in the endosperm transfer cells of maize. Plant Cell;7:747-757.

Hugouvieux, V., Kwak, J. M., Schroeder, J. I., An mRNA cap binding protein, ABH1, modulates early abscisic acid signal transduction in Arabidopsis. Cell 2001,106(4):477-487.

Husselstein, T. Schaller H, Gachotte D, and Benveniste, P. 1999. Delta7-sterol-C5-desaturase: Molecular characterization and functional expression of wild-type and mutant alleles. Plant Mol. Biol;39:891-901.

Hutchison, R S, Groom, Q, Ort, D R. 2000 Differential effects of chilling-induced photooxidation on the redox regulation of photosynthetic enzymes. Biochemistry;39:6679-6688.

Ignarro, L J. 2000 Nitric Oxide : Biology and Pathobiology. Academic Pr; ISBN: 0123704200. Ignatova, L K. Moskvin, O V, Romanova, A K, and Ivanov, B N. 1998 Carbonic anhydrases in the C3-plant leaf cell. Aust. J. Plant Physiol;25:673-677.

Ikeda Y, Koizumi N, Kusano T, Sano H. 2000 Specific binding of a 14-3-3 protein to autophosphorylated WPK4, an SNF1-related wheat protein kinase, and to WPK4-phosphorylated nitrate reductase. J Biol Chem.275:31695-31700.

Ikeda, Y, Koizumi, N, Kusano, T, Sano, H. 1999 Sucrose and Cytokinin Modulation of WPK4, a Gene Encoding a SNF1-Related Protein Kinase from Wheat. Plant Physiol;121:813-820.

Ikuta, N, Souza, M B, Valencia, F F, Castro, M E, Schenberg, A C, Pizzirani-Kleiner, A, Astolfi-Filho, S. The alpha-amylase gene as a marker for gene cloning: direct screening of recombinant clones. Biotechnology (NY) March 1990;8(3):241-242.

Imai, R, Chang, L, Ohta, A, Bray, E A, Takagi, M. 1996 A lea-class gene of tomato confers salt and freezing tolerance when expressed in Saccharomyces cerevisiae. Gene; 1 70:243-248.

Imai, J, Yahara, I. 2000 Role of HSP90 in salt stress tolerance via stabilization and regulation of calcineurin. Mol Cell Biol;20:9262-9270.

Incharoensakdi, A, Matsuda, N, Hibino, T, Meng, Y, Ishikawa, H, Hara, A, Funaguma, T, Takabe, T and Takabe T. Overproduction of spinach betaine aldehyde dehydrogenase in Escerichia coli. European. Journal of Biochemistry 2000;267:7015-7023.

Issakidis, E, Saarinen, M, Decottignies, P, Jacquot, J P, Cretis, C, et al. 1994 Identification and characterization of the second regulatory disulfide bridge of recombinant sorghum leaf NADP-malate dehydrogenase. J. Biol. Chem;269:3511-3517.

Issakidis-Bourguet, E, Mouaheb, N, Meyer, Y, Miginiac-Maslow, M. 2001 Heterologous complementation of yeast reveals a new putative function for chloroplast m-type thioredoxin. Plant J;25:127-135.

Isshiki, T, Mochizuki, N, Maeda T, Yamamoto M. 1992 Characterization of a fission yeast gene, gpa2, that encodes a G alpha subunit involved in the monitoring of nutrition. Genes Dev;6:2455-2462.

Issinger, O. G 1993 Casein kinases: pleiotropic mediators of cellular regulation. Pharmacol Ther;59(1):1-30.

Itoh, R, Takan, H, Ohta, N, Miyagishima S, Kuroiwa, H, Kuroiwa, T. Two ftsH-family genes encoded in the nuclear and chloroplast genomes of the primitive red alga Cyanidioschyzon merolae. Plant Mol Bio October 1999;41(3):321-337.

Iwasaki, T, Shibaoka, H. 1991 Brassinosteroids act as regulators of tracheary-element differentiation in isolated Zinnia mesophyll cells. Plant Cell Physiol;32:1007-1014.

Izawa, T, Oikawa, T, Tokutomi, S, Okuno, K, Shimamoto, K. 2000 Phytochromes confer the photoperiodic control of flowering in rice (a short-day plant). Plant J;22:391-399.

Jackson, C, Dench, J, Moore, A L, Halliwell, B, Foyer C H, Hall, D O. 1978 Subcellular localisation and identification of superoxide dismutase in the leaves of higher plants. Eur J Biochem;91 :339-344.

Jacobs, J S, Anderson, A R, Parker, R P. 1998 The 3′ to 5′ degradation of yeast mRNAs is a general mechanism for mRNA turnover that requires the SK12 DEVH box protein and 3′ to 5′ exonucleases of the exosome complex. EMBO J;17:1497-1506.

Jaglo-Ottosen, K R, Gilmour, S J, Zarka, D G, Schabenberger, O, Thomashow, M F. 1999 Arabidopsis CBF1 overexpression induces COR genes and enhances freezing tolerance. Science;280(5360):104-106.

Jambunathan, R, Kherdekar, M S, Bandyopadhay, R. 1990 Flavan 4-ols concentration in mold susceptible and mold resistant Sorghum at different stages of grain development. J. of Agric. and Food Chem;38(2):545.

Jang, J, Fujioka, S, Tasaka, M, Seto, H, Takatsuto, S, Ishii, A, Aida, M, Yoshida, S, Sheen, J. 2000 A critical role of sterols in embryonic patterning and meristem programming revealed by fackel mutants of Arabidopsis thaliana. Gen. Dev;14:1485-1497.

Jansson, B P, Malandrin, L, Johansson, H E. 2000 Cell cycle arrest in archaea by the hypusination inhibitor N(1)-guanyl-1,7-diaminoheptane. J Bacteriol;182:1158-1161.

Jarmuszkiewicz, W, Almeida, A M, Sluse-Goffart, C M, Sluse, F E, Vercesi, A E. 1998 Linoleic Acid-induced Activity of Plant Uncoupling Mitochondrial Protein in Purified Tomato Fruit Mitochondria during Resting, Phosphorylating and Progressively Uncoupled Respiration. J. of Biol. Chem;273:34882-34886.

Jasinski, M, Stukkens, Y, Degand, H, Purnelle, B, Marchand-Brynaert, J, Boutry, M. A plant plasma membrane ATP binding cassette-type transporter is involved in antifungal terpenoid secretion. Plant Cell May 2001;13(5):1095-1107.

Jefferson, R et al. Glucuronidase from Escherichia coli as a gene-fusion marker. Proc Natl Acad Sci USA 1986;83(22):8447-8451.

Jezek, P, Costa, A D T, Vercesi, A E. 1996 Evidence for Anion-translocating Plant Uncoupling Mitochondrial Protein in Potato Mitochondria J. of Biol. Chem;271 :32743-3248.

Jezek, P, Costa, A D T, Vercesi, A E. 1997. Reconstituted Plant Uncoupling Mitochondrial Protein Allows for Proton Translocation via Fatty Acid Cycling Mechanism. J. of Biol. Chem;272:24272-24278.

Jiang, P, Zucker, P, Atkinson, M R, Kamberov, E S, Tirasophon, W, Chandran, P, Schefke, B R, Ninfa, A J. 1997 Structure/function analysis of the PII signal transduction protein of Escherichia coli: genetic separation of interactions with protein receptors. J Bacteriol;179(13):4342-4353.

Jiang, W, Hou, Y and Inouye, M. 1997 CspA, the major cold-shock protein of Escherichia coli, is an RNA chaperone. J. Biol. Chem;272:196-202.

Jiang, Y, Broach, J R. 1999 Tor proteins and protein phosphatase 2A reciprocally regulate Tap42 in controlling cell growth in yeast. EMBO J;18:2782-2792.

Jin, P, Guo, T, Becraft, P W. 2000 The maize CR4 receptor-like kinase mediates a growth factor-like differentiation response. Genesis;27:104-116.

Jinn, T L, Stone, J M, Walker, J C. 2000 HAESA, an Arabidopsis leucine-rich repeat receptor kinase, controls floral organ abscission. Genes Dev; 14:108-117.

Joazeiro, C A, Hunter, T. 2000 Biochemistry. Ubiquitination—more than two to tango. Science;289:2061-2062.

Joazeiro, C A, Weissman, A M. 2000 RING finger proteins: mediators of ubiquitin ligase activity. Cell;102:549-52.

Joersbo, M, Donaldson, Ikreiberg, J, Petersen S G, Brunstedt, J Okkels, F T. Analysis of mannose selection used for transformation of sugar beet. Molecular Breeding 1998;4(2):111-117.

Johnson, B S, Singh, N K, Cherry, J H, Locy, R D. 1997 Purification and characterization of glutamate decarboxylase from cowpea. Phytochemistry;46:39-44.

Johnston, L H, Eberly, S L, Chapman, J W, Araki, H, Sugino, A. 1990 The product of the Saccharomyces cerevisiae cell cycle gene DBF2 has homology with protein kinases and is periodically expressed in the cell cycle. Molecular and Cellular Biology;10:1358-1366.

Jonak, C, Kiegerl, S, Ligterink, W, Barker, P J, Huskisson, N S, and Hirt, H. Stress signaling in plants: a mitogen-activated protein kinase pathway is activated by cold and drought. Proc. Natl. Acad. Sci USA Oct. 1, 1996;93(20):11274-11279.

Jones, D H, Ley, S, Aitken, A. 1995 Isoforms of 14-3-3 protein can form homo- and heterodimers in vivo and in vitro: implications for function as adapter proteins. FEBS Lett;368:55-58.

Jones, H D, Smith, S J, Desikan, R, Plakidou-Dymock, S, Lovegrove, A, Hooley, R. 1998 Heterotrimeric G proteins are implicated in gibberellin induction of a-amylase gene expression in wild oat aleurone. Plant Cell;10:245-254.

Jones, R J, Roessler, J A and Ouattar, S. 1985 Thermal environment during endosperm cell division in maize: Effect on number of endosperm cells and starch granules. Crop Sc;25:830-834.

Jordan, E T, Hatfield, P M, Hondred, D, Talon, M, Zeevaart, J A, Vierstra, R D. 1995 Phytochrome A overexpression in transgenic tobacco. Correlation of dwarf phenotype with high concentrations of phytochrome in vascular tissue and attenuated gibberellin levels. Plant Physiol;107:797-805.

Josh, C P. An instpection of the domain between putative TATA box and translation start site in 79 plant genes. Nulceic Acids Res. Aug. 25, 1987;15(16):6643-6653.

Junge, W, Lill, H, Engelbrecht, S. 1997 ATP synthase: an electrochemical transducer with rotary mechanics. TIBS;22:420-423.

Kaeppler, H F, Gu, W, Somers, D A, Rines, H W, Cockburn, A F. Silicon carbide fiber-mediated DNA delivery into plant cells. Plant Cell Reports 1990;9(8):415-418.

Kagaya, Y, Ohmiya, K, Hattori, T. RAV1, a novel DNA-binding protein, binds to bipartite recognition sequence through two distinct DNA-binding domains uniquely found in higher plants. Nucleic Acids Res Jan. 15, 1999;27(2):470-478.

Kaim, G, Dimroth, P. 1999 ATP synthase by F-type ATP synthase is obligatorily dependent on the transmembrane voltage. EMBO J;18:4118-4127.

Kakimoto, T. 1996 CKI1, a histidine kinase homolog implicated in cytokinin signal transduction. Science;274:982-985.

Kaldenhoff, R, Grote, K, Zhu, J., and Zimmermann, U. 1998 Significance of plasmalemma aquaporins for water transport in Arabidopsis thaliana. Plant Journal;14:121-128.

Kaldis, P, Sutton, A, Solomon, M J. 1996 The Cdk-activating kinase (CAK) from budding yeast. Cell;86:553-564.

Kaldis, P. 1999 The cdk-activating kinase (CAK): from yeast to mammals. Cell Mol Life Sci;55:284-296.

Kampfenkel, K, Mohlmann, T, Batz, O, van Montagu, M, Inze, D, Neuhaus, H E. 1995 Molecular characterization of an Arabidopsis thaliana cDNA encoding a novel putative adenylate translocator of higher plants. FEBS Lett;374:351-355.

Kang, H G, Jang, S, Chung, J E, Cho, Y G, An, G. 1997 Characterization of two rice MADS box genes that control flowering time. Mol Cells;7:559-566.

Kang, H A and Hershey, J W. 1994 Effect of initiation factor eIF-5A depletion on protein synthesis and proliferation of Saccharomyces cerevisiae. J. Biol. Chem;269:3934-3940.

Karlsson, J, Clarke, A K, Chen, Z -Y, Hugghins, S Y, Park, Y, Husic, H D, Moroney, J V and Samuelsson, G. 1998 A novel a-type carbonic anhydrase associated with the thlakoid membrane in Chlamydomonas reinhardtii is required for growth at ambient CO2. EMBO J;17(5):1208-1216.

Kathiresan, A, Miranda, J, Chinnappa, C, Reid D. 1998 y-aminobutyric acid promotes stem elongation in Stellaria longipes: the role of ethylene. Plant Growth Regul;26:131-137.

Kathiresan, A, Tung, P, Chinnappa, C, Reid, D. 1997 y-aminobutyric acid stimulates ethylene biosynthesis in sunflower. Plant Physiol;115:129-135.

Katz, E, Thompson, C J, Hopwood, D A. Cloning and expression of the tyrosinase gene from Streptomyces antibioticus in Streptomyces lividans. J Gen Microbiol September 1983; 129(Pt9):2703-2714.

Kauschmann, A. A, Jessop, C, Koncz, M, Szekeres, L, Willmitzer, and Altmann T. 1996 Genetic evidence for an essential role of brassinosteroids in plant development. Plant J; 9:701-713.

Kawai M, Pan L, Reed J C, Uchimiya H. 1999 Evolutionally conserved plant homologue of the Bax inhibitor-1 (BI-1) gene capable of suppressing Bax-induced cell death in yeast. FEBS Lett;464: 143-147.

Kawasaki, T, Holt, B F, Nam, J, Boyes, D, Mackey, D, Taylor, C, Dangl, J 2001 RIN2 interacted with an Arabidopsis disease resistance gene, RPM1 encodes and RING finger-type ubiquitin ligase. 12^{th} International Research Conference on Arabidopsis. Abstract 200.

Kay, R et al. Duplication of Ca MV 35S promoter sequences creates a strong enhancer for plant genes. Science 1987;236:1299-1302.

Kaydamov, C, Tewes, A, Adler, K, Manteuffel, R. 2000 Molecular characterization of cDNAs encoding G protein alpha and beta subunits and study of their temporal and spatial expression patterns in Nicotiana plumbaginifolia Viv. Biochim Biophys Acta;1491:143-160.

Keeney, S, Giroux, C N, Kleckner, N. 1997 Meiosis-specific DNA double-strand breaks are catalyzed by Spo11, a member of a widely conserved protein family. Cell;88:375-384.

Kehoe, J W, Bertozzi, C R. 2000 Tyrosine sulfation: a modulator of extracellular protein-protein interactions. Chem Biol;7:R57-61.

Kemper, W M, Berry, K W, Merrick, W C. 1976 Purification and properties of rabbit reticulocyte protein synthesis initiation factors M2B□ and M2B□. J Biol Chem;251:5551-5557.

Khuri, S, Bakker, F T, Dunwell, J M. 2001 Phylogeny, function, and evolution of the cupins, a structurally conserved, functionally diverse superfamily of proteins. Mol Biol Evol;18:593-605.

Kim, K K, Hung, L W, Yokota, H, Kim, R, Kim, S H. 1998 Crystal structures of eukaryotic translation initiation factor 5A from Methanococcus jannaschii at 1.8 A resolution. Proc Natl Acad Sci USA;95:10419-10424.

Kingston-Smith, A H, Foyer, C H. 2000 Bundle sheath proteins are more sensitive to oxidative damage than those of the mesophyll in maize leaves exposed to paraquat or low temperatures. J Exp Bot;51:123-130.

Kinnersley, A M, Lin, F. 2000 Receptor modifiers indicate GABA is a potential modulator of ion transport in plants. Plant Growth Regul;32:65-76.

Kinnersley, A M. 1998 Bioactivity of AuxiGro Plant Metabolic Primer, a formulation containing GABA and glutamic acid. In: Proc. 25^{th} Ann. Mtg. Plant Growth Regulation Soc. Of America, pp. 89-94.

Kinoshita, Y, Jarell, A D, Flaman, J M, Foltz, G, Schuster, J, Sopher, B L, Irvin, D K, Kanning, K, Komblum, H I, Nelson, P S, Hieter, P, Morrison, R S. 2001 Pescadillo, a novel cell cycle regulatory protein abnormally expressed in malignant cells. J Biol Chem; 276(9):6656-6665.

Kinoshita, T, Yadegari, R, Harada, J J, Goldberg, R B, Fische, R L. 1999 Imprinting of the MEDEA polycomb gene in the Arabidopsis endosperm. Plant Cell;11: 1945-1952.

Kirsch, C et al 2001 A highly specific pathogen-responsive promoter element from the immediate-early avtivated CMPG1 gene in Petroselinum crispum. The Plant Journal;26(2):217-227.

Kisselbach, T A. 1949 The Structure and Reproduction of Corn. reprinted by Cold Spring Harbor Laboratory Press 1999.

Kiyosue, T, Wada, M. 2000 LKP1 (LOV kelch protein 1): a factor involved in the regulation of flowering time in arabidopsis. Plant J;23:807-815.

Kiyosue, T, Abe, H, Yamaguchi-Shinozaki, K, Shinozaki, K. 1998 ERD6, a cDNA clone for an early dehydration-induced gene of Arabidopsis, encodes a putative sugar transporter. Biochim. Biophys. Acta;1370:187-191.

Kiyosue, T, Ohad, N, Yadegari, R, Hannon, M, Dinneny, J, Wells, D, Katz, A, Margossian, L, Harada, J J, Goldberg, R B, and Fischer, R L. 1999 Control of fertilization-independent endosperm development by the MEDEA polycomb gene in Arabidopsis. Proc. Natl. Acad. Sci. USA;96:4186-4191.

Kiyosue, T, Yamaguchi-Shinozaki, K, Shinozaki, K. 1994 Characterization of two cDNAs (ERD10 and ERD14) corresponding to genes that respond rapidly to dehydration stress in Arabidopsis thaliana. Plant Cell Physiol;35:225-231.

Klahre, U T, Noguchi, S, Fujioka, S, Takatsuto, T, Yokota, T, Nomura, S, Yoshida, and Chuan, NH. 1998 The Arabidopsis DIMINUTO/DWARF1 gene encodes a protein involved in steroid synthesis. Plant Cell; 10:1677-1690.

Klee, H, Horsch, R, Rogers, S. Agrobacterium-mediated plant transformation and its further applications to plant biology. Annu. Rev. Plant Physiol. Plant Mol Biol. 1987;38:467-486.

Klein, R G, Beeson, W M, Cline, T R, Mertz, E T. 1971 Opaque-2 and floury-2 corn studies with growing swine. J Anim Sci;32(2):256-261.

Klessig, D F, Durner, J, Noad, R, Navarre, D A, Wendehenne, D, Kumar, D, Zhou, J M, Shah, J, Zhang, S, Kachroo, P, Trifa, Y, Pontier, D, Lam, E, Silva, H. 2000 Nitric oxide and salicylic acid signaling in plant defense. Proc Natl Acad Sci USA;97:8849-8855.

Kliebenstein, D J, Monde, R A, Last, R L. 1998 Superoxide dismutase in Arabidopsis: an eclectic enzyme family with disparate regulation and protein localization. Plant Physiol;118:637-650.

Klimczak, L J, Collinge, M A, Farini, D, Giuliano, G, Wlaker, J C and Cashmore, A R. 1995 Reconstitution of Arabidopsis Casein Kinase II from recombinant subunits and phosphorylation of transcription factor GBF1. Plant Cell;7(1):105-115.

Klimczak, L J, Schindler, U and Cashmore, A R. 1992 Reconstitution of Arabidopsis casein kinase II from recombinant subunits and phosphorylation of transcription factor GBF1. Plant Cell;4(1): 87-98.

Knauer, R, Lehle, L. 1999 The oligosaccharyltransferase complex from Saccharomyces cerevisiae. Isolation of the OST6 gene, its synthetic interaction with OST3, and analysis of the native complex. J Biol Chem;274:17249-17256.

Kodaki, T, Yamashita, S. Yeast phosphatidylethanolamine methylation pathway. Cloning and characterization of two distinct methyltransferase genes. J Biol Chem 1987;262:15428-15435.

Kodama, H. Fatty acid desaturation during chilling acclimation is one of the factors involved in conferring low-temperature tolerance to young tobacco leaves. Plant Physiology April 1995; 107(4):1177-1185.

Kodama, O, Miyakawa, J, Akatsuka, T, Kiyosawa, S. 1992 Sakuranetin, a flavonone phytoalexin from ultraviolet-irradiated rice leaves. Phytochem;31:3807-3808.

Kohler, C, Merkle, T, Roby, D, Neuhaus, G. 2001 Developmentally regulated expression of a cyclic nucleotide-gated ion channel from Arabidopsis indicates its involvement in programmed cell death. Planta;213:327-332.

Koizumi, N, Martinez, I M, Kimata, Y, Kohno, K, Sano, H, Chrispeels, M J. 2001 Molecular characterization of two Arabidopsis ire1 homologs, endoplasmic reticulum-located transmembrane protein kinases. Plant Physiol;127:949-962.

Komori, K, Sakae, S, Shinagawa, H, Morikawa, K, Ishino, Y. 1998 A Holliday junction resolvase from Pyrococcus furiosus: functional similarity to Escherichia coli RuvC provides evidence for conserved mechanism of homologous recombination in Bacteria, Eukarya, and Archaea. Proc Natl Acad Sci USA;96:8873-8878.

Koornnef, Maarten, Leon-Kloosterziel, Karen M, Schwartz, Steven H, Zeevaart, Jan A D. The genetic and molecular dissection of abscisic acid biosynthesis and signal transduction in Arabidopsis. Plant Physiol. Biochem;36:83-89, 1998.

Koyama, H, Kawamura, A, Kihara, T, Hara, T, Takita, E, Shibata, D. 2000 Overexpression of mitochondrial citrate synthase in Arabidopsis thaliana improved growth on a phosphorus-limited soil. Plant Cell Physiol;41:1030-1037.

Kramer, P J, Boyer, J S.1995 Water relations of plants and soil. Academic Press.

Kubigsteltig, I, Laudert, D, Weiber, E W. 1999 Structure and regulation of the Arabidopsis thaliana allene oxide synthase gene. Planta;208:463-471.

Kuhlbrandt, W, Wang, D N, Fujiyoshi, Y. Atomic model of plant light-harvesting complex by electron crystallography. Nature Feb. 17, 1994;367(6464):614-621.

Kuihn, C, Franceschi, V R, Schulz, A, Lemoine, R, and Frommer, W B. 1997 Localization and turnover of sucrose transporters in enucleate sieve elements indicate macromolecular trafficking. Science;275:1298-1300.

Kumar, A M, Soll, D. 1992 Arabidopsis alternative oxidase sustains Escherichia coli respiration. Proc. Natl. Acad. Sci. USA;89:10842-10846.

Kunz, J, Loeschmann, A, Deuter-Reinhard, M, Hall, M N. 2000 FAP1, a homologue of human transcription factor NF-X1, competes with rapamycin for binding to FKBP12 in yeast. Mol Microbiol. September 2000;37(6):1480-1493.

Kusaba, M, Dwyer, K, Hendershot, J, Vrebalov, J, Nasrallah, J B, Nasrallah, M E. 2001 Self-Incompatibility in the Genus Arabidopsis. Characterization of the s locus in the outcrossing a. lyrata and its autogamous relative a. thaliana. Plant Cell;13:627-643.

Kusao, T., Berberich, T., Harada, M., Suzuki, N., Sugawara, K. 1995 A maize DNA-binding factor with bZIP motif is induced by low temperature. Mol. Gen. Genet;248:507-517.

Kusnetsov, V, Herrmann, R G, Kulaeva, O N, Oelmuller, R. 1998 Cytokinin stimulates and abscisic acid inhibits greening of etiolated Lupinus luteus cotyledons by affecting the expression of the light-sensitive protochlorophyllide oxidoreductase. Mol Gen Genet;259:21-28.

Kyozuka, J, Konishi, S, Nemoto, K, Izawa, T, Shimamoto, K. 1998 Down-regulation of RFL, the FLO/LFY homolog of rice, accompanied with panicle branch initiation. Proc Natl Acad Sci USA;95:1979-1982.

Kyrpides, N C, Woese, C R. 1998 Universally conserved translation initiation factors. Proc Natl Acad Sci USA;95:224-228.

Kyte, J and Doolittle, R F. 1982 A simple method for displaying the hydropathic character of a protein. J. Mol. Biol;157(6):105-142.

Lahiri, S D, Yao, J, McCumbers, C, Allison, L A. 1999 Tissue-specific and light-dependent expression within a family of nuclear-encoded sigma-like factors from Zea mays. Mol Cell Biol Res Commun;1(1):14-20.

Lai, M H, Bard, M, Pierson, C A, Alexander, J F, Goebl, M, Carter, G T, and Kirsch, D. 1994 The identification of a gene family in the Saccharomyces cerevisiae ergosterol biosynthesis pathway. Gene;140: 41-49.

Laloi, M, Klein, M, Riesmeir, J W, Muller-Rober, B, Fluery, C Bouillaud, F, Ricquier, D. 1997. A plant cold-induced uncoupling protein. Nature;389:135-136.

Lam, E, Kato, N, Lawton, M. 2001 Programmed cell death, mitochondria and the plant hypersensitive response. Nature;411:848-853.

Lam, H M, Chiu, J, Hsieh, M H, Meisel, L, Oliveiria, I C, Shin, M, Corruzzi, G. 1998 Glutamate receptor genes in plants. Nature;396:125-126.

Laudert, D, Pfannschmidt, U, Lottspeich, F, Hollander-Czytka, H, and Weiler, E W. 1996 Cloning, molecular and functional characterization of Arabidopsis thaliana allene oxide synthase (CYP 74), the first enzyme of the octadecanoid pathway to jasmonates. Plant Mol. Biol;31:323-335.

Laudert, D, Schaller, F, Weiler, E. 2000 Transgenic Nicotiana tabacum and Arabidopsi thaliana plants overexpressing allene oxide synthase. Planta;211:163-165.

Laufs, J, Wirtz, U, Kammann, M, Matzeit, V, Schaefer, S, Schell, J, Czemilofsky, A P, Baker, B, Gronenborn, B. Wheat dwarf virus Ac/Ds vectors: expression and excision of transposable elements introduced into various cereals by a viral replicon. Proc Natl Acad Sci USA October 1990; 87(19):7752-7756.

Lee, I, Wolfe, D S, Nilsson, O, Weigel, D. 1997 A LEAFY co-regulator encoded by UNUSUAL FLORAL ORGANS. Curr Biol;7:95-104.

Lee, I J, Foster, K R, Morgan, P W. 1998 Photoperiod control of gibberellin levels and flowering in sorghum. Plant Physiol;116:1003-1011.

Lee, J H, Hubel, A, Schoffl, F. 1995 Derepression of the activity of genetically engineered heat shock factor causes constitutive synthesis of heat shock proteins and increased thermotolerance in transgenic Arabidopsis. Plant J;8:603-612.

Lee, J H, Van Montagu, M, Verbruggen, N. A highly conserved kinase is an essential component for stress tolerance in yeast and plant cells. Proc Natl Acad Sci USA May 1, 1999;96(10):5873-5877.

Lee, K O, Jang, H H, Jung, B G, Chi, Y H, Lee, J Y, Choi, Y O, Lee, J R, Lim, C O, Cho, M J, Lee, S Y. 2000 Rice 1Cys-peroxiredoxin over-expressed in transgenic tobacco does not maintain dormancy but enhances antioxidant activity. FEBS Lett;486:103-106.

Lee, R H, Wang, C H, Huang, L T, Chen, S C. 2001 Leaf senescence in rice plants: cloning and characterization of senescence up-regulated genes. J Exp Bot;52:1117-1121.

Lee, S., Suh, S., Seju, K., Crain, R. C., Kwak, J. M., Nam, H. G., and Lee, Y. 1997 Systemic elevation of phosphatidic acid and lysophospholipid levels in wounded plants. Plant J;12:547-556.

Lee, T M, Lur, H S, Lin, Y H, Chu, C. 1996 Physiological and biochemical changes related to methyl jasmonate-induced chilling tolerance of rice Oryza sativa L. Plant Cell Envir;19:65-74.

Lee, Y, Lloyd, A M and Roux, S J. 1999 Antisense expression of the CK2 σ-subunit gene in Arabidopsis. Effects on light-regulated gene expression and plant growth. Plant Physiology;119:989-1000.

Lee, Y R, Assmann, S M. 1999 Arabidopsis thaliana ‘extra-large GTP-binding protein’ (AtXLG1): a new class of G-protein. Plant Mol Biol 40:55-64.

Lees, N D, Bard, M, and Kirsch, D R. 1999 Biochemistry and molecular biology of sterol synthesis in Saccharomyces cerevisiae. Crit. Rev. Biochem. Mol. Biol;34:33-47.

Legendre, L 1993 Phospholipase activity and phospholipid patterns in tobacco cells treated with fungal elicitor. Plant Sci;107: 17-25.

Leggewie, G, Wilmitze, R L, Riesmeier, J W. 1997 Two cDNAs from potato are able to complement a phosphate uptake-deficient yeast mutant: identification of phosphate transporters from higher plants. Plant Cell;9:381-392.

Lemoine, R, Kühn, C, Thiele, N, Delrot, S, Frommer, WB. 1996 Antisense inhibition of the sucrose transporter: Effects on amount of carrier and sucrose transport activity. Plant Cell Environ; 19:1124-1131.

Leshem, Y Y, Pinchasov, Y. 2000 Non-invasive photoacoustic spectroscopic determination of relative endogenous nitric oxide and ethylene content stoichiometry during the ripening of strawberries Fragaria anannasa (Duch.) and avocados Persea americana (Mill.). J Exp Bot;51:1471-1473.

Leung, J, and Giraudat, J. 1998 Abscisic acid signal transduction. Annu. Rev. Plant Physiol. Plant Mol. Biol;49:199-222.

Leustek, T, Smith, M, Murillo, M, Singh, D P, Smith, A G, Woodcock, S C, Awan, S J, Warren, M J. 1997 Siroheme biosynthesis in higher plants. Analysis of an S-adenosyl-L-methionine-dependent uroporphyrinogen III methyltransferase from Arabidopsis thaliana. J Biol Chem;272:2744-2752.

Li, B D, Liu, L, Dawson, M, De Benedetti, A. 1997 Overexpression of eukaryotic initiation factor 4E (eIF4E) in breast carcinoma. Cancer;79:2385-2390.

Li, Q, Ritzel, R G, McLean, L L T, McIntosh, L, Ko, T, et al. 1996 Cloning and analysis of the alternative oxidase gene of Neurospora crassa. Genetics;142:129-140.

Li, W, Assmann, S M. 1993 Characterization of a G-protein-regulated outward K+ current in mesophyll cells of vicia faba L. Proc Natl Acad Sci USA;90:262-266.

Li, Z, Wurtzel, E T. 1998 The Itk gene family encodes novel receptor-like kinases with temporal expression in developing maize endosperm. Plant Mol Biol;37:749-761.

Liang, F, Cunningham, K. W, Harper, J F, and Sze, H. 1997 ECA1 complements yeast mutants defective in Ca pumps and encodes an ER-type Ca-ATPase in Arabidopsis thaliana. Proc. Natl. Acad. Sci. USA;94:8579-8584.

Lill, N L, Douillard, P, Awwad, R A, Ota, S, Lupher, M L Jr, Miyake, S, Meissner-Lula, N, Hsu, V W, Band, H. 2000 The evolutionarily conserved N-terminal region of Cbl is sufficient to enhance down-regulation of the epidermal growth factor receptor. J Biol Chem;275:367-377.

Lin, C M, Koh, S, Stacey, G, Yu, S M, Lin, T Y, Tsay, Y F. 2000 Cloning and functional characterization of a constitutively expressed nitrate transporter gene, OsNRT1, from rice. Plant Physiol. 2000;122:379-388.

Lincoln, C, Long, J, Yamaguchi, J, Serikawa, K, Hake, S. 1994 A knottedl-like homeobox gene in Arabidopsis is expressed in the vegetative meristem and dramatically alters leaf morphology when overexpressed in transgenic plants. Plant Cell;6: 1859-1876.

Ling, V, Snedden, W A, Shelp, B J, Assmann, S A. 1994 Analysis of a soluble calmodulin binding protein from fava bean roots: identification of glutamate decarboxylase as a calmodulin-activated enzyme. Plant Cell;6:1135-1143.

Lipowsky, G, Bischoff, F R, Schwarzmaier, P, Kraft, R, Kostka, S, Hartmann, E, Kutay, U, Gorlich, D. 2000 Exportin 4: a mediator of a novel nuclear export pathway in higher eukaryotes. EMBO J;19:4362-4371.

Liscovitch, M., and Cantley, L C. 1994 Lipid second messengers. Cell;77:329-334

Liscovitch, M., and Cantley, L C. 1995 Signal transduction and membrane traffic: The PITP/phosphoinositide connection. Cell;81:659-662.

Lisitsky I, and Schuster G. 1995 Phosphorylation of a chloroplast RNA-binding protein changes its affinity to RNA. Nucleic Acids Res. 23:2506-11

Lisse, T, Bartels, D, Kalbitzer, H R, Jaenicke, R. 1996 The recombinant dehydrin-like desiccation stress protein from the resurrection plant Craterostigma plantagineum displays no defined three-dimensional structure in its native state. Biol Chem; 1996 377:555-561.

Liu, D, Bienkowska, J, Petosa, C, Collier, R J, Fu, H, Liddington, R. 1995 Crystal structure of the zeta isoform of the 14-3-3 protein. Natur;376:191-194.

Liu, C., Muchhal, U S, Uthappa, M, Kononowicz, A K, Raghothama, K G. 1998 Tomato Phosphate Transporter Genes Are Differentially Regulated in Plant Tissues by Phosphorus. Plant Physiol;116:91-99.

Liu, K H, Huang, C Y, Tsay, Y F. CHL1 is a dual-affinity nitrate transporter of Arabidopsis involved in multiple phases of nitrate uptake. Plant Cell May 1999;11(5):865-874.

Liu, L, Tewari, R P, and Williamson, P R 1999 Laccase protects Cryptococcu neoformans from antifungal activity of alveolar macrophages. Infection and Immunity;67:6034-6039.

Liu, L, Zeng, M, Hausladen, A, Heitman, J, Stamler, J S. 2000 Protection from nitrosative stress by yeast flavohemoglobin. Proc Nati Acad Sci USA;97:4672-4676.

Liu Q, and Dreyfuss G. 1995 In vivo and in vitro arginine methylation of RNA-binding proteins. Mol Cell Biol. 15:2800-8.

Liu, Y P, Nemeroff, M, Yan, Y P and Chen, K Y. 1997 Interaction of eukaryotic initiation factor 5A with the human immunodeficiency virus type 1 Rev response element RNA and U6 snRNA requires deoxyhypusine or hypusine modification. Biol. Signals; 6:166-174.

Lloyd, A M, Walbot, V and Davis, R W. 1992 Arabidopsis and Nicotiana anthocyanin production activated by maize regulators R and C1. Science;258:1773-1775.

Lo, Samuel C, Yoki K C Butt, Chan, Y S Gilbert. 2000 False nitric oxide synthase immunoreactivity in Asparagus bean. Nitric Oxide;4: 175.

Loggini, B, Scartazza, A, Brugnoli, E, Navari-Izzo, F. 1999 Antioxidative defense system, pigment composition, and photosynthetic efficiency in two wheat cultivars subjected to drought. Plant Physiol;119:1091-1100.

Lopez-Bucio, J, de La Vega, O M, Guevara-Garcia, A, Herrera-Estrella, L. 2000 Enhanced phosphorus uptake in transgenic tobacco plants that overproduce citrate. Nat Biotechnol;1 8:450-453.

Lorbiecke, R, Sauter, M. 1999 Adventitious root growth and cell-cycle induction in deepwater rice. Plant Physiol;119:21-30.

Lorenz, T R. and Parks, L W. 1992 Cloning, sequencing, and disruption of the gene encoding sterol C-14 reductase in Saccharomyces cerevisiae. DNA Cell Biol;11:685-692.

Lorick, K L, Jensen, J P, Fang, S, Ong, A M, Hatakeyama, S, Weissman, A M. 1999 RING fingers mediate ubiquitin-conjugating enzyme (E2)-dependent ubiquitination. Proc Natl Acad Sci USA; 96:11364-11369.

Low, P S, Merida, J R. 1996 The oxidative burst in plant defense: function and signal transduction. Physiol. Plant;10:533-542.

Lu, Y P, Zhen, R G, Rea, P A. 1997 AtPT4: A fourth member of the Arabidopsis phosphate transporter gene family (accession no.U97546). Plant Physiol;114:747-757.

Ludwig, S R, Bowen, B, Beach, L, Wessler, S. 1990 A regulatory gene as a novel visible marker for maize transformation. Science;247:449-450.

Ludwig-Muller, J, Krishna, P, Forreiter, C. A glucosinolate mutant of Arabidopsis is thermosensitive and defective in cytosolic Hsp90 expression after heat stress. Plant Physiol July 2000; 123(3):949-958.

Luo, M, Bilodeau, P, Koltunow, A, Dennis, E. S, Peacock, W J, and Chaudhury, A M. 1999 Genes controlling fertilization-independent seed development in Arabidopsis thaliana. Proc. Natl. Acad. Sci. USA;96:296-301.

Lyznik, L A, Rao, K V, Hodges, T K. FLP-mediated recombination of FRT sites in the maize genome. Nucleic Acids Res. Oct. 1, 1996;24(19):3784-3789.

Ma, H, Yanofsky, M F, Meyerowitz, E M. 1990 Molecular cloning and characterization of GPA1, a G protein alpha subunit gene from Arabidopsis thaliana. Proc Natl Acad Sci USA;87:3821-3825.

Ma, J, Przibilla, E, Hu, J, Bogorad, L, Ptashne, M. Yeast activators stimulate plant gene expression. Nature Aug. 18, 1988;334(6183):631-633.

Maia, I G, Benedetti, C E, Leite, A, Turcinelli, S R, Vercesi, A E, Arruda, P. 1998 AtPUMP: an Arabidopsis gene encoding a plant uncoupling mitochondrial protein. FEBS Lett;429:403-406.

Makishima, T, Yoshimi, M, Komiyama, S, Hara, N, Nishimoto, T. 2000 A subunit of the mammalian oligosaccharyltransferase, DAD1, interacts with Mc1-1, one of the bc1-2 protein family. J Biochem (Tokyo);128:399-405.

Malik, M K, Slovin, J P, Hwang, C H, Zimmerman, J L. 1999 Modified expression of a carrot small heat shock protein gene, hsp17. 7, results in increased or decreased thermotolerance. Plant J;20:89-99.

Mandava, N. 1988 Plant growth-promoting brassinosteroids. Annu. Rev. Plant Physiol. Plant Mol. Biol;39:23-52.

Maniatis, Sambrook. Molecular Cloning: A Laboratory Manual, Cold Spring Harbor Press, 1989. Manival, X, Ghisolfi-Nieto, L, Joseph, G, Bouvet, P and Erard, M, RNA-binding strategies common to cold-shock domain- and RNA recognition motif-containing proteins. NAR 2001,29(11):2223-2233.

Mano, J, Babiychuk, E, Belles-Boix, E, Hiratake, J, Kimura, A, Inze, D, Kushnir, S, Asada, K. 2000 A novel NADPH:diamide oxidoreductase activity in Arabidopsis thaliana P1 zeta-crystallin. Eur J Biochem;267:3661-3671.

Marcireau, Guyonnet, D, and Karst, F. 1992 Construction and growth properties of a yeast strain defective in sterol 14-reductase. Curr. Genet.;22:267-272.

Marcus, A I, Moore, R C, Cyr, R J. 2001 The Role of Microtubules in Guard Cell Function. Plant Physiol;125:387-395.

Marechal, V, Elenbaas, B, Piette, J, Nicolas, J C, Levine, A J. 1994 The ribosomal L5 protein is associated with mdm-2 and mdm-2-p53 complexes. Mol Cell Biol;114:7414-7420.

Markham, K R, Tanner, G J, Cassi-Lit, M, Whitecross, M I, Naidu, M, Mitchell, K A. 1998 Protectant 3′,4′-dihydroxy flavones induced by enhanced UV-B in UV-tolerant rice cultivar. Phytochem;4 (6):2208-2212.

Marrs, K A, Casey, E S, Capitant, S A, Bouchard, R A, Dietrich, P S, Mettler, I J, Sinibaldi, R M. 1993 Characterization of two maize HSP90 heat shock protein genes: expression during heat shock, embryogenesis, and pollen development. Dev Genet;14:27-41.

Marshall, J G and Dumbroff, E B. Turgor regulation via cell wall adjustment in white spruce. Plant Physiol. January 1999;119(1):313-320.

Martin, C, Prescott, A, Mackay, S, Barlett, J, Vrijlandt, E. (1991) Control of anthocyanin biosynthesis in flowers of Antirrhinum majus. Plant J;1:37-49.

Martin, D, Proebsting, W, Hedden, P. The SLENDER gene of pea encodes a gibberellin 2-oxidase. Plant Physiology November 1999;121(3):775-781.

Martin, R C, Mok, M C, Mok D W S. 1999b Isolation of a cytokinin gene, ZOG1, encoding zeatin O-glucosyltransferase from Phaseolus lunatus. Proc. Natl. Acad. Sci. U.S.A;96:284-289.

Martin, R C, Mok, M C, Mok, D W S. 1999a A gene encoding the cytokinin enzyme zeatin O-xyosyltransferase of Phaseolus vulgaris. Plant Physiol;120:553-557.

Martin, W, Mustafa A Z, Henze, K, Schnarrenberger, C. Higher-plant chloroplast and cytosolic fructose-1,6-bisphosphatase isoenzymes: origins via duplication rather than prokaryote-eukaryote divergence. Plant Mol Biol November 1996;32(3):485-491.

Martinez-Garcia, J F, Huq, E, Quail, P H. 2000 Direct targeting of light signals to a promoter element-bound transcription factor. Science;288:859-863.

Martinez-Garcia, J F, Quail, P H. 1999 The HMG-I/Y protein PF1 stimulates binding of the transcriptional activator GT-2 to the PHYA gene promoter. Plant J;18:173-183.

Marty, I, Brugidou, C, Chartier, Y, Meyer, Y. 1993 Growth-related gene expression in Nicotiana tabacum mesophyll protoplasts. Plant J. August 1993;4(2):265-278.

Mason, M G, Botella, J R. 2000 Completing the heterotrimer: isolation and characterization of an arabidopsis thaliana G protein gamma-subunit cDNA. Proc Natl Acad Sci USA;97: 14784-14788.

Massacci, A, Battistelli, A, Loreto, F. 1996 Effect of drought stress on photosynthetic characteristics, growth and sugar accumulation of field-grown sweet sorghum. Aust J Plant Physiol;23:331-340.

Massacci, A, Jannelli, M A, Pietrini, F, Loreto, F. 1995 The effect of growth at low temperature on photosynthetic characteristics and mechanisms of photoprotection of maize leaves. J Exp Bot;46:119-127.

Matsubayashi, Y, Sakagami, Y. 1999 Characterization of specific binding sites for a mitogenic sulfated peptide, phytosulfokine-alpha, in the plasma-membrane fraction derived from Oryza sativa L. Eur J Biochem;262:666-671.

Matsubayashi, Y, Sakagami, Y. 2000 120- and 160-kDa receptors for endogenous mitogenic peptide, phytosulfokine-alpha, in rice plasma membranes. J Biol Chem;275:15520-15525.

Matsubayash,i Y, Takagi, L, Sakagami, Y. 1997 Phytosulfokine-alpha, a sulfated pentapeptide, stimulates the proliferation of rice cells by means of specific high- and low-affinity binding sites. Proc Natl Acad Sci USA.;94:13357-13362.

Matsufuji, S, Miyazaki, Y, Kanamoto, R, Kameji, T, Murakami, Y, Baby, T G, Fujita, K, Ohno, T, Hayashi, S. 1990 Analyses of ornithine decarboxylase antizyme mRNA with a cDNA cloned from rat liver. J Biochem (Tokyo);108:365-371.

Matsukura, C, Saitoh, T, Hirose, T, Ohsugi, R, Perata, P, Yamaguchi, J. 2000 Sugar Uptake and Transport in Rice Embryo. Expression of Companion Cell-Specific Sucrose Transporter (OsSUT1) induced by Sugar and Light. Plant Phys;124:85-93.

Matsumoto, Y, Sarkar, G, Sommer, S S, Wickner, R B. 1993 A yeast antiviral protein, SK18, shares a repeated amino acid sequence pattern with beta-subunits of G proteins and several other proteins. Yeast;9:43-51.

Mattoon, J R, Caravajal, E and Guthrie, D. 1990 Effects of hap mutations on heme and cytochrome formation in yeast. Curr. Genet;17:179-183.

Maural, C, Reizer, J, Schroeder, J L, and Chrispeels, M J. 1993 The vacuolar membrane protein γ-TIP creates specific channels in Xenopus oocytes. EMBO;12:2241-2247.

Maxwelll, D P, Wang, Y, McIntosh, L. The alternative oxidase lowers mitochondrial reactive oxygen production in plant cells. 1999. Proc. Nat Acad Sc;96:8271-8276.

May, T, Soll, J. 2000 14-3-3 proteins form a guidance complex with chloroplast precursor proteins in plants. Plant Cell;12:53-64.

Mayer, R R, Cherry, J H, Rhodes, D. 1990 Effects of heat shock on amino acid metabolism of cowpea cells. Plant Physiol;94:796-810.

Mazin, A V, Zaitseva, E, Sung, P, Kowalczykowski, S C. 2000 Tailed duplex DNA is the preferred substrate for Rad51 protein-mediated homologous pairing. EMBO J;19:1148-1156.

McBride A E, Weiss V H, Kim H K, Hogle J M, Silver P A. 2000 Analysis of the yeast arginine methyltransferase Hmt1p/Rmt1p and its in vivo function. J Biol Chem. 275:3128-36.

McCabe, T C, Finnegan, P M, Millar, A H, Day, D A, Whelan, J. 1998 Differential expression of the alternative oxidase genes in soybean cotyledons during postgerminative development. Plant Physiol;118:675-682.

McCracken, A A, Brodsky, J L. 2000 A molecular portrait of the response to unfolded proteins. Genome Biol.1(2):REVIEWS1013.

McGaw 1987 Cytokinin biosynthesis and metabolism. Plant hormones and their role in plant growth and development. Davies, P J ed. 76-93.

McGee 199) Maize Diseases: A reference source for seed technologists. The American Phytopathological Society, St. Paul, Minn.

McIntosh, L, Eichler, T, Gray, G, Maxwell, D, Nickels, R, Wang, Y. 1998 Biochemical and genetic controls exerted by plant mitochondria. Biochim Biophys. Acta;1365:278-284.

McIhtosh, L. 1994 Molecular biology of the alternative oxidase. Plant Physiol, 105:781-786.

McKenzie, M J, Mett, V, Reynolds, P H S, Jameson, P E. 1998 Controlled cytokinin production in transgenic tobacco using a copper-inducible promoter. Plant Physiol;116:969-977.

McKersie, B D, Bowley, S R, Jones, K S. 1999 Winter survival of transgenic alfalfa overexpressing superoxide dismutase. Plant Physiol; 119:839-848.

McKersie, B D, Bowley, S R. 1997 Active oxygen and freezing tolerance in transgenic plants. Plant Cold Hardiness: New York: Plenum: 68:203-204.

McKersie, B D, Murnaghan, J, Jones, K S, Bowley, S R. 2000 Iron-superoxide dismutase expression in transgenic alfalfa increases winter survival without a detectable increase in photosynthetic oxidative stress tolerance. Plant Physiol;122:1427-1437.

McMullen, M D, Byrne, P F, Snook, M E, Wiseman, B R, Lee, E A, Widstrom, N. W. 1998 Quantitative trait loci and metabolic pathways. Proc. Natl. Acad. Sci;95:1996-2000.

McNeil, S, Rhodes, D, Russell, B, Nuccio, M, Sachar-Hill, Y, and Hanson, A. Metabolic Modeling identifies the key constraints on an engineered glycine betaine syntheis pathway in tobacco. Plant Physiology 2000;124:153-162.

McNeil, S D., Nuccio, M L, and Hanson, A D. Betaines and related osmoprotectants. Targets for metabolic engineering of stress resistance. Plant Physiol. (August 1999),120:945-949.

McNeil, S D, Nuccio M L, Ziemak M J, Hanson A D. Enhanced synthesis of choline and glycine betaine in transgenic tobacco plants that overexpress phosphoethanolamine N-methyltransferase. Proc Natl Acad Sci USA 2001;98:10001-10005.

McWhorter, C G Epicuticular wax on Johnsongrass (Sorghum halepense) leaves. Weed Science 1997;41:475-482.

Meacham, G C, Patterson, C, Zhang, W, Younger, J M, Cyr, D M. 2001 The Hsc70 co-chaperone CHIP targets immature CFTR for proteasomal degradation. Nat Cell Biol;3: 100-5.

Meijer, H. J. G., Divecha, N., van der Ende, H., Musgrave, A., Munnik, T. 1999 Hyperosmotic stress induces rapid synthesis of phosphatidyl-d-inositol 3,5-bisphosphate in plant cells. Planta;208:294-298.

Melis, A. 1991. Dynamics of photosynthetic membrane composition and function. Biochim. Biophys. Acta;1058:87-106.

Membrillo-Hernandez, J, Coopamah, M D, Anjum, M F, Stevanin, T M, Kelly A, Hughes, M N, Poole, R K. 1999 The flavohemoglobin of Escherichia coli confers resistance to a nitrosating agent, a “Nitric oxide Releaser,” and paraquat and is essential for transcriptional responses to oxidative stress. J Biol Chem;274:748-754.

Merkouropoulos, G, Barnett, D, Shirsat, A. 1999 The Arabidopsis extensin gene is developmentally regulated, is induced by wounding, methyl jasmonate, abscisic and salicylic acid, and codes for a protein with unusual motifs. Planta;208:212-219.

Mezitt, L A, Lucas, W J. 1996 Plasmodesmal cell-to-cell transport of proteins and nucleic acids. Plant Mol. Biol;32:251-273.

Michaels, S D, Amasino, R M. 1999 Flowering Locus C encodes a novel MADS domain protein that acts as a repressor of flowering. Plant Cell;11:763-764.

Michelet, B, and Boutry, M. 1995 The plasma membrane ATPase: A highly regulated enzyme with multiple physiological functions. Plant Physiol;108:1-6.

Mikami, K, Katagiri, T, Iuchi, S, Yamaguchi-Shinozaki, K, and Shinozaki, K. 1998 A gene encoding phosphatidylinositol-4-phosphate 5-kinase is induced by water stress and abscisic acid in Arabidopsis thaliana. Plant J;15:563-568.

Millar, A A, Clemens, S, Zachgo, S, Giblin, E M, Taylor, D C, Kunst, L. CUT1, an Arabidopsis gene required for cuticular wax biosynthesis and pollen fertility, encodes a very-long-chain fatty acid condensing enzyme. Plant Cell May 1999;11(5):825-38.PMID: 10330468; UI: 99264304.

Millar, A A, Wrischer, M, Kunst, L. Accumulation of very-long-chain fatty acids in membrane glycerolipids is associated with dramatic alterations in plant morphology. Plant Cell November 1998; 10(11):1889-18902.

Miranda, E J, Hazel, J R. 1996 Temperature-induced changes in the transbilayer distribution of phosphatidylethanol-amine in mitoplasts of rainbow trout (Oncorthynchus mykiss) liver. J. Exp. Zool;274:23-32.

Miranda, L. T. de. 1984 Genetics of environmental resistance and super-genes: latente heat tolerance. Maize genetics cooperation news letter. 58.

Mitchell, P, Tollervey, D. 2000 Musing on the structural organization of the exosome complex. Nat Struct Biol;7:843-846.

Mitsuhara, I, Malik, K A, Miura, M, Ohashi, Y. 1999 Animal cell-death suppressors Bcl-x(L) and Ced-9 inhibit cell death in tobacco plants. Curr Biol;9:775-778.

Mitsukawa, N, Okumura, S, Shirano, Y, Sato, S, Kato, T, Harashima, S, Shibata, D. 1997 Overexpression of an Arabidopsis thaliana high-affinity phosphate transporter gene in tobacco cultured cells enhances cell growth under phosphate-limited conditions. Proc Natl Acad Sci USA;94:7098-7102.

Miyazawa, Y, Sakai, A, Miyagishima, S, Takano, H, Kawano, S, Kuroiwa, T. 1999 Auxin and cytokinin have opposite effects on amyloplast development and the expression of starch synthesis genes in cultured bright yellow-2 tobacco cells. Plant Physiol;121:461-469.

Mizoguchi, T, Irie, K, Hirayama, T, Hayashida, N, Yamaguchi-Shinozaki, K, Matsumoto, K, Shinozaki, K. Proc. Natl. Acad. Sci USA Jan. 23, 1996;93(2):765-769.

Mizukami, Y. 2001 A matter of size: developmental control of organ size in plants. Curr. Opin. Plant Biol;4(6):533-539.

Mizuuchi, K, Kemper, B, Hays, J, Weisberg, R A. 1983 T4 endonuclease VII cleaves holiday structures. Cell;29:357-365.

Mock, J J, Pearce, R B. 1975 An ideotype of maize. Euphytica;24:613-623.

Moffatt, B A, Wang, L, Allen, M S, Stevens, Y Y, Qin, W, Snider, J, von Schwartzenberg, K. 2000 Adenosine kinase of Arabidopsis. Kinetic properties and gene expression. Plant Physiol; 124:1775-1785.

Möhlmann, T, Tjaden, J, Schwöppe, C, Winkler, H H, Kampfenkel, K, Neuhaus, H E. 1998 Occurence of two plastidic ATP/ADP transporters in Arabidopsis thaliana: Molecular characterisation and comparative structural analysis of homologous ATP/ADP translocators from plastids and Rickettsia prowazekii. Eur. J. Biochem;252:353-359.

Momand, J, Zambetti, G P, Olson, D C, George, D, Levine, A J. 1992 The mdm-2 oncogene product forms a complex with the p53 protein and inhibits p53-mediated transactivation. Cell;69: 1237-1245.

Montgomery, B L, Yeh, K C, Crepeau, M W, Lagarias, J C. 1999 Modification of distinct aspects of photomorphogenesis via targeted expression of mammalian biliverdin reductase in transgenic Arabidopsis plants. Plant Physiol;121:629-639.

Moore, A L, Siedow, J N. 1991 The regulation and nature of the cyanide-resistant alternative oxidase of plant mitochondria. Biochim. Biophys. Acta; 1059:121-140.

Moorhead, G, Douglas, P, Cotelle, V, Harthill, J, Morrice, N, Meek, S, Deiting, U, Stitt, M, Scarabel, M, Aitken, A, MacKintosh, C. 1999 Phosphorylation-dependent interactions between enzymes of plant metabolism and 14-3-3 proteins. Plant J; 18:1-12.

Moose, S P, Sisco, P H. Glossy15 an APETALA2-loke gene from maize that regulates leaf epidermal cell identity. Genes and Development. 1996;10:3018-3027.

Moose, S P, Sisco, P H. Glossyl 5 controls the epidermal juvenile-to-adult phase transition in Maize. The Plant Cell. 1994;6:1343-1355.

Morelli, G, Ruberti, I. 2000 Shade avoidance responses. Driving auxin along lateral roots. Plant Physiol;122:621-626.

Moreno, M A, Harper, L C, Krueger, R W, Dellaporta, S L, Freeling, M. 1997 liguleless1 encodes a nuclear-localized protein required for induction of ligules and auricles during maize leaf organogenesis. Genes Dev;11:616-628.

Morgan, D O. 1995 Principles of CDK regulation. Nature;374:131-134.

Mori, H, Inoue, M, Yano, M, Wakabayashi, H, Kido, H. 2000 14-3-3tau associates with a translational control factor FKBP12-rapamycin-associated protein in T-cells after stimulation by pervanadate. FEBS Lett;467:61-64.

Morrissey P J, Glick R E and Melis A 1989 Supramolecular assembly and function of subunits associated with the chlorophyll a-b light-hatvesting complex II (LHCII) in soybean chloroplast. Plant Cell Physiol. 30: 335-344.

Morsomme, P, Dambly, S, Maudoux, O, and Boutry, M. 1998 Single point mutations distributed in 10 soluble and membrane regions of the Nicotiana plumbaginifolia plasma membrane PMA2 H⁺-ATPase activate the enzyme and modify the structure of the C-terminal region. J. Biol. Chem; 273:34837-34842.

Morsomme, P, de Kerchove d'Exaerde, A, De Meester, S, Thines, D, Goffeau, A, and Boutry, M. 1996 Single point mutations in various domains of a plant plasma membrane H⁺-ATPase expressed in S. cerevisiae increase H⁺-pumping and permit yeast growth at low pH. EMBO J;20:5513-5526.

Mothe-Satney, I, Brunn, G J, McMahon, L P, Capaldo, C T, Abraham, R T, Lawrence, J C Jr. 2000 Mammalian target of rapamycin-dependent phosphorylation of PHAS-I in four (S/T)P sites detected by phospho-specific antibodies. J Biol Chem;275:33836-33843.

Muehlbauer, G J, Fowler, J E, Girard, L, Tyers, R, Harper, L, Freeling, M. 1999 Ectopic expression of the maize homeobox gene liguleless3 alters cell fates in the leaf. Plant Physiol;119:651-662.

Muller, V, Basset, G, Nelson, D R, Klingenberg, M. 1996 Probing the role of positive residues in the ADP/ATP carrier from yeast. The effect of six arginine mutations on oxidative phosphorylation and AAC expression. Biochem;35:16132-16143.

Müller, V, Heidkämper, D, Nelson, D R, Klingenberg, M. 1997 Mutagenesis of some positive and negative residues occuring in repeat triad residues in the ADP/ATP carrier from yeast. Biochemistry;36: 16008-16018.

Munnik, T. 2001 Phosphatidic acid: an emerging plant lipid second messenger. Trds PI. Sci;6:227-233.

Munnik, T, de Vrije, T, Irvine, R F, and Musgrave, A. 1996 Identification of diacylglycerol pyrophosphate as a novel metabolic product of phosphatidic acid during G-protein activation in plants. J. Biol. Chem;271:15708-15715

Munnik, T, Irvine, R F, and Musgrave, A. 1998a Phospholipid signaling in plants. Biochim. Biophys. Acta;1389:222-272

Munnik, T, Vanhimbergen, J A., Terriet, B, Braun, F J, Irvine, R F, Vandenende, H, and Musgrave, A. 1998b Detailed analysis of the turnover of polyphosphoinositides and phosphatidic acid upon activation of phospholipases C and D in Chlamydomonas cells treated with non-permeabilizing concentrations of mastoparan. Planta;207:133-145.

Murakami, S, Kondo, Y, Nakano, T, Sato, F. 2000 Protease activity of CND41, a chloroplast nucleoid DNA-binding protein, isolated from cultured tobacco cells. FEBS Lett;468:15-18.

Murakami, T, Anzai, H, Imai, S, Satoh, A, Nagaoka, K, Thompson, C J. Mol Gen Genet 1986;205:42-50.

Murakami, Y, Ichiba, T, Matsufuji, S, Hayashi, S. 1996 Cloning of antizyme inhibitor, a highly homologous protein to ornithine decarboxylase. J Biol Chem;271 :3340-3342.

Murakami, Y, Matsufuji, S, Miyazaki, Y, Hayashi, S. 1994 Forced expression of antizyme abolishes omithine decarboxylase activity, suppresses cellular levels of polyamines and inhibits cell growth. Biochem J;304:183-187.

Muramoto, T, Kohchi, T, Yokota, A, Hwang, I, Goodman, H M. 1999 The Arabidopsis photomorphogenic mutant hy1 is deficient in phytochrome chromophore biosynthesis as a result of a mutation in aplastid heme oxygenase. Plant Cell;11:335-348.

Murchie, E H and Horton, P. 1997. Acclimation of photosynthesis to irradiance and spectral quality in British plant species: Chlorophyll content, photosynthetic capacity and habitat preference. Plant Cell Environment;20:438-448.

Mussig, C, Biesgen, C, Janina, L, Uwer, U, Weiler, E, Altmann, T. 2000 A novel stress-inducible 12-oxophytodienoate reductase from Arabidopsis thaliana provides a potential link between Brassinosteroid-action and Jasmonic-acid synthesis. J Plant Physiol;157:143-152.

Muyrers, J P, Zhang, Y, Buchholz, F, Stewart, A F. 2000 RecE/RecT and Redalpha/Redbeta initiate double-stranded break repair by specifically interacting with their respective partners. Genes Dev;14:1971-1982.

Nagano, Y, Okada, Y, Narita, H, Asaka, Y, Sasaki, Y. 1995 Location of light-repressible, small GTP-binding protein of the YPT/rat family in the growing zone of eliolated pea stems. Proc. Natl. Acad. Sci;92:6314-6318.

Nakafuku, M, Obara, T, Kaibuchi, K Miyajima, I, Miyajima, A, Itoh, H, Nakamura, S, Arai, K, Matsumoto, K, Kaziro, Y. 1988 Isolation of a second yeast Saccharomyces cerevisiae gene (GPA2) coding for guanine nucleotide-binding regulatory protein: studies on its structure and possible functions. Proc. Natl. Acad. Sci USA;85:1374-1378.

Nakano, T, Murakami, S, Shoji, T, Yoshida, S, Yamada, Y, Sato, F. 1997 A novel protein with DNA binding activity from tobacco chloroplast nucleoids. Plant Cell;9:1673-1682.

Nakashima, T, Sekiguchi, T, Kuraoka, A, Fukushima, K, Shibata, Y, Komiyama, S, Nishimoto, T. 1993 Molecular cloning of a human cDNA encoding a novel protein, DAD1, whose defect causes apoptotic cell death in hamster BHK21 cells. Mol Cell Biol;13:6367-74.

Napoli, C A, Beveridge, C A, Snowden, K C. 1999 Reevaluating concepts of apical dominance and the control of axillary bud outgrowth. Curr Top Dev Biol;44: 127-169.

Nathan, D F, Vos, M H, Lindquist, S. 1997 In vivo functions of the Saccharomyces cerevisiae Hsp90 chaperone. Proc Natl Acad Sci USA;94:12949-12956.

Nathan, D F, Vos, M H, Lindquist, S. 1999 Identification of SSF1, CNS1, and HCH1 as multicopy suppressors of a Saccharomyces cerevisiae Hsp90 loss-of-function mutation. Proc Natl Acad Sci USA;96:1409-1414.

Neff, M, Nguyen, S, Malancharuvil, E, Fujioka, S, Noguchi, T, Seto ,H, Tsubuke, M, Honda, T, Takatsuto, S, Yoshida, S, Chory, J. 1999. BAS1: A gene regulating brassinosteroid levels and light responsiveness in Arabidopsis. Proc. Nat. Acad. Sci;96(26):15316-15323.

Neff, M M, Frankhauser, C, Chory, J. 2000 Light: an indicator of time and place. Genes Devel;14:257-271.

Nelson, D C, Lasswell, J, Rogg, L E, Cohen, M A, Bartel, B. 2000 FKF1, a clock-controlled gene that regulates the transition to flowering in Arabidopsis. Cell;101:331-340.

Nelson, D R, Lawson, J E, Klingenberg, M, Douglas, M G. 1993 Site-directed mutagenesis of the yeast mitochondrial ADP/ATP translocator. J. Mol. Biol;230:1159-1170.

Nelson, O E Jr and Chang, M T. 1974. Effect of multiple aleurone layers on the protein and amino acid content of maize endosperm. Crop Sci;14:374-376.

Neuhaus, H E, Thom, E, Mohlmann, T, Steup, M, Kampfenkel, K. Characterization of a novel eukaryotic ATP/ADP translocator located in the plastid envelope of Arabidopsis thaliana L. Plant J January 1997;11(1):73-82.

Ni, M, Tepperman, J M, Quail, P H. 1999 Binding of phytochrome B to its nuclear signalling partner PIF3 is reversibly induced by light. Nature;400:781-784.

Nicholson, R L, Kollipara, S S, Vincent, J R, Lyons, P C, Cadena-Gomez, G. 1987 Phytoalexin synthesis by the sorghum mesocotyl in response to infection by pathogenic and nonpathogenic fungi. Proc. Natl. Acad. Sci. USA;84:5520-5524.

Nieto-Sotelo, J, Ichida, A, Quail, P H. 1994 PF1: an A-T hook-containing DNA binding protein from rice that interacts with a functionally defined d(AT)-rich element in the oat phytochrome A3 gene promoter. Plant Cell;6:287-301.

Nieto-Sotelo, J, Kannan, K B, Martinez, L M, Segal, C. 1999 Characterization of a maize heat-shock protein 101 gene, HSP101, encoding a ClpB/Hsp100 protein homologue. Gene;230: 187-195.

Nieto-Sotelo, J, Martinez, L M and Meely, R. 2000 Maize Genetics Conference Abstracts 42:P126.

Nigg, E A. 1996 Cyclin-dependent kinase 7: at the cross-roads of transcription, DNA repair and cell cycle control? Curr Opin Cell Biol;8:312-317.

Nikawa, J, Tsukagoshi, Y, and Yamashita, S. 1986. Cloning of a gene encoding choline transport in Saccharomyces cerevisiae. J Bacteriol;166:328-330.

Nishimura, A, Tamaoki, M, Sakamoto, T, Matsuoka, M. 2000 Over-expression of tobacco knottedl-type class1 homeobox genes alters various leaf morphology. Plant Cell Physiol;41 :583-590.

Nishizawa, A N, Buchanan, B B. Enxyme regulation in C4 photosynthesis. Purification and properties of thioredoxin-linked fructose bisphosphatase and sedoheptulose bisphosphatase from corn leaves. J Biol Chem Jun. 25, 1981;256(12):6119-6126.

Niyogi, K K. 1999. Photoprotection revisited: Genetic and molecular approaches. Ann. Rev. Plant Physio. Plant Mol. Biol:50;333-359.

Nuccio, M., Ziemak, M., Henry, S., Weretilnyk, E. and Hanson A. 2000 cDNA cloning of phosphoethanolomine N-methyltransferase from spinach by Schizosaccharomyces pombe and characterization of the recombinant enzyme. The Journal of Biological Chemistry;275,19:14095-14101.

Nuccio, M L, McNeiol, S D, Ziemak, M J, Hanson, A D, Jain, R K, and Selvaraj, G. Choline import into chloroplasts limits glycine betaine synthesis in tobacco: Analysis of plants engineered with a chloroplastic or a cytosolic pathway. Metabolic Engineering; 2000;2:300-311.

O'Regan, S, Traiffort, E, Ruat, M, Cha, N, Compaore, D, and Meunier, M F. An electric lobe suppressor for a yeast choline transport mutation belongs to a new family of transporter-like proteins. Proc. Natl Acad Sci USA Feb. 15, 2000;97(4):1835-1840.

Ober, D, Hartmann, T. 1999a Deoxyhypusine synthase from tobacco. cDNA isolation, characterization, and bacterial expression of an enzyme with extended substrate specificity. J Biol Chem;274:32040-32047.

Ober, D, Hartmann, T. 1999b Homospermidine synthase, the first pathway-specific enzyme of pyrrolizidine alkaloid biosynthesis, evolved from deoxyhypusine synthase. Proc Natl Acad Sci USA;96:14777-14782.

Odell, J T, Nagy, F, Chua, N H. Promoter sequences from the caluiflower mosaic virus genome. Nature 1985;313:810-812.

Ogawa, H, Imai, S, Shimizu, T, Satoh, A, Kojima, M. Cosynthesis and protoplast fusion by mutants of bialaphos (AMPBA) producing Streptomyces hygroscopicus. J Antibiot (Tokyo) August 1983;36(8):1040-1044.

Oh, S A, Park, J H, Lee, G I, Paek, K H, Park, S K, Nam, H G. 1997 Identification of three genetic loci controlling leaf senescence in Arabidopsis thaliana. Plant J;12:527-535.

Ohad, N, Margossian, L, Hsu, Y. -C, Williams, C, Repetti, P, and Fischer, R L. 1996 A mutation that allows endosperm development without fertilization. Proc. Natl. Acad. Sci. USA;93:5319-5324.

Ohad, N, Yadegari, R, Margossian, L, Hannon, M, Michaeli, D, Harada, J J, Goldberg, R B, Fischer, R L. 1999 Mutations in FIE, a WD polycomb group gene, allow endosperm development without fertilization. Plant Cell;11:407-416.

Oka et al., Mol Gen Genet, May 4, 1979, 172(2):151-9

Okamuro, J K, Caster, B, Villarroel, R, Van Montagu, M, Jofuku, K D. The AP2 domain of APETALA2 defines a large new family of DNA binding proteins in Arabidopsis. Proc Natl Acad Sci USA Jun. 24, 1997;94(13):7076-7081.

Olien, C R, Smith, M N. 1977 Ice adhesions in relation to freeze stress. Plant Physiol;60:499-503.

Omata, T, Price, G D, Badger, M R, Okamura, M, Gohta, S and Ogawa, T. 1999 Identification of an ATP-binding cassette transporter involved in bicarbonate uptake in the cyanobacterium Synechococcus sp. Strain PCC 7942. Proc. Natl. Acad. Sci. USA;96(23):13571-13576.

Omirulleh, S, Abraham, M, Golovkin, M, Stefanov, I, Karabaev, M K, Mustardy, L, Morocz, S, Dudits, D. Activity of a chimeric promoter with the doubled CaMV 35S enhancer element in protoplast-derived cells and transgenic plants in maize. Plant Mol Biol February 1993;21(3):415-428.

Oparka, K J, Duckett, C M, Prior, D A M, and Fisher, D B. 1994 Real-time imaging of phloem unloading in the root tip of Arabidopsis. Plant J;6:759-766.

Opsahl-Ferstad, H -G, Le Deunff, E, Dumas, C, Rogowsky, P M. 1997 ZmEsr, a novel endosperm-specific gene expressed in a restricted region around the maize embryo. Plant J;12:235-246.

Oram, M, Keeley, A, Tsaneva, I. 1998 Holliday junction resolvase in Schizosaccharomyces pombe has identical endonuclease activity to the CCE1 homologue YDC2. Nucleic Acids Res;26:594-601.

Ori, N, Juarez, M T, Jackson, D, Yamaguchi, J, Banowetz, G M, Hake, S. 1999 Leaf senescence is delayed in tobacco plants expressing the maize homeobox gene knotted1 under the control of a senescence-activated promoter. Plant Cell;11: 1073-1080.

Orzaez, D, de Jong, A J, Woltering, E J. 2001 A tomato homologue of the human protein PIRIN is induced during programmed cell death. Plant Mol Biol;46:459-468.

Oster, U, Tanaka, R, Tanaka, A, Rudiger, W. Cloning and functional expression of the gene encoding key enzyme for chlorophyll b biosynthesis (CAO) from Arabidopsis thaliana. Plant J February 2000;21(3):305-310.

Osterlund, M T, Hardtke, C S, Wei, N, Deng, X W. 2000 Targeted destabilization of HY5 during light-regulated development of Arabidopsis. Nature;405:462-466.

Oufattole, M, Arango, M, Boutry, M. 2000 Identification and expression of three new Nicotiana plumbaginifolia genes which encode isoforms of a plasma-membrane H+-ATPase, and one of which is induced by mechanical stress. Planta;210:715-722.

Ourisson, G. 1994 Pecularities of sterol biosynthesis in plants. J. Plant Physiol;143: 434-439.

Ouyang, Y b, Lane, W S, Moore, K L. 1998 Tyrosylprotein sulfotransferase: purification and molecular cloning of an enzyme that catalyzes tyrosine 0-sulfation, a common posttranslational modification of eukaryotic proteins. Proc Natl Acad Sci USA;95:2896-2901.

Overall, R L, Blackman, L M. 1996 A model for macromolecular structure of plasmodesmata. Trends Plant Sci;1:207-211.

Ow, D W, Wood, K V, DeLuca, M, De Wet, J R, Helinski, D R, Howell, S H. Transient and stable expression of the firefly luciferase gene in plant calls and transgenic plants. Science Nov. 14, 1986; 234:856-859.

Ozcan, S, and Johnston, M. 1995 Three different regulatory mechanisms enable yeast hexose transporter (HXT) genes to be induced by different levels of glucose. Mol Cell Biol;15:1564-1572.

Padmanabhan, V, Dias, D M, Newton, R J. 1997 Expression analysis of a gene family in loblolly pine (Pinus taeda L.) induced by water deficit stress. Plant Mol Biol;35:801-807.

Padmavati, M, Reddy, A R. 1999 Flavonoid biosynthetic pathway and cereal defence response: an emerging trend in crop biotechnology. Plant Biochem. Biotechnol;8:15-20.

Padmavati, M, Sakthivel, N, Thara, K V, Reddy, A R. 1997 Differential sensitivity of rice pathogens to growth inhibition by flavonoids. Phytochem;46(3):499-502.

Palmgren, M G. 1998 Proton gradients and plant growth: Role of the plasma membrane H⁺-ATPase. Adv. Bot. Res;28:1-70.

Palmgren, M G., and Christensen, G. 1994 Functional comparisons between plant plasma membrane H⁺-ATPase isoforms expressed in yeast. J. Biol. Chem;269:3027-3033.

Pan, L, Kawai, M, Yu, L H, Kim, K M, Hirata, A, Umeda, M, Uchimiya, H 2001 The Arabidopsis thaliana ethylene-responsive element binding protein (AtEBP) can function as a dominant suppressor of Bax-induced cell death of yeast. FEBS Lett;508:375-378.

Pan, L, Kawai, M, Yano, A, Uchimiya, H. 200) Nucleoside diphosphate kinase required for coleoptile elongation in rice. Plant Physiol;122:447-452.

Pan, R C, Gu, H. 1995 Effect of methyl jasmonate in the growth and drought resistance in peanut seedlings. Acta Phytophysiologica Simica;21:215-220.

Pare, P W, Albom, H T, Tumlinson, J H. 1998 Concerted biosynthesis of an insect elicitor of plant volatiles. Proc Natl Acad Sci USA;95:13971-13975.

Park, M H, Cooper, H L and Folk, J E. 1982 The biosynthesis of protein-bound hypusine (N-(4-amino-2-hydroxybutyl)lysine). Lysine as the amino acid precursor and the intermediate role of deoxyhypusine (N-(4-aminobutyl)lysine). J. Biol. Chem.;257:7217-7222.

Park, M H, Lee, Y B, Joe, Y A. 1997 Hypusine is essential for eukaryotic cell proliferation. Biol Signals;6:115-123.

Park, M H, Wolff, E C, Folk, J E. 1993 Hypusine: its post-translational formation in eukaryotic initiation factor 5A and its potential role in cellular regulation. Biofactors;4:95-104.

Park, Y, Karlsson, J, Rojdestvensk,i I, Pronina, N, Klimov, V, Oquist, G and Samuelsson, G. 1999 Role of a novel photosystem II-associated carbonic anhydrase in photosynthetic carbon assimilation in Chlamydomonas reinhardtii. FEBS Letters;444:102-105.

Parks, B M, Quail, P H, Hangarter, R P. 1996 Phytochrome A regulates red-light induction of phototropic enhancement in Arabidopsis. Plant Physiol;110:155-162.

Parsons, C A, Baumann, P, Van Dyck, E, West, S C. 2000 Precise binding of single-stranded DNA termini by human RAD52 protein. EMBO J. 2000;19:4175-4181.

Partaledis, J A and Berlin, V. 1993 The FKB2 gene of Saccharomyces cerevisiae, encoding the immunosuppressant-binding protein FKBP-13, is regulated in response to accumulation of unfolded proteins in the endoplasmic reticulum. PNAS (USA);90:5450-5454.

Paszkowski, J, Shillito, R D, Saul, M, Mandak, V, Hohn, T, Hohn, B, Potrykus, I. Direct gene transfer to plants. EMBO Journal 1984;3(12):2717-2722.

Patel, S., Morris, S. A., Adkins, C. E., O'beirne, G. and Taylor, C. W. 1997 Ca²⁺-independent inhibition of inositol trisphosphate receptors by calmodulin. Redistribution of calmodulin as a possible means of regulating Ca²⁺ mobilization. Proc. Natl Acad Sci. USA;94:11627-11632.

Patil, C, Walter, P. 2001 Intracellular signaling from the endoplasmic reticulum to the nucleus: the unfolded protein response in yeast and mammals. Curr Opin Cell Biol;13(3):349-355.

Patrick, J W. 1997 Phloem unloading: Sieve element unloading and post-sieve element transport. Annu. Rev. Plant Physiol. Plant Mol. Biol.;48:191-222.

Patterson, G W, Hugly, S, and Harrison, D. 1993 Sterols and phytyl esters of Arabidopsis thaliana under normal and chilling temperatures. Phytochemistry;33:1381-1383.

Pearce, G, Strydom, D, Johnson, S, Ryan C. A polypeptide from tomato leaves induces wound-inducible proteinase inhibitor proteins. Science Aug. 23, 1991;253:895-898.

Peat, T S, Newman, J, Waldo, G S, Berendzen, J, Terwilliger, T C. 1998 Structure of translation initiation factor 5A from Pyrobaculum aerophilum at 1.75 A resolution. Structure. 1998;6:1207-1214.

Pechan, T, Jiang, B, Steckler, D, Ye, L, Lin, L, Luthe, D S, Williams, W P. 1999 Characterization of three distinct cDNA clones encoding cysteine proteinases from maize (Zea mays L.) callus. Plant Mol Biol;40:111-119.

Pechan, T, Ye, L, Chang, Y m, Mitra, A, Lin, L, Davis, F M, Williams, W P, Luthe, D S. 2000 A unique 33-kD cysteine proteinase accumulates in response to larval feeding in maize genotypes resistant to fall armyworm and other lepidoptera. Plant Cell; 12:1031-1040.

Pelletier, M K, Burbulis, I E, Winkel-Shirely, B. Disruption of specific flavonoid genes enhances the accumulation of flavonoid enzymes and end-products in Arabidopsis seedlings. Plant Mol Biol May 1999;40(1):45-54.

Peng, J, Richards, D E, Hartley, N M, Murphy, G P, Devos, K M, Flintham, J E, Beales, J, Fish, L J, Worland, A J, Pelica, F, Sudhakar, D, Christou, P, Snape, J W, Gale, M D, Harberd, N P. 1999 ‘Green revolution’ genes encode mutant gibberellin response modulators. Nature;400:256-261.

Penninckx, I A M A, Thomma, B P H J, Buchala, A, Metraux, J. -P, and Broekaert, W F. 1998 Concomitant activation of a jasmonate and ethylene response pathways is required for induction of a plant defensin gene in Arabidopsis. Plant Cell;10:2103-2113.

Peskan, T, Oelmuller, R. 2000 Heterotrimeric G-protein beta-subunit is localized in the plasma membrane and nuclei of tobacco leaves. Plant Mol Biol;42:915-922.

Petit, J M, van Wuytswinkel, O, Briat, J -F, Lobreaux, S. Characterization of an Iron-Dependent Regulatory Sequence (IDRS) involved in the transcriptional control of AtFer1 and ZmFer1 plant ferritin genes by iron. J. Biol.Chem. Feb. 23, 2001;276(8):5584-5590.

Petukhova, G, Sung, P, Klein, H. 2000 Promotion of Rad5 1-dependent D-loop formation by yeast recombination factor Rdh54/Tid1. Genes Dev;14:2206-2215.

Petzold, U, Peschel, S, Dahse, I, Adam, G. 1992. Stimulation of source-applied 14-C-Sucrose export in Vicia faba plants by brassinosteroids, GA3 and IAA. Acta Bot Neerl;41 :469-479.

Pical, C, Westergren, T, Dove, S K, Larsson, C, Sommarin, M. 1999 Salinity and hyperosmotic stress induce rapid increases in phosphatidylinositol 4,5-bisphosphate, diacylglycerol pyrophosphate, and phosphatidylcholine in Arabidopsis thaliana cells. J. Biol. Chem;274:38232-38240.

Picard, D, Khursheed, B, Garabedian, M J, Fortin, M G, Lindquist, S, Yamamoto, K R. 1990 Reduced levels of hsp90 compromise steroid receptor action in vivo. Nature;348:166-168.

Pichon, O, Desbiez, M -O. 1994 Is cytoplasmic pH involved in the regulation of cell cycle in plants? Physiol Plant;92:261-265.

Pickart, C M. 2001 Mechanisms underlying ubiquitination. Annu Rev Biochem;70:503-533.

Piotrowski, M, Morsomme, P, Boutry, M, and Oecking, C. 1998 Complementation of the Saccharomyces cerevisiae plasma membrane H⁺-ATPase by a plant H⁺-ATPase generates a highly abundant fusicoccin binding site. J. Biol. Chem;273:30018-30023.

Pirrotta, V. 1998 Polycombing the genome: PcG, trxG, and chromatin silencing. Cell;93:333-336.

Pittman, R H, Andrews, M T, Setzer, D R. 1999 A feedback loop coupling 5 S rRNA synthesis to accumulation of a ribosomal protein. J Biol Chem;274:33198-33201.

Plakidou-Dymock, S, Dymock, D, Hooley, R. 1998 A higher plant seven-transmembrane receptor that influences sensitivity to cytokinins. Curr. Biol;8:315-324.

PlantCARE database: PlantCARE, a plant cis-acting regulatory element database. S. Rombauts, P. Dehais, M. Van Montagu and P. Rouze. Nucleic Acids Res (1999). Vol:27-(1) p:295-296

Plaxton, W C. 1996 The organization and regulation of plant glycolysis. Annu. Rev. Plant Physiol. Plant Mol. Biol;47: 185-214.

Ponting, C P, Blake, D J, Davies, K E. 1996 ZZ and TAZ: new putative zinc fingers in dystrophin and other proteins. Trends Biochem Sci;21:11-13.

Poole, R K, Hughes, M N. 2000 New functions for the ancient globin family: bacterial responses to nitric oxide and nitrosative stress. Mol Microbiol; 36:775-783.

Pozuelo, M, MacKintosh, C, Galvan, A, Fernandez, E. 2001 Cytosolic glutamine synthetase and not nitrate reductase from the green alga Chlamydomonas reinhardtii is phosphorylated and binds 14-3-3 proteins. Planta;212:264-269.

Prandl, R, Hinderhofer, K, Eggers-Schumacher, G, Schoffl, F. 1998 HSF3, a new heat shock factor from Arabidopsis thaliana, derepresses the heat shock response and confers thermotolerance when overexpressed in transgenic plants. Mol Gen Genet;258:269-278.

Prasher, D, McCann, R O, Cormier, M J. Cloning and expression of the cDNA coding for aequorin, a bioluminescent calcium-binding protein. Biochem Biophys Res Commun 1985;126(3):1259-1268.

Pratt, W B, Silverstein, A M, Galigniana, M D. 1999 A model for the cytoplasmic trafficking of signalling proteins involving the hsp90-binding immunophilins and p50cdc37. Cell Signal;11:839-851.

Premachandra, G S, Hahn, D T, Axtell, J D and Joly, R J. Epicuticular wax load and water-use efficiency on Bloomless and sparse-bloom mutants of Sorghum bicolor. L. Environmental and Experimental Botany 1994;34(3):293-301.

Preston, G M, Carroll, T P, Guggino, W B, and Agre, P. 1992 Appearance of water channels in Xenopus oocytes expressing red cell CHIP28 protein. Science;256:385-387.

Preuss, D. 1999 Chromatin silencing and Arabidopsis development: A role for polycomb proteins. Plant Cell;11:765-768.

Pringa, E, Martinez-Noel, G, Muller, U, Harbers, K. 2001 Interaction of the ring finger-related u-box motif of a nuclear dot protein with ubiquitin-conjugating enzymes. J Biol Chem;276:19617-19623.

Prioul, J L. 1992 Source-sink manipulations and carbohydrate metabolism in maize. Crop Science;32:751-756.

Prodromou, C, Siligardi, G, O'Brien, R, Woolfson, D N, Regan, L, Panaretou, B, Ladbury, J E, Piper, P W, Pearl, L H. 1999 Regulation of Hsp90 ATPase activity by tetratricopeptide repeat (TPR)-domain co-chaperones. EMBO J;18:754-762.

Provencher, Laurel Mezitt, Miao, Long, Sinha, Neelima and Lucas William J. 2001 Sucrose Export Defective 1 Encodes a Novel Protein Implicated in Chloroplast-to-Nucleus Signaling. Plant Cell, 2001;13(5):1127-1141.

Pysh, L D, Wysocka-Diller, J W, Camilleri, C, Bouchez, D, Benfey, P N. 1999 The GRAS gene family in Arabidopsis: sequence characterization and basic expression analysis of the SCARECROW-LIKE genes. Plant J;18:111-119.

Qin, M, Kuhn, R, Moran, S, Quail, P H. 1997 Overexpressed phytochrome C has similar photosensory specificity to phytochrome B but a distinctive capacity to enhance primary leaf expansion. Plant J;12:1163-1172.

Queitsch, C, Hong, S W, Vierling, E, Lindquist, S. 2000 Heat shock protein 101 plays a crucial role in thermotolerance in Arabidopsis. Plant Cell;12:479-492.

Quirino, B F, Reiter, W D, Amasino, R D. 2001 One of two tandem Arabidopsis genes homologous to monosaccharide transporters is senescence-associated. Plant Mol Biol;46:447-457.

Raghothama, K G. 1999 Phosphate Acquisition. Annu. Rev. Plant Physiol;50:665-693. Raines, C, Lloyd, J, Dyer, T. Review article. New insights into the structure and function of sedoheptulose-1,7-biphosphatase; an important but neglected Calvin cycle enzyme. J Experimental Botany 1999;50:1-8.

Raines, C A, Lloyd, J C, Willingham, N M, Potts, S, Dyer T A. cDNA and gene sequences of wheat chloroplast sedoheptulose-1,7-bisphosphatase homology with fructose-1,6-bisphosphatases. Eur J Biochem May 1, 1992;205(3):1053-1059.

Rajashekar, C B, Burke, M J. Freezing characteristics of rigid plant tissues: Development of cell tension during extracellular freezing. Plant Physiol 1996; 111: 597-603.

Rajashekar, C B, Lafta, A. 1996 Cell-wall changes and cell tension in repsonse to cold acclimation and exogenous abscisic acid in leaves and cell cultures. Plant Physiol;111:605-612.

Ramaratnam, N, Osawa, T, Namiki, M, Kawakishi, S. 1989 Chemical studies on novel rice hull antioxidants. 2. Identification of isovitexin, a C-glycosyl flavonoid. J. Agric. Food Chem;37:316-319.

Ramirez-Parra, E, Gutierrez, C. 2000 Characterization of wheat DP, a heterodimerization partner of the plant E2F transcription factor which stimulates E2F-DNA binding. FEBS Lett;486:73-78.

Ramirez-Parra, E, Xie, Q, Boniotti, M, and Gutierrez, C. 1999. The cloning of plant E2F, a retinoblastoma-binding protein reveals unique and conserved features with animal G₁/S regulators. Nucleic Acids Research;27(17):3527-3533.

Ramputh, A -I, Bown, A W. 1996 Rapid-aminobutyric acid synthesis and the inhibition of the growth and development of oblique-banded leaf-roller larvae. Plant Physiol; 111:1349-13 54.

Rao, M V, Lee, H, Creelman, R A, Mullet, J E, Davis, K R. 2000 Jasmonic Acid Signaling Modulates Ozone-Induced Hypersensitive Cell Death. Plant Cell;12:1633-1646.

Raskin, I, Turner, I M, Melander, W R. 1989 Regulation of heat production in the inflorescences of an Arum lily by endogenous salicyclic acid. Pro. Natl. Acad. Sci. USA;86:2214-2218.

Ratcliffe O J, Nadzan G C, Reuber T L, Riechmann J L. 2001 Regulation of flowering in Arabidopsis by an FLC homologue. Plant Physiol;126(1):122-132.

Rathinasabapathi, B, Burnet, M, Russell, B, Gage, D, Liao, P., Nyes, G, Scott, P, Golbeck, J and Hanson A. 1997. Choline momooxygenase, an unusual iron-sulfur enzyme catalyzing the first step of glycone betaine synthesis in plants: prosthetic group characterization and cDNA cloning. Proc. Natl. Acad. Sci;94:3454-3458.

Ravanko, K, Jarvinen, K, Paasinen-Sohns, A, Holtta, E. 2000 Loss of p27Kip1 from cyclin E/cyclin-dependent kinase (CDK) 2 but not from cyclin D1/CDK4 complexes in cells transformed by polyamine biosynthetic enzymes. Cancer Res;60:5244-5253.

Raven, J A. 1997 CO2-concentrating mechanisms: a direct role for thylakoid lumen acidification. Plant Cell and Environment;20:147-154.

Ravetch, J V, Horiuchi, K, Zinder, N D. Nucleotide sequences near the origin of replication of bacteriophage f1. Proc Natl Acad Sci USA October 1977;74(10):4219-4222.

Ravindra, R, Kunapuli, S P, Forman, L J, Nagele, R G, Foster, K A, Patel, S A. 1996 Effect of transient overexpression of Gq alpha on soluble and polymerized tubulin pools in GH3 and AtT-20 cells. J Cell Biochem;61:392-401.

Rea, P A 1999. MRP subfamily ABC transporters from plants and yeast. J.Exp.Bot;50, Special Issue:895-913.

Reddy, V S, Goud, K V, Sharma, R P, Reddy, A R. 1994 The ultraviolet-B responsive anthocyanin production is associated with a specific phase of phenylalanine ammonialyase biosynthesis. Plant Physiol;105:1059-1066.

Reed, Andrew, J, Below, Fred E., Hageman, Richard H. Grain protein accumulation and the relationship between leaf nitrate reductase and protease activities during grain development in maize (Zea mays L.). Plant Physiol. (1980);66:164-170.

Reed, J W, Nagpal, P, Poole, D S, Furuya, M, Chory, J. 1993 Mutations in the gene for the red/far-red light receptor phytochrome B alter cell elongation and physiological responses throughout Arabidopsis development. Plant Cell;5:147-157.

Regenberg, B, Villalba, J M, Lanfermeijer, F C, and Palmgren, M G. 1995 C terminal deletion analysis of plant plasma membrane H⁺-ATPase: Yeast as a model system for solute transport across the plant plasma membrane. Plant Cell;7:1655-1666.

Reichel, C, Mathur, J, Eckes, P, Langenkemper, K, Koncz, C, Schell, J, Reiss, B, Maas, C. Enhanced green fluorescence by the expression of an Aequorea victoria green fluorescent protein mutant in mono- and dicotyledonous plant cells. Proc Natl Acad Sci USA Jun. 11, 1996; ;93(12):5888-5893.

Reinbothe, C, Parthier, B, Reinbothe, S. 1997 Temporal pattern of jasmonate-induced alterations in gene expression of barley leaves. Planta;201:281-287.

Reintanz, B., Lehnen, M., Reichelt, M., Gershenzon, J., Kowalczyk, M., Sandberg, G., Godde, M., Uhl, R., and Palme, K. 2001. bus, a Bushy Arabidopsis CYP79F1 knockout mutant with abolished synthesis of short-chain aliphatic glucosinolates. Plant Cell;13:351-3674.

Reiss, B, Klemm, M, Kosak, H, Schel, J. 1996 RecA protein stimulates homologous recombination in plants. Proc Natl Acad Sci USA.;93:3094-3098.

Reiss, B, Schubert, I, Kopchen, K, Wendeler, E, Schell, J, Puchta, H. 2000 RecA stimulates sister chromatid exchange and the fidelity of double-strand break repair, but not gene targeting, in plants transformed by Agrobacterium. Proc Natl Acad Sci USA.;97:3358-3363.

Reuber, S, Bornman, J F, Weissenbock, G. 1996 Phenylpropanoid compounds in primary leaf tissues in rye (Secale cereale). Light response of their metabolism and the possible role in UV-B protection. Physiol. Plantarum;97:160-168.

Rhee, S K, Icho, T, Wickner, R B. 1989 Structure and nuclear localization signal of the SKI3 antiviral protein of Saccharomyces cerevisiae. Yeast;5:149-158.

Rhoads, D M, McIntosh, L. 1991 Isolation and characterization of a cDNA clone encoding an alternative oxidase protein of Sauromatum guttatum (Schott). Proc. Natl. Acad. Sci. USA;88:2122-2126.

Rhoads, D M, McIntosh, L. 1992 Salicyclic acid regulation of respiration in higher plants: alternative oxidase expression. Plant Cell;4:1131-1139.

Rhoads, D M, McIntosh, L. 1993 The salicyclic acid-inducible alternative oxidase gene aox1 and genes encoding pathogenesis-related proteins share regions of sequence similarity in their promoters. Plant Mol. Biol;21:615-624.

Ribas-Carbo, M, Wiskich, J T, Berry, J A, Siedow, J N. 1995 Ubiquinone redox behavior in plant mitochondria during electron transport. Arch. Biochem. Biophys;317:156-160.

Riccardi F, Gazeau P, de Vienne D, Zivy M. 1998 Protein changes in response to progressive water deficit in maize. Quantitative variation and polypeptide identification. Plant Physiol.;117:1253-1263.

Rice, Evans C A, Miller, N J, Paganga, G 1997. Antioxidant properties of phenolic compounds. Trends in Plant Science;2(4):152-159.

Richard, S, Drevet, C, Jouanin, L, Seguin, A. Isolation and characterization of a cDNA clone encoding a putative white spruce glycine-rich RNA binding protein. Genel999, 240(2):379-388.

Riedl, S J, Renatus, M, Schwarzenbacher, R, Zhou, Q, Sun, C, Fesik, S W, Liddington, R C, Salvesen, G S. 2001 Structural basis for the inhibition of caspase-3 by XIAP. Cell; 104:791-800.

Riesmeier, J W, Willmitzer, L, and Frommer, W B. 1992 Isolation and characterization of a sucrose carrier cDNA from spinach by functional expression in yeast. EMBO J;11:4705-4713.

Riesmeier, J W, Willmitzer, L, and Frommer, W B. 1994 Evidence for an essential role of the sucrose transporter in phloem loading and assimilate partitioning. EMBO J;13:1-7.

Rinke, J, Steitz, J A. 1982 Precursor molecules of both human 5S ribosomal RNA and transfer RNAs are bound by a cellular protein reactive with anti-La lupus antibodies. Cell; 29:149-159.

Riou-Khamlichi, C, Huntley, R, Jacqmard, A, Murray, J A. 1999 Cytokinin activation of Arabidopsis cell division through a D-type cyclin. Science;283:1541-1544.

Roberts, M R. 2000 Regulatory 14-3-3 protein-protein interactions in plant cells. Curr Opin Plant Biol 2000;3:400-405.

Roberts, M A, Wray, J L. 1996 Cloning and characterization of an Arabidopsis thaliana cDNA clone encoding an organellar isoform of serine acetyltransferase. Plant Mol Biol;30:1041-1049.

Robinson, L C, Tatchell, K. 1991 TFS1: a suppressor of cdc25 mutations in Saccharomyces cerevisiae. Mol Gen Genet;230:241-250.

Robson, P R, McCormac, A C, Irvine, A S, Smith, H. 1996 Genetic engineering of harvest index in tobacco through overexpression of a phytochrome gene. Nat Biotechnol;14:995-998.

Roessner, C A, Scott, A I. Fluorescence-based method for selection of recombinant plasmids. BioTechniques 1995;19(5):760-764.

Roitsch, T, Bittner, M, and Godt, D E. 1995 Induction of apoplastic invertase of Chenopodium rubrum by D-glucose and a glucose analog and tissue-specific expression suggest a role in sink-regulation. Plant Physiol;108:285-294.

Ross, K E, Kaldis, P, Solomon, M J. 2000 Activating phosphorylation of the Saccharomyces cerevisiae cyclin-dependent kinase, cdc28p, precedes cyclin binding. Mol Biol Cell;1:1597-1609.

Roth, D, Birkenfeld, J, Betz, H. 1999 Dominant-negative alleles of 14-3-3 proteins cause defects in actin organization and vesicle targeting in the yeast Saccharomyces cerevisiae. FEBS Lett.460:411-6.

Roth, G. 1998 High oil corn gets mixed reviews in 1997. Penn State. Department of Crop and Soil Sciences. Available at www.agronomy.psu.edu/Extension/CornManagement/articles/CA2.

Roychowdhury, S, Rasenick, M M. 1994 Tubulin-G protein association stabilizes GTP binding and activates GTPase: cytoskeletal participation in neuronal signal transduction. Biochemistry. 1994;33:9800-9805.

Rudt, F and Pieler, T. 1996 Cytoplasmic retention and nuclear import of 5S ribosomal RNA containing RNPs. EMBO J;15:1383-1391.

Ruegger, M, Dewey, E, Gray, W M, Hobbie, L, Turner, J, Estelle, M. 1998 The TIR1 protein of Arabidopsis functions in auxin response and is related to human SKP2 and yeast grr1p. Genes Dev;12:198-207.

Running, M P, Hake S. 2001 The role of floral meristems in patterning. Curr Opin Plant Biol. February 2001;4(1):69-74.

Rushton, P J et al. 1995 Members of a new family of DNA binding proteins bind to a conserved cis-elements in the promoters of alpha-Amy2 genes. Plant Mol. Biol;29:691.

Russell, B, Rathinasabapathi, and Hanson, A. 1998 Osmotic stress induces expression of choline monooxygenase in sugar beet and amaranth. Plant Physiology;116:859-865.

Russell, W A. 1991 Genetic improvement of maize yields. Adv. Agronomy;46:245-298.

Rutherford, S L, Lindquist, S. 1998 Hsp90 as a capacitor for morphological evolution. Nature;396:336-342.

Saalbach, G, Schwerdel, M, Natura, G, Buschmann, P, Christov, V, Dahse, I. 1997 Over-expression of plant 14-3-3 proteins in tobacco: enhancement of the plasmalemma K+ conductance of mesophyll cells. FEBS Lett:413:294-298.

Saibil, H. 2000 Molecular chaperones: containers and surfaces for folding, stabilising or unfolding proteins. Curr Opin Struct Biol;10:251-258.

Sairam, R. 1994 Effects of homobrassinolide application on plant metabolism and grain yield under irrigated and moisture-stress conditions of two wheat varieties. Plant Growth Regul;14:173-181.

Saisho, D, Nambara, E, Naifo, S, Tsufsumi, N, Hirai, A, Nakazono, M. 1997 Characterization of the gene family for alternative oxidase from Arabidopsis thaliana. Plant Mol.Biol;35:585-596.

Sakakibara, H, Takei, K, Sugiyama, T. Isolation and characterization of a cDNA that encodes maize uroporphyrinogen III methyltransferase, an enzyme involved in the synthesis of siroheme, which is prosthetic group of nitrite reductase. The Plant Journal November 1996;10(5):883-892.

Sakamato, A, Valverde, R, Alia, Chen T H and Murata, N. Transformation of Arabidopsis with the codA gene for choline oxidase enhances freezing tolerance of plants. Plant J. June 2000; 22(5):449-453.

Sakamoto, T, Kobayashi, M, Itoh, H, Tagiri, A, Kayano, T, Tanaka, H, Iwahori, S and Matsuoka, M. 2001 Expression of a Gibberellin 2-Oxidase gene around the shoot apex is related to phase transition in rice. Plant Physiology;125:1508-1516.

Sakurai, A, Fujioka, S. 1997 Studies on biosynthesis of brassinosteroids. Biosci. Biotechnol. Biochem;61:757-762.

Sales, K, Brandt, W, Rumbak, E, Lindsey, G. 2000 The LEA-like protein HSP 12 in Saccharomyces cerevisiae has a plasma membrane location and protects membranes against desiccation and ethanol-induced stress. Biochim Biophys Acta;1463:267-278.

Samach, A, Klenz, J E, Kohalmi, S E, Risseeuw, E, Haughn, G W, Crosby, W L. 1999 The UNUSUAL FLORAL ORGANS gene of Arabidopsis thaliana is an F-box protein required for normal patterning and growth in the floral meristem. Plant J.;20:433-445.

Sanchez, J P, Chua, N H. 2001 Arabidopsis plc1 is required for secondary responses to abscisic acid signals. Plant Cell.13:1143-1154.

Sanchez, P, de Torres, Zabala M, Grant, M. 2000 AtBI-1, a plant homologue of Bax inhibitor-1, suppresses Bax-induced cell death in yeast and is rapidly upregulated during wounding and pathogen challenge. Plant J;21:393-399.

Sanchez-Fernandez, R, Davies, T G, Coleman, J O, Rea, P A. 2001 The Arabidopsis thaliana ABC protein superfamily, a complete inventory. J Biol Chem;276:30231-30244.

Sanders, D, Brownlee, C, Harper, J F. 1999 Communicating with calcium. The Plant Cell;11:691-706.

Sarkar, G, Turner, R T, Bolander, M E. Restriction-site PCR: a direct method of unknown sequence retrieval adjacent to a known locus by using universal primers. PCR Methods Appl May 1993;2(4):318-322.

Sasaki, K, Abid, M R, Miyazaki, M. 1996 Deoxyhypusine synthase gene is essential for cell viability in the yeast Saccharomyces cerevisiae. FEBS Lett;384:151-154.

Sasse, J. 1990 Brassinolide-induced elongation and auxin. Physiol. Plant 80:401-408.

Sauer, N, Caspari, T, Klebl, F, Tanner, W. 1990a Functional expression of the Chlorella hexose transporter in S. pombe. Proc. Natl. Acad. Sci. USA;87:7949-7950.

Sauer, N, Friedlander, K, Graiml-Wicke, U. 1990b Primary structure, genomic organization and heterologous expression of a glucose transporter from A. thaliana. EMBO J;9:3045-3050.

Sauer, N, Stadler, R. 1993 A sink-specific H⁺/monosaccharide co-transporter from Nicotiana tabacum: Cloning and heterologous expression in baker's yeast. Plant J;4:601-610.

Sauer, N, Stolz, J. 1994 SUC1 and SUC2: Two sucrose transporters from Arabidopsis thaliana: Expression and characterization in baker's yeast and identification of the histidine-tagged protein. Plant J;6:67-77.

Sauer, N, Tanner, W. 1989 The hexose carrier from Chlorella. cDNA cloning of a eucaryotic H⁺-cotransporter. FEBS Lett;259:43-46.

Sauter, M. 1997 Differential expression of a CAK (cdc2-activating kinase)-like protein kinase, cyclins and cdc2 genes from rice during the cell cycle and in response to gibberellin. Plant J;11:181-190.

Savino, G, Briat, J -F, and Lobreaux, S. Inhibition of the Iron-induced ZmFer1 Maize Ferritin Gene Expression by Antioxidants and Serine/Threonine Phosphatase Inhibitors. J. Biol. Chem. 1997;272:33319-33326.

Scandalios, J G. 1990 Response of plant antioxidant defense genes to environment stress. Adv. Genet;28:1-41.

Scanlon, M J and Myers, A M. 1998 Phenotypic analysis and molecular cloning of discolored-1 (dsc1), a maize gene required for early kernel development. Plant Molecular Biology;37:483-493.

Schachtman, D P, Reid, R J, Ayling, S M. 1998 Phosphorus Uptake by Plants: From Soil to Cell. Plant Physiol;116:447-453.

Schaller, F, Biesgen, C, Mussig, C, Altmann, T. Weiler, E. 2000 12-Oxophytodienoate reductase 3 (OPR3) is the isoenzyme involved in jasmonate biosynthesis. Planta;210:979-984.

Schatz, O, Oft, M, Dascher, C, Schebesta, M, Rosorius, O, Jaksche, H, Dobrovnik, M, Bevec, D, Hauber, J. 1998 Interaction of the HIV-1 rev cofactor eukaryotic initiation factor 5A with ribosomal protein L5. Proc Natl Acad Sci USA;95:1607-1612.

Scheibe, R. 1991 Redox-modulation of chloroplast enzymes. A common principle for individual control. Plant Physiol;96:1-3.

Schimkat, D, Heineke, D, Heldt, H. 1990 Regulation of sedoheptulose-1,7-bisphosphatase by sedoheptulose-7-phosphate and glycerate, and of fructose-1,6-bisphosphatase by glycerate in spinach chloroplasts. Planta;181(1):97-103.

Schnall, R, Mannhaupt, G, Stucka, R, Tauer, R, Ehnle, S, Schwarzlose, C, Vetter, I, Feldmann, H. 1994 Identification of a set of yeast genes coding for a novel family of putative ATPases with high similarity to constituents of the 26S protease complex. Yeast;10:1141-1155.

Schneeberger, R G, Becraft, P W, Hake, S, Freeling, M. 1995 Ectopic expression of the knox homeo box gene rough sheathl alters cell fate in the maize leaf. Genes Dev;9:2292-2304.

Schnier, J, Schwelberger, H G, Smit-McBride, Z, Kang, H A, Hershey, J W. 1991 Translation initiation factor 5A and its hypusine modification are essential for cell viability in the yeast Saccharomyces cerevisiae. Mol Cell Biol. 1991;11:3105-3114.

Schopfer, C R, Nasrallah, M E, Nasrallah, J B. 1999 The male determinant of self-incompatibility in Brassica. Science;286:1697-1700.

Schrammeijer B, Risseeuw E, Pansegrau W, Regensburg-Tuink T J, Crosby W L, Hooykaas P J. 2001 Interaction of the virulence protein VirF of Agrobacterium tumefaciens with plant homologs of the yeast Skp1 protein. Curr Biol;11:258-262.

Schrick, K, Mayer, U, Horrichs, A, Kuhut, C, Bellini, C, Dangl, J, Schmitt, J, Jurgens, G. 2000 FACKEL is a sterol C-14 reductase required for organized cell division and expansion in Arabidopsis embryogenesis. Gen Dev;14:1471-1484.

Schroder, M, Chang, J S, Kaufman, R J. 2000 The unfolded protein response represses nitrogen-starvation induced developmental differentiation in yeast. Genes Dev;14(23):2962-2975.

Schroeder, Julian, Kwak, June and Allen, Gethyn. 2001 Guard cell abscisic acid signaling and engineering drought hardiness in plants. Nature;410:327-330.

Schule, T, Rose, M, Entian, K D, Thumm, M, Wolf, D H. 2000 Ubc8p functions in catabolite degradation of fructose-1,6-bisphosphatase in yeast. EMBO J;19:2161-2167.

Schuler, I, Milon, A, Nakatani, Y, Ourisson, G, Albrecht, A M, Benveniste, P, and Hartmann, M A. 1991 Differential effects of plant sterols on water permeability and on acyl chain ordering of soybean phosphatidylcholine bilayers. Proc. Natl. Acad. Sci;88: 6926-6930.

Schulman, B A, Carrano, A C, Jeffrey, P D, Bowen, Z, Kinnucan, E R, Finnin, M S, Elledge, S J, Harper, J W, Pagano, M, Pavletich, N P. 2000 Insights into SCF ubiquitin ligases from the structure of the Skp1-Skp2 complex. Nature;408:381-386.

Schultz, J, Copley, R R, Doerks, T, Ponting, C P, Bork, P. 2000 SMART: a web-based tool for the study of genetically mobile domains. Nucleic Acids Res;28:231-234.

Schultz, T F, Medina, J, Hill, A, Quatrano, R S. 1998 14-3-3 proteins are part of an abscisic acid-VIVIPAROUS1 (VP1) response complex in the Em promoter and interact with VP1 and EmBP1. Plant Cell;10:837-847.

Schumacher, K, Chory, J. 2000 Brassinosteroid signal transduction: still casting the actors. Curr. Opin. Plant Biol;3:79-84.

Schuinemann, D, Borchert, S, Fluigge, U I, Heldt, H W. 1993 ATP/ADP translocator from pea root plastids. Comparison with translocators from spinach chloroplasts and pea leaf mitochondria. Plant Physiol;103:131-137.

Schwacke, R., Grallath, S., Breitkreuz, K. E., Stransky, E., Stransky, H., Frommer, W. B., Rentsch, D., LeProT1, a transporter for proline, glycine betaine, and gamma-amino butyric acid in tomato pollen. Plant Cell (1999), March 11(3):377-392.

Schwartz, S H, Tan, B C, Gage, D A, Zeevaart, J A, McCarty, D R. 1997 Specific oxidative cleavage of carotenoids by VP14 of maize. Science;276(5320):1872-1874.

Schwechheimer, C, Deng, X W. 2000 The COP/DET/FUS proteins-regulators of eukaryotic growth and development. Semin Cell Dev Biol;11:495-503.

Schwechheimer, C, Serino, G, Callis, J, Crosby, W L, Lyapina, S, Deshaies, R J, Gray, W M, Estelle, M, Deng, X W. 2001 Interactions of the COP9 signalosome with the E3 ubiquitin ligase SCFTIRI in mediating auxin response. Science;292:1379-1382.

Schweizer, D B, Buchala, A, and Metraux, J P. 1997 Gene expression patterns and levels of jasmonic acid in rice treated with the resistance inducer 2,6-dichloro isonicotinic acid. Plant Physiol;115:61-70.

Searfoss, A M, Wickner, R B. 2000 3′ poly(A) is dispensable for translation. Proc Natl Acad Sci USA;97:9133-9137.

Sehnke, P C, Chung, H J, Wu, K, Ferl, R J. 2001 Regulation of starch accumulation by granule-associated plant 14-3-3 proteins. Proc Natl Acad Sci USA;98:765-770.

Sentoku, N, Sato, Y, Matsuoka, M. 2000 Overexpression of rice OSH genes induces ectopic shoots on leaf sheaths of transgenic rice plants. Dev Biol;220:358-364.

Seo, S, Sano, H, and Ohashi, Y. 1997 Jasmonic acid in wound signal transduction pathways. Physiol. Plant;101:740-745.

Seymour, R S. 1997 Plant That Warm Themselves. Sci. Amer;276:104-105.

Shakya, R, Sturm, A. 1998 Characterization of source- and sink-specific sucrose/H⁺ symporters from carrot. Plant Physiol;118:1473-1480.

Shalev, G, Sitrit, Y, Avivi-Ragolski, N, Lichtenstein, C, Levy, A A. 1999 Stimulation of homologous recombination in plants by expression of the bacterial resolvase ruvC. Proc Natl Acad Sci USA;96:7398-7402.

Shank K J, Su P, Brglez I, Boss W F, Dewey R E, Boston R S. 2001 Induction of lipid metabolic enzymes during the endoplasmic reticulum stress response in plants. Plant Physiol;126(1):267-277.

Sharkey, T D, Vassey, T L, Vanderveer, P J, Vierstra, R D. 1991 Carbon metabolism enzymes and photosynthesis in trangenic tobacco (Nicotiana tabacum L.) having excess phytochrome. Planta;185:287-296.

Shaw, R J, Wilson, J L, Smith, K T, Reines, D. 2001 Regulation of an IMP dehydrogenase gene and its overexpression in drug-sensitive transcription elongation mutants of yeast. J Biol Chem; 276(35)32905-32916.

Shaw, R H. 1983 Estimates of yield reductions in corn caused by water and temperature stress. In CD Ruper, P J Kramer, eds, Crop Relations to Water and Temperature Stress in Humid Temperate Climates. Westview Press, Boulder, CO 49-66.

Shaw, R H. 1988 Climate Requirements. In: Corn and corn improvement Spragure G F, Dudley J W eds. Pg 609-638.

Sheen, J, Hwang, S, Niwa, Y, Kobayashi, H, Galvraith, D W. Green-fluorescent protein as a new vital marker in plant cells. Plant J 1995 Nov;8(5):777-784.

Shelp, B J, Bown, A W, McLean, M D. 1999 Metabolism and functions of y-aminobutyric acid. Trends Plant Sci;41 :446-452.

Shen, B, Zheng, Z, Dooner, H K. 2000 A maize sesquiterpene cyclase gene induced by insect herbivory and volicitin: characterization of wild-type and mutant alleles. Proc Natl Acad Sci USA December 19;97(26):14807-14812.

Shen E C, Henry M F, Weiss V H, Valentini S R, Silver P A, Lee M S. 1998 Arginine methylation facilitates the nuclear export of hnRNP proteins. Genes Dev. 12:679-91.

Sheridan, W F, Clark, J K. 1993 Mutational analysis of morphogenesis of the maize embryo. Plant J;3:347-358.

Shi, X P, Yin, K C, Waxman, L. 1997 Effects of inhibitors of RNA and protein synthesis on the subcellular distribution of the eukaryotic translation initiation factor, eIF-5A, and the HIV-1 Rev protein. Biol Signals;6:143-149.

Shiba, T, Mizote, H, Kaneko, T, Nakajima, T, Kakimoto, Y. 1971 Hypusine, a new amino acid occurring in bovine brain. Isolation and structural determination. Biochim Biophys Acta;244:523-531.

Shibamoto, T, Hagiwara, Y, Hagiwara, H, Osawa, T. 1994 Flavonoid with strong antioxidative activity isolated from young barley leaves. Am. Chem. Soc Symp. Ser;547:153-163.

Shikanai, T, Takeda, T, Yamauch,i H, Sano, S, Tomizawa, K I, Yokota, A, Shigeoka, S. 1998 Inhibition of ascorbate peroxidase under oxidative stress in tobacco having bacterial catalase in chloroplasts. FEBS Lett;428:47-51.

Shinohara, A, Ogawa, T. 1999 Rad51/RecA protein families and the associated proteins in eukaryotes. Mutat Res; 435:13-21.

Shinomura, T, Uchida, K, Furuya, M. 2000 Elementary processes of photoperception by phytochrome A for high-irradiance response of hypocotyl elongation in Arabidopsis. Plant Physiol;122:147-156.

Shinozaki, K., and Yamaguchi-Shinozaki, K. 1997 Gene expression and signal transduction in water-stress response. Plant Physiol;115:327-334.

Shinozaki, K., Yamaguchi-Shinozaki, K. 2000 Molecular responses to dehydration and low temperature: differences and cross-talk between two stress signaling pathways. Curr. Opin. Plant Biol;3 :217-223.

Shirley, B W, Kubasek, W L, Storz, G, Bruggemann, E, Kepperneef, M, Ausubel, F M, Goodman, H M. 1995. Analysis of Arabidopsis mutants deficient in flavonoid biosynthesis. The Plant Journal;8(5):659-671.

Siedow, J N, Bickett, D M. 1981 Structural features required for inhibition of cyanide-insensitive electron transfer by propyl galate. Arch. Biochem. Biophys;207:32-39.

Siedow, J N, Umbach, A L, Moore, A L. 1995 The active site of the cyanide resistant oxidase from plant mitochondria contains a binuclear iron center. FEBS Lett;362:10-14.

Siedow, J N, Umbach, A L. 1995 Plant mitochondrial electron transfer and molecular biology. Plant Cell;7:821-831.

Siedow, J N, Whelan, J, Kearns, A, Wiskich, J T, Day, D A. 1992 Topology of the alternative oxidase in soybean mitochondria Molecular, Biochemical and Physiological Aspects of Plant Respiration (Amsterdam:Academic) pp. 19-27.

Silberstein, S, Collins, P G, Kellehe,r D J, Gilmore, R. 1995 The essential OST2 gene encodes the 16-kD subunit of the yeast oligosaccharyltransferase, a highly conserved protein expressed in diverse eukaryotic organisms. J Cell Biol;131:371-383.

Simons, B H, Millenaar, F F, Mulder, L, Van Loon, L C, Lambers, H. 1999 Enhanced expression and activation of the alternative oxidase during infection of Arabidopsis with Pseudomonal syringae pv tomato. Plant Physiol;120:529-538.

Singh, J, Nakamura, S, Ota, Y. 1993 Effect of epibrassinolide on gram (Cicer arietinum) plants grown under water stress in the juvenile stage. Indian J. Agric. Sci;63:395-397.

Sinha, N R, Williams, R E, Hake, S. 1993 Overexpression of the maize homeo box gene, KNOTTED-1, causes a switch from determinate to indeterminate cell fates. Genes Dev;7:787-795.

Sivamani, E, Bahieldinl, A, Wraith, J M, Al-Niemi, T, Dye, W E, Ho, T D, Qu, R. 2000 Improved biomass productivity and water use efficiency under water deficit conditions in transgenic wheat constitutively expressing the barley HVA1 gene. Plant Sci;155:1-9.

Sjolund, R D. 1997 The phloem sieve element: A river runs through it. Plant Cell;9:1137-1146.

Smith, F W, Ealing, P M, Dong, B, Delhaize, E. 1997 The cloning of two Arabidopsis genes belonging to a phosphate transporter family. Plant J;11: 83-92.

Snedden W. A. and Blumwald E. 2000 Alternative splicing of a novel diacylglycerol kinase in tomato leads to a calmodulin-binding isoform. Plant J;24:317-326.

Snedden, W A, Koutsia, N, Baum, G, Fromm, H. 1996 Activation of a petunia glutamate decarboxylase by calcium/calmodulin or by a monoclonal antibody which recognizes the calmodulin binding domain. J Biol Chem;271;4148-4153.

Snyder, B A, Nicholson, R L. 1990 Synthesis of phytoalexins in sorghum as a site specific response to fungal ingress. Science;248:1637.

Somers, D E, Schultz, T F, Milnamow, M, Kay, S A. 2000 ZEITLUPE encodes a novel clock-associated PAS protein from Arabidopsis. Cell;101:319-329.

Somerville, C. Direct tests of the role of membrane lipid composition in low-temperature-induced photoinhibition and chilling sensitivity in plants and cyanobacteria. Proc Natl Acad Sci USA Jul. 3, 1995; 92(14):6219-6225.

Somssich, I. E et al, 1989: Differential early activation of defense-related genes in elicitor-treated parsley cells. Plant Mol Biol;12:227-234.

Sonenberg, N, Newgard, C B. 2001 Protein synthesis. The perks of balancing glucose. Science;293(5531):818-819.

Sonoda, E, Sasaki, M S, Morrison, C, Yamaguchi-Iwai, Y, Takata, M, Takeda, S. 1999 Sister chromatid exchanges are mediated by homologous recombination in vertebrate cells. Mol Cell Biol;19:5166-5169.

Sowa, A W, Duff, S M G, Guy, P A, Hill, R D. 1998 Altering hemoglobin levels changes energy status in maize cells under hypoxia. Proc. Natl. Acad. Sci. USA;95:10317-10321.

Spear E, Ng D T. 2001 The unfolded protein response: no longer just a special teams player. Traffic;2(8):515-523.

Spencer, T M, O'Brien, J V, Start, W G, Adams, T R, Gordon-Kamm, W J, Lemaux, P G. Segregation of transgenes in maize. Plant Molecular Biology 1992;18:201-210.

Sperling, U, van Cleve, B, Frick, G, Apel, K, Armstrong, G A. 1997 Overexpression of light-dependent PORA or PORB in plants depleted of endogenous POR by far-red light enhances seedling survival in white light and protects against photooxidative damage. Plant J;12:649-658.

Stacey, M G, Kopp, O R, Kim, T H, von Arnim, A G. 2000 Modular domain structure of Arabidopsis COP1. Reconstitution of activity by fragment complementation and mutational analysis of a nuclear localization signal in planta. Plant Physiol;124:979-990.

Stacy, R A, Aalen, R B. 1998 Identification of sequence homology between the internal hydrophilic repeated motifs of group 1 late-embryogenesis-abundant proteins in plants and hydrophilic repeats of the general stress protein GsiB of Bacillus subtilis. Planta; 1998 206:476-478.

Stadler, R, Brandner, J, Schulz, A, Gahrtz, M, Sauer, N. 1995 Phloem loading by the PmSUC2 sucrose carrier from Plantago major occurs into companion cells. Plant Cell;7:1545-1554.

Stadler, R, Sauer, N. 1996 The Arabidopsis thaliana AtSUC2 gene is specifically expressed in companion cells. Bot. Acta; 109:299-308.

Stalker, D M, Malyj, L D, McBride, K E. Purification and properties of a nitrilase specific for the herbicide bromoxynil and corresponding nucleotide sequence analysis of the bxn gene. J Biol Chem May 5, 1988; 263(13):6310-6314.

Staswick, P E, Yuen, G Y, and Lehman, C C. 1998 Jasmonate signaling mutants of Arabidopsis are susceptible to the soil fungus Pythium irregulare. Plant J;15:747-754.

Steindler, C, Matteucci, A, Sessa, G, Weimar, T, Ohgishi, M, Aoyama, T, Morelli, G, Ruberti, I. 1999 Shade avoidance responses are mediated by the ATHB-2 HD-zip protein, a negative regulator of gene expression. Development;126:4235-4245.

Steitz, J A, Berg. C, Hendrick, J P, La Branche-Chabot, H, Metspalu, A, Rinke, J, and Yario, T. (1988) A 5S rRNA/L5 complex is a precursor to ribosome assembly in mammalian cells. J. Cell Biol;106.545-556.

Stemler, A J. 1997 The case of chloroplast thylakoid carbonic anhydrase. Physiol. Plantarum;99:348-353.

Steudle, E, and Henzler, T. 1995. Water channels in plants: do basic concepts of water transport change? Journal of Experimental Botany;46(290):1067-1076.

Stormo, G D. Gene-finding approaches for eukaryotes. Gene Res April 2000;10(4):394-397.

Subbaiah, C C, Bush, D S, Sacks, M M 1998 Mitochondrial contribution to the anoxic Ca2+ signal in maize suspension-cultured cells. Plant Physiol;118:759-771.

Subbaiah, C C, Sachs, M M. 2000 Maize cap1 encodes a novel SERCA-type calcium-ATPase with a calmodulin-binding domain. J.Bio.Chem;275 :21678-21687.

Sugano, S., Andronis, C., Green, R. M., Wang, Z -H. and Tobin, E. M.1998. Proc. Natl. Acad Sci. USA;95:11020-11025.

Sugano, S., Andronis, C., Ong, M. S., Green, R. M. and Tobin, E. M 1999. Proc. Natl. Acad Sci. USA;96:12362-12366.

Sugimoto, A, Hozak, R R, Nakashima, T, Nishimoto, T, Rothman, J H. 1995 dad-1, an endogenous programmed cell death suppressor in Caenorhabditis elegans and vertebrates. EMBO J;14:4434-4441.

Sugiyama, T, New, J H, Kowalczykowski, S C. 1998 DNA annealing by RAD52 protein is stimulated by specific interaction with the complex of replication protein A and single-stranded DNA. Proc Natl Acad Sci USA;95:6049-6054.

Sung han park, Su Hyun Park, Hang Gyeong Chin, Moo Je Cho, Robert A. Martienssen, and Chang-deok Han 2000 Inhibitor of striate conditionally suppresses cell proliferation in variegated maize. Genes and Development;14:1005-1016.

Sung, P, Trujillo, K M, Van Komen, S. 2000 Recombination factors of Saccharomyces cerevisiae. Mutat Res;451 :257-275.

Sutcliffe, J G. Nucleotide sequence of the ampicillin resistance gene of Escherichia coli plasmid pBR322. Proc Natl Acad Sci USA August 1978;75(8):3737-3741.

Swire-Clark, G A, Marcotte, W R. 1999 The wheat LEA protein Em functions as an osmoprotective molecule in Saccharomyces cerevisiae. Plant Mol Biol;39:117-128.

Synetos, D, Frantziou, C P, Alksne, L E. 1996 Mutations in yeast ribosomal proteins S28 and S4 affect the accuracy of translation and alter the sensitivity of the ribosomes to paromomycin. Biochim Biophys Acta;1309:156-166.

Szczegielniak, J, Liwosz, A, Jurkowski, I, Loog, M, Dobrowolska, G Y, E k, P, Harmon, A C, Muszynska, G. 2000 Calcium dependent protein kinase from maize seedlings activated by phospholipids. Eur. J. Biochem;267:3818-3827.

Sze, H, Li, X, Palmgren, M G. 1999 Energization of plant cell membranes by H+-pumping ATPases:regulation and biosynthesis. Plant Cell;11:677-689.

Ta, C T, Weiland, R T. Nitrogen partitioning in maize during ear development. Crop Science March-April1992:32(2):443-451.

Tacke, E, Korfhage, C, Michel, D, Maddaloni, M, Motto, M, Lanzini, S, Salamini, F, Doring, H P. Transposon tagging of the maize Glossy2 locus with the transposable element En/Spm. Plant J. December 1995;8(6):907-17. PMID: 8580961; UI: 96132147.

Takahashi, R, Deveraux, Q, Tamm, I, Welsh, K, Assa-Munt, N, Salvesen, G S, Reed, J C. 1998 A single BIR domain of XIAP sufficient for inhibiting caspases. J Biol Chem;273:7787-7790.

Takahashi, M, Nakanishi, H., Kawasaki, S, Nishizawa, N K, Mori, S. Enhanced tolerance of rice to low iron availability in alkaine soils using barley nicotianamine aminotransferase genes. Nature Biotechnology, 2001;19:466-469.

Takahashi, M, Yamaguchi, H, Nakanishi, H, Shioiri, T, Nishizawa, N, Mori, S. Cloning two genes for nicotianamine aminotransferase, a critical enzyme in iron acquisition (Strategy II) in graminaceous plants. Plant Physiol 1999; 121:947-956.

Takahashi, Y., Shomura, A., Sasaki, T. and Yano, M. 2001 Hd6, a rice quantitative trait locus involved in photoperiod sensitivity, encodes the alpha subunit of protein kinase CK2. Proc. Natl. Acad Sci. USA;98:7922-7927.

Takano, M, Kanegae, H, Shinomura, T, Miyao, A, Hirochika, H, Furuya, M. 2001 Isolation and characterization of rice phytochrome A mutants. Plant Cell; 13:521-534.

Takayama, S, Shiba, H, Iwano, M, Shimosato, H, Che, F S, Kai, N, Watanabe, M, Suzuki, G, Hinata, K, Isogai, A. 2000 The pollen determinant of self-incompatibility in Brassica campestris. Proc Natl Acad Sci USA; 97:1920-1925.

Takeuchi, Y, Ogasawara, M, Konnai, M, Takematsu, T. 1992 Application of brassinosteroids in agriculture in Japan. Proc. Plant Growth Regul. Soc. Am; 19:343-352.

Tan, S and Troxler, R. 1999 Characterization of two chloroplast RNA polymerase sigma factors from Zea mays: Photoregulation and differential expression. PNAS (USA); 96 :5316-5321.

Tanaka, H, Masuta, C, Uehara, K, Kataoka, J, Koiwai, A, Noma, M. 1997 Morphological changes and hypomethylation of DNA in transgenic tobacco expressing antisense RNA of the S-adenosyl-L-homocysteine hydrolase gene. Plant Mol Biol; 35:981-986.

Tanaka, Y, Makishima, T, Sasabe, M, Ichinose, Y, Shiraishi, T, Nishimoto, T, Yamada, T. 1997 dad-1, A putative programmed cell death suppressor gene in rice. Plant Cell Physiol;38:379-383.

Tanaka, A, Ito, H, Tanaka, R, Tanaka, N K. Chlorophyll a oxygenase (CAO) is involved in chlorophyll b formation from chlorophyll a. Proc. Natl.Acad.Sci USA Oct. 13, 1998; 95(21):12719-12723.

Tanaka, H, Masuta, C, Uehara, K, Kataoka, J, Koiwai, A, Noma, M. Morphological changes and hypomethylation of DNA in transgenic tobacco expressing antisense RNA of the S-adenosyl-L-homocysteine hydrolase gene. Plant Mol Biol December 1997;35(6):981-986.

Tanaka, N, Ogura, T, Noguchi, T, Hirano, H, Yabe, N, Hasunuma, K. 1998 Phytochrome-mediated light signals are transduced to nucleoside diphosphate kinase in Pisum sativum L. cv. Alaska. J Photochem Photobiol B; 45:113-121.

Tantikanjana, T, Yong, J. W. H., Letham, D. S., Griffith, M., Hussain, M., Ljung, K, Sandberg, G., and Sundaresan, V. 2001. Control of axillary bud initiation and shoot architecture in Arabidopsis through the SUPERSHOOT gene. Genes and Dev;15:1577-1588.

Tao, Y, Kassatly, R F, Cress, W D, Horowitz, J M. 1997 Subunit composition determines E2F DNA-binding site specificity. Mol Cell Biol;17:6994-7007.

Tavernier, E, Pugin, A. 1995 Transbilayer distribution of phosphathidylcholine and phosphathidylethanolamine in vacuolar membrane of Acer pseudoplatanus cells. Biochimie;77: 174-181.

Taylor, S, Hofer, J, Murfet, 1. 2001 Stamina pistilloida, the Pea Ortholog of Fim and UFO, Is Required for Normal Development of Flowers, Inflorescences, and Leaves. Plant Cell; 13:31-46.

Telfer, A, Dhami, S, Bishop, S M, Phillips, D, Barber, J. 1994 Beta-Carotene quenches singlet oxygen formed by isolated photosystem II reaction centers. Biochemistry;33:14469-14474.

Tenkouano, A, Miller, F R, Hart, G E, Frederiksen, R A, Nicholson, R. L 1993 Phytoalexin assay in juvenile Sorghum: An aid to breeding for anthracnose resistance. Crop Science;33:243-248.

Tepperman, J M, Zhu, T, Chang, H S, Wang, X, Quail, P H. 2001 Multiple transcription-factor genes are early targets of phytochrome A signaling. Proc Natl Acad Sci USA;98:9437-9442.

Tevini, M, Braun, J, Fieser, G. 1991 The protective function of the epidermal layer of rye seedlings against ultraviolet-B radiation. Photochem. and Photobiol;53:329-333.

Theodoulou, F L. 2000. Plant ABC transporters. Biochem. Biophys. Acta; 1465(1-2):79-103.

Thiele, A, Herold, M, Lenk, I, Quail, P H, Gatz, C. 1999 Heterologous expression of Arabidopsis phytochrome B in transgenic potato influences photosynthetic performance and tuber development. Plant Physiol; 120:73-82.

Thillet, J, Absil, J, Stone, S R, Pictet, R. Site-directed mutagenesis of mouse dihydrofolate reductase. Mutants with increased resistance to methotrexate and trimethoprim. J Biol Chem Sep. 5, 1988;263(25):12500-12508.

Thomas, C, Sun, Y, Naus, K, Lloyd, A, Roux, S. 1999 Apyrase Functions in Plant Phosphate Nutrition and Mobilizes Phosphate from Extracellular ATP. Plant Physiol;119:543-552.

Thomas, D J, Avenson, T J, Thomas, J B, Herbert, S K. 1998 A cyanobacterium lacking iron superoxide dismutase is sensitized to oxidative stress induced with methyl viologen but Is not sensitized to oxidative stress induced with norflurazon. Plant Physiol;116:1593-1602.

Thomas, D J, Thomas, J B, Prier, S D, Nasso, N E, Herbert, S K. 1999 Iron superoxide dismutase protects against chilling damage in the cyanobacterium synechococcus species PCC7942. Plant Physiol;120:275-282.

Thomas, S G, Phillips, A L, Hedden, P. Molecular cloning and functional expression of gibberellin 2-oxidases, multifunctional enzymes involved in gibberellin deactivation. Proc. Natl. Acad. Sci USA Apr. 13, 1999;96(8):4698-4703.

Thompson, C K, Movva, N R, Tizard, R, Crameri, R, Davies, J E, Lauwereys, M, Botterman, J. EMBO J 1987;6:2519-2623.

Thompson, J F, Stewart, C R, Morris, C J. 1966 Changes in amino acid content of excised leaves during incubation. I. The effect of water content of leaves and atmospheric oxygen level. Plant Physiol;41:1578-1582.

Thorsness, P E, White, K H, Fox T D. Inactivation of YME1, a member of the ftsH-SEC18-PAS 1-CDC48 family of putative ATPase-encoding genes, causes increased escape of DNA from mitochondria in Saccharomyces cerevisiae. Mol Cell Biol September 1993; 13(9):5418-5426.

Tian, Lining, Sequin, Armand, Charest. Expression of the green fluorescent protein gene in conifer tissues. Plant Cell Reports 1997;16(5):267-271.

Timmermans, M C, Hudson, A, Becraft, P W, Nelson, T. 1999 ROUGH SHEATH2: a Myb protein that represses knox homeobox genes in maize lateral organ primordia. Science;284: 151-153.

Timmermans, M C, Maliga, P, Vieira, J, Messing, J. The pFF plasmids: cassettes utilising CaMV sequences for expression of foreign genes in plants. J Biotechnol June 1990;14(3-4):333-344.

Tinland, B, Schoumacher, F, Gloeckler, V, Bravo-Angel, A M, Hohn, B. The Agrobacterium tumefaciens virulence D2 protein is responsible for precise integration of T-DNA into plant genome. EMBO Jounral 1995;14(14):3585-3595.

Tjaden, J, Mohlmann, T, Kampfenkel, K, Henrichs, G, Neuhaus, H E. (1998) Altered plastidic ATP/ADP-transporter activity influences potato (Solanum tuberosum) morphology, amount and composition of tuber starch, and tuber morphology. Plant J; 16:531-540.

Tjaden, J, Schwoppe, C, Mohlmann, T, Neuhaus, H E. 1998 Expression of the plastidic ATP/ADP transporter gene in Escherichia coli lead to the presence of a functional adenine nucleotide transport system in the bacterial cytoplasmic membrane. J. Biol. Chem;273:9630-9636.

Toh-E, A, Guerry, P, Wickner, R B. 1978 Chromosomal superkiller mutants of Saccharomyces cerevisiae. J Bacteriol;136:1002-1007.

Toh-E, A, Wickner, R B. 1980 “Superkiller” mutations suppress chromosomal mutations affecting double-stranded RNA killer plasmid replication in Saccharomyces cerevisiae. Proc Natl Acad Sci USA;77:527-530.

Tome, M E, Fiser, S M, Payne, C M, Gerne,r E W. 1997 Excess putrescine accumulation inhibits the formation of modified eukaryotic initiation factor 5A (eIF-5A) and induces apoptosis. Biochem J;328:847-854.

Tome, M E, Gerner, E W. 1996 Hypusine modification in eukaryotic initiation factor 5A in rodent cells selected for resistance to growth inhibition by ornithine decarboxylase-inhibiting drugs. Biochem J;320:55-60.

Tomitani, A, Okada, K, Miyashita, H, Matthijs, H C, Ohno, T, Tanada, A. Chlorophyll b and phycobilins in the common ancestor of cyanobacteria and chloroplasts. Nature July 8, 1999; 400(6740):159-162.

Tongaonkar, P, Chen, L, Lambertson, D, Ko, B, Madura, K. 2000 Evidence for an interaction between ubiquitin-conjugating enzymes and the 26S proteasome. Mol CellBiol; 20:4691-4698.

Topham, M. K. and Prescott, S. M. 1999 Mammalian diacylglycerol kinases, a family of lipid kinases with signaling functions. J. Biol. Chem;274:11447-11450.

Topham, M. K., Bunting, M., Zimmerman, G. A. McIntyre, T. M. Blackshear, P. J. and Prescott, S. M. (1998) Protein kinase C regulates the nuclear localization of diacylglycerol kinase-zeta. Nature;394:697-700.

Torii, K U, Mitsukawa, N, Oosumi, T, Matsuura, Y, Yokoyama, R, Whittier, R F, Komeda, Y. 1996 The Arabidopsis ERECTA gene encodes a putative receptor protein kinase with extracellular leucine-rich repeats. Plant Cell;8:735-746.

Toroser, D, Athwal, G S, Huber, S C. 1998 Site-specific regulatory interaction between spinach leaf sucrose-phosphate synthase and 14-3-3 proteins. FEBS Lett;435:110-114.

Toyn, J H, Araki, H, Sugino, A,. Johnston, L H. 1991 The cell-cycle-regulated budding yeast gene DBF2, encoding a putative protein kinase, has a homologue that is not under cell cycle control. Gene;104:63-70.

Tozawa et. al. 1998 Nuclear encoding of a plastid a factor in rice and its tissue- and light-dependent expression. Nucleic Acid Res; 26:415-419.

Travers, K J, Patil, C K, Wodicka, L, Lockhart, D J, Weissman, J S, Walter, P. 2000 Functional and genomic analyses reveal an essential coordination between the unfolded protein response and ER-associated degradation. Cell;101(3):249-58.

Tripp, M L, Bouchard, R A, Pinon, R. 1989 Cloning and characterization of NSP1, a locus encoding a component of a CDC25-dependent, nutrient-responsive pathway in Saccharomyces cerevisiae. Mol Microbiol;3:1319-1327.

Trotochaud, A E, Jeong, S, Clark, S E. 2000 CLAVATA3, a multimeric ligand for the CLAVATA1 receptor-kinase. Science;289:613-617.

Truernit, E, Sauer, N. 1995 The promoter of the Arabidopsis thaliana SUC2 sucrose-H⁺ symporter gene directs expression of β-glucuronidase to the phloem: Evidence for phloem loading and unloading by SUC2. Planta;196:564-570.

Trull, M C, Deikman, J. An Arabidopsis mutant missing one acid phosphatase isoform. Planta November 1998;206(4):544-550.

Tsiantis, M, Schneeberger, R, Golz, J F, Freeling, M, Langdale, J A. The maize rough sheath2 gene and leaf development programs in monocot and dicot plants. Science 1999;284:154-156.

Tsonev, T D, Lazova, G N, Stoinova, Z G, Popova, L P. A Possible Role for Jasmonic Acid Adaptation of Barley Seedlings to Salinity Stress. J. Plant Growth Regul. 1998 Summer;17(3):153-159.

Tsuge, T, Inagaki, N, Yoshizumi, T, Shimada, H, Kawamoto, T, Matsuki, R, Yamamoto, N, Matsui, M. 2001 Phytochrome-mediated control of COP1 gene expression in rice plants. Mol Genet Genomics;265:43-50.

Tubbe, A, Buckhout, T J. 1992 In vitro analysis of the H⁺-hexose symporter on the plasma membrane of sugarbeets (Beta vulgaris L.). Plant Physiol;99:945-951.

Turano, F J, Fang, T K. 1998 Characterization of two glutamate decarboxylase cDNA clones from Arabidopsis thaliana. Plant Physiol;117:1411-1421.

Turlings, T C, Fritzsche, M E. 1999 Attraction of parasitic wasps by caterpillar-damaged plants. Novartis Found Symp. 1999;223:21-32.

Turlings, T C J, Scheepmaker, J W A, Vet, L E M, Tumlinson, J H. & Lewis, W J J. How contact foraging experiences affect preferences for host-related odors in the larval parasitoid Cotesia marginiventris (Cresson) (Hymenopters: Braconidae). J Chemical Ecology May 1990; 16(5):1577-1589.

Ueguchi-Tanaka, M, Fujisawa, Y, Kobayashi, M, Ashikari, M, Iwasaki, Y, Kitano, H, Matsuoka, M. 2000 Rice dwarf mutant d1, which is defective in the alpha subunit of the heterotrimeric G protein, affects gibberellin signal transduction. Proc Natl Acad Sci USA;97:11638-11643.

Ugaki, M, Ueda, T, Timmermans, M C, Vieira, J, Elliston, K O, Messing, J. Replication of a geminivirus derived shuttle vector in maize endosperm cells. Nucl Acids Res 1991;19:371-377.

Umbach, A L, Siedow, J N. 1993 Covalent and noncovalent dimers of the cyanide-resistant alternative oxidase protein in higher plant mitochondria and their relationship to enzyme activity. Plant Physiol;103:845-854.

Umeda, M, Bhalerao, R P, Schell, J, Uchimiya, H, Koncz, C. 1998 A distinct cyclin-dependent kinase-activating kinase of Arabidopsis thaliana. Proc Natl Acad Sci USA;95:5021-5026.

Umeda, M, Umeda-Hara, C, Yamaguchi, M, Hashimoto, J, Uchimiya, H. 1999 Differential expression of genes for cyclin-dependent protein kinases in rice plants. Plant Physiol;119:31-40.

Urao, T B, Yakubov, R, Satoh, K, Yamaguchi-Shinozaki, M, Seki, T, Hirayama, and K. Shinozaki, A. Transmembrane Hybrid-Type Histidine Kinase in Arabidopsis Functions as an Osmosensor. The Plant Cell;11, 1999:1743-1754.

Uren, A G, Beilharz, T, O'Connell, M J, Bugg, S J, van Driel, R, Vaux, D L, Lithgow, T. 1999 Role for yeast inhibitor of apoptosis (IAP)-like proteins in cell division. Proc Natl Acad Sci USA; 96:10170-10175.

Uren, A G, O'Rourke, K, Aravind, L A, Pisabarro, M T, Seshagiri, S, Koonin, E V, Dixit, V M. 2000 Identification of paracaspases and metacaspases: two ancient families of caspase-like proteins, one of which plays a key role in MALT lymphoma. Mol Cell;6:96-97.

Vaarala, M H, Porvari, K, Kyllonen, A, Vihko, P. 2000 Differentially expressed genes in two LNCaP prostate cancer cell lines reflecting changes during prostate cancer progression. Lab Invest;80: 1259-1268.

Vaidyanathan, R, Kuruvilla, S, Thomas, G. 1999 Characterization and expression pattern of an abscisic acid and osmotic stress responsive gene from rice. Plant Science;140:21-30.

van Bel, A J E. 1993 Strategies of phloem loading. Annu. Rev. Plant Physiol. Plant Mol. Biol; 44:253-281.

Van Breusegem, F, Slooten, L, Stassart, J M, Moens, T, Botterman, J, Van Montagu, M, Inze, D. 1999 Overproduction of Arabidopsis thaliana FeSOD confers oxidative stress tolerance to transgenic maize. Plant Cell Physiol;40:515-523.

Van Camp, W, Capiau, K, Van Montagu, M, Inze, D, Slooten, L. 1996 Enhancement of oxidative stress tolerance in transgenic tobacco plants overproducing Fe-superoxide dismutase in chloroplasts. Plant Physiol;112:1703-1714.

van den Wijngaard P W, Bunney T D, Roobeek I, Schonknecht G, de Boer A H. 2000 Slow vacuolar channels from barley mesophyll cells are regulated by 14-3-3 proteins. FEBS Lett;488:100-104.

Van der Knaap, E, Kim, J H, Kende, H. 2000 A novel gibberellin-induced gene from rice and its potential regulatory role in stem growth. Plant Physiology;122:695-704.

Van Dyck, E, Stasiak, A Z, Stasiak, A, West, S C. 1999 Binding of double-strand breaks in DNA by human Rad52 protein. Nature;398:728-731.

van Heeswijk, W C, Wen, D, Clancy, P, Jaggi, R, Ollis, D L, Westerhoff, H V, Vasudevan, S G. 2000 The Escherichia coli signal transducers PII (GlnB) and GlNK form heterotrimers in vivo: fine tuning the nitrogen signal cascade. Proc Natl Acad Sci USA; 97(8):3942-3947.

van Heusden, G P, Griffiths, D J, Ford, J C, Chin-A-Woeng, T F, Schrader, P A, Carr, A M, Steensma, H Y. 1995 The 14-3-3 proteins encoded by the BMH1 and BMH2 genes are essential in the yeast Saccharomyces cerevisiae and can be replaced by a plant homologue. Eur J Biochem;229:45-53.

van Himbergen, J. A. J., ter Riet, B., Meijer, H. J. G., van den Ende, H., Musgrave, A., Munnik, T. 1999 Mastoparan analogues activate phospholipase C- and phospholipase D activity in Chlamydomonas: a comparative study. J. Exp. Bot;50:1735-1742.

van Hoof, A, Staples, R R, Baker, R E, Parker, R. 2000 Function of the ski4p (Csl4p) and ski7p proteins in 3′-to-5′ degradation of mRNA. Mol Cell Biol;20:8230-8243.

Vanlerberghe, G C, Day, D A, Wiskich, J T, Vanlerberghe, A E, McIntosh, L. 1995 Alternative oxidase activity in tobacco leaf mitochondria: dependence on tricarboxylic acid cycle-mediated redox regulation and pyruvate activation. Plant Physiol;109:353-361.

Vanlerberghe, G C, McIntosh, L. 1994 Mitochondrial electron transport regulation of nuclear gene expression: studies with the alternative gene of tobacco. Plant Physiol;105:867-874.

Vanlerberghe, G C, McIntosh, L. 1999 Signals regulating the expression of the nuclear gene encoding alternative oxidase of plant mitochondria. Plant Physiol;111:589-595.

Veena, Reddy, V S, Sopory, S K. 1999 Glyoxalase I from Brassicajuncea: molecular cloning, regulation and its over-expression confer tolerance in transgenic tobacco under stress. Plant J;17:385-395.

Venema, J, Tollervey, D. 1996 RRP5 is required for formation of both 18S and 5.8S rRNA in yeast. EMBO J;15:5701-15714.

Vercesi, A E, Martins, I S, Silva, M A P, Leite, H M F, Cuccovia, I M, Chaimovich, H. PUMPing plants. Nature May 4, 1995;375:24.

Vincent, A, Liebman, S W. 1992 The yeast omnipotent suppressor SUP46 encodes a ribosomal protein which is a functional and structural homolog of the Escherichia coli S4 ram protein. Genetics;132:375-386.

Vollbrecht, E, Veit, B, Sinha, N, Hake, S 1991 The developmental gene Knotted-1 is a member of a maize homeobox gene family. Nature;350:241-243.

Von Schaewen, A, Stitt, M, Schmidt, R, Sonnewald, U, Willmitzer, L. Expression of a yeast-derived invertase in the cell wall of tobacco and Arabidopsis plants leads to accumulation of carbohydrate and inhibition of photosynthesis and strongly influences growth and phenotype of transgenic tobacco plants. EMBO J October 1990;9(10):3033-3044.

Wagner, A M. 1995 A role for active oxygen species as second messengers in the induction of alternative oxidase gene expression in Petunia hybrida cells. FEBS Lett;368:339-342.

Wagner, A M, Moore, A L. 1997 Structure and function of the plant alternative oxidase: its putative role in the oxygen defence mechanism. Biosci. Rep;17:319-333.

Walker, A R, Davison, P A, Bolognesi-Winfield, A C, James, C M, Srinivasan, N, Blundell, T L, Esch, J J, Marks, M D, Gray, J C. (1999) The TRANSPARENT TESTA GLABRA1 locus, which regulates trichome differentiation and anthocyanin biosynthesis in Arabidopsis, encodes a WD40 repeat protein. Plant Cell;11:1337-50.

Wallace, W, Secor, J, Schrader, E 1984 Rapid accumulation of -aminobutyric acid and alanine in soybean leaves in response to an abrupt transfer to lower temperature, darkness or mechanical manipulation. Plant Physiol;75:170-175.

Walsh, J, Freeling, M. 1999 The liguleless2 gene of maize functions during the transition from the vegetative to the reproductive shoot apex. Plant J;19:489-495.

Wang, X Z, Harding, H P, Zhang, Y, Jolicoeur, E M, Kuroda, M, Ron, D. 1998 Cloning of mammalian Irel reveals diversity in the ER stress responses. EMBO J;17(19):5708-5717.

Wang, Z Y, Kenigsbuch, D, Sun, L, Harel, E, Ong, M S, Tobin, E M. 1997 A Myb-related transcription factor is involved in the phytochrome regulation of an Arabidopsis Lhcb gene. Plant Cell;9:491-507.

Wang et al., Molecular and Cellular Biology, August 1992, p. 3399-3406

Wang, B, Zeng, G. 1993 Effect of epibrassinolide on the resistance of rice seedlings to chilling injury. Zhiwu Shengli Xuebao,19:53-60.

Wang, C Y, Buta, J G 1994 Methyl jasmonate reduces chilling injury in Cucurbita pepo through its regulation of abscisic acid and polyamine levels. Environ Exp Bot;34:427-432.

Wang, J, Zhang, H, Allen, R D. 1999 Overexpression of an Arabidopsis peroxisomal ascorbate peroxidase gene in tobacco increases protection against oxidative stress. Plant Cell Physiol;40:725-732.

Wang, N, Yan, K, Rasenick, M M. 1990 Tubulin binds specifically to the signal-transducing proteins, Gs alpha and Gi alpha 1. J Biol Chem;265:1239-1242.

Wang, Z Y, Seto, H, Fujioka, S, Yoshida, S, Chory, J. 2001 BRI1 is a critical component of a plasma-membrane receptor for plant steroids. Nature. 2001;410:380-383.

Ward, J, Kuhn, C, Tegeder, M, and Frommer, W B. 1998 Sucrose transport in plants. Int. Rev. Cytol;178:41-71.

Wardrop, A J, Wicks, R E, Entsch, B. Occurrence and expression of members of the ferritin gene family in cowpeas. Biochemical Journal Feb. 1, 1999;337(pt.3):523-530.

Warren, M J, Bolt, E L; Rossner, C A; Scott, A I; Spenser, J B; Woodcock, S C. Gene dissection demonstrates that the Escherichia coli cysG gene encodes a multifunctional protein. BioChemical Journal 1994;302(Pt 3):837-844.

Wasternack, C, Parthier, B. 1997 Jasmonate signalled plant gene expression. Trends Plant Sci;3:302-307.

Watanabe, T, Sakai, S. 1998 Effects of active oxygen species and methyl j asmonate on expression of the gene for a wound-inducible 1-aminocyclopropane-1-carboxylate synthase in winter squash (Cucurbita maxima). Planta;206:570-576.

Weaver et al. A comparison of the expression patterns of several senescence-associated genes in response to stress and hormone treatment. Plant Mol Biol. 1998,37:455-469

Weber, E J. 1987 Lipids of the Kernel. Pages 311-349 in: Corn: Chemistry and Technology. Watson and Ramstad ed. Am. Assoc. of Cereal Chem, Inc., St. Paul, Minn.

Weber, H, Borisjuk, L, Heim, U, Sauer, N, Wobus, U. 1997 A role for sugar transporters during seed development: Molecular characterization of a hexose and a sucrose carrier in fava bean seeds. Plant Cell;9:895-908.

Weber, K L, Bolander, M E, Sarkab, G. Rapid acquisition of unknown DNA sequence adjacent to a known segment by multiplex restriction site PCR. Biotechniques Sep. 1998;25(3):415-419.

Wegrzyn T et al. 2000 A novel alpha amylase gene is transiently upregulated during low temperature exposure in apple fruit. Eur. J. Biochem;267:1313.

Weige, I D, Alvarez, J, Smyth, D R, Yanofsky, M F, Meyerowitz, E M. 1992 LEAFY controls floral meristem identity in Arabidopsis. Cell;69:843-859.

Weise, A, Barker, L, Kuhn, C, Lalonde, S, Buschmann, H, Frommer, W, Ward, J, 2000 A New Subfamily of Sucrose Transporters, SUT4, with Low Affinity/High Capacity Localized in Enucleate Sieve Elements of Plants. Plant Cell;12:1345-1355.

Weiss, C A, Huang, H, Ma, H. 1993 Immunolocalization of the G protein alpha subunit encoded by the GPA1 gene in Arabidopsis. Plant Cell;5:1513-1528.

Wendler, W M, Kremmer, E, Forster, R, Winnacker, E L. 1997 Identification of pirin, a novel highly conserved nuclear protein. J Biol Chem;272:8482-8489.

Weretilnyk, E A, Alexander, K J, Drebenstedt, M, Snider, J D, Summers, P S, Moffatt, B A. 2001. Maintaining methylation activities during salt stress. the involvement of adenosine kinase. Plant Physiol;125:856-865.

Werstuck, G H, Lentz, S R, Dayal, S, Hossain, G S, Sood, S K, Shi, Y Y, Zhou, J, Maeda, N, Krisans, S K, Malinow, M R, Austin, R C. 2001 Homocysteine-induced endoplasmic reticulum stress causes dysregulation of the cholesterol and triglyceride biosynthetic pathways. J Clin Invest;107(10):1263-1273.

Whelan, J, Millar, A H, Day, D A. 1996 The alternative oxidase is encoded in a multigene family in soybean. Planta;198:197-201.

Wickner, R B. 1996 Double-stranded RNA viruses of Saccharomyces cerevisiae. Microbiol Rev;60:250-265.

Wildt, S, & Deuschle, U. cobA, a red fluorescent transcriptional reporter for Escherichia coli, yeast, and mammalian cells. Nature Biotechnology, December 1999;17(12):1175-1178.

Wilhelm, E P, Mullen, R E, Keeling, P L, and Singletary, G W 1999 Heat stress during grain filling in maize: Effects on kernel growth and metabolism. Crop Sci;39:1733-1741.

Wilkinson, M, de Andrade Silva, E, Zachgo, S, Saedler, H, Schwarz-Sommer, Z. 2000 CHORIPETALA and DESPENTEADO: general regulators during plant development and potential floral targets of FIMBRIATA-mediated degradation. Development;127:3725-3734.

Williams, R. L., and Katan, M. 1996 Structural views of phosphoinositide-specific phospholipase C: Signalling the way ahead. Structure;4:1387-1394

Willingham, N M, Lloyd, J C, Raines, C A. Molecular cloning of the Arabidopsis thaliana sedoheptulose-1,7-biphosphatase gene and expression studies in wheat and Arabidopsis thaliana. Plant Mol Biol November 1994;26(4):1191-1200.

Wilmink, A, Dons, J J M. Selective agents and marker genes for use in transformation of monocotyledonous plants. Plant Molecular Biology Reporter 1993;11(2):165-185.

Wingender, E, Chen, X, et al. TRANSFAC: an integrated system for gene expression regulation. Nucleic Acids Res;28:316-319 (2000).

Winkler, H H, Neuhous, H E. Non-mitochondrial ATP transport. Trends Biochem Sci February 1999; 24(2):64-68.

Wise, A, Thomas, P G, White, I R, Millner, P A. 1994 Isolation of a putative receptor from Zea mays microsomal membranes that interacts with the G-protein, GP alpha 1. FEBS Lett;356:233-237.

Wohl, T, Klier, H, Ammer, H, Lottspeich, F, Magdolen, V. 1993 The HYP2 gene of Saccharomyces cerevisiae is essential for aerobic growth: characterization of different isoforms of the hypusine-containing protein Hyp2p and analysis of gene disruption mutants. Mol Gen Genet;241:305-311.

Wojtaszek, P. 2000 Nitric oxide in plants to NO or not to NO. Phytochemistry;54:1-4.

Wolf, M J et al. 1972. Maize with multilayer aleurone of high protein content. Crop Sci 12:440-442

Woo, E J, Dunwell, J M, Goodenough, P W, Marvier, A C, Pickersgill, R W. 2000 Germin is a manganese containing homohexamer with oxalate oxidase and superoxide dismutase activities. Nat Struct Biol;7:1036-1040.

Woo, H R, Chung, K M, Park, J H, Oh, S A, Ahn, T, Hong, S H, Jang, S K, Nam, H G. 2001 ORE9, an F-Box Protein That Regulates Leaf Senescence in Arabidopsis. Plant Cell;13:1779-1790.

Woodrow, I E. Sedpheptulose-1,7-bisphosphatase from wheat chloroplasts. Methods Enzymol 1982;90(Pt E):392-396.

Wright, S Y, Suner, M M, Bell, P J, Vaudin, M, Greenland, A J. Isolation and characterization of male flower cDNAs from maize. Plant J January 1993;3(1):41-49.

Wu, J, and Seliskar, DM. 1998 Salinity adaptation of plasma membrane H+-ATPase in the salt marsh plant Spartina patens: ATP hydrolysis and enzyme kinetics. J. Exp. Bot;49: 1005-1013.

Wu, K, Lu, G, Sehnke, P, Fer,l R J. 1997 The heterologous interactions among plant 14-3-3 proteins and identification of regions that are important for dimerization. Arch Biochem Biophys;339:2-8.

Wu, T C, Lichten, M. 1994 Meiosis-induced double-strand break sites determined by yeast chromatin structure. Science;263 :515-518.

Wu, W H, Assmann, S M. 1994 A membrane-delimited pathway of G-protein regulation of the guard-cell inward K+ channel. Proc Natl Acad Sci USA;91 :6310-6314.

Xiao, B, Smerdon, S J, Jones, D H, Dodson, G G, Soneji, Y, Aitken, A, Gamblin, S J. 1995 Structure of a 14-3-3 protein and implications for coordination of multiple signalling pathways. Nature;376:188-191.

Xiao, W, Kang, S G, Jang, J -C. 2001 The role of F-box protein AtFBL3 in sugar signal transduction in Arabidopsis thaliana. 12^{th} International Conference on Arabidopsis Research, abstract 159.

Xie, D X, Feys, B F, James, S, Nieto-Rostro, M, and Turner, J G. COI1: An Arabidopsis gene required for jasmonate-regulated defense and fertility. Science. May 15, 1998;280(5366):1091-1094.

Xie, Q, Suarez-Lopez, P, and Gutierrez, C. 1995 Identification and analysis of a retinoblastoma binding motif in a replication protein of a plant DNA virus: requirement for efficient viral DNA replication. EMBO Journal. August 15;14(16): 4073-4082.

Xie, Y, Varshavsky, A. 2000 Physical association of ubiquitin ligases and the 26S proteasome. Proc Natl Acad Sci USA;97:2497-2502.

Xin, Z, Browse, J. Eskimo 1 mutants of Arabidopsis are constitutively freezing-tolerant. Proc Natl Acad Sci USA Jun. 23, 1998;95(13):7799-7804.

Xing, W, Rajashekar, C B, Glycine betaine involvement in freezing tolerance and water stress in Arabidopsis thaliana. Environ. Expt. Bot. August 2001,46(1):21-28

Xu, W, He, Y, Wang, Y, Zhao, Y. 1994 Preliminary study of brassinosterome binding sites from mung bean epicotlyls. Acta. Phytophysiol. Sin;20:298-302.

Xu, X, Dietrich, C R, Delledonne, M, Xia, Y, Wen, T J, Robertson, D S, Nikolau, B J, Schnable, P S. Sequence analysis of the cloned glossy8 gene of maize suggests that it may code for a beta-ketoacyl reductase required for the biosynthesis of cuticular waxes. Plant Physiol October 1997; 115(2):501-510.

Yaffe, M B, Rittinger, K, Volinia, S, Caron, P R, Aitken, A, Leffers, H, Gamblin, S J, Smerdon, S J, Cantley, L C. 1997 The structural basis for 14-3-3:phosphopeptide binding specificity. Cell; 91:961-971.

Yamada, T, Ohta, H, Shinohara, A, Iwamatsu, A, Shimada, H, Tsuchiya, T, Masuda, T, Takamiya, K. 2000 A cysteine protease from maize isolated in a complex with cystatin. Plant Cell Physiol. February 2000;41(2):185-191.

Yamaguchi, M, Umeda, M, Uchimiya, H. 1998 A rice homolog of Cdk7/MO15 phosphorylates both cyclin-dependent protein kinases and the carboxy-terminal domain of RNA polymerase II. Plant J;16:613-619.

Yamaguchi, M, Fabian, T, Sauter, M, Bhalerao, R P, Schrader, J, Sandberg, G, Umeda, M, Uchimiya, H. 2000 Activation of CDK-activating kinase is dependent on interaction with H-type cyclins in plants. Plant J;24: 11-20.

Yamakawa, S, Matsubayashi, Y, Sakagami, Y, Kamada, H, Satoh, S. 1999 Promotive effects of the peptidyl plant growth factor, phytosulfokine-alpha, on the growth and chlorophyll content of Arabidopsis seedlings under high night-time temperature conditions. Biosci Biotechnol Biochem;63 :2240-2243.

Yamamoto, T., Lin, H., Sasaki, T and Yano, M. 2000. Identification of heading date quantitative trait locus Hd6 and characterization of its epistatic interactions with Hd2 in rice using advanced backcross progeny. Genetics,154(2):885-891.

Yamamuro, C, Ihara, Y, Wu, X, Noguchi, T, Fujioka, S, Takatsuto, S, Ashikari, M, Kitano, H, Matsuoka, M. 2000 Loss of Function of a Rice brassinosteroid insensitive I Homolog Prevents Internode Elongation and Bending of the Lamina Joint. Plant Cell;12:1591-1606.

Yamanaka, K, Tanaka, M, Tsutsui, H, Kupper, T S, Asahi, K, Okamura, H, Nakanish,i K, Suzuki, M, Kayagaki, N, Black, R A, Miller, D K, Nakashima, K, Shimizu, M, Mizutani, H. 2000 Skin-specific caspase-1-transgenic mice show cutaneous apoptosis and pre-endotoxin shock condition with a high serum level of IL-18. J Immunol;165:997-1003.

Yamasaki, H, Sakihama, Y. 2000 Simultaneous production of nitric oxide and peroxynitrite by plant nitrate reductase: in vitro evidence for the NR-dependent formation of active nitrogen species. FEBS Lett;468:89-92.

Yang, H, Matsubayashi, Y, Nakamura, K, Sakagami, Y. 1999 Oryza sativa PSK gene encodes a precursor of phytosulfokine-alpha, a sulfated peptide growth factor found in plants. Proc Natl Acad Sci USA;96:13560-13565.

Yang, T, Poovaiah, B W. 2000 An early ethylene up-regulated gene encoding a calmodulin-binding protein involved in plant senescence and death. J Biol Chem;275:38467-38473.

Yang, W, Nadolska-Orcyk, A, Wood, K, Hahn, D, Rich, P, Wood, A, Saneoka, Premachandra, G, Bonham, C and Rhodes, J. 1995 Near-iosgenic lines of maize differing for glycine beatine. Plant Physiology; February;107(2):621-630.

Yankulov, K Y, Bentley, D L. 1997 Regulation of CDK7 substrate specificity by MAT1 and TFIIH. EMBO J;16:1638-1646.

Yanofsky, M F, Ma, H, Bowman, J L, Drews, G N, Feldmann, K A, Meyerowitz, E M. 1990 The protein encoded by the Arabidopsis homeotic gene agamous resembles transcription factors. Nature;346:35-39.

Yaropolov, A I, Skorobogato'ko, S S, and Varfolomeyev, S D 1994 Laccase properties, catalytic mechanism, and applicability. Applied Biochemistry and Biotechnology;49:257-280.

Yokokura, H, Picciotto, M R, Nairn, A C, Hidaka, H. 1995. The regulatory region of calcium/calmodulin-dependent protein kinase I contains closely associated autoinhibitory and calmodulin-binding domains. J Biol Chem;270:23851-23859.

Yoo, J G, Bowien, B. Analysis of the cbbF genes from Alcaligenes eutrophus that encode fructose-1,6-/sedoheptulose-1,7-bisphosphatase. Curr Microbiol July 1995;31(1):55-61.

Yoshida, S, Ito, M, Nishida, I, Watanabe, A. 2001 Isolation and RNA gel blot analysis of genes that could serve as potential molecular markers for leaf senescence in Arabidopsis thaliana. Plant Cell Physiol;42:170-178.

You, J, Pickart, C M. 2001 A hect domain e3 enzyme assembles novel polyubiquitin chains. J Biol Chem;276:19871-19878.

Young, T E, Gallie, D R 1999 Analysis of programmed cell death in wheat endosperm reveals differences in endosperm development between cereals. Plant Mol Biol;39:915-926.

Young, J C, DeWitt, N D, and Sussman, M R. 1998 A transgene encoding a plasma membrane H⁺-ATPase that confers acid resistance inArabidopsis thaliana seedlings. Genetics;149:501-507.

Young, J C, Hartl, F U. 2000 Polypeptide release by hsp90 involves ATP hydrolysis and is enhanced by the co-chaperone p23. EMBO J;19:5930-5940.

Young, J C, Obermann, W M, Hartl, F U. 1998 Specific binding of tetratricopeptide repeat proteins to the C-terminal 12-kDa domain of hsp90. J Biol Chem;273:18007-18010.

Young, S. A., Wang, X. and Leach, J. E. 1996 Changes in the plasma membrane distribution of rice phospholipase C during resistant interactions with Xanthomonas oryzae pv oryzae. Plant Cell;8:1079-1090.

Young, T E, Gallie, D R 2000 Programmed cell death during endosperm development. Plant Mol Biol;44: 283-301.

Young, T E, Gallie, D R. 2000 Regulation of programmed cell death in maize endosperm by abscisic acid. Plant Mol Biol;42:397-414.

Yu, D, Chen, C, Chen, Z. 2001 Evidence for an Important Role of WRKY DNA Binding Proteins in the Regulation of NPR1 Gene Expression. Plant Cell;13:1527-1540.

Yu, O, Jung, W, Shi, J, Croes, R A, Fader, G M, McGonigle, B, Odell, J T. 2000 Production of the isoflavones genistein and daidzein in non-legume dicot and monocot tissues. Plant Physiol;124:781-793.

Yu, X, Sukumaran, S, Mrton, L. 1998 Differential expression of the arabidopsis nia1 and nia2 genes. cytokinin-induced nitrate reductase activity is correlated with increased nial transcription and mrna levels. Plant Physiol;116:1091-1096.

Yukioka, H, Inagaki, S, Tanaka, R, Katoh, K, Miki, N, Mizutani, A, Masuko, M. 1998 Transcriptional activation of the alternative oxidase gene of the fungus Magnaporthe grisea by a respiratory-inhibiting fungicide and hydrogen peroxide. Biochim. Biophys. Acta;1442:161-169.Yun, S J, Oh, S_H. 1998 Cloning and characterization of tobacco cDNA encoding calcium/calmodulin dependent glutamate decarboxylase. Mol. Cells;8:125-129.

Zambryski et al., J. Mol. Appl. Genet., 1, 361-370 (1982); GenBank Accession AJ237588

Zarsky, V, Cvrckova, F, Bischoff, F, Palme, K. 1997 At-GDI1 from Arabidopsis thaliana encodes a rab-specific GDP dissociation inhibitor that complements the sec19 mutation of Saccharomyces cerevisiae. FEBS Lett;403:303-308.

Zeng, Y, and Elbein, A D. 1995 UDP-N-acetylglucosamine: dolichyl-phosphate N-acetylglucosamine-1-phosphate transferase is amplified in tunicamycin-resistant soybean cells. Eur. J. Biochem;233:458-466.

Zhang, C, Cai, Y, Adachi, M T, Oshiro, S, Aso, T, Kaufman, R J, Kitajima, S. 2001 Homocysteine induces programmed cell death in human vascular endothelial cells through activation of the unfolded protein response. J Biol Chem;276(38):35867-35874.

Zhang, H and Blumwald, E. 2001. Transgenic salt-tolerant tomato plants accumulate salt in foliage but not in fruit. Nature Biotechnolgy. August;19:765-768.

Zhao, R, Dielen, V, Kinet, J -M, Boutry, M. 2000 Cosuppression of a plasma membrane H+-ATPase isoform impairs sucrose translocation, stomatal opening, plant growth, and male fertility. Plant Cell;12:535-546.

Zheng, N, Wang, P, Jeffrey, P D, Pavletich, N P. 2000 Structure of a c-Cbl-UbcH7 complex: RING domain function in ubiquitin-protein ligases. Cell;102:533-539.

Zhong, X, Malhotra, R, Guidotti, G. 2000 Regulation of yeast ectoapyrase ynd1p activity by activator subunit vma13p of vacuolar H+-ATPase. J Biol Chem;275:35592-35599.

Zhou, H, Stiff, C M, Konzak, C F. Stably transformed callus of wheat by electroporation-induced direct gene transfer. Plant Cell Reports 1993;12(11):612-616.

Zhou, J J, Theodolou, F, Sauer, N, Sanders, D, and Miller, A J. 1997 A kinetic model with ordered cytoplasmic dissociation for SUC1, an Arabidopsis H⁺/sucrose cotransporter expressed in Xenopus oocytes. J. Membr. Biol;159:113-125.

Zhu, D, Scandalios, J G. 1992 Expression of the maize MnSod (Sod3) gene in MnSOD-deficient yeast rescues the mutant yeast under oxidative stress. Genetics;131:803-809.

Zhu, Y, Tepperman, J M, Fairchild C D, Quail, P H. 2000 Phytochrome B binds with greater apparent affinity than phytochrome A to the basic helix-loop-helix factor PIF3 in a reaction requiring the PAS domain of PIF3. Proc Natl Acad Sci USA;97:13419-13424.

Zielinskie, R E. Calmodulin and calmodulin-binding proteins in plants. Ann. Rev. Plant Physiol. Plant Mol. Biol;1998;49:697-725.

Zik, M, Arazi, T, Snedden, W A, Fromm, H. 1998 Two isoforms of glutamate decarboxylase in Arabidopsis are regulated by calcium/calmodulin and differ in organ distribution. Plant Mol. Biol 37:967-1075.

Zimmarman et al., IMPDH: regulation of expression and role in cellular proliferation and T lymphocyte activation. Prog Nucleic Acid Res Mol Biol 61:181-209.

Zimmer, S G, DeBenedetti, A, Graff, J R. 2000 Translational control of malignancy: the mRNA cap-binding protein, eIF-4E, as a central regulator of tumor formation, growth, invasion and metastasis. Anticancer Res;20:1343-1351.

Zinselmeier, C, Jeong, B -R, Boyer, J S. 1999 Starch and the control of kernel number in maize at low water potentials. Plant Physiol;121:25-35.

Zuk, D and Jacobson, A. 1998 A single amino acid substitution in yeast eIF-5A results in mRNA stabilization. EMBO J;17:2914-2925.

Zukowski, M M, Gaffney, D F, Speck D, Kauffmann, M, Findeli, A, Wisecup A, Lecocq, J P. Chromogenic identification of genetic regulatory signals in Bacillus subtilis based on expression of a cloned Pseudomonas gene. Proc Natl Acad Sci USA February 1983;80(4):1101-1105.

What is claimed is:

1. An isolated polynucleotide comprising a nucleic acid sequence selected from the group consisting of SEQ ID NO: 1 through SEQ ID NO: 368 or complements thereof.

2. A recombinant DNA construct comprising a polynucleotide according to claim 1.

3. A recombinant DNA construct according to claim 2, wherein said polynucleotide is operably linked to a promoter functional in a plant cell.

4. A recombinant DNA construct according to claim 2, wherein said polynucleotide is operably linked to a 3′ untranslated region functional in a plant cell.

5. A transformed cell or organism comprising a polynucleotide according to claim 1.

6. The transformed cell or organism according to claim 5, wherein the cell is a plant cell or plant.

7. The transformed cell or organism according to claim 6, wherein the organism is a plant selected from the group consisting of cotton, wheat, soybean, maize, rice, canola, teosinte and Arabidopsis.

8. A substantially purified polypeptide comprising an amino acid sequence selected from the group consisting of SEQ ID NO: 369 through SEQ ID NO: 736.

9. An isolated polynucleotide encoding a polypeptide having an amino acid sequence selected from the group consisting of SEQ ID NO: 369 through SEQ ID NO: 736.

10. An isolated polynucleotide encoding a polypeptide having at least 70% amino acid sequence identity with a polypeptide having an amino acid sequence selected from the group consisting of SEQ ID NO: 369 through SEQ ID NO: 736.

11. A recombinant DNA construct comprising a polynucleotide selected from the group consisting of:
(a) a polynucleotide comprising a nucleic acid sequence selected from the group consisting of SEQ ID NO: 1 through SEQ ID NO: 368;
(b) a polynucleotide encoding a polypeptide having an amino acid sequence selected from the group consisting of SEQ ID NO: 369 through SEQ ID NO: 736;
(c) a polynucleotide comprising a nucleic acid sequence complementary to a nucleic acid sequence selected from the group consisting of SEQ ID NO: 1 through SEQ ID NO: 368;
(d) a polynucleotide having at least 60% sequence identity to a polynucleotide of (a), (b) or (c);
(e) a polynucleotide encoding a polypeptide having at least 70% sequence identity to a polypeptide having an amino acid sequence selected from the group consisting of SEQ ID NO: 369 through SEQ ID NO: 736;
(f) an oligonucleotide comprising from about 15 to 100 nucleotide bases, wherein the oligonucleotide hybridizes under low stringency conditions to a polynucleotide of (a), (b) or (c);
(g) a polynucleotide comprising a promoter functional in a plant cell, operably joined to a coding sequence for a polypeptide having at least 70% sequence identity to a polypeptide having an amino acid sequence selected from the group consisting of SEQ ID NO: 369 through SEQ ID NO: 736, wherein the encoded polypeptide is a functional homolog of said polypeptide having an amino acid sequence selected from the group consisting of SEQ ID NO: 369 through SEQ ID NO: 736; and
(h) a polynucleotide comprising a promoter functional in a plant cell, operably joined to a coding sequence for a polypeptide having an amino acid sequence selected from the group consisting of SEQ ID NO: 369 through SEQ ID NO: 736, wherein transcription of the coding sequence produces an RNA molecule having sufficient complementarity to a polynucleotide encoding said polypeptide to result in decreased expression of said polypeptide when the construct is expressed in a plant cell.

12. A transformed plant comprising a recombinant DNA construct, wherein said construct comprises a promoter region functional in a plant cell operably joined to a polynucleotide comprising coding sequence for a polypeptide having an amino acid sequence selected from the group consisting of SEQ ID NO: 369 through SEQ ID NO: 736.

13. A transformed plant of claim 12 wherein said polynucleotide is oriented with respect to said promoter such that transcription of said polynucleotide produces an mRNA encoding said polypeptide.

14. A transformed plant of claim 12 wherein said polynucleotide is oriented with respect to said promoter such that transcription from said polynucleotide produces an RNA complementary to the mRNA encoding said polypeptide.

15. The transformed plant according to claim 12 wherein said plant is maize.

16. The transformed plant according to claim 12 wherein said plant is soybean.

17. A method of producing a plant having an improved property, wherein said method comprises transforming a plant with a recombinant construct comprising a promoter region functional in a plant cell operably joined to a polynucleotide comprising coding sequence for a polypeptide associated with said property, and growing said transformed plant, wherein said polynucleotide is selected from the group consisting of:
(a) a polynucleotide encoding a polypeptide useful for manipulating protein quality in plant cells by modification of amino acid transporters, wherein said polynucleotide comprises a sequence selected from the group consisting of SEQ ID NO: 1 through SEQ ID NO: 9;
(b) a polynucleotide encoding a polypeptide useful for improving plants by providing protection against osmotic stress, wherein said polynucleotide comprises a sequence selected from the group consisting of SEQ ID NO: 10 through SEQ ID NO: 12;
(c) a polynucleotide encoding a polypeptide useful for improving yield in a plant by altering sugar transport and/or metabolism, wherein said polynucleotide comprises a sequence selected from the group consisting of SEQ ID NO: 10 through SEQ ID NO: 12;
(d) a polynucleotide encoding a polypeptide useful for providing increased vigor to a plant by modification of plant steroid biosynthesis, wherein said polynucleotide comprises a sequence selected from the group consisting of SEQ ID NO: 13 through SEQ ID NO: 19;
(e) a polynucleotide encoding a polypeptide useful for improving crop productivity or grain composition by manipulating plant growth rate, wherein said polynucleotide comprises a sequence selected from the group consisting of SEQ ID NO: 20 through SEQ ID NO: 24;
(f) a polynucleotide encoding a polypeptide useful for providing improved plants having cercosporin resistance, wherein said polynucleotide comprises SEQ ID NO: 25;
(g) a polynucleotide encoding a polypeptide useful for improving plant growth and grain quality by modification of nitrogen/nitrate levels in a plant, wherein said polynucleotide comprises a sequence selected from the group consisting of SEQ ID NO: 26 through SEQ ID NO: 27;
(h) a polynucleotide encoding a polypeptide useful for altering nitrogen utilization in a plant by modification of siroheme synthesis, wherein said polynucleotide comprises a sequence selected from the group consisting of SEQ ID NO: 28 through SEQ ID NO: 30;
(i) a polynucleotide encoding a polypeptide useful for improving plant cold tolerance, wherein said polynucleotide comprises sequence of a cold induced gene selected from the group consisting of SEQ ID NO: 31 through SEQ ID NO: 42;
(j) a polynucleotide encoding a polypeptide useful for improving plant yield by modification of the cell cycle pathway, wherein said polynucleotide comprises a sequence selected from the group consisting of SEQ ID NO: 43 through SEQ ID NO: 61;
(k) a polynucleotide encoding a polypeptide for improving plant growth by manipulating cytokinin levels or signaling pathways, wherein said polynucleotide comprises a sequence selected from the group consisting of SEQ ID NO: 62 through SEQ ID NO: 76;
(l) a polynucleotide encoding a polypeptide useful for alteration of oil content of a seed by manipulating embryo size, wherein said polynucleotide comprises a sequence selected from the group consisting of SEQ ID NO: 77 through SEQ ID NO: 79;
(m) a polynucleotide encoding a polypeptide useful for improving plant growth as the result of manipulating the cell cycle by modification of E2F activity, wherein said polynucleotide comprises a sequence selected from the group consisting of SEQ ID NO: 80 through SEQ ID NO: 82;
(n) a polynucleotide encoding a polypeptide useful for reduction of senescence, wherein said polynucleotide comprises a sequence selected from the group consisting of SEQ ID NO: 83 through SEQ ID NO: 93;
(o) a polynucleotide encoding a polypeptide useful for improving insect resistance, wherein said polynucleotide comprises a sequence selected from the group consisting of SEQ ID NO: 94 through SEQ ID NO: 96;
(p) a polynucleotide encoding a polypeptide useful for improving cold tolerance in a plant, wherein said polynucleotide comprises SEQ ID NO: 97;
(q) a polynucleotide encoding a polypeptide useful for improving cold or heat tolerance of a plant by alteration of fatty acid composition, wherein said polynucleotide comprises a sequence selected from the group consisting of SEQ ID NO: 98 through SEQ ID NO: 101;
(r) a polynucleotide encoding a polypeptide useful for improving stress resistance in a plant by modification of ferritin levels, wherein said polynucleotide comprises a sequence selected from the group consisting of SEQ ID NO: 102 through SEQ ID NO: 104;
(s) a polynucleotide encoding a polypeptide useful for improving plant herbicide resistance, wherein said polynucleotide comprises a sequence selected from the group consisting of SEQ ID NO: 105 through SEQ ID NO: 107;
(t) a polynucleotide encoding a transcription factor polypeptide useful for improving fungal resistance, wherein said polynucleotide comprises SEQ ID NO: 108;
(u) a polynucleotide encoding a polypeptide useful for improving plant growth and stress resistance by increasing levels of GABA, wherein said polynucleotide comprises SEQ ID NO: 109;
(v) a polynucleotide encoding a polypeptide useful for improving cold tolerance in a plant by heat generation resulting from modification of alternatative oxidase activity, wherein said polynucleotide comprises a sequence selected from the group consisting of SEQ ID NO: 110 through SEQ ID NO: 111;
(w) a polynucleotide encoding a polypeptide useful for improving heat tolerance in a plant, wherein said polynucleotide comprises a sequence selected from the group consisting of SEQ ID NO: 112 through SEQ ID NO: 119;
(x) a polynucleotide encoding a polypeptide useful for improving plant growth and disease resistance by altering the activity of heterotrimeric G proteins, wherein said polynucleotide comprises a sequence selected from the group consisting of SEQ ID NO: 120 through SEQ ID NO: 124;
(y) a polynucleotide encoding a polypeptide useful for improving homologous recombination in a plant, wherein said polynucleotide comprises a sequence selected from the group consisting of SEQ ID NO: 125 through SEQ ID NO: 126;
(z) a polynucleotide encoding a polypeptide useful for improving tolerance to heat shock by modification of hsp90 protein function, wherein said polynucleotide comprises a sequence selected from the group consisting of SEQ ID NO: 127 through SEQ ID NO: 147;
(aa) a polynucleotide encoding a polypeptide useful for generating improved plants having increased seed or kernel yield under drought conditions, wherein said polynucleotide comprises SEQ ID NO: 148;
(bb) a polynucleotide encoding a polypeptide useful for improving plant growth by modification of jasmonate activity, wherein said polynucleotide comprises a sequence selected from the group consisting of SEQ ID NO: 149 through SEQ ID NO: 162;
(cc) a polynucleotide encoding a polypeptide useful for improving plant growth by modification of nitric oxide signaling pathways, wherein said polynucleotide sequence comprises SEQ ID NO: 163;
(dd) a polynucleotide encoding a polypeptide useful for improving plant vigor by modification of photomorphogenic response, wherein said polynucleotide comprises SEQ ID NO: 164;
(ee) a polynucleotide encoding a polypeptide useful for improving plant growth by modification of phosphorous utilization, wherein said polynucleotide comprises a sequence selected from the group consisting of SEQ ID NO: 165 through SEQ ID NO: 180;
(ff) a polynucleotide encoding a polypeptide useful for reducing shade avoidance in a plant by modification of phytochrome activity, wherein said polynucleotide comprises a sequence selected from the group consisting of SEQ ID NO: 181 through SEQ ID NO: 210;
(gg) a polynucleotide encoding a polypeptide useful for improving nitrogen assimilation by modification of the TOR pathway, wherein said polynucleotide comprises a sequence selected from the group consisting of SEQ ID NO: 211 through SEQ ID NO: 225;
(hh) a polynucleotide encoding a polypeptide useful for enhancing seed germination and growth by modification of plant hemoglobin levels, wherein said polynucleotide comprises a sequence selected from the group consisting of SEQ ID NO: 226 through SEQ ID NO: 227;
(ii) a polynucleotide encoding a polypeptide useful for controlling apomixis or altering grain composition in a seed by manipulating polycomb proteins, wherein said polynucleotide comprises a sequence selected from the group consisting of SEQ ID NO: 228 through SEQ ID NO: 232;
(jj) a polynucleotide encoding a polypeptide useful for improving plant growth by modification of retinoblastoma-like proteins in a plant, wherein said polynucleotide comprises a sequence selected from the group consisting of SEQ ID NO: 233 through SEQ ID NO: 234;
(kk) a polynucleotide encoding a polypeptide useful for increasing root mass in a plant, wherein said polynucleotide comprises a sequence selected from the group consisting of SEQ ID NO: 235 through SEQ ID NO: 239;
(ll) a polynucleotide encoding a polypeptide useful for generating early flowering in a plant, wherein said polynucleotide comprises a sequence selected from the group consisting of SEQ ID NO: 240 through SEQ ID NO: 246;
(mm) a polynucleotide encoding a polypeptide useful for improving stalk strength in a plant, wherein said polynucleotide comprises a sequence selected from the group consisting of SEQ ID NO: 247 through SEQ ID NO: 248;
(nn) a polynucleotide encoding a polypeptide useful for conferring virus resistance in a plant, wherein said polynucleotide comprises a sequence selected from the group consisting of SEQ ID NO: 249 through SEQ ID NO: 257;
(oo) a polynucleotide encoding a polypeptide useful for improving yield in a plant, wherein said polynucleotide comprises a sequence selected from the group consisting of SEQ ID NO: 258 through SEQ ID NO: 272;
(pp) a polynucleotide encoding a polypeptide useful for improving water stress tolerance in a plant by modification of wax biosynthesis, wherein said polynucleotide comprises a sequence selected from the group consisting of SEQ ID NO: 273 through SEQ ID NO: 277;
(qq) a polynucleotide encoding a polypeptide useful for improving plant growth by altering development, wherein said polynucleotide comprises a sequence selected from the group consisting of SEQ ID NO: 278 and SEQ ID NO: 282;
(rr) a polynucleotide encoding a polypeptide useful for improving plant yield by reducing plant height in high density populations, wherein said polynucleotide encodes a growth regulating factor and comprises a sequence selected from the group consisting of SEQ ID NO: 279 through SEQ ID NO: 281;
(ss) a polynucleotide encoding a polypeptide useful for improving photosynthetic carbon fixation in a plant, wherein said polynucleotide comprises a sequence selected from the group consisting of SEQ ID NO: 283 through SEQ ID NO: 286;
(tt) a polynucleotide encoding a polypeptide useful for improving plant growth by modification of F-box proteins, wherein said polynucleotide comprises a sequence selected from the group consisting of SEQ ID NO: 287 through SEQ ID NO: 291;
(uu) a polynucleotide encoding a polypeptide useful for improving biotic and abiotic stress resistance in a plant, by modification of the mevalonate pathway, wherein said polynucleotide comprises a sequence selected from the group consisting of SEQ ID NO: 292 through SEQ ID NO: 293;
(vv) a polynucleotide encoding a polypeptide useful for improving resistance to oxidative stress in a plant, wherein said polynucleotide comprises a sequence selected from the group consisting of SEQ ID NO: 294 through SEQ ID NO: 331;
(ww) a polynucleotide encoding a polypeptide useful for improving plant growth by modification of ATP production in a plant, wherein said polynucleotide comprises a sequence selected from the group consisting of SEQ ID NO: 332 through SEQ ID NO: 336;
(xx) a polynucleotide encoding a polypeptide useful for improving plant growth by modification of ATP or ADP transport in a plant, wherein said polynucleotide comprises a sequence selected from the group consisting of SEQ ID NO: 337 through SEQ ID NO: 341;
(yy) a polynucleotide encoding a polypeptide useful for altering fatty acid or amino acid composition of a plant seed by modification of AAA ATPase activity in a plant, wherein said polynucleotide comprises a sequence selected from the group consisting of SEQ ID NO: 342 through SEQ ID NO: 343;
(zz) a polynucleotide encoding a polypeptide useful for improving plant yield by modification of plant architecture, wherein said polynucleotide comprises a sequence selected from the group consisting of SEQ ID NO: 344 through SEQ ID NO: 349;
(aaa) a polynucleotide encoding a polypeptide useful for improving yield in a plant by modification of carbohydrate transport, wherein said polynucleotide comprises a sequence selected from the group consisting of SEQ ID NO: 350 through SEQ ID NO: 361;
(bbb) a polynucleotide encoding a polypeptide useful for improving photosynthetic carbon fixation by modification of sedoheptulose 1,7-bisphosphatase activity in a plant, wherein said polynucleotide comprises a sequence selected from the group consisting of SEQ ID NO: 362 through SEQ ID NO: 366;
(ccc) a polynucleotide encoding a polypeptide useful for improving plant resistance to biotic and abiotic stress by modification of flavonoid biosynthesis, wherein said polynucleotide comprises a sequence selected from the group consisting of SEQ ID NO: 367 through SEQ ID NO: 368.

18. The method according to claim 17 wherein said polynucleotide is oriented with respect to said promoter such that transcription of said polynucleotide produces an mRNA encoding said polypeptide, and wherein said mRNA is translated to express said polypeptide in said plant.

19. The method according to claim 17 wherein said polynucleotide is oriented with respect to said promoter such that transcription from said polynucleotide produces an RNA complementary to the mRNA encoding said polypeptide, and wherein the level of said polypeptide in said plant is decreased as the result of the presence of said complementary RNA.

20. A method of producing a plant having an improved property, wherein said method comprises transforming a plant with a recombinant construct comprising a promoter region functional in a plant cell operably joined to a polynucleotide comprising coding sequence for a polypeptide associated with said property, and growing said transformed plant, wherein said polypeptide is selected from the group consisting of:
(a) a polypeptide useful for manipulating protein quality in plant cells by modification of amino acid transporters, wherein said polypeptide comprises a sequence selected from the group consisting of SEQ ID NO: 369 through SEQ ID NO: 377;
(b) a polypeptide useful for improving plants by providing protection against osmotic stress, wherein said polypeptide comprises a sequence selected from the group consisting of SEQ ID NO: 378 through SEQ ID NO: 380;
(c) a polypeptide useful for improving yield in a plant by altering sugar transport and/or metabolism, wherein said polypeptide comprises a sequence selected from the group consisting of SEQ ID NO: 378 through SEQ ID NO: 380;
(d) a polypeptide useful for providing increased vigor to a plant by modification of plant steroid biosynthesis, wherein said polypeptide comprises a sequence selected from the group consisting of SEQ ID NO: 381 through SEQ ID NO: 387;
(e) a polypeptide useful for improving crop productivity or grain composition by manipulating plant growth rate, wherein said polypeptide comprises a sequence selected from the group consisting of SEQ ID NO: 388 through SEQ ID NO: 392;
(f) a polypeptide useful for providing improved plants having cercosporin resistance, wherein said polypeptide comprises SEQ ID NO: 393;
(g) a polypeptide useful for improving plant growth and grain quality by modification of nitrogen/nitrate levels in a plant, wherein said polypeptide comprises a sequence selected from the group consisting of SEQ ID NO: 394 through SEQ ID NO: 395;
(h) a polypeptide useful for altering nitrogen utilization in a plant by modification of siroheme synthesis, wherein said polypeptide comprises a sequence selected from the group consisting of SEQ ID NO: 396 through SEQ ID NO: 398;
(i) a polypeptide useful for improving plant cold tolerance, wherein said polypeptide comprises a sequence selected from the group consisting of SEQ ID NO: 399 through SEQ ID NO: 410;
(j) a polypeptide useful for improving plant yield by modification of the cell cycle pathway, wherein said polypeptide comprises a sequence selected from the group consisting of SEQ ID NO: 411 through SEQ ID NO: 429;
(k) a polypeptide for improving plant growth by manipulating cytokinin levels or signaling pathways, wherein said polypeptide comprises a sequence selected from the group consisting of SEQ ID NO: 430 through SEQ ID NO: 444;
(l) a polypeptide useful for alteration of oil content of a seed by manipulating embryo size, wherein said polypeptide comprises a sequence selected from the group consisting of SEQ ID NO: 445 through SEQ ID NO: 447;
(m) a polypeptide useful for improving plant growth as the result of manipulating the cell cycle by modification of E2F activity, wherein said polypeptide comprises a sequence selected from the group consisting of SEQ ID NO: 448 through SEQ ID NO: 450;
(n) a polypeptide useful for reduction of senescence, wherein said polypeptide comprises a sequence selected from the group consisting of SEQ ID NO: 451 through SEQ ID NO: 461;
(o) a polypeptide useful for improving insect resistance, wherein said polypeptide comprises a sequence selected from the group consisting of SEQ ID NO: 462 through SEQ ID NO: 464;
(p) a polypeptide useful for improving cold tolerance in a plant, wherein said polypeptide comprises SEQ ID NO: 465;
(q) a polypeptide useful for improving cold or heat tolerance of a plant by alteration of fatty acid composition, wherein said polypeptide comprises a sequence selected from the group consisting of SEQ ID NO: 466 through SEQ ID NO: 469;
(r) a polypeptide useful for improving stress resistance in a plant by modification of ferritin levels, wherein said polypeptide comprises a sequence selected from the group consisting of SEQ ID NO: 470 through SEQ ID NO: 472;
(s) a polypeptide useful for improving plant herbicide resistance, wherein said polypeptide comprises a sequence selected from the group consisting of SEQ ID NO: 473 through SEQ ID NO: 475;
(t) a transcription factor polypeptide useful for improving fungal resistance, wherein said polypeptide comprises SEQ ID NO: 476;
(u) a polypeptide useful for improving plant growth and stress resistance by increasing levels of GABA, wherein said polypeptide comprises SEQ ID NO: 477;
(v) a polypeptide useful for improving cold tolerance in a plant by heat generation resulting from modification of alternatative oxidase activity, wherein said polypeptide comprises a sequence selected from the group consisting of SEQ ID NO: 478 through SEQ ID NO: 479;
(w) a polypeptide useful for improving heat tolerance in a plant, wherein said polypeptide comprises a sequence selected from the group consisting of SEQ ID NO: 480 through SEQ ID NO: 487;
(x) a polypeptide useful for improving plant growth and disease resistance by altering the activity of heterotrimeric G proteins, wherein said polypeptide comprises a sequence selected from the group consisting of SEQ ID NO: 488 through SEQ ID NO: 492;
(y) a polypeptide useful for improving homologous recombination in a plant, wherein said polypeptide comprises a sequence selected from the group consisting of SEQ ID NO: 493 through SEQ ID NO: 494;
(z) a polypeptide useful for improving tolerance to heat shock by modification of hsp90 protein function, wherein said polypeptide comprises a sequence selected from the group consisting of SEQ ID NO: 495 through SEQ ID NO: 515;
(aa) a polypeptide useful for generating improved plants having increased seed or kernel yield under drought conditions, wherein said polypeptide comprises SEQ ID NO: 516;
(bb) a polypeptide useful for improving plant growth by modification of jasmonate activity, wherein said polypeptide comprises a sequence selected from the group consisting of SEQ ID NO: 517 through SEQ ID NO: 530;
(cc) a polypeptide useful for improving plant growth by modification of nitric oxide signaling pathways, wherein said polypeptide sequence comprises SEQ ID NO: 531;
(dd) a polypeptide useful for improving plant vigor by modification of photomorphogenic response, wherein said polypeptide comprises SEQ ID NO: 532;
(ee) a polypeptide useful for improving plant growth by modification of phosphorous utilization, wherein said polypeptide comprises a sequence selected from the group consisting of SEQ ID NO: 533 through SEQ ID NO: 548;
(ff) a polypeptide useful for reducing shade avoidance in a plant by modification of phytochrome activity, wherein said polypeptide comprises a sequence selected from the group consisting of SEQ ID NO: 549 through SEQ ID NO: 578;
(gg) a polypeptide useful for improving nitrogen assimilation by modification of the TOR pathway, wherein said polypeptide comprises a sequence selected from the group consisting of SEQ ID NO: 579 through SEQ ID NO: 593;
(hh) a polypeptide useful for enhancing seed germination and growth by modification of plant hemoglobin levels, wherein said polypeptide comprises a sequence selected from the group consisting of SEQ ID NO: 594 through SEQ ID NO: 595;
(ii) a polypeptide useful for controlling apomixis or altering grain composition in a seed by manipulating polycomb proteins, wherein said polypeptide comprises a sequence selected from the group consisting of SEQ ID NO: 596 through SEQ ID NO: 600;
(jj) a polypeptide useful for improving plant growth by modification of retinoblastoma-like proteins in a plant, wherein said polypeptide comprises a sequence selected from the group consisting of SEQ ID NO: 601 through SEQ ID NO: 602;
(kk) a polypeptide useful for increasing root mass in a plant, wherein said polypeptide comprises a sequence selected from the group consisting of SEQ ID NO: 603 through SEQ ID NO: 607;
(ll) a polypeptide useful for generating early flowering in a plant, wherein said polypeptide comprises a sequence selected from the group consisting of SEQ ID NO: 608 through SEQ ID NO: 614;
(mm) a polypeptide useful for improving stalk strength in a plant, wherein said polypeptide comprises a sequence selected from the group consisting of SEQ ID NO: 615 through SEQ ID NO: 616;
(nn) a polypeptide useful for conferring virus resistance in a plant, wherein said polypeptide comprises a sequence selected from the group consisting of SEQ ID NO: 617 through SEQ ID NO: 625;
(oo) a polypeptide useful for improving yield in a plant, wherein said polypeptide comprises a sequence selected from the group consisting of SEQ ID NO: 626 through SEQ ID NO: 640;
(pp) a polypeptide useful for improving water stress tolerance in a plant by modification of wax biosynthesis, wherein said polypeptide comprises a sequence selected from the group consisting of SEQ ID NO: 641 through SEQ ID NO: 645;
(qq) a polypeptide useful for improving plant growth by altering development, wherein said polypeptide comprises a sequence selected from the group consisting of SEQ ID NO: 646 and SEQ ID NO: 650;
(rr) a polypeptide useful for improving plant yield by reducing plant height in high density populations, wherein said polypeptide is a growth regulating factor and comprises a sequence selected from the group consisting of SEQ ID NO: 647 through SEQ ID NO: 649;
(ss) a polypeptide useful for improving photosynthetic carbon fixation in a plant, wherein said polypeptide comprises a sequence selected from the group consisting of SEQ ID NO: 651 through SEQ ID NO: 654;
(tt) a polypeptide useful for improving plant growth by modification of F-box proteins, wherein said polypeptide comprises a sequence selected from the group consisting of SEQ ID NO: 655 through SEQ ID NO: 659;
(uu) a polypeptide useful for improving biotic and abiotic stress resistance in a plant by modification of the mevalonate pathway, wherein said polypeptide comprises a sequence selected from the group consisting of SEQ ID NO: 660 through SEQ ID NO: 661;
(vv) a polypeptide useful for improving resistance to oxidative stress in a plant, wherein said polypeptide comprises a sequence selected from the group consisting of SEQ ID NO: 662 through SEQ ID NO: 699;
(ww) a polypeptide useful for improving plant growth by modification of ATP production in a plant, wherein said polypeptide comprises a sequence selected from the group consisting of SEQ ID NO: 700 through SEQ ID NO: 704;
(xx) a polypeptide useful for improving plant growth by modification of ATP or ADP transport in a plant, wherein said polypeptide comprises a sequence selected from the group consisting of SEQ ID NO: 705 through SEQ ID NO: 709;
(yy) a polypeptide useful for altering fatty acid or amino acid composition of a plant seed by modification of AAA ATPase activity in a plant, wherein said polypeptide comprises a sequence selected from the group consisting of SEQ ID NO: 710 through SEQ ID NO: 711;
(zz) a polypeptide useful for improving plant yield by modification of plant architecture, wherein said polypeptide comprises a sequence selected from the group consisting of SEQ ID NO: 712 through SEQ ID NO: 717;
(aaa) a polypeptide useful for improving yield in a plant by modification of carbohydrate transport, wherein said polypeptide comprises a sequence selected from the group consisting of SEQ ID NO: 718 through SEQ ID NO: 729;
(bbb) a polypeptide useful for improving photosynthetic carbon fixation by modification of sedoheptulose 1,7-bisphosphatase activity in a plant, wherein said polypeptide comprises a sequence selected from the group consisting of SEQ ID NO: 730 through SEQ ID NO: 734;
(ccc) a polypeptide useful for improving plant resistance to biotic and abiotic stress by modification of flavonoid biosynthesis, wherein said polypeptide comprises a sequence selected from the group consisting of SEQ ID NO: 735 through SEQ ID NO: 736.

21. The method according to claim 20 wherein said polynucleotide is oriented with respect to said promoter such that transcription of said polynucleotide produces an mRNA encoding said polypeptide, and wherein said mRNA is translated to express said polypeptide in said plant.

22. The method according to claim 20 wherein said polynucleotide is oriented with respect to said promoter such that transcription from said polynucleotide produces an RNA complementary to the mRNA encoding said polypeptide, and wherein the level of said polypeptide in said plant is decreased as the result of the presence of said complementary RNA.

23. A method for introducing into a maize line an enhanced phenotype as compared to a phenotype in parental units of said maize line, said method comprising
(a) generating a population of transgenic plants comprising a variety of heterologous DNA for the transcription of which there is no known phenotype in corn,
(b) observing phenotypes for said transgenic plants,
(c) selecting seeds from transgenic plants having an unexpected enhanced phenotype, and
(d) optionally, repeating a cycle of germinating transgenic seed, growing subsequent generation plants from said transgenic seed, observing phenotypes of said subsequent generation plants and collecting seeds from subsequent generation plants having an enhanced phenotype.

24. A method according to claim 23 wherein said population of transgenic plants is produced by generating a plurality of transgenic events for a plurality of unique transgenic DNA constructs wherein each of said transgenic events comprises introducing into the genome of a parental maize line a single transgenic DNA construct comprising a promoter operably linked to heterologous DNA, wherein said transgenic DNA construct is introduced into said genome in sufficient quantity to produce transgenic cells which can be cultured into plants of transgenic maize comprising said population culturing said transgenic cells into a population of transgenic plants producing progeny transgenic seed,

25. A method according to claim 24 wherein said plurality of transgenic events is at least 2 and said plurality of unique transgenic DNA constructs is at least 20.

26. A method according to claim 24 wherein said plurality of transgenic events is at least 2 and said plurality of unique transgenic DNA constructs is at least 50.

27. A method according to claim 24 wherein said DNA construct comprises heterologous DNA operably linked to the 5′ end of a promoter region comprising a rice actin promoter and rice actin intron.

28. A method according to claim 24 further comprising crossing transgenic plants from said population with at least one other maize line to produce a hybrid population, observing phenotypes in said hybrid population and selecting seed from plants having an unexpected phenotype.
